(19) United States
(12) Patent Application Publication
(10) Pub. No.: US 2025/0034177 A1
(43) Pub. Date: Jan. 30, 2025

(54) Organic Compound, Light-Emitting Device, Light-Emitting Apparatus, Electronic Device, and Lighting Device

(71) Applicant: Semiconductor Energy Laboratory Co., Ltd., Kanagawa-ken (JP)

(72) Inventors: Harue OSAKA, Atsugi (JP); Satoko NUMATA, Atsugi (JP); Nobuharu OHSAWA, Zama (JP); Yuko KUBOTA, Atsugi (JP); Kyoko TAKEDA, Atsugi (JP); Hiromitsu KIDO, Atsugi (JP)

(73) Assignee: Semiconductor Energy Laboratory Co., Ltd., Kanagawa-ken (JP)

(21) Appl. No.: 18/763,512

(22) Filed: Jul. 3, 2024

(30) Foreign Application Priority Data
Jul. 7, 2023 (JP) 2023-112138

(51) Int. Cl. C07F 7/08; C09K 11/06; H10K 85/40; H10K 85/60

(52) U.S. Cl. CPC C07F 7/0812; C09K 11/06; H10K 85/40; H10K 85/631; H10K 85/6572

(57) ABSTRACT

An organic compound having high heat resistance is provided. An organic compound represented by a general formula (G1) is provided. In the formula, Q represents carbon or silicon; each of R¹ and R² independently represents an alkyl group, a cycloalkyl group, or an aryl group; n represents an integer from 1 to 3; each of α¹ to α⁴ independently represents a phenylene group or a biphenylene group; at least one of R¹⁵, R²⁵, R³¹ and at least one of R⁴⁵, R⁵⁵, and R⁶¹ represent single bonds and the others thereof represent H, an alkyl group, or an aryl group; and R¹¹ to R¹⁴, R²¹ to R²⁴, R³² to R³⁵, R⁴¹ to R⁴⁴, R⁵¹ to R⁵⁴, and R⁶² to R⁶⁵ represent H, an alkyl group, a cycloalkyl group, or an aryl group.

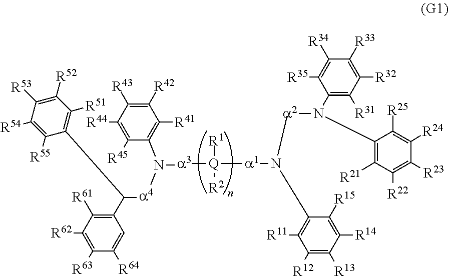

### BACKGROUND OF THE INVENTION

### 1. Field of the Invention

One embodiment of the present invention relates to an organic compound, a light-emitting device, a light-emitting apparatus, a display device, an electronic device, a lighting device, and an electronic device. Note that one embodiment of the present invention is not limited to the above technical field. The technical field of one embodiment of the present invention disclosed in this specification and the like relates to an object, a method, or a manufacturing method. One embodiment of the present invention relates to a process, a machine, manufacture, or a composition of matter. Specifically, examples of the technical field of one embodiment of the present invention disclosed in this specification include a semiconductor device, a display device, a liquid-crystal device, a light-emitting apparatus, a lighting device, a power storage device, a memory device, an imaging device, a driving method thereof, and a manufacturing method thereof.

### 2. Description of the Related Art

Light-emitting devices (also referred to as organic EL devices) including organic compounds and utilizing electroluminescence (EL) have been put to more practical use. In the basic structure of such light-emitting devices, an organic compound layer containing a light-emitting material (an EL layer) is located between a pair of electrodes. Carriers are injected by application of voltage to the device, and recombination energy of the carriers is used, whereby light emission can be obtained from the light-emitting material.

Such light-emitting devices are of self-luminous type and thus have advantages over liquid crystal devices, such as high visibility and no need for backlight when used in pixels of a display, and are suitable as devices used in flat panel displays. Displays that include such light-emitting devices are also highly advantageous in that they can be thin and lightweight. Moreover, such light-emitting devices also have a feature that response speed is extremely fast.

Since light-emitting layers of such light-emitting devices can be successively formed two-dimensionally, planar light emission can be achieved. This feature is difficult to realize with point light sources typified by incandescent lamps or LEDs or linear light sources typified by fluorescent lamps; thus, such light-emitting devices also have great potential as planar light sources, which can be applied to lighting devices and the like.

Displays or lighting devices including light-emitting devices can be used for a variety of electronic devices as described above, and research and development of light-emitting devices have progressed for more favorable characteristics.

Many problems for the improvement of light-emitting device characteristics depend on the substances included in light-emitting devices, such as organic compounds, metals, and metal compounds. Substance development, device structure modification, and the like have been performed with the aim of overcoming the problems. Patent Document 1 discloses a hole-transport material, which is a kind of organic compound capable of increasing the emission efficiency of a light-emitting device when used in the light-emitting device.

The heat resistance of light-emitting devices has also been problematic because the luminance of a light-emitting device might be degraded by crystallization or the like if an organic compound in the light-emitting device has an inadequate glass transition temperature (T_{g}).

### REFERENCES

### Patent Document

[Patent Document 1] Japanese Published Patent Application No. 2009-298767

### Non-Patent Documents

[Non-Patent Document 1] Nicholas J. Turro, V. Ramamurthy, J. C. Scaiano, “MODERN MOLECULAR PHOTOCHEMISTRY OF ORGANIC MOLECULES”, UNIVERSITY SCIENCE BOOKS, 2010/02/10, pp. 204-208
[Non-Patent Document 2] Daisaku TANAKA et al., “Ultra High Efficiency Green Organic Light-Emitting Devices”, Japanese Journal of Applied Physics, Vol. 46, No. 1, 2007, pp. L10-L12

### SUMMARY OF THE INVENTION

One way of improving the heat resistance of an organic compound is to increase the molecular weight. Considering an effect on the carrier-transport property and the like of the organic compound, introduction of a substituent such as an aryl group or a heteroaryl group is a possible way of increasing the molecular weight; however, this is also problematic because the S₁ level and the T₁ level are lowered.

In view of the above, an object of one embodiment of the present invention is to provide an organic compound having high heat resistance. Another object is to provide an organic compound whose T_{g}, which is an indicator of the heat resistance of organic compounds, is high. Another object is to provide an organic compound which has a high S₁ level and a high T₁ level. Another object to provide an organic compound having a high carrier-transport property.

An object of one embodiment of the present invention is to provide a light-emitting device with favorable light-emitting characteristics. Another object is to improve the reliability of a light-emitting device. Another object is to improve the emission efficiency of a light-emitting device.

Another object of one embodiment of the present invention is to provide a light-emitting apparatus with low power consumption. Another object is to provide a display device with low power consumption. Another object is to provide a lighting device with low power consumption.

Another object of one embodiment of the present invention is to provide a novel organic compound, a novel light-emitting device, a novel light-emitting apparatus, a novel display device, or a novel lighting device.

Note that the description of these objects does not preclude the existence of other objects. In one embodiment of the present invention, there is no need to achieve all the objects. Other objects will be apparent from and can be derived from the descriptions of the specification, the drawings, the claims, and the like.

One embodiment of the present invention is an organic compound represented by a general formula (G1) below.

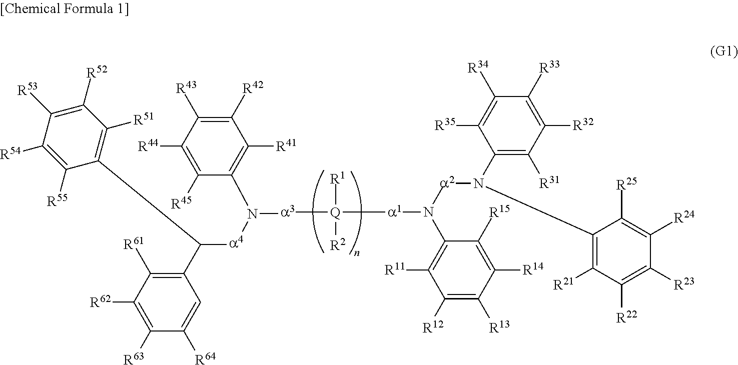

In the general formula (G1), Q represents carbon or silicon; each of R¹ and R² independently represents an alkyl group having 1 to 6 carbon atoms, a substituted or unsubstituted cycloalkyl group having 6 to 8 carbon atoms, or a substituted or unsubstituted aryl group having 6 to 12 carbon atoms; n represents an integer greater than or equal to 1 and less than or equal to 3; each of α¹ to α⁴ independently represents a substituted or unsubstituted phenylene group or a substituted or unsubstituted biphenylene group; at least one of R¹⁵, R²⁵, R³¹, R⁴⁵, R⁵⁵, and R⁶¹ represents a single bond; each of the others of R¹⁵, R²⁵, R³¹, R⁴⁵, R, and R⁶¹ independently represents hydrogen, an alkyl group having 1 to 6 carbon atoms, or a substituted or unsubstituted aryl group having 6 to 12 carbon atoms; and each of R¹¹ to R¹⁴, R²¹ to R²⁴, R³² to R³⁵, R⁴¹ to R⁴⁴, R⁵¹ to R⁵⁴, and R⁶² to R⁶⁵ independently represents hydrogen, an alkyl group having 1 to 6 carbon atoms, a substituted or unsubstituted cycloalkyl group having 6 to 8 carbon atoms, or a substituted or unsubstituted aryl group having 6 to 12 carbon atoms. When R¹⁵ is a single bond, R¹⁵ is bonded to α² to form a ring. When R⁴⁵ is a single bond, R⁴⁵ is bonded to α⁴ to form a ring. When both R²⁵ and R³¹ are single bonds, R²⁵ and R³¹ are bonded to each other to form a ring. When both R⁵⁵ and R⁶¹ are single bonds, R⁵⁵ and R⁶¹ are bonded to each other to form a ring. When the one of R²⁵ and R³¹ is a single bond and the other of R²⁵ and R³¹ is hydrogen, an alkyl group having 1 to 6 carbon atoms, or a substituted or unsubstituted aryl group having 6 to 12 carbon atoms, α² is a substituted or unsubstituted biphenylene group and bonded to one of R²⁵ and R³¹ to form a ring. When one of R⁵⁵ and R⁶¹ is a single bond and the other of R⁵⁵ and R⁶¹ is hydrogen, an alkyl group having 1 to 6 carbon atoms, or a substituted or unsubstituted aryl group having 6 to 12 carbon atoms, α⁴ is a substituted or unsubstituted biphenylene group and bonded to one of R⁵⁵ and R⁶¹ to form a ring.

Another embodiment of the present invention is the organic compound with the above structure in which each of α¹ and α³ independently represents a substituted or unsubstituted m-phenylene group.

Another embodiment of the present invention is the organic compound represented by any one of a general formula (G2-1), a general formula (G1-2-2), and a general formula (G2-3) below.

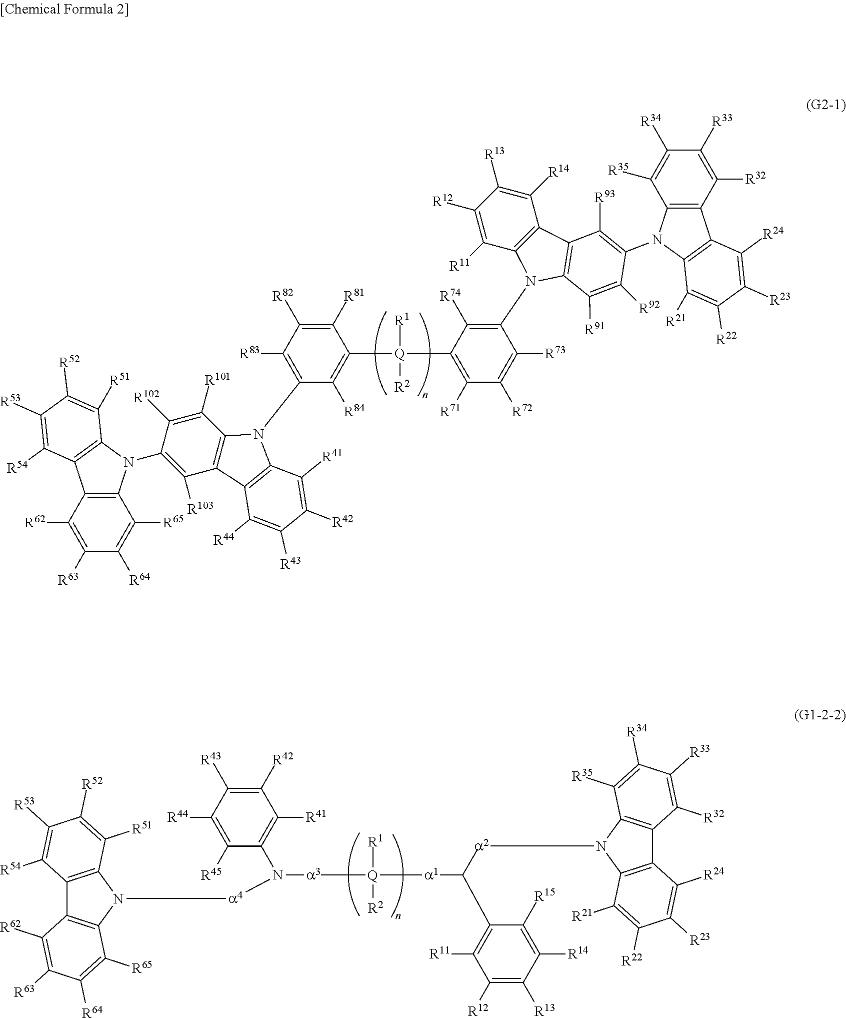

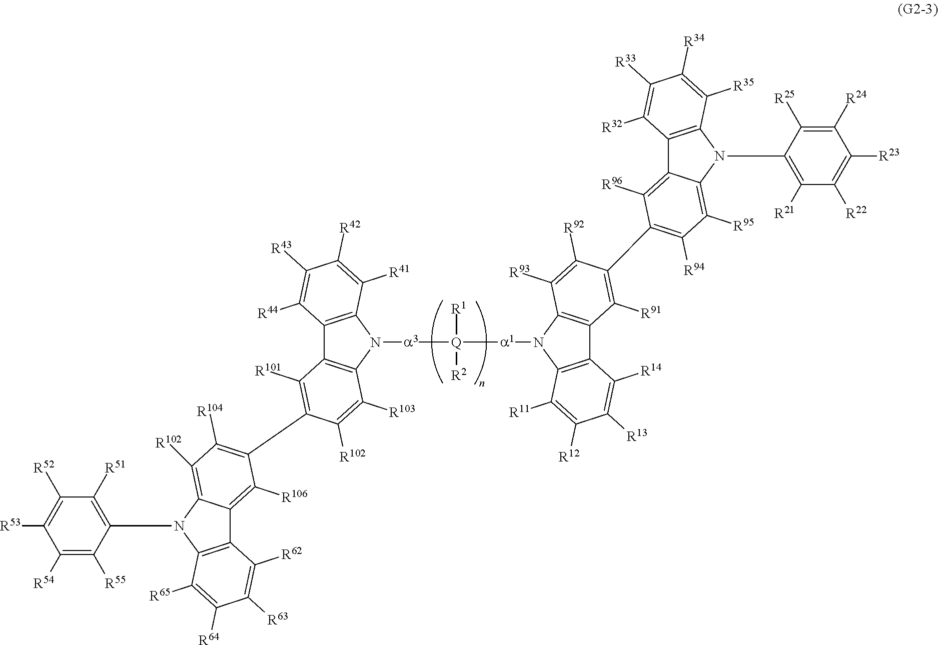

In the general formulae (G2-1), (G1-2-2), and (G2-3), Q represents carbon or silicon; each of R¹ and R² independently represents an alkyl group having 1 to 6 carbon atoms, a substituted or unsubstituted cycloalkyl group having 6 to 8 carbon atoms, or a substituted or unsubstituted aryl group having 6 to 12 carbon atoms; n represents an integer greater than or equal to 1 and less than or equal to 3; each of α¹ to α⁴ independently represents a substituted or unsubstituted phenyl group or a substituted or unsubstituted biphenyl group; and each of R¹¹ to R¹⁵, R²¹ to R²⁵, R³¹ to R³⁵, R⁴¹ to R⁴⁵, R¹ to R⁵⁵, R⁶¹ to R⁶⁵, R⁷¹ to R⁷⁴, R⁸¹ to R⁸⁴, R⁹¹ to R⁹⁶, and R¹⁰¹ to R¹⁰⁶ independently represents hydrogen, an alkyl group having 1 to 6 carbon atoms, or a substituted or unsubstituted aryl group having 6 to 12 carbon atoms.

Another embodiment of the present invention is the organic compound with the above structure in which the T_{g} is higher than or equal to 100° C.

Another embodiment of the present invention is an organic compound represented by any one of a structural formula (100), a structural formula (101), a structural formula (102), and a structural formula (114) below.

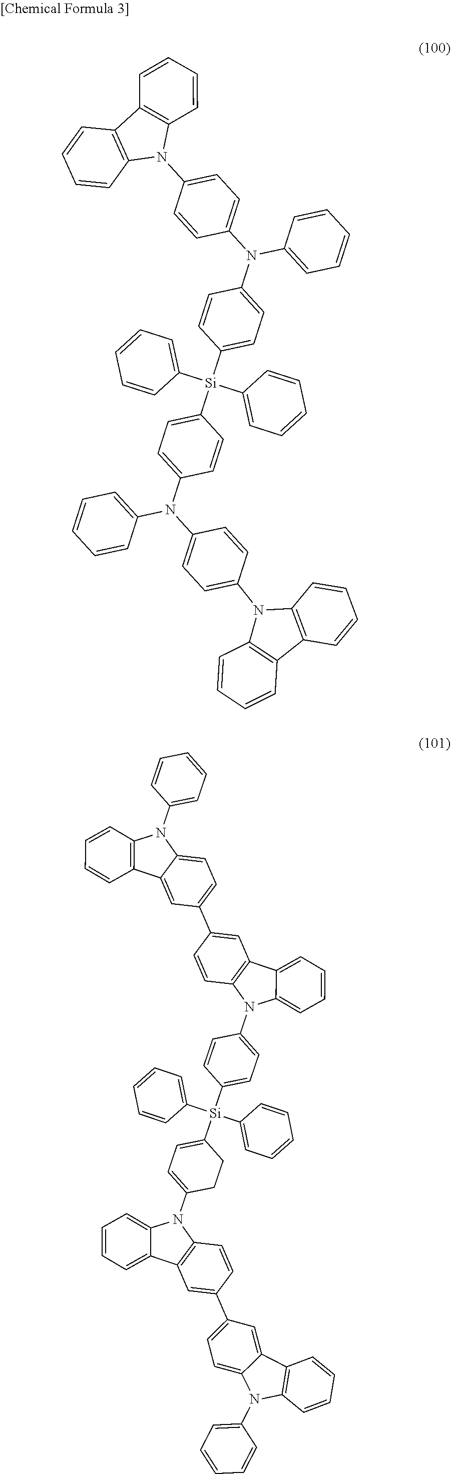 [SMILES: c1ccc(N(c2ccc(-n3c4ccccc4c4ccccc43)cc2)c2ccc([Si](c3ccccc3)(c3ccccc3)c3ccc(N(c4ccccc4)c4ccc(-n5c6ccccc6c6ccccc65)cc4)cc3)cc2)cc1 ; C1=C(n2c3ccccc3c3cc(-c4ccc5c(c4)c4ccccc4n5-c4ccccc4)ccc32)CCC([Si](c2ccccc2)(c2ccccc2)c2ccc(-n3c4ccccc4c4cc(-c5ccc6c(c5)c5ccccc5n6-c5ccccc5)ccc43)cc2)=C1]

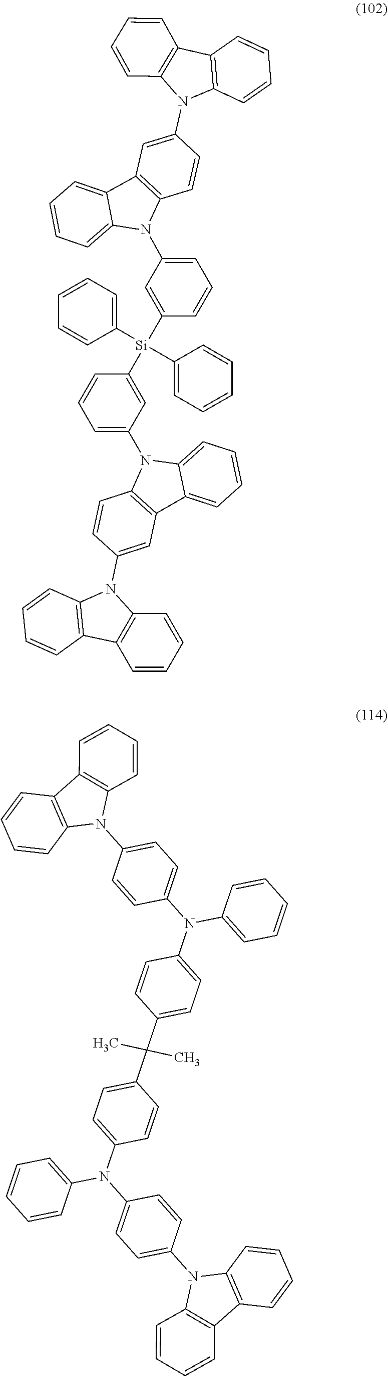 [SMILES: c1ccc([Si](c2ccccc2)(c2cccc(-n3c4ccccc4c4cc(-n5c6ccccc6c6ccccc65)ccc43)c2)c2cccc(-n3c4ccccc4c4cc(-n5c6ccccc6c6ccccc65)ccc43)c2)cc1 ; CC(C)(c1ccc(N(c2ccccc2)c2ccc(-n3c4ccccc4c4ccccc43)cc2)cc1)c1ccc(N(c2ccccc2)c2ccc(-n3c4ccccc4c4ccccc43)cc2)cc1]

Another embodiment of the present invention is a light-emitting device including a first electrode, a second electrode, and an organic compound layer (an EL layer); the organic compound layer is positioned between the first electrode and the second electrode; and the organic compound layer includes the organic compound of one embodiment of the present invention (the organic compound represented by the general formula (G1)).

Another embodiment of the present invention is a light-emitting device including a first electrode, a second electrode, and an organic compound layer (an EL layer); the organic compound layer is positioned between the first electrode and the second electrode; the organic compound layer includes a plurality of layers each including an organic compound and includes at least one light-emitting layer; and among the plurality of layers each including the organic compound, any one of layers from a layer in contact with the first electrode to at least the light-emitting layer includes the organic compound of one embodiment of the present invention (the organic compound represented by the general formula (G1)).

Another embodiment of the present invention is a light-emitting device including a first electrode, a second electrode, and an organic compound layer (an EL layer); the organic compound layer is positioned between the first electrode and the second electrode; the organic compound layer includes a plurality of layers each including an organic compound and includes at least one light-emitting layer; the light-emitting compound layer includes the organic compound of one embodiment of the present invention (the organic compound represented by the general formula (G1)) and a second organic compound; and the second organic compound includes a π-electron deficient heteroaromatic ring.

Another embodiment of the present invention is a light-emitting device in which an organic compound layer (an EL layer) between a pair of electrodes has a stacked-layer structure of a plurality of layers including a light-emitting layer, end portions of the organic compound layer are aligned or substantially aligned with each other, the organic compound layer includes a first partial structure, a second partial structure, and a third partial structure, the first partial structure and the second partial structure are connected through the third partial structure, at least one of the first partial structure and the second partial structure includes any one or more of a π-electron rich heteroaromatic ring, an aromatic amine skeleton, and a π-electron deficient heteroaromatic ring, and the third partial structure includes a first organic compound represented by a general formula (L1) below. Note that the first organic compound includes the above-described organic compound of one embodiment of the present invention (the organic compound represented by the general formula (G1)).

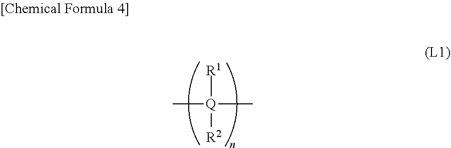

In general formula (L1), Q represents carbon or silicon; each of R¹ and R² independently represents an alkyl group having 1 to 6 carbon atoms, a substituted or unsubstituted cycloalkyl group having 6 to 8 carbon atoms, or a substituted or unsubstituted aryl group having 6 to 12 carbon atoms; and n is an integer greater than or equal to 1 and less than or equal to 3.

Another embodiment of the present invention is the light-emitting device with any of the above structures, in which the first organic compound is included in a light-emitting layer or a layer in contact with the light-emitting layer.

Another embodiment of the present invention is the light-emitting device with any of the above structures, in which any one of the first partial structure and the second partial structure is represented by a general formula (T1) below.

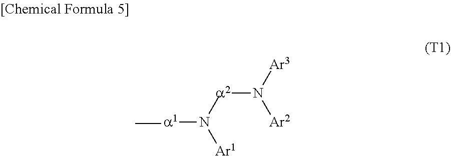

In the general formula (T1), each of α¹ and α² independently represents a substituted or unsubstituted arylene group having 6 to 12 carbon atoms, and each of Ar¹ to Ar³ independently represents a substituted or unsubstituted aryl group having 6 to 12 carbon atoms. Note that Ar¹ and α² may be bonded to each other to form a ring but are not necessarily bonded to each other. Furthermore, α² and Ar² may be bonded to each other to form a ring but are not necessarily bonded to each other. Ar² and Ar³ may be bonded to each other to form a ring but are not necessarily bonded to each other.

Another embodiment of the present invention is the light-emitting device with any of the above structures, in which the T_{g} of the first organic compound is higher than or equal to 100° C.

Another embodiment of the present invention is a light-emitting apparatus that includes a light-emitting device with any of the above structures and a transistor or a substrate.

Another embodiment of the present invention is an electronic device including the above light-emitting apparatus; and a sensor unit, an input unit, or a communication unit.

Another embodiment of the present invention is a lighting device including the above light-emitting apparatus and a housing.

Another embodiment of the present invention can provide an organic compound having high heat resistance. Another embodiment can provide an organic compound having a high T_{g}. Another embodiment can provide an organic compound having a high S₁ level and a high T₁ level. Another embodiment can provide an organic compound having a carrier-transport property. An organic compound that can be used for a light-emitting layer and a layer in contact with the light-emitting layer in a light-emitting device exhibiting blue fluorescence or phosphorescence can be provided.

Another embodiment of the present invention can provide a light-emitting device with favorable characteristics. Another embodiment can improve the reliability of a light-emitting device. Another embodiment can improve the emission efficiency of a light-emitting device. In addition, a light-emitting device having heat resistance high enough to withstand heating during a manufacturing process, storage, driving, and the like can be provided.

Another embodiment of the present invention can provide a light-emitting apparatus with low power consumption. Another embodiment can provide a display device with low power consumption. Another embodiment can provide a lighting device with low power consumption.

Another embodiment of the present invention can provide a novel organic compound, a novel light-emitting device, a novel light-emitting apparatus, a novel display device, or a novel lighting device.

Note that the description of these effects does not preclude the existence of other effects. One embodiment of the present invention does not necessarily have all of these effects. Other effects can be derived from the description of the specification, the drawings, and the claims.

### BRIEF DESCRIPTION OF THE DRAWINGS

FIGS. 1A to 1F each illustrate a structure of a light-emitting device according to an embodiment.

FIG. 2 illustrates a structure of light-emitting devices according to an embodiment.

FIGS. 3A to 3D illustrate a light-emitting apparatus according to an embodiment.

FIGS. 4A to 4C illustrate a method for manufacturing a light-emitting apparatus according to an embodiment.

FIGS. 5A to 5C illustrate a method for manufacturing a light-emitting apparatus according to an embodiment.

FIGS. 6A to 6D illustrate a method for manufacturing a light-emitting apparatus according to an embodiment.

FIGS. 7A to 7C illustrate a light-emitting apparatus according to an embodiment.

FIGS. 8A to 8F illustrate an apparatus and pixel arrangements according to an embodiment.

FIGS. 9A to 9C illustrate pixel circuits and a transistor according to an embodiment.

FIG. 10 illustrates a light-emitting apparatus according to an embodiment.

FIGS. 11A to 11E illustrate electronic devices according to an embodiment.

FIGS. 12A to 12E illustrate electronic devices according to an embodiment.

FIGS. 13A and 13B illustrate electronic devices according to an embodiment.

FIGS. 14A and 14B illustrate a lighting device according to an embodiment.

FIG. 15 illustrates lighting devices according to an embodiment.

FIG. 16 shows a ¹H NMR spectrum of PSiYGA2.

FIG. 17 shows an MS spectrum of PSiYGA2.

FIG. 18 shows a ¹H NMR spectrum of YGABmP.

FIG. 19 shows an MS spectrum of YGABmP.

FIG. 20 shows emission spectra of toluene solutions of PSiYGA2, YGABmP, YGA1BP, and YGABP.

FIG. 21 shows a ¹H NMR spectrum of PCCz2PSi.

FIG. 22 shows emission spectra of toluene solutions of PCCz2PSi and PCCP.

FIG. 23 shows a ¹H NMR spectrum of mCzCz2PSi.

FIG. 24 shows an MS spectrum of mCzCz2PSi.

FIG. 25 shows an absorption spectrum and an emission spectrum of a toluene solution of mCzCz2PSi.

FIG. 26 shows an absorption spectrum and an emission spectrum of a thin film of mCzCz2PSi.

FIG. 27 shows emission spectra of toluene solutions of mCzCz2PSi and PSiCzCz.

FIG. 28 shows phosphorescence spectra of thin films of mCzCz2PSi and PSiCzCz.

FIG. 29 shows luminance-current density characteristics of a light-emitting device 1 and a comparative light-emitting device 2.

FIG. 30 shows current efficiency-luminance characteristics of the light-emitting device 1 and the comparative light-emitting device 2.

FIG. 31 shows power efficiency-luminance characteristics of the light-emitting device 1 and the comparative light-emitting device 2.

FIG. 32 shows external quantum efficiency-luminance characteristics of the light-emitting device 1 and the comparative light-emitting device 2.

FIG. 33 shows electroluminescence spectra of the light-emitting device 1 and the comparative light-emitting device 2.

FIG. 34 shows luminance-current density characteristics of a light-emitting device 3 and a comparative light-emitting device 4.

FIG. 35 shows current efficiency-luminance characteristics of the light-emitting device 3 and the comparative light-emitting device 4.

FIG. 36 shows electroluminescence spectra of the light-emitting device 3 and the comparative light-emitting device 4.

FIG. 37 shows luminance-current density characteristics of a light-emitting device 5 and a comparative light-emitting device 6.

FIG. 38 shows current efficiency-luminance characteristics of the light-emitting device 5 and the comparative light-emitting device 6.

FIG. 39 shows external quantum efficiency-luminance characteristics of the light-emitting device 5 and the comparative light-emitting device 6.

FIG. 40 shows electroluminescence spectra of the light-emitting device 5 and the comparative light-emitting device 6.

FIG. 41 shows luminance-current density characteristics of a light-emitting device 7 and a comparative light-emitting device 8.

FIG. 42 shows current efficiency-luminance characteristics of the light-emitting device 7 and the comparative light-emitting device 8.

FIG. 43 shows the external quantum efficiency-luminance characteristics of the light-emitting device 7 and the comparative light-emitting device 8.

FIG. 44 shows the electroluminescence spectra of the light-emitting device 7 and the comparative light-emitting device 8.

FIG. 45 shows luminance changes over driving time of the light-emitting device 7 and the comparative light-emitting device 8.

FIG. 46 shows luminance-current density characteristics of a light-emitting device 9 and a comparative light-emitting device 10.

FIG. 47 shows current efficiency-luminance characteristics of the light-emitting device 9 and a comparative light-emitting device 10.

FIG. 48 shows the power efficiency-luminance characteristics of the light-emitting device 9 and a comparative light-emitting device 10.

FIG. 49 shows the electroluminescence spectra of the light-emitting device 9 and a comparative light-emitting device 10.

FIG. 50 shows luminance-current density characteristics of a light-emitting device 11 and a comparative light-emitting device 12.

FIG. 51 shows current efficiency-luminance characteristics of the light-emitting device 11 and the comparative light-emitting device 12.

FIG. 52 shows the luminance-voltage characteristics of the light-emitting device 11 and the comparative light-emitting device 12.

FIG. 53 shows the current density-voltage of the light-emitting device 11 and the comparative light-emitting device 12.

FIG. 54 the power efficiency-luminance characteristics of the light-emitting device 11 and the comparative light-emitting device 12.

FIG. 55 shows external quantum efficiency-luminance characteristics of the light-emitting device 11 and the comparative light-emitting device 12.

FIG. 56 shows energy efficiency-luminance characteristics of the light-emitting device 11 and the comparative light-emitting device 12.

FIG. 57 shows the electroluminescence spectra of the light-emitting device 11 and a comparative light-emitting device 12.

FIG. 58 shows bonding energy of a Q-R₁ bond of each of organic compounds.

### DETAILED DESCRIPTION OF THE INVENTION

Embodiments of the present invention will be described in detail below with reference to the drawings. Note that the present invention is not limited to the following description, and the modes and details of the present invention can be modified in various ways without departing from the spirit and scope of the present invention. Thus, the present invention should not be construed as being limited to the description in the following embodiments.

Note that the position, size, range, or the like of each component illustrated in drawings and the like is not accurately represented in some cases for easy understanding. Thus, the disclosed invention is not necessarily limited to the position, size, range, or the like disclosed in the drawings and the like.

Ordinal numbers such as “first” and “second” in this specification and the like are used for convenience and do not denote the order of steps or the stacking order of layers in some cases. Thus, for example, description can be made even when “first” is replaced with “second” or “third”, as appropriate. In addition, the ordinal numbers in this specification and the like are not necessarily the same as those used to specify one embodiment of the present invention.

In the description of structures of the present invention in this specification and the like with reference to the drawings, the same components in different drawings are denoted by the same reference numeral in some cases.

In this specification and the like, the terms “film” and “layer” can be interchanged with each other. For example, the term “conductive layer” can be changed to the term “conductive film” in some cases. For another example, the term “insulating film” can be changed into the term “insulating layer” in some cases.

In this specification and the like, the term “substituted” in the expression “substituted or unsubstituted” means that the group has a substituent.

Note that in this specification and the like, “hydrogen” includes protium and deuterium. Thus, unless otherwise specified, “hydrogen” simply shown in a formula or a sentence or hydrogen omitted in a chemical formula may be deuterium in some cases.

In this specification and the like, a singlet excited state (S*) refers to a singlet state having excitation energy. An S₁ level means the lowest level of the singlet excitation energy level, that is, the excitation energy level of the lowest singlet excited state. A triplet excited state (T*) refers to a triplet state having excitation energy. A T₁ level means the lowest level of the triplet excitation energy level, that is, the excitation energy level of the lowest triplet excited state.

In this specification and the like, a fluorescent material or a fluorescent compound refers to a material or a compound that emits light during the relaxation from the singlet excited state to the ground state. A phosphorescent material or a phosphorescent compound refers to a material or a compound that emits light in the visible light region at room temperature during the relaxation from the triplet excited state to the ground state. In other words, a phosphorescent material or a phosphorescent compound refers to a material or a compound that can convert triplet excitation energy into visible light.

Note that in this specification and the like, “room temperature” refers to a temperature in the range of 0° C. to 40° C.

Note that when the ν=0→ν=0 transition (0→0 band) between vibrational levels of the ground state and the excited state is clearly observed from a fluorescence spectrum or a phosphorescence spectrum, the S₁ level or the T₁ level of an organic compound is preferably calculated using the 0→0 band (see Non-Patent Document 1, for example). When the 0→0 band is unclear, the S₁ level can be energy of the wavelength at the intersection of the horizontal axis (wavelength) or the base line and a tangent to the fluorescence spectrum at a point where the slope of the spectrum at a peak on the shorter wavelength side has a maximum value, and the T₁ level can be energy of the wavelength at the intersection of the horizontal axis (wavelength) or the base line and a tangent to the phosphorescence spectrum at a point where the slope of the spectrum at a peak on the shorter wavelength side has a maximum value (see Non-Patent Document 2, for example). In the case where the levels are compared with each other, those calculated by the same method are used.

In this specification and the like, a blue range refers to the range of wavelengths greater than or equal to 400 nm and less than 490 nm, and blue light emission has at least one emission spectrum peak in that range. A green wavelength range refers to the range of wavelengths greater than or equal to 490 nm and less than 580 nm, and green light emission has at least one emission spectrum peak in that range. A red wavelength range refers to the range of wavelengths greater than or equal to 580 nm and less than or equal to 680 nm, and red light emission has at least one emission spectrum peak in that range. A near infrared light wavelength range refers to the range of wavelengths greater than or equal to 700 nm and less than or equal to 2500 nm, and near infrared light emission has at least one emission spectrum peak in that range.

In this specification and the like, when an aromatic ring (including a heteroaromatic ring) A is described, the description of the aromatic ring A can be applied to a fused aromatic ring generated by fusion of the aromatic ring A to another aromatic ring or a heteroaromatic ring, unless otherwise specified. For example, in the case where an organic compound having a carbazole ring is described, the description of the organic compound having a carbazole ring can be applied to an organic compound having a fused aromatic ring such as a benzocarbazole ring or a dibenzocarbazole ring, which is generated by fusion of a carbazole ring to a benzene ring(s), unless otherwise specified.

The values of HOMO and LUMO levels used in this specification can be obtained by electrochemical measurement. Typical examples of the electrochemical measurement include cyclic voltammetry (CV) measurement and differential pulse voltammetry (DPV) measurement.

The value of T_{g} used in this specification can be obtained with a differential scanning calorimetry device (DSC). In the case where the values of T_{g} of different compounds are compared with each other, the values calculated by the same method are used. In this specification, with the rate of temperature rise set to approximately between 40° C./min and 50° C./min, the measured value of the T_{g} endotherm rising (onset) temperature is regarded as a read value.

### Embodiment 1

In this embodiment, an organic compound of one embodiment of the present invention will be described.

An organic compound of one embodiment of the present invention is represented by the general formula (G1). In the general formula (G1) below, lines are drawn around each of a first partial structure (P1), a second partial structure (P2), and a third partial structure (L1) for description. The organic compound represented by the general formula (G1) has a structure in which the first partial structure (P1) and the second partial structure (P2) are connected to each other through the third partial structure (L1). The main chain of the third partial structure (L1) connecting the first partial structure (P1) and the second partial structure (P2) is composed of an sp³ hybrid orbital (a sigma bond). Such a structure can increase the S₁ level and the T₁ level of the organic compound.

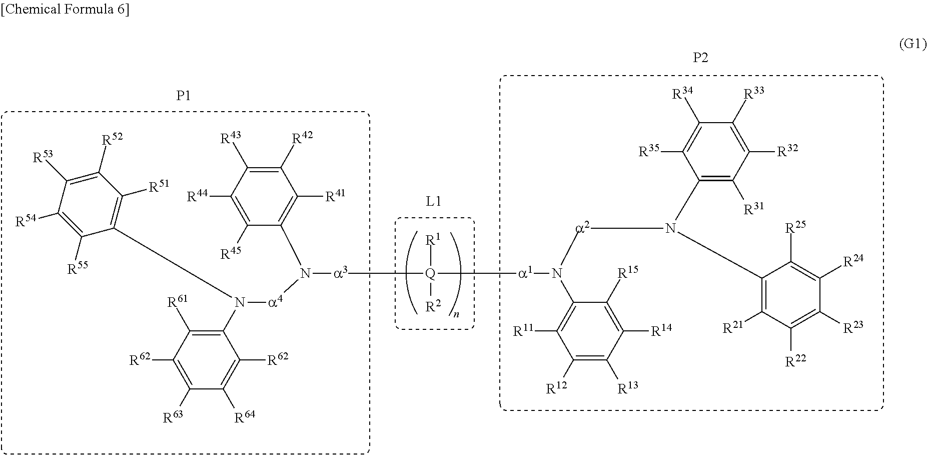

In the general formula (G1), Q represents carbon or silicon; each of R¹ and R² independently represents an alkyl group having 1 to 6 carbon atoms, a substituted or unsubstituted cycloalkyl group having 6 to 8 carbon atoms, or a substituted or unsubstituted aryl group having 6 to 12 carbon atoms; n represents an integer greater than or equal to 1 and less than or equal to 3; each of α¹ to α⁴ independently represents a substituted or unsubstituted phenylene group or a substituted or unsubstituted biphenylene group; at least one of R¹⁵, R²⁵, R³¹, R⁴⁵, R⁵⁵, and R⁶¹ represents a single bond; each of the others of R¹⁵, R²⁵, R³¹, R⁴⁵, R⁵⁵, and R⁶¹ independently represents hydrogen, an alkyl group having 1 to 6 carbon atoms, or a substituted or unsubstituted aryl group having 6 to 12 carbon atoms; and each of R¹¹ to R¹⁴, R²¹ to R²⁴, R³² to R³⁵, R⁴¹ to R⁴⁴, R⁵¹ to R⁵⁴, and R⁶² to R⁶⁵ independently represents hydrogen, an alkyl group having 1 to 6 carbon atoms, a substituted or unsubstituted cycloalkyl group having 6 to 8 carbon atoms, or a substituted or unsubstituted aryl group having 6 to 12 carbon atoms. When R¹⁵ is a single bond, R¹⁵ is bonded to α² to form a ring. When R⁴⁵ is a single bond, R⁴⁵ is bonded to α⁴ to form a ring. When both R²⁵ and R³¹ are single bonds, R²⁵ and R³¹ are bonded to each other to form a ring. When both R⁵⁵ and R⁶¹ are single bonds, R⁵⁵ and R⁶¹ are bonded to each other to form a ring. When one of R²⁵ and R³¹ is a single bond and the other of R²⁵ and R³¹ is hydrogen, an alkyl group having 1 to 6 carbon atoms, or a substituted or unsubstituted aryl group having 6 to 12 carbon atoms, α² is a substituted or unsubstituted biphenylene group and bonded to one of R²⁵ and R³¹ to form a ring. When one of R⁵⁵ and R⁶¹ is a single bond and the other of R⁵⁵ and R⁶¹ is hydrogen, an alkyl group having 1 to 6 carbon atoms, or a substituted or unsubstituted aryl group having 6 to 12 carbon atoms, α⁴ is a substituted or unsubstituted biphenylene group and bonded to one of R⁵⁵ and R⁶¹ to form a ring.

Note that the T_{g} of the organic compound can also be increased by a method in which a chain substituent is simply introduced into the organic compound. Note that a chain substituent does not have a greater effect of increasing heat resistance than an aromatic group such as an aryl group or a heteroaryl group. In addition, introducing a plurality of chain substituents into an organic compound may impair the carrier-transport property of the organic compound. Meanwhile, the organic compound of one embodiment of the present invention can have a high T_{g}, a high hole-transport property, a high S₁ level, and a high T₁ level because of its structure in which the first partial structure (P1) and the second partial structure (P2) each having an aromatic amine skeleton having a high hole-transport property are connected through the third partial structure (L1) including the main chain composed of the sigma bond.

When R¹⁵ in the general formula (G1) is a single bond, R¹⁵ can be bonded to α² to form a ring as shown in a general formula (G1-1-1) below. When R⁴⁵ is also a single bond, R⁴⁵ can be bonded to α⁴ to form a ring as shown in a general formula (G1-1-2) below. When such a ring(s) is/are formed, the organic compound can have a higher S₁ level and a higher T₁ level than when such a ring(s) is/are not formed.

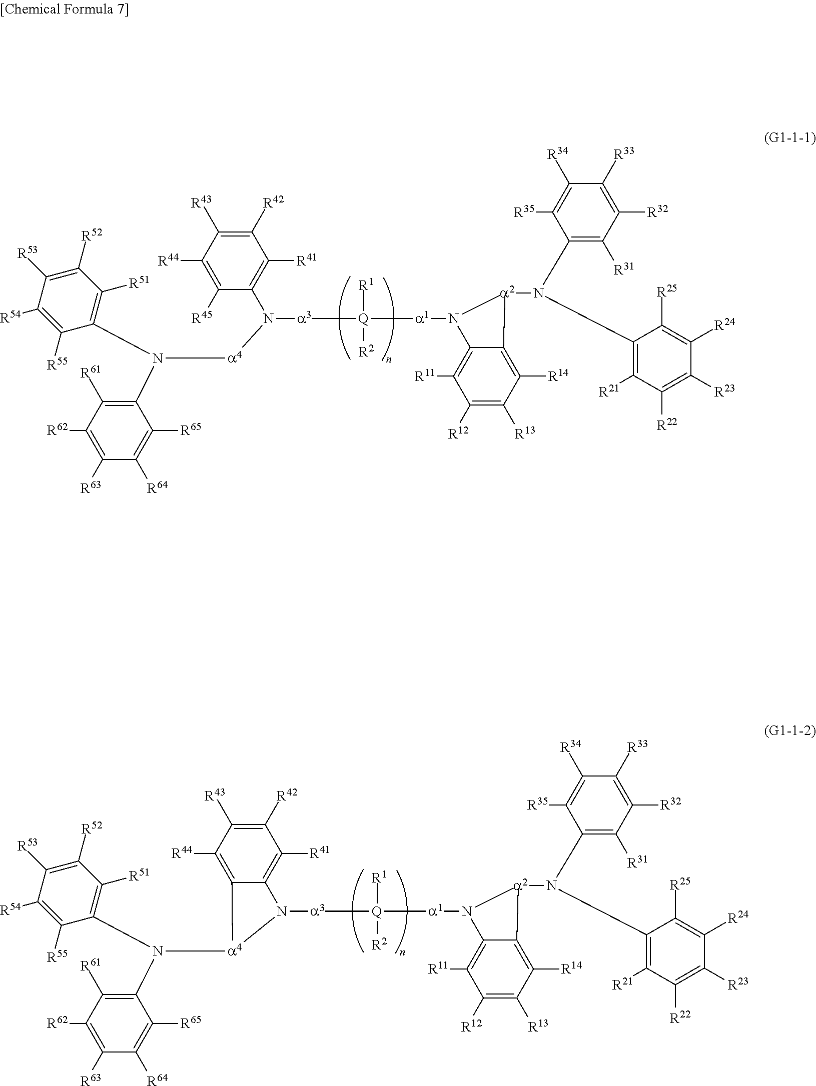

When each of R²⁵ and R³¹ in the general formula (G1) is a single bond, R²⁵ and R³¹ can be bonded to each other to form a ring as shown in a general formula (G1-2-1) below. When each of R⁵⁵ and R⁶¹ is also a single bond, R⁵⁵ and R⁶¹ can be bonded to each other to form a ring as shown in a general formula (G1-2-2) below. When such a ring(s) is/are formed, the organic compound can have a higher S₁ level and a higher T₁ level than when such a ring(s) is/are not formed. In addition, the formation of the ring(s) results in a molecular structure including a carbazole skeleton. The carbazole skeleton contributes to an improvement in hole-transport property, whereby the organic compound can have a high hole-transport property.

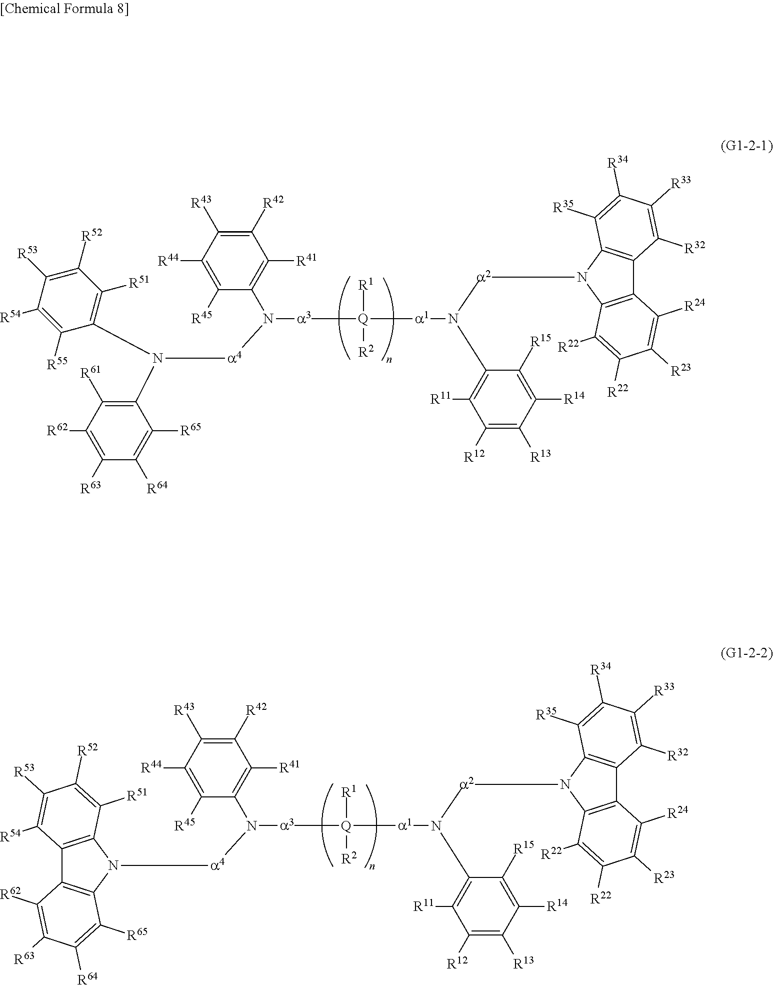

In the general formula (G1), when one of R²⁵ and R³¹ is a single bond and the other thereof is hydrogen, an alkyl group having 1 to 6 carbon atoms, or a substituted or unsubstituted aryl group having 6 to 12 carbon atoms, α² is a substituted or unsubstituted biphenylene group and one of R²⁵ and R³¹ can be bonded to α² to form a ring. In a general formula (G1-3-1) shown below, R²⁵ is a single bond. Furthermore, when one of R⁵⁵ and R⁶¹ is a single bond and the other thereof is hydrogen, an alkyl group having 1 to 6 carbon atoms, or a substituted or unsubstituted aryl group having 6 to 12 carbon atoms, α⁴ is a substituted or unsubstituted biphenylene group and one of R⁵⁵ and R⁶¹ can be bonded to α⁴ to form a ring. In the general formula (G1-3-2) shown below, R⁵⁵ is a single bond. Such structures increase the molecular weight and accordingly the organic compound can have a higher T_{g}. When such a ring(s) is/are formed, the organic compound can have a higher S₁ level and a higher T₁ level than when such a ring(s) is/are not formed.

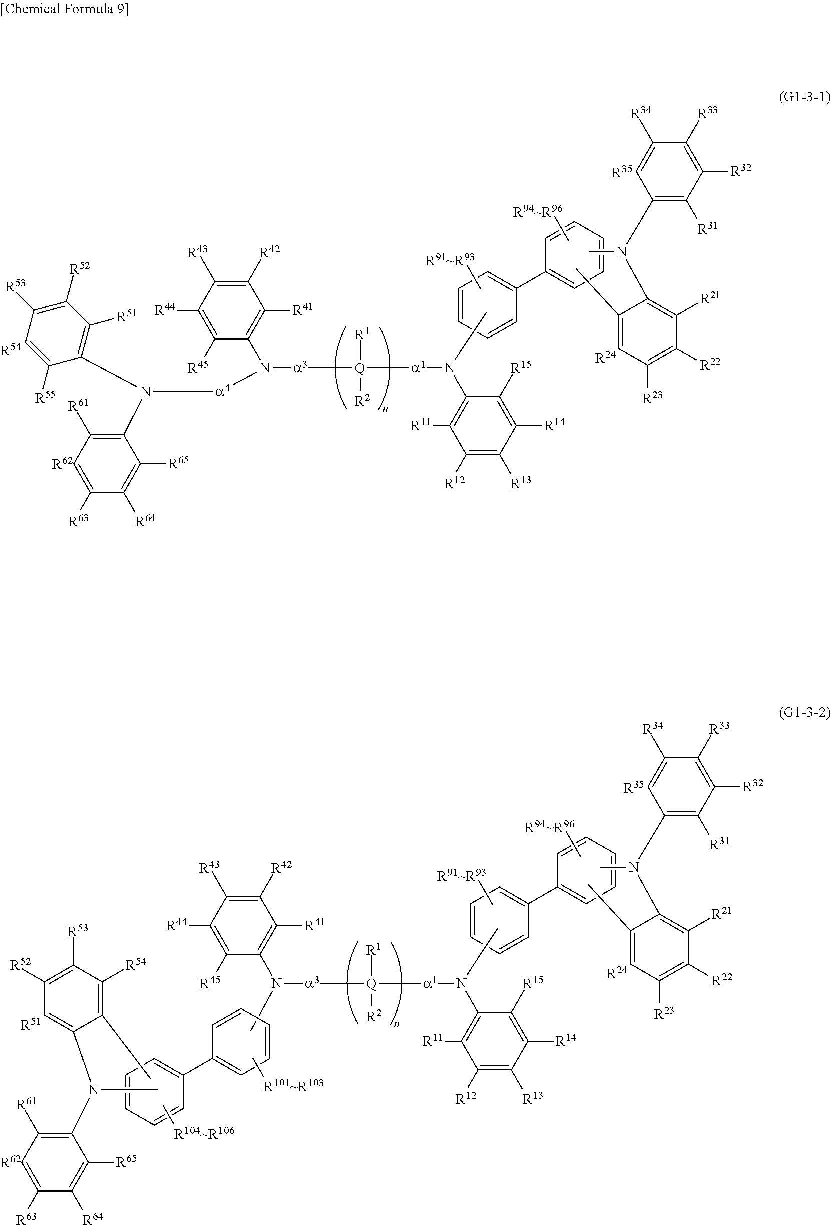

In the general formulae (G1-3-1) and (G1-3-2), each of R⁹¹ to R⁹⁶ and R¹⁰¹ to R¹⁰⁶ independently represents hydrogen, an alkyl group having 1 to 6 carbon atoms, or a substituted or unsubstituted aryl group having 6 to 12 carbon atoms.

In the case where each of α¹ and α³ in the general formula (G1) is independently a substituted or unsubstituted m-phenylene group, the organic compound has a bulky molecular structure and has a low sublimation temperature accordingly, so that the organic compound is not easily decomposed thermally during sublimation.

Another embodiment of the present invention is an organic compound represented by any of the general formulae (G2-1) and (G2-3) and a general formula (G2-2) below.

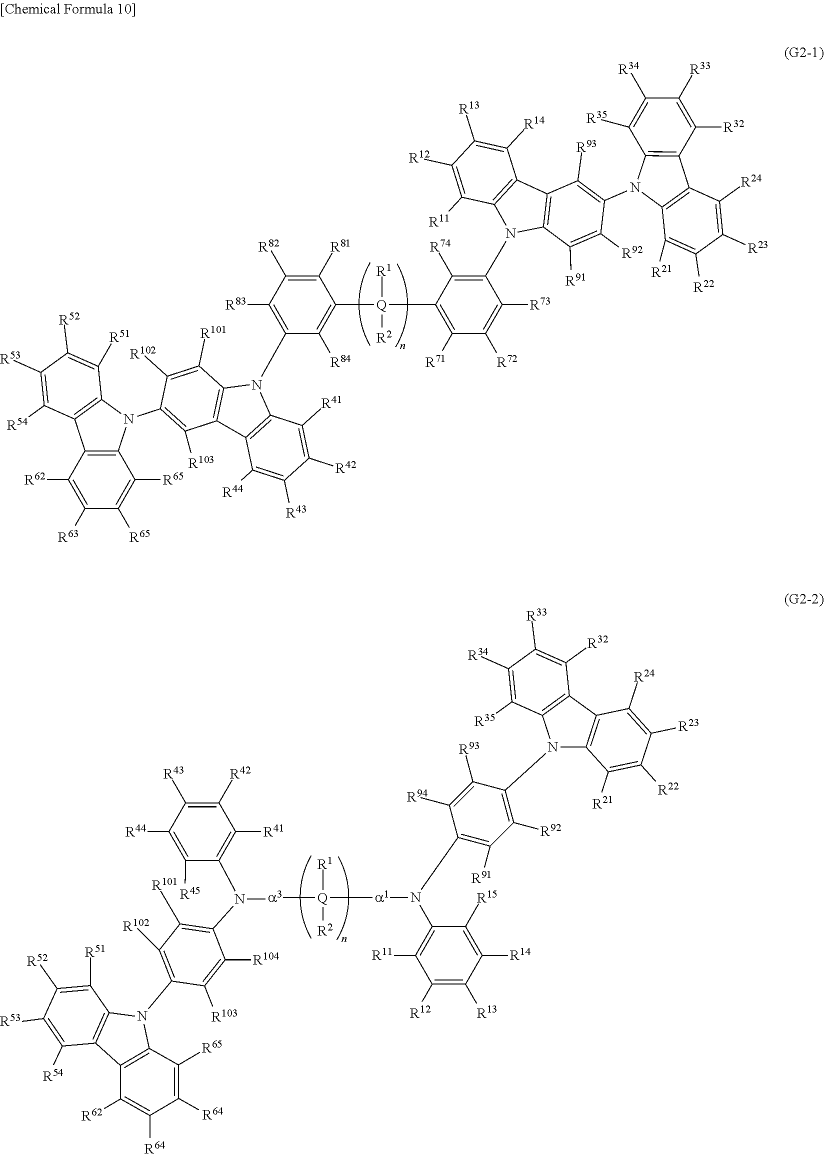

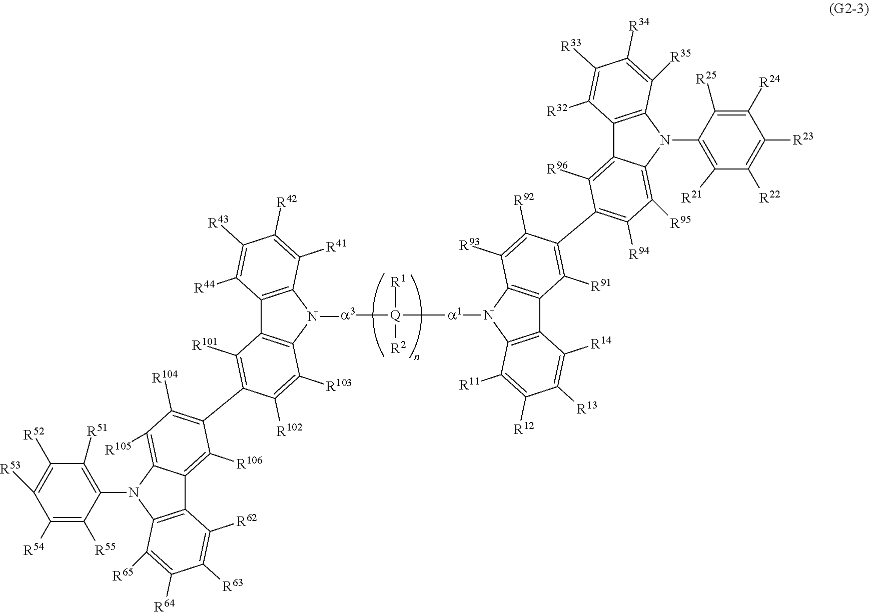

In the general formulae (G2-1), (G2-2), and (G2-3), Q represents carbon or silicon; each of R¹ and R² independently represents an alkyl group having 1 to 6 carbon atoms, a substituted or unsubstituted cycloalkyl group having 6 to 8 carbon atoms, or a substituted or unsubstituted aryl group having 6 to 12 carbon atoms; n represents an integer greater than or equal to 1 and less than or equal to 3; each of α¹ to α⁴ independently represents a substituted or unsubstituted phenyl group or a substituted or unsubstituted biphenyl group; and each of R¹¹ to R¹⁵, R²¹ to R²⁵, R³¹ to R³⁵, R⁴¹ to R⁴⁵, R⁵¹ to R⁵⁵, R⁶¹ to R⁶⁵, R⁷¹ to R⁷⁴, R⁸¹ to R⁸⁴, R⁹¹ to R⁹⁶, and R¹⁰¹ to R¹⁰⁶ independently represents hydrogen, an alkyl group having 1 to 6 carbon atoms, or a substituted or unsubstituted aryl group having 6 to 12 carbon atoms.

The general formula (G2-1) shows a structure where, in the general formula (G1), R¹⁵, R⁴⁵, R²⁵, R³¹, R⁵⁵, and R⁶¹ are each a single bond, α² and α⁴ are substituted or unsubstituted phenylene groups, R¹⁵ is bonded to α² to form a ring, R⁴⁵ is bonded to α⁴ to form a ring, R²⁵ and R³¹ are bonded to each other to form a ring, and R⁵⁵ and R⁶¹ are bonded to each other to form a ring. When such a ring(s) is/are formed, the organic compound can have a higher S₁ level and a higher T₁ level than when such a ring(s) is/are not formed. Furthermore, the organic compound has a structure including a carbazole ring having an excellent hole-transport property and thus can have a high hole-transport property.

In addition to the above, each of α¹ and α³ in the general formula (G1) is independently a substituted or unsubstituted m-phenylene group in the general formula (G2-1). Such a structure enables the organic compound to be bulky, thereby reducing the intermolecular interaction. Consequently, the organic compound can have a low sublimation temperature and is not easily decomposed thermally during sublimation.

The general formula (G2-2) shows a structure in which R²⁵ and R³¹ in the general formula (G1) are each a single bond and are bonded to each other to form a ring, and R⁵⁵ and R⁶¹ are each a single bond and are bonded to each other to form a ring. When such a ring(s) is/are formed, the organic compound can have a higher S₁ level and a higher T₁ level than when such a ring(s) is/are not formed. Furthermore, the organic compound has a structure including a carbazole ring having an excellent hole-transport property and thus can have a high hole-transport property.

The general formula (G2-3) is a structure where, in the general formula (G1), R³¹ is a single bond, α² is a substituted or unsubstituted biphenylene group, R³¹ is bonded to α² to form a ring, R⁶¹ is a single bond, α⁴ is a substituted or unsubstituted biphenylene group, and R⁶¹ is bonded to α⁴ to form a ring. Such structures increase the molecular weight and accordingly the organic compound can have a higher T_{g}. When such a ring(s) is/are formed, the organic compound can have a higher S₁ level and a higher T₁ level than when such a ring(s) is/are not formed. Furthermore, two carbazole rings having an excellent hole-transport property are bonded to each other at their 3-positions, whereby the organic compound can have a further higher hole-transport property.

Another embodiment of the present invention is an organic compound represented by a general formula (G3-1) or a general formula (G3-2) below.

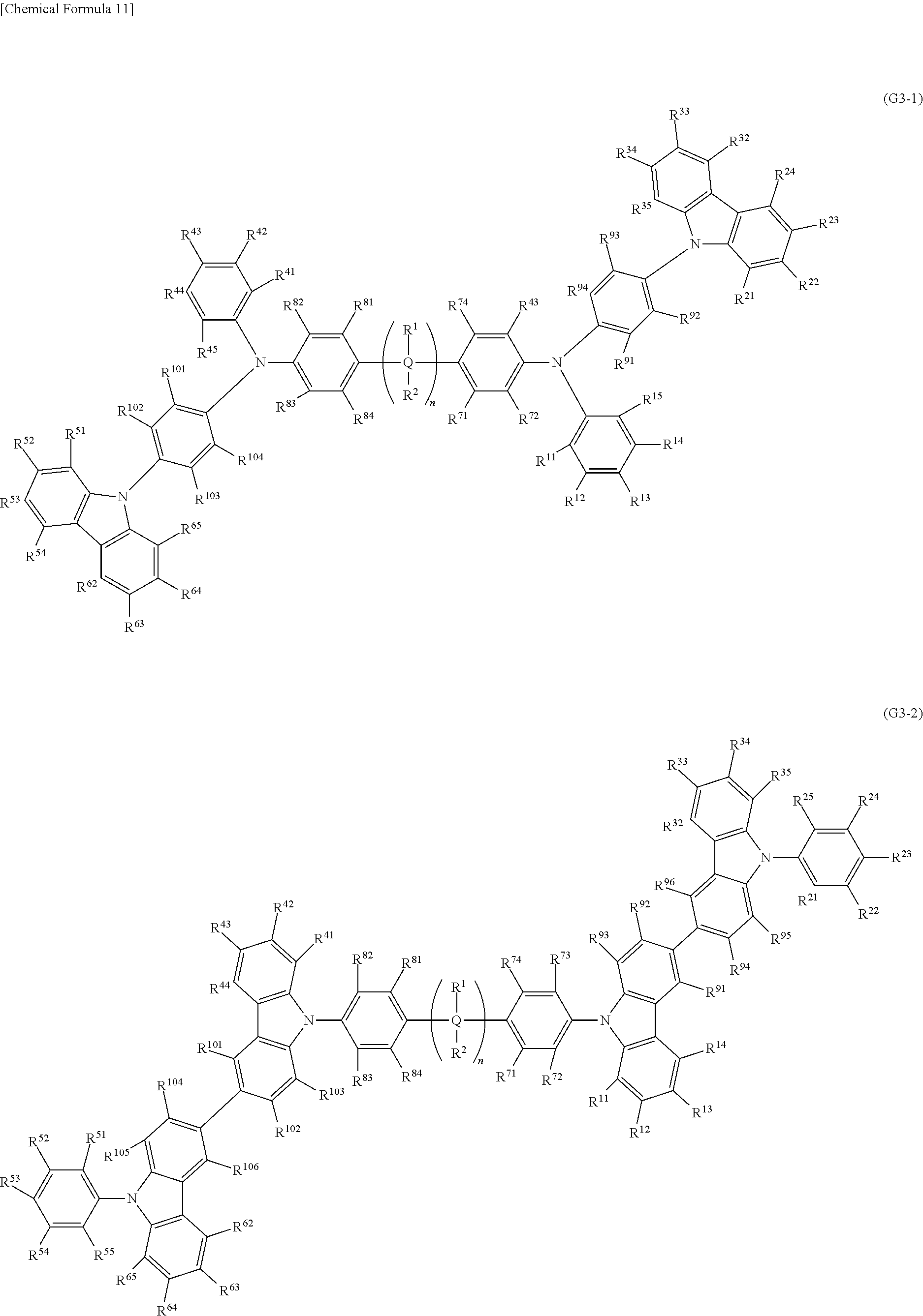

In the general formulae (G3-1) and (G3-2), Q represents carbon or silicon; each of R¹ and R² independently represents an alkyl group having 1 to 6 carbon atoms, a substituted or unsubstituted cycloalkyl group having 6 to 8 carbon atoms, or a substituted or unsubstituted aryl group having 6 to 12 carbon atoms; n represents an integer greater than or equal to 1 and less than or equal to 3; each of α¹ to α⁴ independently represents a substituted or unsubstituted phenyl group or a substituted or unsubstituted biphenyl group; and each of R¹¹ to R¹⁵, R²¹ to R²⁵, R³¹ to R³⁵, R⁴¹ to R⁴⁵, R¹ to R⁵⁵, R⁶¹ to R⁶⁵, R⁷¹ to R⁷⁴, R⁸¹ to R⁸⁴, R⁹¹ to R⁹⁶, and R¹⁰¹ to R¹⁰⁶ independently represents hydrogen, an alkyl group having 1 to 6 carbon atoms, or a substituted or unsubstituted aryl group having 6 to 12 carbon atoms.

In the general formula (G3-1), each of α¹ and α³ in the general formula (G2-2) is a substituted or unsubstituted p-phenylene group. In the general formula (G3-2), each of α¹ and α³ in the general formula (G2-3) is a substituted or unsubstituted p-phenylene group.

Since a silicon atom has a larger atomic radius than a carbon atom, in each of the above general formulae, steric hindrance between the substituents bonded to Q can be effectively inhibited in the case of silicon as Q, as compared with the case of carbon as Q. Such inhibition can improve the chemical stability of the organic compound against excitation. A light-emitting device using the organic compound should have light-emitting device characteristics with a long lifetime.

When Q is silicon in each of the above general formulae, the carrier-transport property can be improved. A light-emitting device using the organic compound should have a lower driving voltage.

When R¹ and R² are not bulky substituents in each of the above general formulae where Q is carbon, the steric hindrance between the substituents (i.e., R¹, R², and the first and second partial structures) bonded to Q can be more inhibited. Hence, when Q is carbon, it is preferable that each of R¹ and R² be independently a chain group such as an alkyl group having 1 to 6 carbon atoms or a cyclic group such as a cycloalkyl group, for example.

When R¹ and R² in each of the above general formulae have increased molecular weights, the organic compound as a whole has a higher molecular weight. However, the organic compound as a whole having an excessively high molecular weight might have a too high sublimation temperature (or evaporation temperature) to be easily thermally decomposed during evaporation. When each of R¹ and R² is an alkyl group having 1 to 6 carbon atoms, a substituted or unsubstituted cycloalkyl group having 6 to 8 carbon atoms, or a substituted or unsubstituted aryl group having 6 to 12 carbon atoms, an excessive increase in the sublimation temperature (or evaporation temperature) of the organic compound can be inhibited. When each of R¹ and R² is independently a methyl group or a phenyl group, the stability of the organic compound can be improved.

As described above, increasing the molecular weight raises the T_{g} of the organic compound of one embodiment of the present invention. Meanwhile, if the organic compound of one embodiment of the present invention has an excessively high molecular weight, the organic compound might have a too high sublimation temperature (or evaporation temperature) to be easily thermally decomposed during evaporation. Hence, the molecular weight of the organic compound of one embodiment of the present invention is preferably higher than or equal to 600 and lower than or equal to 1500, further preferably higher than or equal to 700 and lower than or equal to 1300. With that structure, the organic compound can have an increased T_{g} and is not easily decomposed thermally.

In each of the above general formulae, in the case where n is 2 or 3, a plurality of Qs may be the same or different from each other, a plurality of R¹s may be the same or different from each other, and a plurality of R²s may be the same or different from each other.

Next, an alkyl group having 1 to 6 carbon atoms, a substituted or unsubstituted cycloalkyl group having 6 to 8 carbon atoms, and an aryl group having 6 to 12 carbon atoms, which are substituents that can be used in the above general formulae, are described and specific examples thereof are given. Note that the substituents that can be used in the above general formulae are not limited to the specific examples given below.

### <<Alkyl Group Having 1 to 6 Carbon Atoms>>

In this specification and the like, an alkyl group having 1 to 6 carbon atoms refers to a monovalent group obtained by eliminating one hydrogen atom from a straight-chain or branched alkane (CₙH₂ₙ₊₂ (n=1 to 6)) having 1 to 6 carbon atoms. Specific examples of the alkyl group having 1 to 6 carbon atoms include a methyl group, an ethyl group, an n-propyl group, an isopropyl group, an n-butyl group, a sec-butyl group, an isobutyl group, a tert-butyl group, an n-pentyl group, an isopentyl group, a sec-pentyl group, a tert-pentyl group, a neopentyl group, an n-hexyl group, an isohexyl group, a sec-hexyl group, a tert-hexyl group, a neohexyl group, a 3-methylpentyl group, and a 2-methylpentyl group.

### <<Cycloalkyl Group Having 6 to 8 Carbon Atoms>>

In this specification and the like, a cycloalkyl group having 6 to 8 carbon atoms refers to a monovalent group obtained by eliminating one hydrogen atom from one of carbon atoms forming the ring of a cycloalkane (CₙH₂ₙ (n=6 to 8)) having 6 to 8 carbon atoms forming the ring. Specific examples of the cycloalkyl group having 6 to 8 carbon atoms include a cyclohexyl group, a cyclooctyl group, and a cycloheptyl group. In the case where the cycloalkyl group having 6 to 8 carbon atoms includes a substituent, a specific example of the substituent is an alkyl group having 1 to 6 carbon atoms.

### <<Aryl Group Having 6 to 12 Carbon Atoms>>

In this specification and the like, an aryl group having 6 to 12 carbon atoms refers to a monovalent group obtained by eliminating one hydrogen atom from one of carbon atoms forming the ring(s) of a monocyclic or polycyclic aromatic compound having 6 to 12 carbon atoms forming the ring(s). Note that in this specification and the like, a polycyclic aromatic compound refers to an aromatic compound having two or more cyclic structures in a molecule, which corresponds to both a material including a plurality of rings bonded by a single bond, such as biphenyl, and a material in which a plurality of rings are fused, such as naphthalene. Specific examples of the aryl group having 6 to 12 carbon atoms include a phenyl group, a naphthyl group, a fluorenyl group, a biphenyl group, and the like. In the case where the aryl group having 6 to 12 carbon atoms includes a substituent, a specific example of the substituent is an alkyl group having 1 to 6 carbon atoms.

The above are examples of the substituents that can be employed in the above general formulae.

Specific examples of the organic compounds of embodiments of the present invention represented by the above general formulae include organic compounds represented by the structural formulae (100) to a structural formula (129) below.

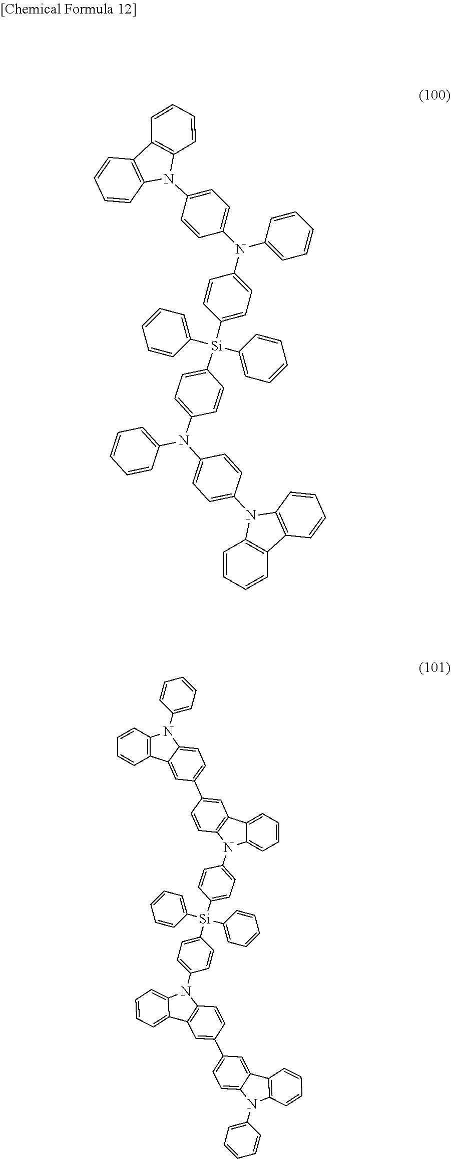

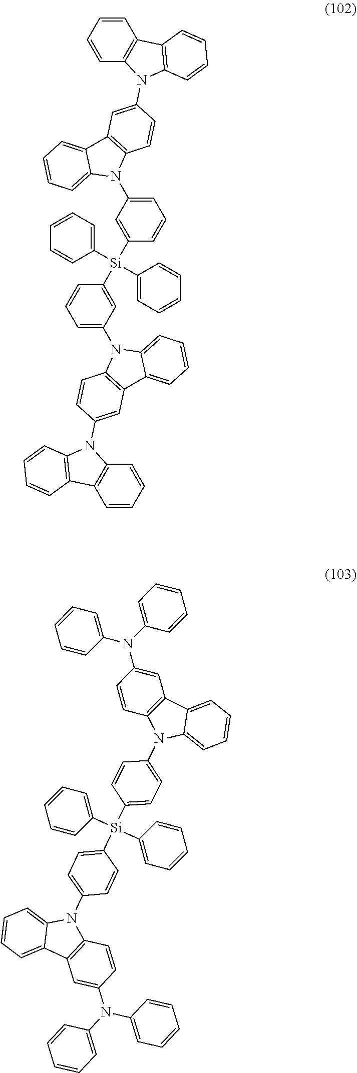

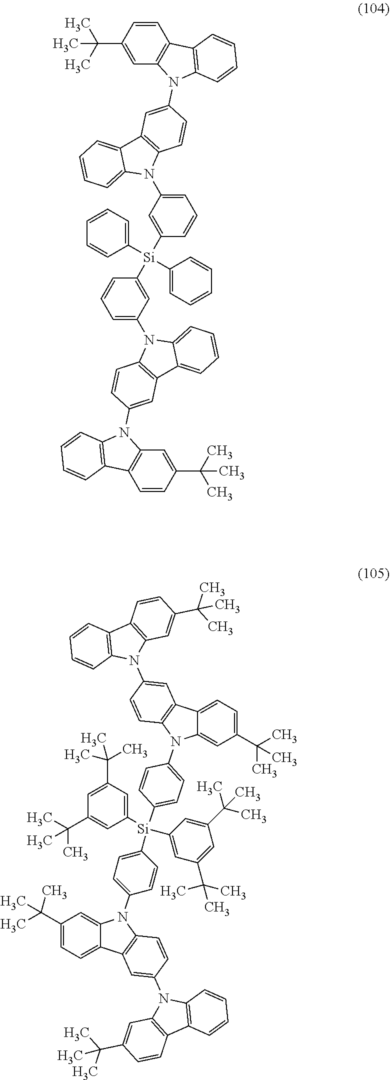

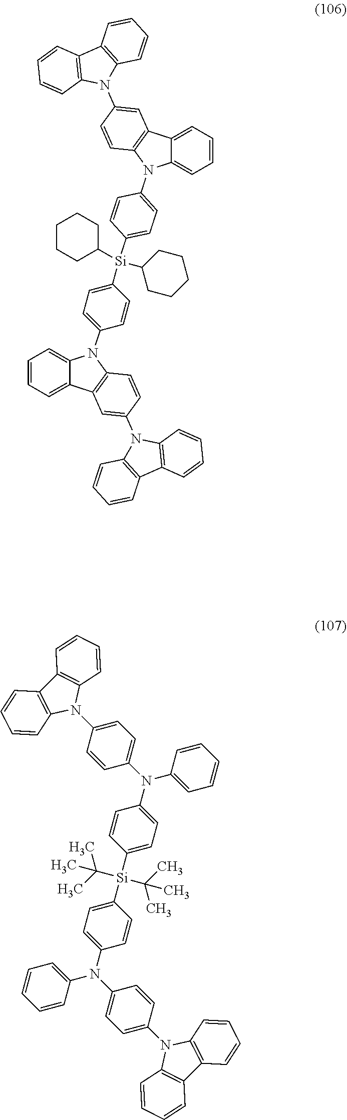

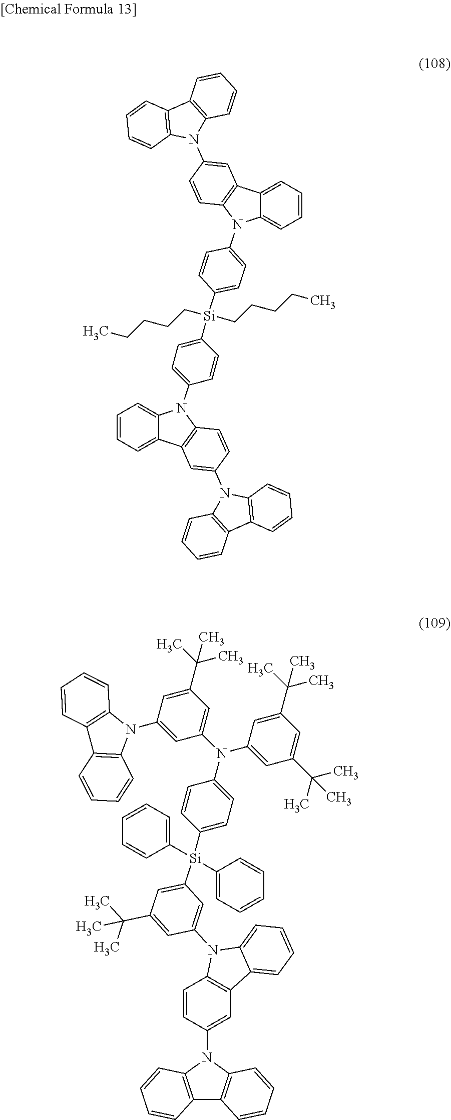

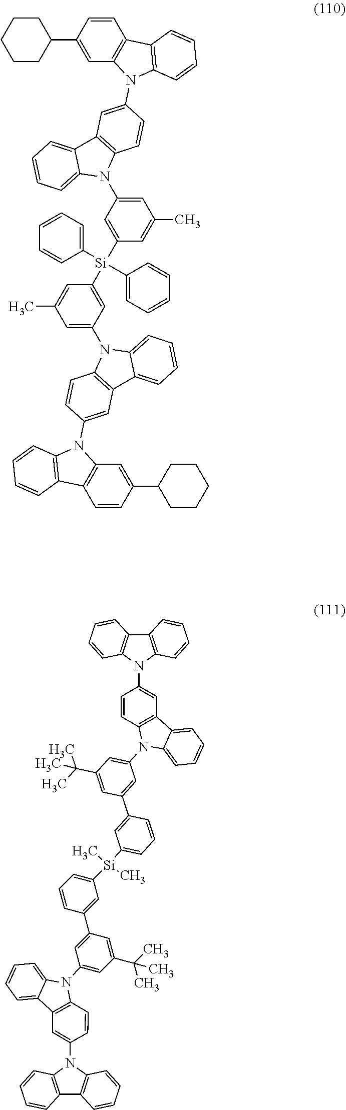

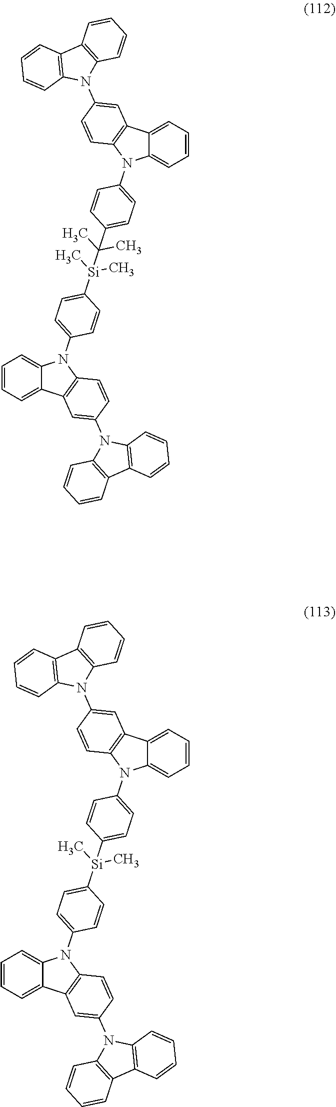

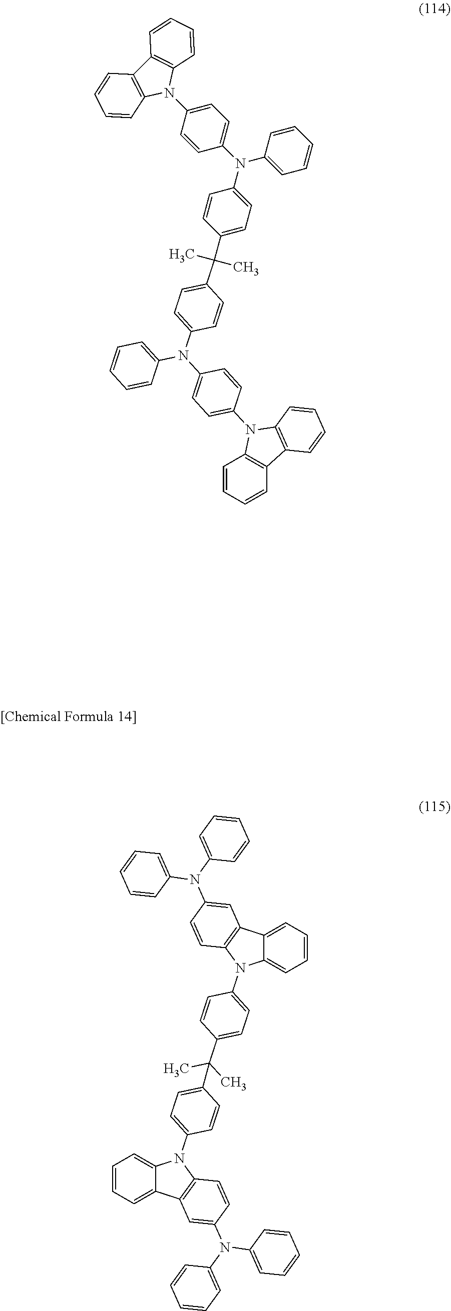

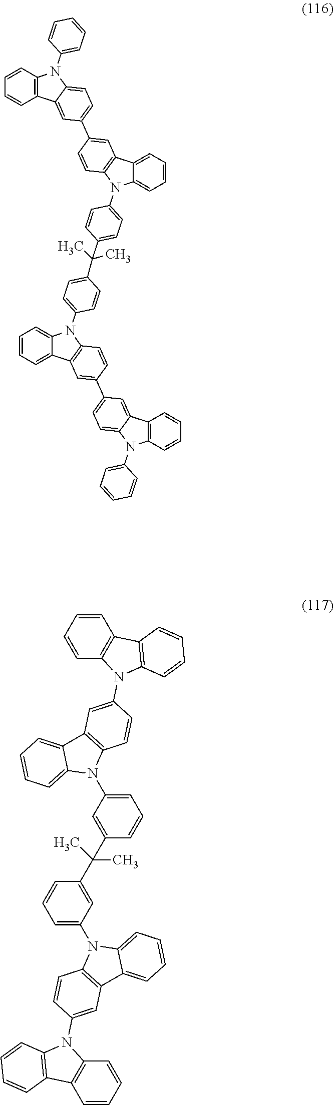

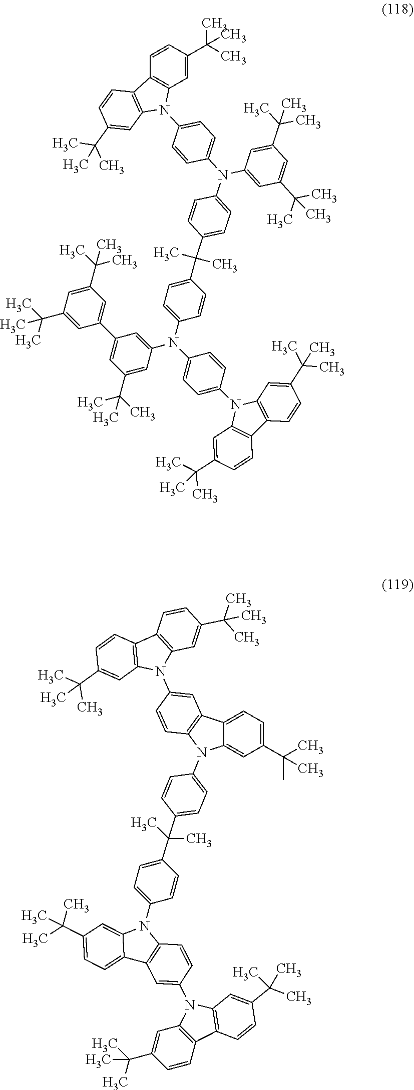

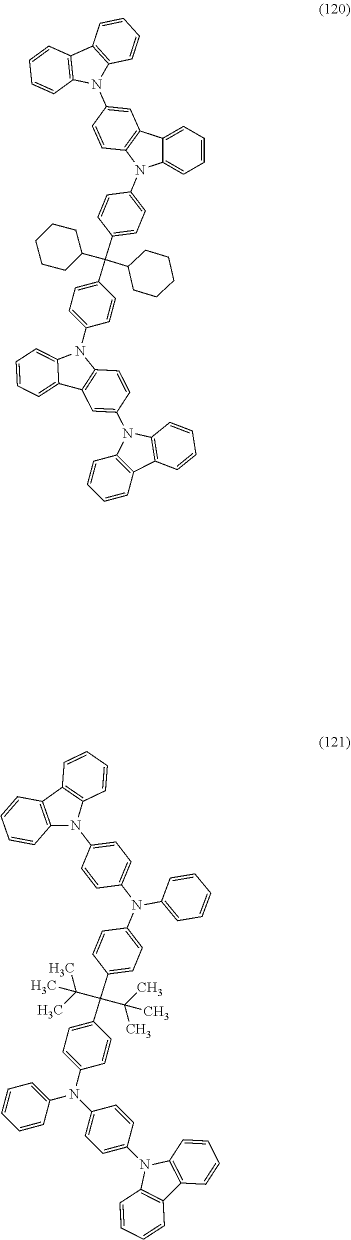

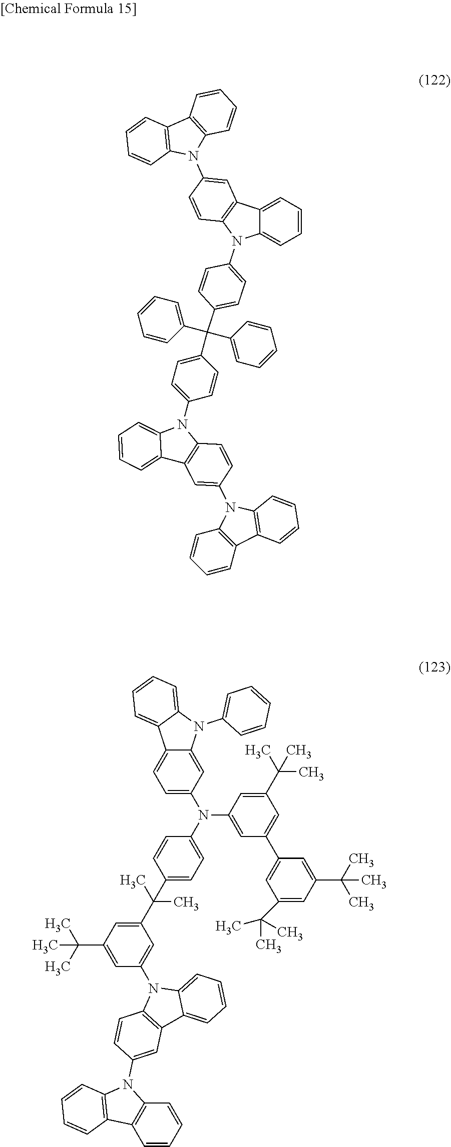

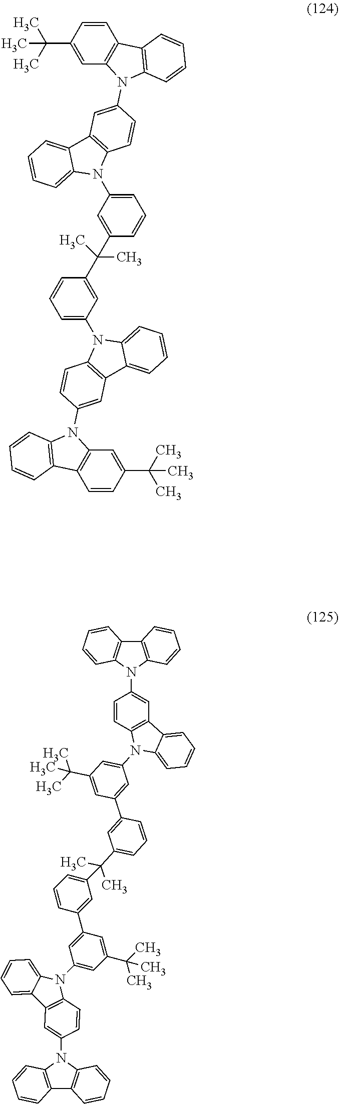

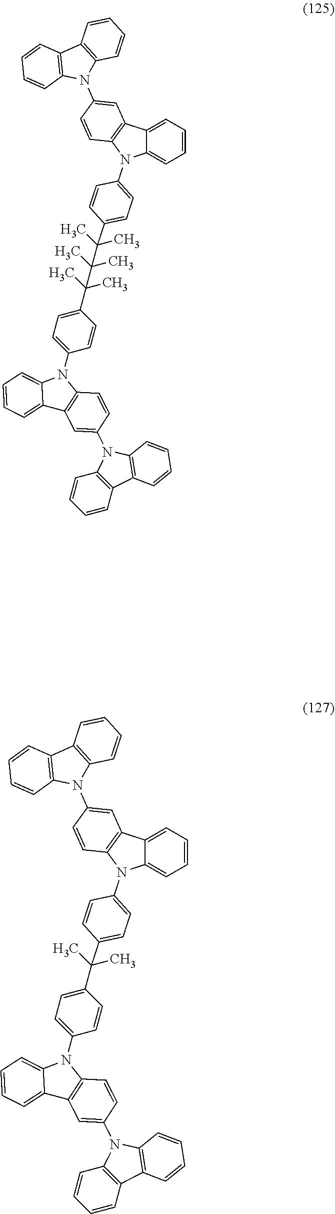

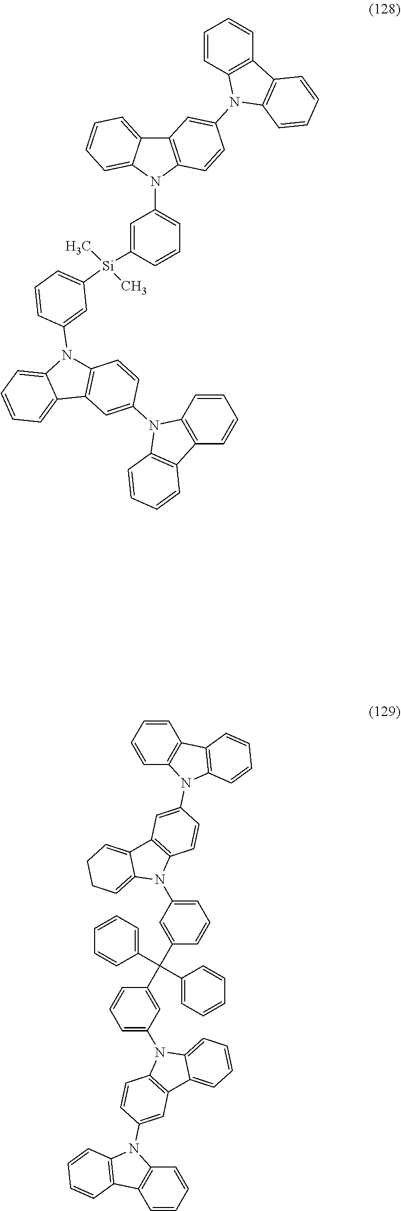

The organic compounds represented by structural formulae (100) to (129) are examples of the organic compound of one embodiment of the present invention; however, one embodiment of the present invention is not limited thereto.

When an alkyl group such as a tert-butyl group or a methyl group is introduced into an aromatic ring or a heteroaromatic ring included in the organic compound of one embodiment of the present invention, the organic compound becomes bulky and accordingly can have a low sublimation temperature and is not easily decomposed thermally. Examples of the structure in which an alkyl group such as a tert-butyl group or a methyl group is introduced into the aromatic ring or the heteroaromatic ring are represented by structural formulae (104), (105), (107), (109) to (111), (118), (119), (123), (124), and (125).

An alkyl group such as a tert-butyl group or a methyl group is bonded to carbon or silicon included in the third partial structure (L1), as in the structural formulae (109) to (111) and (114) to (128) and structural formulae (130) to (135), to adjust the molecular weight, and thus the sublimation temperature can be adjusted.

Next, as an example of a synthesis method of the organic compound of one embodiment of the present invention, a synthesis method of the organic compound represented by the general formula (G1) below is described. Note that the synthesis method of the organic compound represented by the general formula (G1) can employ a variety of reactions and is not limited to the following synthesis methods.

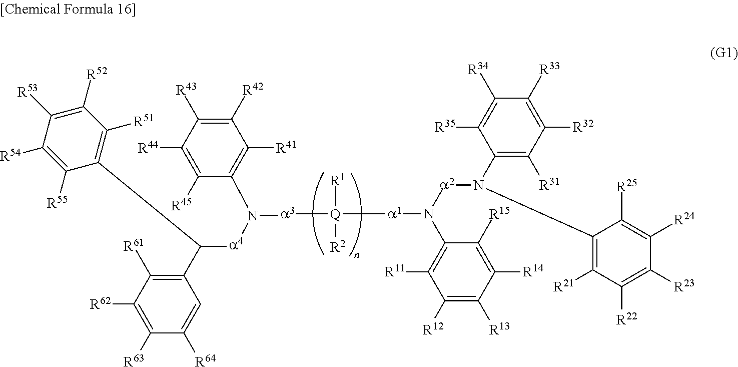

In the general formula (G1), Q represents carbon or silicon; each of R¹ and R² independently represents an alkyl group having 1 to 6 carbon atoms, a substituted or unsubstituted cycloalkyl group having 6 to 8 carbon atoms, or a substituted or unsubstituted aryl group having 6 to 12 carbon atoms; n represents an integer greater than or equal to 1 and less than or equal to 3; each of α¹ to α⁴ independently represents a substituted or unsubstituted phenylene group or a substituted or unsubstituted biphenylene group; at least one of R¹⁵, R²⁵, R³¹, R⁴⁵, R⁵⁵, and R⁶¹ represents a single bond; each of the others of R¹⁵, R²⁵, R³¹, R⁴⁵, R⁵⁵, and R⁶¹ independently represents hydrogen, an alkyl group having 1 to 6 carbon atoms, or a substituted or unsubstituted aryl group having 6 to 12 carbon atoms; and each of R¹¹ to R¹⁴, R²¹ to R²⁴, R³² to R³⁵, R⁴¹ to R⁴⁴, R⁵¹ to R⁵⁴, and R⁶² to R⁶⁵ independently represents hydrogen, an alkyl group having 1 to 6 carbon atoms, a substituted or unsubstituted cycloalkyl group having 6 to 8 carbon atoms, or a substituted or unsubstituted aryl group having 6 to 12 carbon atoms. When R¹⁵ is a single bond, R¹⁵ is bonded to α² to form a ring. When R⁴⁵ is a single bond, R⁴⁵ is bonded to α⁴ to form a ring. When both R²⁵ and R³¹ are single bonds, R²⁵ and R³¹ are bonded to each other to form a ring. When both R⁵⁵ and R⁶¹ are single bonds, R⁵⁵ and R⁶¹ are bonded to each other to form a ring. When one of R²⁵ and R³¹ is a single bond and the other of R²⁵ and R³¹ is hydrogen, an alkyl group having 1 to 6 carbon atoms, or a substituted or unsubstituted aryl group having 6 to 12 carbon atoms, α² is a substituted or unsubstituted biphenylene group and bonded to one of R²⁵ and R³¹ to form a ring. When one of R⁵⁵ and R⁶¹ is a single bond and the other of R⁵⁵ and R⁶¹ is hydrogen, an alkyl group having 1 to 6 carbon atoms, or a substituted or unsubstituted aryl group having 6 to 12 carbon atoms, α⁴ is a substituted or unsubstituted biphenylene group and bonded to one of R⁵⁵ and R⁶¹ to form a ring.

A halogen compound (a1), an amine compound (a2), and an amine compound (a3) are subjected to cross coupling reaction, so that the organic compound represented by the general formula (G1) can be obtained. A synthesis scheme (A-1) is shown below.

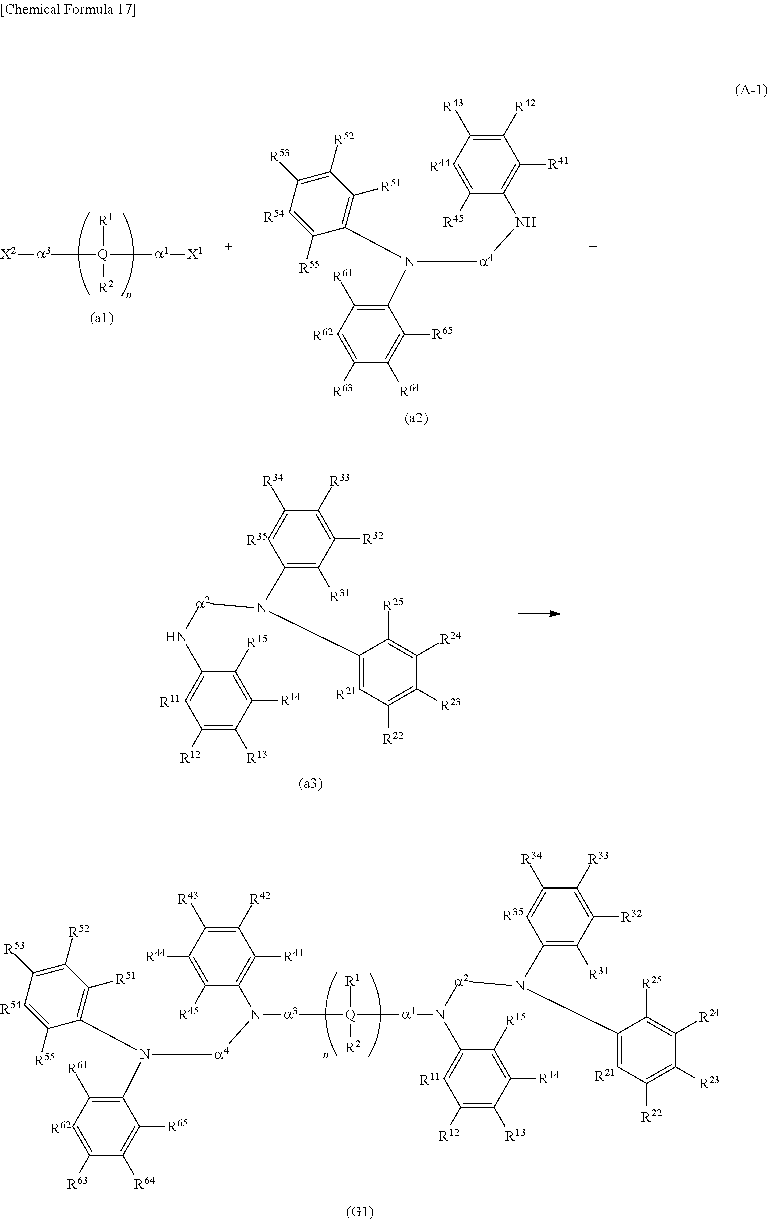

In the synthesis scheme (A-1), Q, R¹, R², α¹ to α⁴, R¹⁵, R²⁵, R³¹, R⁴⁵, R⁵⁵, R⁶¹, R¹¹ to R¹⁴, R²¹ to R²⁴, R³² to R³⁵, R⁴¹ to R⁴⁵, R⁵¹ to R⁵⁵, and R⁶¹ to R⁶⁵ are each the atom or group described above. In addition, X¹ and X² each represent a halogen.

In the synthesis scheme (A-1), specific examples of the halogen include chlorine, bromine, and iodine; X¹ and X² are preferably bromine or iodine, further preferably iodine owing to its high reactivity. This reaction can proceed under various conditions. For example, a synthesis method in which a metal catalyst is used under the presence of a base can be employed. Specifically, the Ullmann coupling, the Buchwald-Hartwig reaction, or the like can be used.

In the case where the amine compounds (a2) and (a3) have different molecular structures, the amine compounds (a2) and (a3) are preferably reacted successively with the halogen compound (a1) for higher-purity and higher-yield synthesis. By contrast, in the case where the amine compounds (a2) and (a3) have the same molecular structure, preferably, two equivalents of the amine compound (a2) are reacted with one equivalent of the halogen compound (a1), in which case reaction steps and synthesis cost can be reduced.

The structures described in this embodiment can be used in combination with any of the structures described in the other embodiments as appropriate.

### Embodiment 2

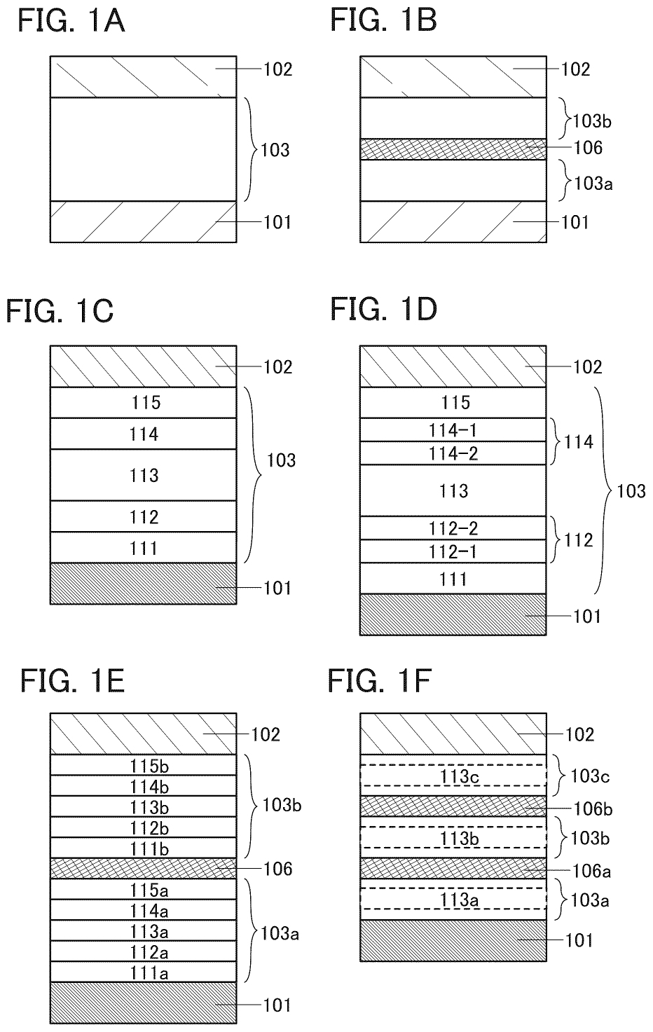

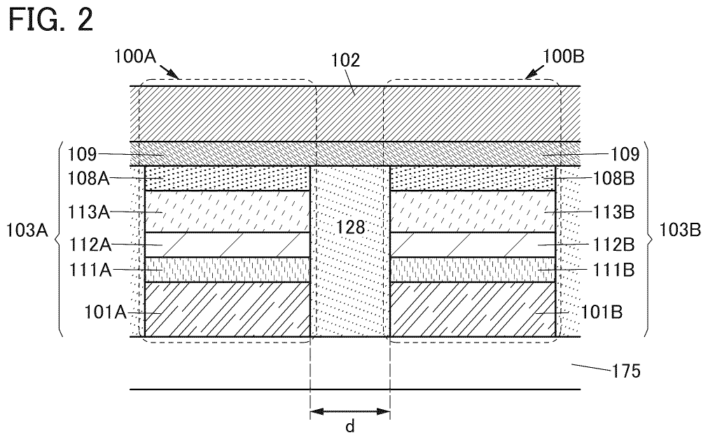

In this embodiment, a structure of a light-emitting device of one embodiment of the present invention is described with reference to FIGS. 1A to 1F and FIG. 2.

For the light-emitting device of one embodiment of the present invention, an organic compound (referred to as a first organic compound) described below can be used. Note that the first organic compound includes the organic compound represented by the general formula (G1) described in Embodiment 1.

The first organic compound includes a first partial structure, a second partial structure, and a third partial structure. The first partial structure and the second partial structure are connected through the third partial structure. At least one of the first and second partial structures includes a carrier-transport (hole-transport and/or electron-transport) skeleton. The third partial structure is represented by the general formula (L1).

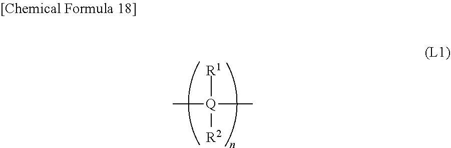

In general formula (L1), Q represents carbon or silicon; each of R¹ and R² independently represents an alkyl group having 1 to 6 carbon atoms, a substituted or unsubstituted cycloalkyl group having 6 to 8 carbon atoms, or a substituted or unsubstituted aryl group having 6 to 12 carbon atoms; and n is greater than or equal to 1 and less than or equal to 3.

Examples of the hole-transport skeleton include a π-electron rich heteroaromatic ring and an aromatic amine skeleton, and examples of the electron-transport skeleton include a π-electron deficient heteroaromatic ring. In other words, at least one of the first and second partial structures preferably includes one or more of a π-electron rich heteroaromatic ring, an aromatic amine skeleton, and a π-electron deficient heteroaromatic ring. In that case, the first organic compound can be a carrier-transport material.

As the π-electron rich heteroaromatic ring, a fused aromatic ring including at least one of a pyrrole ring, a furan ring, and a thiophene ring as a ring is preferably used, for example. Specifically, a carbazole ring, a dibenzofuran ring, a dibenzothiophene ring, and a fused aromatic ring in which an aromatic ring or a heteroaromatic ring is further fused to a carbazole ring, a dibenzofuran ring, or a dibenzothiophene ring is preferred because of its chemical stability. In particular, a 9-substituted carbazolyl group is preferably used to increase the S₁ level and the T₁ level.

As the aromatic amine skeleton, a tertiary amine having no NH bond is preferred, and a triarylamine skeleton is particularly preferred because of its excellent chemical stability in excitation and favorable hole-transport property. As an aryl group of the triarylamine skeleton, a substituted or unsubstituted aryl group having 6 to 13 carbon atoms forming a ring is preferred and examples thereof include a phenyl group, a naphthyl group, and a fluorenyl group. In particular, a phenyl group is preferably used to increase the S₁ level and the T₁ level.

Further preferably, at least one of the first and second partial structures have both a π-electron rich heteroaromatic ring and an aromatic amine skeleton. In that case, the organic compound can have an excellent hole-transport property and be stable and highly reliable.

Examples of the π-electron deficient heteroaromatic ring include an oxadiazole ring, a triazole ring, a benzimidazole ring, a quinoxaline ring, a dibenzoquinoxaline ring, a quinazoline ring, a phenanthroline ring, a pyridine ring, a diazine ring (a pyrimidine ring, a pyrazine ring, and a pyridazine ring), a triazine ring, and a furodiazine ring. A skeleton including a triazine ring or a diazine ring is particularly preferred because of its chemical stability in excitation.

The first and second partial structures may each include a carrier-transport skeleton; however, one of the first and second partial structures does not necessarily include a carrier-transport skeleton.

Note that the first organic compound may have a bipolar property. In other words, the first partial structure may include one or more of a π-electron rich heteroaromatic ring and an aromatic amine skeleton while the second partial structure may include a π-electron deficient heteroaromatic ring.

The structure where the first and second partial structures each include a carrier-transport skeleton can further increase the carrier-transport property of the organic compound. This structure also increases the molecular weight, resulting in a higher T_{g}. The structure where the first and second partial structures include carrier-transport skeletons having the same structure is preferred because the synthesis of the organic compound can be facilitated.

The one of the first and second partial structures that does not include a carrier-transport skeleton preferably includes, as the partial structure, a structure that can increase the molecular weight of the first organic compound. Examples of the structure that can increase the molecular weight of the first organic compound include a substituted or unsubstituted aryl group having 6 to 12 carbon atoms, preferably a phenyl group. The structure can increase the heat resistance of the first organic compound.

The first and second partial structures are connected through the third partial structure represented by the general formula (L1) described in Embodiment 1 as described above, whereby the organic compound can have high heat resistance, a high S₁ level, and a high T₁ level.

When the first organic compound having a high S₁ level and a high T₁ level is used for a carrier-transport layer of a light-emitting device, the characteristics of the light-emitting device can be improved.

A specific example of the carrier-transport skeleton that is preferably used for at least one of the first and second partial structures is a partial structure represented by the general formula (T1).

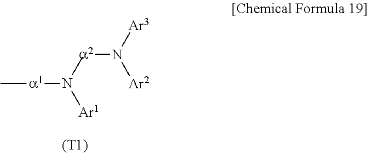

In the general formula (T1), each of α¹ and α² independently represents a substituted or unsubstituted arylene group having 6 to 12 carbon atoms, and each of Ar¹ to Ar³ independently represents a substituted or unsubstituted aryl group having 6 to 12 carbon atoms. Note that Ar¹ and α² may be bonded to each other to form a ring but are not necessarily bonded to each other. Furthermore, α² and Ar² may be bonded to each other to form a ring but are not necessarily bonded to each other. Ar² and Ar³ may be bonded to each other to form a ring but are not necessarily bonded to each other.

The partial structure represented by the general formula (T1) includes one or more of a π-electron rich heteroaromatic ring and an aromatic amine skeleton. Accordingly, the first organic compound can be a hole-transport material by using the partial structure represented by the general formula (T1).

The first organic compound can have higher heat resistance and a further improved carrier-transport property by employing carrier-transport skeletons for both the first and second partial structures. A specific example of the first organic compound is the organic compound represented by a general formula (G0).

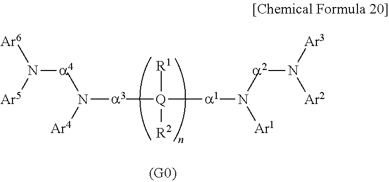

In the general formula (G0), Q represents carbon or silicon; each of R¹ and R² independently represents an alkyl group having 1 to 6 carbon atoms, a substituted or unsubstituted cycloalkyl group having 6 to 8 carbon atoms, or a substituted or unsubstituted aryl group having 6 to 12 carbon atoms; and n represents an integer greater than or equal to 1 and less than or equal to 3. Each of α¹ to α⁴ independently represents a substituted or unsubstituted phenylene group or a substituted or unsubstituted biphenylene group; and each of Ar¹ to Ar⁶ independently represents a substituted or unsubstituted aryl group having 6 to 12 carbon atoms. Note that Ar¹ and α², α² and Ar², and/or Ar² and Ar³ may be bonded to each other to form a ring(s) but are not necessarily bonded to each other. Ar⁴ and α⁴, α⁴ and Ar⁵, and/or Ar⁵ and Ar⁶ may be bonded to each other to form a ring(s) but are not necessarily bonded to each other.

The organic compound represented by the general formula (G0) employs the carrier-transport skeletons for both the first and second partial structures, and these are connected through the partial structure represented by the general formula (L1). Accordingly, the first organic compound can have higher heat resistance and a further improved carrier-transport property.

Preferably in the general formulae (T1) and (G0), each of α¹ to α⁴ is independently a substituted or unsubstituted phenylene group or a substituted or unsubstituted biphenylene group. Further preferably in the general formula (T1), each of Ar¹ to Ar⁶ is independently a substituted or unsubstituted phenyl group. With such a structure, an organic compound that has a low sublimation temperature and is not easily decomposed thermally during sublimation can be provided.

Further preferably in the general formulae (T1) and (G0), Ar¹ and α², α² and Ar², and/or Ar² and Ar³ is/are bonded to each other to form a ring(s). Further preferably, Ar⁴ and α⁴, α⁴ and Ar⁵, and/or Ar⁵ and Ar⁶ is/are bonded to each other to form a ring(s). Such a structure is preferred because a π-electron rich heteroaromatic ring such as a carbazole ring can be formed in the general formula (T1) to improve the hole-transport property of the first organic compound.

Next, the arylene group having 6 to 12 carbon atoms that can be applied to the general formulae (T1) and (G0) is described, and specific examples are given. Note that the alkyl group having 1 to 6 carbon atoms, the substituted or unsubstituted cycloalkyl group having 6 to 8 carbon atoms, and the aryl group having 6 to 12 carbon atoms, which are substituents that can be used in the general formulae (T1) and (G0), are similar to those in Embodiment 1. Note that the substituents that can be used in the above general formulae are not limited to the specific examples given below.

### <<Arylene Group Having 6 to 12 Carbon Atoms>>

In this specification and the like, an arylene group having 6 to 12 carbon atoms refers to a divalent group obtained by elimination of one hydrogen atom from one of carbon atoms forming the ring(s) in an aryl group having 6 to 12 carbon atoms. Specific examples of the arylene group having 6 to 12 carbon atoms include a 1,4-phenylene group, a 1,3-phenylene group, and a 4,4′-biphenylene group. In the case where the arylene group having 6 to 12 carbon atoms includes a substituent, specific examples of the substituent include an alkyl group having 1 to 6 carbon atoms.

The above-described first organic compound (including the organic compound represented by the general formula (G1) described in Embodiment 1) has a carrier-transport property. Thus, a light-emitting device can have improved characteristics by using the first organic compound for one or more of the layers (a hole-injection layer, a hole-transport layer, a light-emitting layer, an electron-transport layer, and an electron-injection layer) described later and included in the light-emitting device.

In the case where the above-described first organic compound has a hole-transport property, holes can be smoothly transported in an EL layer 103 with the use of the first organic compound for one or more of a hole-injection layer, a hole-transport layer, and a light-emitting layer described later, whereby the emission efficiency of a light-emitting device 100 can be improved.

In the case where the above-described first organic compound has an electron-transport property, electrons can be smoothly transported in the EL layer with the use of the first organic compound for one or more of the light-emitting layer, an electron-transport layer, and an electron-injection layer described later, whereby the emission efficiency of the light-emitting device can be improved.

The first organic compound may be a bipolar substance (a substance having a high electron-transport property and a high hole-transport property).

The first organic compound preferably has high heat resistance. This can prevent crystallization of the EL layer in the case where the crystallization of the EL layer or the like would be problematic; thus, the light-emitting device can have higher luminance and higher reliability.

Since the first organic compound can have high heat resistance, a high S₁ level, and a high T₁ level at the same time, it is a material effective in the case where the emission wavelength of a light-emitting substance included in a light-emitting layer is short (e.g., blue light emission), in particular. The first organic compound can supply high energy to the light-emitting substance having a short emission wavelength, so that the emission efficiency of the light-emitting device can be increased.

### <<Basic Structure of Light-Emitting Device>>

A basic structure of a light-emitting device is described. FIG. 1A illustrates a light-emitting device including, between a pair of electrodes, an organic compound layer (an EL layer) including a light-emitting layer. Specifically, the EL layer 103 is positioned between the first electrode 101 and the second electrode 102.

FIG. 1B illustrates a light-emitting device that has a stacked-layer structure (tandem structure) in which a plurality of EL layers (two EL layers 103a and 103b in FIG. 1B) are provided between a pair of electrodes and a charge-generation layer 106 is provided between the EL layers. A light-emitting device having a tandem structure enables fabrication of a light-emitting apparatus that has high efficiency without changing the amount of current.

The charge-generation layer 106 has a function of injecting electrons into one of the EL layers 103a and 103b and injecting holes into the other of the EL layers 103a and 103b when a potential difference is caused between the first electrode 101 and the second electrode 102. Thus, when voltage is applied in FIG. 1B such that the potential of the first electrode 101 can be higher than that of the second electrode 102, electrons are injected into the EL layer 103a from the charge-generation layer 106 and holes are injected into the EL layer 103b from the charge-generation layer 106.

Note that in terms of light extraction efficiency, the charge-generation layer 106 preferably has a property of transmitting visible light (specifically, the charge-generation layer 106 preferably has a visible light transmittance of 40% or more). The charge-generation layer 106 functions even if it has lower conductivity than the first electrode 101 or the second electrode 102.

FIG. 1C illustrates a stacked-layer structure of the EL layer 103 in the light-emitting device of one embodiment of the present invention. In this case, the first electrode 101 is regarded as functioning as an anode and the second electrode 102 is regarded as functioning as a cathode. The EL layer 103 has a structure in which the hole-injection layer 111, the hole-transport layer 112, the light-emitting layer 113, the electron-transport layer 114, and the electron-injection layer 115 are stacked in this order over the first electrode 101. Note that the light-emitting layer 113 may have a stacked-layer structure of a plurality of light-emitting layers that emit light of different colors. For example, a light-emitting layer containing a light-emitting substance that emits red light, a light-emitting layer containing a light-emitting substance that emits green light, and a light-emitting layer containing a light-emitting substance that emits blue light may be stacked with or without a layer containing a carrier-transport material therebetween. Alternatively, a light-emitting layer containing a light-emitting substance that emits yellow light and a light-emitting layer containing a light-emitting substance that emits blue light may be used in combination. Note that the stacked-layer structure of the light-emitting layer 113 is not limited to the above. For example, the light-emitting layer 113 may have a stacked-layer structure of a plurality of light-emitting layers that emit light of the same color. For example, a first light-emitting layer containing a light-emitting substance that emits blue light and a second light-emitting layer containing a light-emitting substance that emits blue light may be stacked with or without a layer containing a carrier-transport material therebetween. The structure in which a plurality of light-emitting layers that emit light of the same color are stacked can achieve higher reliability than a single-layer structure in some cases. In the case where a plurality of EL layers are provided as in the tandem structure illustrated in FIG. 1B, the layers in each EL layer are sequentially stacked from the anode side as described above. When the first electrode 101 is the cathode and the second electrode 102 is the anode, the stacking order of the layers in the EL layer 103 is reversed. Specifically, the layer 111 over the first electrode 101 serving as the cathode is an electron-injection layer; the layer 112 is an electron-transport layer; the layer 113 is a light-emitting layer; the layer 114 is a hole-transport layer; and the layer 115 is a hole-injection layer.

The light-emitting layer 113 included in each of the EL layers (103, 103a, and 103b) includes a light-emitting substance and a plurality of substances in an appropriate combination. The plurality of EL layers (103a and 103b) in FIG. 1B may exhibit their respective emission colors. In that case, the light-emitting substances and other substances can differ between the light-emitting layers.

The light-emitting device of one embodiment of the present invention can have a micro optical resonator (microcavity) structure when, for example, the first electrode 101 is a reflective electrode and the second electrode 102 is a transflective electrode in FIG. 1C. Thus, light from the light-emitting layer 113 in the EL layer 103 can be resonated between the electrodes and light emitted through the second electrode 102 can be intensified.

Note that when the first electrode 101 of the light-emitting device is a reflective electrode having a stacked-layer structure of a reflective conductive material and a light-transmitting conductive material (transparent conductive film), optical adjustment can be performed by adjusting the thickness of the transparent conductive film. Specifically, when the wavelength of light obtained from the light-emitting layer 113 is λ, the optical path length between the first electrode 101 and the second electrode 102 (the product of the thickness and the refractive index) is preferably adjusted to be mλ/2 (m is an integer of 1 or more) or close to mλ/2.

To amplify desired light (wavelength: λ) obtained from the light-emitting layer 113, it is preferable to adjust each of the optical path length from the first electrode 101 to a region where the desired light is obtained in the light-emitting layer 113 (light-emitting region) and the optical path length from the second electrode 102 to the region where the desired light is obtained in the light-emitting layer 113 (light-emitting region) to be (2m′+1)λ/4 (m′ is an integer of 1 or more) or close to (2m′+1)λ/4. Here, the light-emitting region means a region where holes and electrons are recombined in the light-emitting layer 113.

By such optical adjustment, the spectrum of specific monochromatic light obtained from the light-emitting layer 113 can be narrowed and light emission with high color purity can be obtained.

In the above case, the optical path length between the first electrode 101 and the second electrode 102 is, to be exact, the total thickness from a reflective region in the first electrode 101 to a reflective region in the second electrode 102. However, it is difficult to precisely determine the reflective regions in the first electrode 101 and the second electrode 102; thus, it is assumed that the above effect can be sufficiently obtained wherever the reflective regions may be set in the first electrode 101 and the second electrode 102. Furthermore, the optical path length between the first electrode 101 and the light-emitting layer that emits the desired light is, to be exact, the optical path length between the reflective region in the first electrode 101 and the light-emitting region in the light-emitting layer that emits the desired light. However, it is difficult to precisely determine the reflective region in the first electrode 101 and the light-emitting region in the light-emitting layer that emits the desired light; thus, it is assumed that the above effect can be sufficiently obtained wherever the reflective region and the light-emitting region may be set in the first electrode 101 and the light-emitting layer that emits the desired light, respectively.

FIG. 1D illustrates a modification example of the stacked-layer structure illustrated in FIG. 1C. Also in this case, the first electrode 101 is regarded as functioning as an anode, and the second electrode 102 is regarded as functioning as a cathode. In this modification example, the hole-transport layer 112 and the electron-transport layer 114 each have a stacked-layer structure of two layers. In other words, the EL layer 103 has a structure in which a hole-injection layer 111, a first hole-transport layer 112-1, a second hole-transport layer 112-2, a light-emitting layer 113, a second electron-transport layer 114-2, a first electron-transport layer 114-1, and an electron-injection layer 115 are stacked in this order over the first electrode 101. Note that the light-emitting layer 113 is positioned between the first electrode 101 and the second electrode 102. The first hole-transport layer 112-1 is positioned between the first electrode 101 and the light-emitting layer 113. The first electron-transport layer 114-1 is positioned between the light-emitting layer 113 and the second electrode 102. The hole-injection layer 111 is positioned between the first electrode 101 and the hole-transport layer 112. The electron-injection layer 115 is positioned between the electron-transport layer 114 and the second electrode 102. The second hole-transport layer 112-2 is positioned between the first hole-transport layer 112-1 and the light-emitting layer 113. In other words, the second electron-transport layer 114-2 is positioned between the light-emitting layer 113 and the first electron-transport layer 114-1. In the case where the EL layer 103 has such a stacked-layer structure, one or more of the hole-injection layer 111, the hole-transport layer 112, and the light-emitting layer 113 preferably contains the first organic compound.

The second hole-transport layer 112-2 is provided to prevent passing of electrons from the light-emitting layer 113 to the first electrode 101 side, for example. Thus, the second hole-transport layer 112-2 can also be referred to as an electron-blocking layer. The second electron-transport layer 114-2 is provided to prevent passing of holes from the light-emitting layer 113 to the second electrode 102 side, for example. Thus, the second electron-transport layer 114-2 can also be referred to as a hole-blocking layer.

The light-emitting device illustrated in FIG. 1E is a light-emitting device having a tandem structure. Owing to a microcavity structure, light (monochromatic light) with different wavelengths from the EL layers (103a and 103b) can be extracted. Thus, separate coloring for obtaining a plurality of emission colors (e.g., R, G, and B) is not necessary. Therefore, high resolution can be easily achieved. A combination with coloring layers (color filters) is also possible. Furthermore, the emission intensity of light with a specific wavelength in the front direction can be increased, whereby power consumption can be reduced.

The light-emitting device illustrated in FIG. 1F is an example of the light-emitting device having the tandem structure illustrated in FIG. 1B, and includes three EL layers (103a, 103b, and 103c) stacked with charge-generation layers (106a and 106b) positioned therebetween, as illustrated in FIG. 1F. The three EL layers (103a, 103b, and 103c) include respective light-emitting layers (113a, 113b, and 113c), and the emission colors of the light-emitting layers can be selected freely. For example, the light-emitting layer 113a can emit blue light, the light-emitting layer 113b can emit red light, green light, or yellow light, and the light-emitting layer 113c can emit blue light, or the light-emitting layer 113a can emit red light, the light-emitting layer 113b can emit blue light, green light, or yellow light, and the light-emitting layer 113c can emit red light.

FIG. 2 is a schematic view of a light-emitting apparatus including a light-emitting device 100A and a light-emitting device 100B of one embodiment of the present invention. In each of the light-emitting devices 100A and 100B, part of the organic compound layer (EL layer) is formed through processing by a lithography method.

The light-emitting device 100A is located over an insulating layer 175 and includes a first electrode 101A that includes an anode, the second electrode 102 that includes a cathode, and an EL layer 103A. The EL layer 103A is located between the first electrode 101A and the second electrode 102. The EL layer 103A includes at least a light-emitting layer 113A. The light-emitting layer 113A includes at least a light-emitting substance. In the example illustrated in FIG. 2, the EL layer 103A includes a hole-injection layer 111A, a hole-transport layer 112A, an electron-transport layer 108A, and an electron-injection layer 109 in addition to the light-emitting layer 113A. In other words, the EL layer 103A illustrated in FIG. 2 includes the hole-injection layer 111A, the hole-transport layer 112A, the light-emitting layer 113A, the electron-transport layer 108A, and the electron-injection layer 109 in this order.

The light-emitting device 100B is located over the insulating layer 175 and includes a first electrode 101B that includes an anode, the second electrode 102 that includes a cathode, and an EL layer 103B. The EL layer 103B is located between the first electrode 101B and the second electrode 102. The EL layer 103B includes at least a light-emitting layer 113B. The light-emitting layer 113B includes at least a light-emitting substance. In the example illustrated in FIG. 2, the EL layer 103B includes a hole-injection layer 111B, a hole-transport layer 112B, an electron-transport layer 108B, and an electron-injection layer 109 in addition to the light-emitting layer 113B. In other words, the EL layer 103B illustrated in FIG. 2 includes the hole-injection layer 111B, the hole-transport layer 112B, the light-emitting layer 113B, the electron-transport layer 108B, and the electron-injection layer 109 in this order.

The use of the first organic compound for one or both of the EL layer 103A of the light-emitting device 100A and the EL layer 103B of the light-emitting device 100B achieves an effect similar to that of the use of the first organic compound for the EL layer 103 of the light-emitting device 100 illustrated in FIG. 1A.

In the EL layer 103A of the light-emitting device 100A, layers other than the electron-injection layer 109 are formed through processing by a lithography method. Thus, the layers other than the electron-injection layer 109 in the EL layer 103A are separate from those in the organic compound layer of the adjacent light-emitting device 100B. End portions (contours) of the layers other than the electron-injection layer 109 in the EL layer 103A are aligned or substantially aligned with each other in a direction perpendicular to a substrate. Thus, end portions of the hole-injection layer 111A, the hole-transport layer 112A, the light-emitting layer 113A, and the electron-transport layer 108A are separate from the hole-injection layer 111B, the hole-transport layer 112B, the light-emitting layer 113B, and the electron-transport layer 108B. The end portions (outlines) of the hole-injection layer 111A, the hole-transport layer 112A, the light-emitting layer 113A, and the electron-transport layer 108A are aligned or substantially aligned with each other in the direction perpendicular to the substrate.

In the EL layer 103B of the light-emitting device 100B, layers other than the electron-injection layer 109 are formed through processing by a lithography method. Thus, the layers other than the electron-injection layer 109 in the EL layer 103B are separate from those in the organic compound layer of the adjacent light-emitting device 100A. End portions (contours) of the layers other than the electron-injection layer 109 in the EL layer 103B are aligned or substantially aligned with each other in a direction perpendicular to a substrate. Thus, end portions of the hole-injection layer 111B, the hole-transport layer 112B, the light-emitting layer 113B, and the electron-transport layer 108B are separate from the hole-injection layer 111A, the hole-transport layer 112A, the light-emitting layer 113A, and the electron-transport layer 108A. The end portions (outlines) of the hole-injection layer 111A, the hole-transport layer 112A, the light-emitting layer 113A, and the electron-transport layer 108A are aligned or substantially aligned with each other in the direction perpendicular to the substrate.

The electron-injection layer 109 and the second electrode 102 are preferably formed after the layers of the EL layer 103A other than the electron-injection layer 109 and the layers of the EL layer 103B other than the electron-injection layer 109 are formed through processing by a lithography method. In other words, the electron-injection layer 109 and the second electrode 102 are each preferably a continuous layer shared by the light-emitting devices 100A and 100B.

The electron-injection layer 109 is preferably formed using a donor substance (also referred to as an electron donor), in which case the driving voltages of the light-emitting devices can be reduced. Typical examples of the donor substance include alkali metals such as lithium (Li), which have a low work function, and compounds of the alkali metals.

In the case where processing by a lithography method is performed in a state where an electron-injection layer including such a donor substance serves as an uppermost surface (interface) of an EL layer, the influence of oxygen or water in the air and a chemical solution or water used during the process sometimes causes a light-emitting device to have a significantly increased driving voltage or greatly reduced current efficiency. However, in one embodiment of the present invention, since the electron-injection layer 109 is formed after the layers other than the electron-injection layer 109 are formed through processing by a lithography method, using a donor substance for the electron-injection layer 109 does not cause deterioration of the characteristics of the light-emitting devices.

In the case where the EL layers are formed through processing by a lithography method, a distance d between the layers of the EL layer 103a other than the electron-injection layer 109 and the layers of the EL layer 103b other than the electron-injection layer 109 can be shorter than the distance d in the case of employing mask vapor deposition. Specifically, the distance d can be reduced to less than 10 μm, less than or equal to 8 μm, less than or equal to 5 μm, less than or equal to 3 μm, less than or equal to 2 μm, less than or equal to 1.5 μm, less than or equal to 1 μm, or less than or equal to 0.5 μm. Using a light exposure apparatus for LSI can further shorten the distance d to less than or equal to 500 nm, less than or equal to 200 nm, less than or equal to 100 nm, or even less than or equal to 50 nm, for example, in a process over a Si wafer.

The partition 128 is preferably provided in the gap between the layers of the EL layer 103a other than the electron-injection layer 109 and the layers of the EL layer 103b other than the electron-injection layer 109. The layers of the EL layer 103A other than the electron-injection layer 109 can be separated from the layers of the EL layer 103B other than the electron-injection layer 109. In this structure, the partition 128 can be regarded as being provided in a region surrounded by the insulating layer 175, the end portions of the layers of the EL layer 103A other than the electron-injection layer 109, the electron-injection layer 109, and the end portions of the layers of the EL layer 103B other than the electron-injection layer 109. It can also be said that, in that case, there is a region where the partition 128 is in contact with the electron-injection layer 109 or the second electrode 102. Although the partition 128 in FIG. 2 is in contact with the end portions of the layers of the EL layers 103A and 103B other than the electron-injection layer 109, the structure is not limited thereto. For example, a structure may be employed in which an insulating layer is provided at each of the end portions of the layers of the EL layers 103A and 103B other than the electron-injection layer 109 and the partition 128 is positioned at the end portions of the layers of the EL layers 103A and 103B other than the electron-injection layer 109 with the insulating layers therebetween.

As in the case of the light-emitting devices 100A and 100B, in processing of the EL layers by a lithography method, surfaces to be processed of the layers of the EL layers other than the electron-injection layer 109 are exposed to oxygen or water in the air, a chemical solution or water used during the processing, heat treatment, and the like. This causes a problem such as crystallization of the EL layers, which may decrease the reliability and luminance of the light-emitting devices. This tendency is observed when the heat resistance of the organic compound used for the light-emitting devices is low; the first organic compound with a high T_{g} is therefore preferably used for the EL layers in the case where the processing is performed by a lithography method. The use of the first organic compound with a high T_{g} can prevent thickness change and film quality change such as crystallization of the EL layers which is attributed to heating, heat generation, or the like, whereby the luminance and reliability of the light-emitting devices 100A and 100B can be increased.

The T_{g} of the first organic compound is preferably higher than or equal to 100° C., further preferably higher than or equal to 110° C., higher than or equal to 120° C., higher than or equal to 130° C., or higher than or equal to 140° C. In that case, even in the case where the surfaces of the EL layers to be processed are exposed to oxygen or water in the air, change in film quality is inhibited. This increases the reliability of the light-emitting device 100.

In the light-emitting device of one embodiment of the present invention, at least one of the first electrode 101 and the second electrode 102 is a light-transmitting electrode (e.g., a transparent electrode or a transflective electrode). In the case where the light-transmitting electrode is a transparent electrode, the transparent electrode has a visible light transmittance higher than or equal to 40%. In the case where the light-transmitting electrode is a transflective electrode, the transflective electrode has a visible light reflectance higher than or equal to 20% and lower than or equal to 80%, preferably higher than or equal to 40% and lower than or equal to 70%. These electrodes preferably have a resistivity of 1×10⁻² Ωcm or less.

When one of the first electrode 101 and the second electrode 102 is a reflective electrode in the light-emitting device of one embodiment of the present invention, the visible light reflectance of the reflective electrode is higher than or equal to 40% and lower than or equal to 100%, preferably higher than or equal to 70% and lower than or equal to 100%. This electrode preferably has a resistivity of 1×10⁻² Ωcm or less.

### <<Specific Structure of Light-Emitting Device>>

Next, a specific structure of the light-emitting device of one embodiment of the present invention will be described. Here, the description is made using FIG. 1E illustrating the tandem structure. Note that the structure of the EL layer applies also to the structure of the light-emitting devices having a single structure in FIGS. 1A and 1C. When the light-emitting device in FIG. 1E has a microcavity structure, the first electrode 101 is formed as a reflective electrode and the second electrode 102 is formed as a transflective electrode. Thus, a single-layer structure or a stacked-layer structure can be formed using one or more kinds of desired electrode materials. Note that the second electrode 102 is formed after formation of the EL layer 103b, with the use of a material selected as appropriate.

### <First Electrode and Second Electrode>

As materials for the first electrode 101 and the second electrode 102, any of the following materials can be used in an appropriate combination as long as the above functions of the electrodes can be fulfilled. For example, a metal, an alloy, an electrically conductive compound, a mixture of these, and the like can be used as appropriate. Specifically, an In—Sn oxide (also referred to as ITO), an In—Si—Sn oxide (also referred to as ITSO), an In—Zn oxide, or an In—W—Zn oxide can be used. In addition, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals. It is also possible to use a Group 1 element or a Group 2 element in the periodic table that is not described above (e.g., lithium (Li), cesium (Cs), calcium (Ca), or strontium (Sr)), a rare earth metal such as europium (Eu) or ytterbium (Yb), an alloy containing an appropriate combination of any of these elements, graphene, or the like.

In the light-emitting device in FIG. 1E, when the first electrode 101 is the anode, a hole-injection layer 111a and a hole-transport layer 112a of the EL layer 103a are sequentially stacked over the first electrode 101 by a vacuum evaporation method. After the EL layer 103a and the charge-generation layer 106 are formed, a hole-injection layer 111b and a hole-transport layer 112b of the EL layer 103b are sequentially stacked over the charge-generation layer 106 in a similar manner.

The light-emitting device of one embodiment illustrated in FIG. 1E can have a micro optical resonator (microcavity) structure when the first electrode 101 is a reflective electrode and the second electrode 102 is a transflective electrode. Thus, light from the light-emitting layer 113 in the EL layer 103 can be resonated between the electrodes and light emitted through the second electrode 102 can be intensified.

Note that when the first electrode 101 of the light-emitting device is a reflective electrode having a stacked-layer structure of a reflective conductive material and a light-transmitting conductive material (transparent conductive film), optical adjustment can be performed by adjusting the thickness of the transparent conductive film. Specifically, when the wavelength of light obtained from the light-emitting layer 113 is λ, the optical path length between the first electrode 101 and the second electrode 102 (the product of the thickness and the refractive index) is preferably adjusted to be mλ/2 (m is an integer of 1 or more) or close to mλ/2.

To amplify desired light (wavelength: λ) obtained from the light-emitting layer 113, it is preferable to adjust each of the optical path length from the first electrode 101 to a region where the desired light is obtained in the light-emitting layer 113 (light-emitting region) and the optical path length from the second electrode 102 to the region where the desired light is obtained in the light-emitting layer 113 (light-emitting region) to be (2m′+1)λ/4 (m′ is an integer of 1 or more) or close to (2m′+1)λ/4. Here, the light-emitting region means a region where holes and electrons are recombined in the light-emitting layer 113.

By such optical adjustment, the spectrum of specific monochromatic light obtained from the light-emitting layer 113 can be narrowed and light emission with high color purity can be obtained.

In the above case, the optical path length between the first electrode 101 and the second electrode 102 is, to be exact, the total thickness from a reflective region in the first electrode 101 to a reflective region in the second electrode 102. However, it is difficult to precisely determine the reflective regions in the first electrode 101 and the second electrode 102; thus, it is assumed that the above effect can be sufficiently obtained wherever the reflective regions may be set in the first electrode 101 and the second electrode 102. Furthermore, the optical path length between the first electrode 101 and the light-emitting layer that emits the desired light is, to be exact, the optical path length between the reflective region in the first electrode 101 and the light-emitting region in the light-emitting layer that emits the desired light. However, it is difficult to precisely determine the reflective region in the first electrode 101 and the light-emitting region in the light-emitting layer that emits the desired light; thus, it is assumed that the above effect can be sufficiently obtained wherever the reflective region and the light-emitting region may be set in the first electrode 101 and the light-emitting layer that emits the desired light, respectively.

In the light-emitting device of one embodiment of the present invention, at least one of the first electrode 101 and the second electrode 102 is a light-transmitting electrode (e.g., a transparent electrode or a transflective electrode). In the case where the light-transmitting electrode is a transparent electrode, the transparent electrode has a visible light transmittance higher than or equal to 40%. In the case where the light-transmitting electrode is a transflective electrode, the transflective electrode has a visible light reflectance higher than or equal to 20% and lower than or equal to 80%, preferably higher than or equal to 40% and lower than or equal to 70%. These electrodes preferably have a resistivity of 1×10⁻² Ωcm or less.

When one of the first electrode 101 and the second electrode 102 is a reflective electrode in the light-emitting device of one embodiment of the present invention, the visible light reflectance of the reflective electrode is higher than or equal to 40% and lower than or equal to 100%, preferably higher than or equal to 70% and lower than or equal to 100%. This electrode preferably has a resistivity of 1×10⁻² 2 Ωcm or less.

### <Hole-Injection Layer>

The hole-injection layers (111, 111a, and 111b) inject holes from the first electrode 101 serving as the anode and the charge-generation layers (106, 106a, and 106b) to the EL layers (103, 103a, and 103b) and contain an organic acceptor material or a material having a high hole-injection property.

The organic acceptor material allows holes to be generated in another organic compound whose HOMO level is close to the LUMO level of the organic acceptor material when charge separation is caused between the organic acceptor material and the organic compound. Thus, as the organic acceptor material, a compound having an electron-withdrawing group (e.g., a halogen group or a cyano group), such as a quinodimethane derivative, a chloranil derivative, and a hexaazatriphenylene derivative, can be used. Examples of the organic acceptor material include 7,7,8,8-tetracyano-2,3,5,6-tetrafluoroquinodimethane (abbreviation: F₄-TCNQ), 3,6-difluoro-2,5,7,7,8,8-hexacyanoquinodimethane, chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F6-TCNNQ), and 2-(7-dicyanomethylen-1,3,4,5,6,8,9,10-octafluoro-7H-pyren-2-ylidene)malononitrile. Note that among organic acceptor materials, a compound in which electron-withdrawing groups are bonded to fused aromatic rings each having a plurality of heteroatoms, such as HAT-CN, is particularly preferred because it has a high acceptor property and stable film quality against heat. Besides, a [3]radialene derivative having an electron-withdrawing group (particularly a cyano group or a halogen group such as a fluoro group), which has a very high electron-accepting property, is preferred; specific examples include α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile].

As the material having a high hole-injection property, an oxide of a metal belonging to Group 4 to Group 8 in the periodic table (e.g., a transition metal oxide such as molybdenum oxide, vanadium oxide, ruthenium oxide, tungsten oxide, or manganese oxide) can be used. Specific examples include molybdenum oxide, vanadium oxide, niobium oxide, tantalum oxide, chromium oxide, tungsten oxide, manganese oxide, and rhenium oxide. Among the above oxides, molybdenum oxide is preferable because it is stable in the air, has a low hygroscopic property, and is easily handled. Other examples include a perylenetetracarboxylic acid derivative such as diquinoxalino[2,3-a:2′,3′-c]phenazine (abbreviation: HATNA), 2,3,8,9,14,15-hexafluorodiquinoxalino[2,3-a:2′,3′-c]phenazine (abbreviation: HATNA-F6), 3,4,9,10-perylenetetracarboxylic diimide (abbreviation: PTCDI), or 3,4,9,10-perylenetetracarboxyl-bis-benzimidazole (abbreviation: PTCBI); (C₆₀—Iₕ) [5,6]fullerene (abbreviation: C₆₀); (C70-D5h) [5,6]fullerene (abbreviation: C₇₀); an organic compound such as phthalocyanine (abbreviation: H₂Pc); and a metal phthalocyanine containing copper, zinc, cobalt, iron, chromium, nickel, or the like or a derivative thereof, such as copper phthalocyanine (abbreviation: CuPc), zinc phthalocyanine (abbreviation: ZnPc), cobalt phthalocyanine (abbreviation: CoPc), iron phthalocyanine (abbreviation: FePc), tin phthalocyanine (abbreviation: SnPc), tin oxide phthalocyanine (abbreviation: SnOPc), titanium oxide phthalocyanine (abbreviation: TiOPc), or vanadium oxide phthalocyanine (abbreviation: VOPc). A phthalocyanine-based metal complex such as CuPc or ZnPc and 2,3,8,9,14,15-hexafluorodiquinoxalino[2,3-a:2′,3′-c]phenazine are especially preferable. Among these materials, CuPc and ZnPc are preferable because they are inexpensive and have favorable characteristics. Using ZnPc, which has a low diffusion coefficient with respect to silicon, reduces the probability that metal diffusion to a semiconductor adversely affects the semiconductor characteristics; accordingly, ZnPc is particularly suitable for a display device manufactured using a silicon semiconductor.

Other examples are aromatic amine compounds, which are low-molecular compounds, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), N,N′-bis[4-bis(3-methylphenyl)aminophenyl]-N,N′-diphenyl-4,4′-diaminobiphenyl (abbreviation: DNTPD), 1,3,5-tris[N-(4-diphenylaminophenyl)-N-phenylamino]benzene (abbreviation: DPA3B), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), and 3-[N-(1-naphthyl)-N-(9-phenylcarbazol-3-yl)amino]-9-phenylcarbazole (abbreviation: PCzPCN1).

Other examples are high-molecular compounds (e.g., oligomers, dendrimers, and polymers) such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide] (abbreviation: PTPDMA), and poly[N,N′-bis(4-butylphenyl)-N,N′-bis(phenyl)benzidine] (abbreviation: Poly-TPD). Alternatively, it is possible to use a high-molecular compound to which acid is added, such as poly(3,4-ethylenedioxythiophene)/poly(styrenesulfonic acid) (abbreviation: PEDOT/PSS) or polyaniline/poly(styrenesulfonic acid) (abbreviation: PAni/PSS), for example.

As the material having a high hole-injection property, a mixed material containing a hole-transport material and the above-described organic acceptor material (electron-accepting material) can be used. In that case, the organic acceptor material extracts electrons from the hole-transport material, so that holes are generated in the hole-injection layer 111 and the holes are injected into the light-emitting layer 113 through the hole-transport layer 112. Note that the hole-injection layer 111 may be formed to have a single-layer structure using a mixed material containing a hole-transport material and an organic acceptor material (electron-accepting material), or a stacked-layer structure of a layer containing a hole-transport material and a layer containing an organic acceptor material (electron-accepting material).

The hole-transport material preferably has a hole mobility higher than or equal to 1×10⁻⁶ cm²/Vs in the case where the square root of the electric field strength [V/cm] is 600. Note that other substances can also be used as long as the substances have hole-transport properties higher than electron-transport properties.

As the hole-transport material, materials having a high hole-transport property, such as a compound having a π-electron rich heteroaromatic ring (e.g., a carbazole derivative, a furan derivative, and a thiophene derivative) and an aromatic amine (an organic compound having an aromatic amine skeleton), are preferable. In the case where the first organic compound has a hole-transport property, it can be used as a hole-transport material.

Examples of the carbazole derivative (an organic compound having a carbazole ring) include a bicarbazole derivative (e.g., a 3,3′-bicarbazole derivative) and an aromatic amine having a carbazolyl group.

Specific examples of the above bicarbazole derivative (e.g., a 3,3′-bicarbazole derivative) include 9,9′-diphenyl-9H,9′H-3,3′-bicarbazole (abbreviation: PCCP), 9,9′-bis(biphenyl-4-yl)-3,3′-bi-9H-carbazole (abbreviation: BisBPCz), 9,9′-bis(biphenyl-3-yl)-3,3′-bi-9H-carbazole (abbreviation: BismBPCz), 9-(biphenyl-3-yl)-9′-(biphenyl-4-yl)-9H,9′H-3,3′-bicarbazole (abbreviation: mBPCCBP), and 9-(2-naphthyl)-9′-phenyl-9H,9′H-3,3′-bicarbazole (abbreviation: βNCCP).

Specific examples of the aromatic amine having a carbazolyl group include 4-phenyl-4′-(9-phenyl-9H-carbazol-3-yl)triphenylamine (abbreviation: PCBA1BP), N-(biphenyl-4-yl)-N-(9,9-dimethyl-9H-fluoren-2-yl)-9-phenyl-9H-carbazol-3-amine (abbreviation: PCBiF), N-(biphenyl-4-yl)-N-[4-(9-phenyl-9H-carbazol-3-yl)phenyl]-9,9-dimethyl-9H-fluoren-2-amine (abbreviation: PCBBiF), N-[4-(9-phenyl-9H-carbazol-3-yl)phenyl]bis(9,9-dimethyl-9H-fluoren-2-yl)amine (abbreviation: PCBFF), N-(1,1′-biphenyl-4-yl)-N-[4-(9-phenyl-9H-carbazol-3-yl)phenyl]-9,9-dimethyl-9H-fluoren-4-amine, N-[4-(9-phenyl-9H-carbazol-3-yl)phenyl]-(9,9-dimethyl-9H-fluoren-2-yl)-9,9-dimethyl-9H-fluoren-4-amine, N-(1,1′-biphenyl-4-yl)-N-[4-(9-phenyl-9H-carbazol-3-yl)phenyl]-9,9-diphenyl-9H-fluoren-2-amine, N-(1,1′-biphenyl-4-yl)-N-[4-(9-phenyl-9H-carbazol-3-yl)phenyl]-9,9-diphenyl-9H-fluoren-4-amine, N-(1,1′-biphenyl-4-yl)-N-[4-(9-phenyl-9H-carbazol-3-yl)phenyl]-9,9′-spirobi(9H-fluoren)-2-amine, N-(1,1′-biphenyl-4-yl)-N-[4-(9-phenyl-9H-carbazol-3-yl)phenyl]-9,9′-spirobi(9H-fluoren)-4-amine, N-[4-(9-phenyl-9H-carbazol-3-yl)phenyl]-N-(1,1′: 3′,1″-terphenyl-4-yl)9,9-dimethyl-9H-fluoren-2-amine, N-[4-(9-phenyl-9H-carbazol-3-yl)phenyl]-N-(1,1′: 4′,1″-terphenyl-4-yl)-9,9-dimethyl-9H-fluoren-2-amine, N-[4-(9-phenyl-9H-carbazol-3-yl)phenyl]-N-(1,1′: 3′,1″-terphenyl-4-yl)-9,9-dimethyl-9H-fluoren-4-amine, N-[4-(9-phenyl-9H-carbazol-3-yl)phenyl]-N-(1,1′: 4′,1″-terphenyl-4-yl)-9,9-dimethyl-9H-fluoren-4-amine, 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 4-phenyldiphenyl-(9-phenyl-9H-carbazol-3-yl)amine (abbreviation: PCA1BP), N,N′-bis(9-phenylcarbazol-3-yl)-N,N′-diphenylbenzene-1,3-diamine (abbreviation: PCA2B), N,N′,N″-triphenyl-N,N′,N″-tris(9-phenylcarbazol-3-yl)benzene-1,3,5-triamine (abbreviation: PCA3B), 9,9-dimethyl-N-phenyl-N-[4-(9-phenyl-9H-carbazol-3-yl)phenyl]fluoren-2-amine (abbreviation: PCBAF), N-phenyl-N-[4-(9-phenyl-9H-carbazol-3-yl)phenyl]-9,9′-spirobi[9H-fluoren]-2-amine (abbreviation: PCBASF), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), 3-[N-(4-diphenylaminophenyl)-N-phenylamino]-9-phenylcarbazole (abbreviation: PCzDPA1), 3,6-bis[N-(4-diphenylaminophenyl)-N-phenylamino]-9-phenylcarbazole (abbreviation: PCzDPA2), 3,6-bis[N-(4-diphenylaminophenyl)-N-(1-naphthyl)amino]-9-phenylcarbazole (abbreviation: PCzTPN2), N-(9,9-spirobi[9H-fluoren]-2-yl)-N,9-diphenylcarbazol-3-amine (abbreviation: PCASF), N-(4-biphenyl)-4-(carbazol-9-yl)phenylaniline (abbreviation: YGA1BP), N,N′-bis[4-(carbazol-9-yl)phenyl]-N,N′-diphenyl-9,9-dimethylfluorene-2,7-diamine (abbreviation: YGA2F), and 4,4′,4″-tris(carbazol-9-yl)triphenylamine (abbreviation: TCTA).

Other examples of the carbazole derivative include 9-[4-(9-phenyl-9H-carbazol-3-yl)phenyl]phenanthrene (abbreviation: PCPPn), 3-[4-(1-naphthyl)phenyl]-9-phenyl-9H-carbazole (abbreviation: PCPN), 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 1,3,5-tris[4-(N-carbazolyl)phenyl]benzene (abbreviation: TCPB), and 9-[4-(10-phenyl-9-anthracenyl)phenyl]-9H-carbazole (abbreviation: CzPA).

Specific examples of the furan derivative (an organic compound having a furan ring) include 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II).

Specific examples of the thiophene derivative (an organic compound having a thiophene ring) include organic compounds having a thiophene ring, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV).

Specific examples of the aromatic amine include 4,4′-bis[N-(1-naphthyl)-N-phenylamino]biphenyl (abbreviation: NPB or α-NPD), N,N′-diphenyl-N,N′-bis(3-methylphenyl)-4,4′-diaminobiphenyl (abbreviation: TPD), N,N′-bis(9,9′-spirobi[9H-fluoren]-2-yl)-N,N′-diphenyl-4-4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), N-(9,9-dimethyl-9H-fluoren-2-yl)-N-{9,9-dimethyl-2-[N′-phenyl-N′-(9,9-dimethyl-9H-fluoren-2-yl)amino]-9H-fluoren-7-yl}phenylamine (abbreviation: DFLADFL), N-(9,9-dimethyl-2-diphenylamino-9H-fluoren-7-yl)diphenylamine (abbreviation: DPNF), N-(9,9-spirobi[9H-fluoren]-2-yl)-N,N′,N′-triphenyl-1,4-phenyldiamine (abbreviation: DPASF), N,N′-diphenyl-N,N′-bis(4-diphenylaminophenyl)spirobi[9H-fluorene]-2,7-diamine (abbreviation: DPA2SF), 4,4′,4″-tris[N-(1-naphthyl)-N-phenylamino]triphenylamine (abbreviation: 1′-TNATA), 4,4′,4″-tris(N,N-diphenylamino)triphenylamine (abbreviation: TDATA), 4,4′,4″-tris[N-(3-methylphenyl)-N-phenylamino]triphenylamine (abbreviation: m-MTDATA), N,N′-di(p-tolyl)-N,N′-diphenyl-p-phenylenediamine (abbreviation: DTDPPA), 4,4′-bis[N-(4-diphenylaminophenyl)-N-phenylamino]biphenyl (abbreviation: DPAB), DNTPD, 1,3,5-tris[N-(4-diphenylaminophenyl)-N-phenylamino]benzene (abbreviation: DPA3B), N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-(6;1′-binaphthyl-2-yl)triphenylamine (abbreviation: BBAαNβNB), 4,4′-diphenyl-4″-(7;1′-binaphthyl-2-yl)triphenylamine (abbreviation: BBAαNβNB-03), 4,4′-diphenyl-4″-(7-phenyl)naphthyl-2-yltriphenylamine (abbreviation: BBAPβNB-03), 4,4′-diphenyl-4″-(6;2′-binaphthyl-2-yl)triphenylamine (abbreviation: BBA(βN2)B), 4,4′-diphenyl-4″-(7;2′-binaphthyl-2-yl)triphenylamine (abbreviation: BBA(βN2)B-03), 4,4′-diphenyl-4″-(4;2′-binaphthyl-1-yl)triphenylamine (abbreviation: BBAβNαNB), 4,4′-diphenyl-4″-(5;2′-binaphthyl-1-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)-triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiβNB), N-[4-(9-phenyl-9H-carbazol-3-yl)phenyl]-N-[4-(1-naphthyl)phenyl]-9,9′-spirobi[9H-fluoren]-2-amine (abbreviation: PCBNBSF), N,N-bis(biphenyl-4-yl)-9,9′-spirobi[9H-fluoren]-2-amine (abbreviation: BBASF), N,N-bis(biphenyl-4-yl)-9,9′-spirobi[9H-fluoren]-4-amine (abbreviation: BBASF(4)), N-(biphenyl-2-yl)-N-(9,9-dimethyl-9H-fluoren-2-yl)-9,9′-spirobi[9H-fluoren]-4-amine (abbreviation: oFBiSF), N-(biphenyl-4-yl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-[4-(9-phenylfluoren-9-yl)phenyl]triphenylamine (abbreviation: BPAFLBi), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, and N,N-bis(9,9-dimethyl-9H-fluoren-2-yl)-9,9′-spirobi-9H-fluoren-1-amine.

Other examples of the hole-transport material include high-molecular compounds (e.g., oligomers, dendrimers, and polymers) such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide](abbreviation: PTPDMA), and poly[N,N′-bis(4-butylphenyl)-N,N′-bis(phenyl)benzidine](abbreviation: Poly-TPD). Alternatively, it is possible to use a high-molecular compound to which acid is added, such as poly(3,4-ethylenedioxythiophene)/poly(styrenesulfonic acid) (abbreviation: PEDOT/PSS) or polyaniline/poly(styrenesulfonic acid) (abbreviation: PAni/PSS), for example.

Note that the hole-transport material is not limited to the above examples, and any of a variety of known materials may be used alone or in combination as the hole-transport material.

The hole-injection layers (111, 111a, and 111b) can be formed by any of known film formation methods, and for example, a vacuum evaporation method can be employed.

### <Hole-Transport Layer>

The hole-transport layers (112, 112a, and 112b) transport the holes, which are injected from the first electrodes 101 by the hole-injection layers (111, 111a, and 111b), to the light-emitting layers (113, 113a, and 113b). Note that the hole-transport layers (112, 112a, and 112b) each contain a hole-transport material. Thus, the hole-transport layers (112, 112a, and 112b) can be formed using hole-transport materials that can be used for the hole-injection layers (111, 111a, and 111b).

Note that in the light-emitting device of one embodiment of the present invention, the organic compound used for the hole-transport layers (112, 112a, and 112b) can also be used for the light-emitting layers 113, 113a, and 113b). The use of the same organic compound for the hole-transport layers (112, 112a, and 112b) and the light-emitting layers (113, 113a, and 113b) is preferable, in which case holes can be efficiently transported from the hole-transport layers (112, 112a, and 112b) to the light-emitting layers (113, 113a, and 113b).

### <Light-Emitting Layer>

The light-emitting layers (113, 113a, and 113b) contain a light-emitting substance. Note that as a light-emitting substance that can be used in the light-emitting layers (113, 113a, and 113b), a substance whose emission color is blue, violet, bluish violet, green, yellowish green, yellow, orange, red, or the like can be used as appropriate. When a plurality of light-emitting layers are provided, the use of different light-emitting substances for the light-emitting layers enables a structure that exhibits different emission colors (e.g., white light emission obtained by a combination of complementary emission colors). When a plurality of light-emitting layers are provided, the light-emitting layers can exhibit the same color. The structure in which a plurality of light-emitting layers that emit light of the same color are stacked can sometimes achieve higher reliability than a single-layer structure. Furthermore, one light-emitting layer may have a stacked-layer structure including different light-emitting substances.

The light-emitting layers (113, 113a, and 113b) may each contain one or more kinds of organic compounds (e.g., a host material) in addition to a light-emitting substance (a guest material).

In the case where a plurality of host materials are used in the light-emitting layers (113, 113a, and 113b), a second host material that is additionally used is preferably a substance having a larger energy gap than those of a known guest material and a first host material. Preferably, the lowest singlet excitation energy level (S₁ level) of the second host material is higher than that of the first host material, and the lowest triplet excitation energy level (T₁ level) of the second host material is higher than that of the guest material. Preferably, the lowest triplet excitation energy level (T₁ level) of the second host material is higher than that of the first host material. With such a structure, an exciplex can be formed by the two kinds of host materials. To form an exciplex efficiently, it is particularly preferable to combine a compound that easily accepts holes (hole-transport material) and a compound that easily accepts electrons (electron-transport material). With the above structure, high efficiency, low voltage, and a long lifetime can be achieved at the same time.

As an organic compound used as the host material (including the first host material and the second host material), organic compounds such as the hole-transport materials usable for the hole-transport layers (112, 112a, and 112b) described above and electron-transport materials usable for electron-transport layers (114, 114a, and 114b) described later can be used as long as they satisfy requirements for the host material used in the light-emitting layer. Another example is an exciplex formed by two or more kinds of organic compounds (the first host material and the second host material). An exciplex whose excited state is formed by two or more kinds of organic compounds has an extremely small difference between the S₁ level and the T₁ level and functions as a thermally activated delayed fluorescent (TADF) material capable of converting triplet excitation energy into singlet excitation energy. In an example of a preferable combination of two or more kinds of organic compounds forming an exciplex, one compound of the two or more kinds of organic compounds has a π-electron deficient heteroaromatic ring and the other compound has a π-electron rich heteroaromatic ring. A phosphorescent substance such as an iridium-, rhodium-, or platinum-based organometallic complex or a metal complex may be used as one compound of the combination for forming an exciplex. The first organic compound can be used as the host material.

Note that in the case where the first organic compound having a hole-transport property is used as the first host material, an organic compound having a π-electron deficient heteroaromatic ring (second organic compound) is preferably used as the second host material in combination to form an exciplex.

There is no particular limitation on the light-emitting substances that can be used for the light-emitting layers (113, 113a, and 113b), and a light-emitting substance that converts singlet excitation energy into light in the visible light range or a light-emitting substance that converts triplet excitation energy into light in the visible light range can be used.

<<Light-Emitting Substance that Converts Singlet Excitation Energy into Light>>

The following substances that emit fluorescent light (fluorescent substances) can be given as examples of the light-emitting substance that converts singlet excitation energy into light and can be used in the light-emitting layers (113, 113a, and 113b): a pyrene derivative, an anthracene derivative, a triphenylene derivative, a fluorene derivative, a carbazole derivative, a dibenzothiophene derivative, a dibenzofuran derivative, a dibenzoquinoxaline derivative, a quinoxaline derivative, a pyridine derivative, a pyrimidine derivative, a phenanthrene derivative, and a naphthalene derivative. A pyrene derivative is particularly preferable because it has a high emission quantum yield. Specific examples of the pyrene derivative include N,N′-bis(3-methylphenyl)-N,N′-bis[3-(9-phenyl-9H-fluoren-9-yl)phenyl]pyrene-1,6-diamine (abbreviation: 1,6mMemFLPAPrn), N,N′-diphenyl-N,N′-bis[4-(9-phenyl-9H-fluoren-9-yl)phenyl]pyrene-1,6-diamine (abbreviation: 1,6FLPAPrn), N,N′-bis(dibenzofuran-2-yl)-N,N′-diphenylpyrene-1,6-diamine (abbreviation: 1,6FrAPrn), N,N′-bis(dibenzothiophen-2-yl)-N,N′-diphenylpyrene-1,6-diamine (abbreviation: 1,6ThAPrn), N,N′-(pyrene-1,6-diyl)bis[(N-phenylbenzo[b]naphtho[1,2-d]furan)-6-amine] (abbreviation: 1,6BnfAPrn), N,N′-(pyrene-1,6-diyl)bis[(N-phenylbenzo[b]naphtho[1,2-d]furan)-8-amine](abbreviation: 1,6BnfAPrn-02), and N,N′-(pyrene-1,6-diyl)bis[(6,N-diphenylbenzo[b]naphtho[1,2-d]furan)-8-amine] (abbreviation: 1,6BnfAPrn-03).

In addition, it is possible to use, for example, 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N′-bis[4-(9H-carbazol-9-yl)phenyl]-N,N′-diphenyl-4,4′-stilbenediamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), 4-(10-phenyl-9-anthryl)-4′-(9-phenyl-9H-carbazol-3-yl)triphenylamine (abbreviation: PCBAPA), 4-[4-(10-phenyl-9-anthryl)phenyl]-4′-(9-phenyl-9H-carbazol-3-yl)triphenylamine (abbreviation: PCBAPBA), perylene, 2,5,8,11-tetra-tert-butylperylene (abbreviation: TBP), N,N′-(2-tert-butylanthracene-9,10-diyldi-4,1-phenylene)bis(N,N′,N′-triphenyl-1,4-phenylenediamine) (abbreviation: DPABPA), N,9-diphenyl-N-[4-(9,10-diphenyl-2-anthryl)phenyl]-9H-carbazol-3-amine (abbreviation: 2PCAPPA), and N-[4-(9,10-diphenyl-2-anthryl)phenyl]-N,N′,N′-triphenyl-1,4-phenylenediamine (abbreviation: 2DPAPPA).

It is also possible to use, for example, N-[9,10-bis(biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(biphenyl-2-yl)-2-anthryl]-N,N′,N′-triphenyl-1,4-phenylenediamine (abbreviation: 2DPABPhA), 9,10-bis(biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone (abbreviation: DPQd), rubrene, 5,12-bis(biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), 1,6BnfAPrn-03, 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02), and 3,10-bis[N-(dibenzofuran-3-yl)-N-phenylamino]naphtho[2,3-b;6,7-b′]bisbenzofuran (abbreviation: 3,10FrA2Nbf(IV)-02). In particular, pyrenediamine compounds such as 1,6FLPAPrn, 1,6mMemFLPAPrn, and 1,6BnfAPrn-03 can be used, for example.

<<Light-Emitting Substance that Converts Triplet Excitation Energy into Light>>

Examples of the light-emitting substance that converts triplet excitation energy into light and can be used in the light-emitting layer 113 include substances that emit phosphorescent light (phosphorescent substances) and TADF materials that exhibit thermally activated delayed fluorescence.

A phosphorescent substance is a compound that emits phosphorescent light but does not emit fluorescent light at a temperature higher than or equal to a low temperature (e.g., 77 K) and lower than or equal to room temperature (i.e., higher than or equal to 77 K and lower than or equal to 313 K). The phosphorescent substance preferably contains a metal element with large spin-orbit interaction, and can be an organometallic complex, a metal complex (platinum complex), or a rare earth metal complex, for example. Specifically, the phosphorescent substance preferably contains a transition metal element. It is preferable that the phosphorescent substance contain a platinum group element (ruthenium (Ru), rhodium (Rh), palladium (Pd), osmium (Os), iridium (Ir), or platinum (Pt)), especially iridium, in which case the probability of direct transition between the singlet ground state and the triplet excited state can be increased.

<<Phosphorescent Substance (from 450 nm to 570 nm: Blue or Green)>>

As examples of a phosphorescent substance which emits blue or green light and whose emission spectrum has a peak wavelength in the wavelength ranging from 450 nm to 570 nm, inclusive, the following substances can be given.

Examples include organometallic complexes having a 4H-triazole ring, such as tris{2-[5-(2-methylphenyl)-4-(2,6-dimethylphenyl)-4H-1,2,4-triazol-3-yl-κN²]phenyl-κC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), tris[4-(3-biphenyl)-5-isopropyl-3-phenyl-4H-1,2,4-triazolato]iridium(III) (abbreviation: [Ir(iPrptz-3b)₃]), and tris[3-(5-biphenyl)-5-isopropyl-4-phenyl-4H-1,2,4-triazolato]iridium(III) (abbreviation: [Ir(iPr5btz)₃]); organometallic complexes having a 1H-triazole ring, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); organometallic complexes having an imidazole ring, such as fac-tris[1-(2,6-diisopropylphenyl)-2-phenyl-1H-imidazole]iridium(III) (abbreviation: [Ir(iPrpim)₃]) and tris[3-(2,6-dimethylphenyl)-7-methylimidazo[1,2-f]phenanthridinato]iridium(III) (abbreviation: [Ir(dmpimpt-Me)₃]); and organometallic complexes in which a phenylpyridine derivative having an electron-withdrawing group is a ligand, such as bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), and bis[2-(4′,6′-difluorophenyl)pyridinato-N,C^{2′}]iridium(III) acetylacetonate (abbreviation: FIr(acac)).

<<Phosphorescent Substance (from 495 nm to 590 nm: Green or Yellow)>>

As examples of a phosphorescent substance which emits green or yellow light and whose emission spectrum has a peak wavelength in the wavelength ranging from 495 nm to 590 nm, inclusive, the following substances can be given.

Examples of the phosphorescent substance include organometallic iridium complexes having a pyrimidine ring, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), (acetylacetonato)bis{4,6-dimethyl-2-[6-(2,6-dimethylphenyl)-4-pyrimidinyl-κN³]phenyl-κC}iridium(III) (abbreviation: [Ir(dmppm-dmp)₂(acac)]), and (acetylacetonato)bis(4,6-diphenylpyrimidinato)iridium(III) (abbreviation: [Ir(dppm)₂(acac)]); organometallic iridium complexes having a pyrazine ring, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine ring, such as tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), bis(2-phenylquinolinato-N,C^{2′})iridium(III) acetylacetonate (abbreviation: [Ir(pq)₂(acac)]), bis[2-(2-pyridinyl-κN)phenyl-κC][2-(4-phenyl-2-pyridinyl-κN)phenyl-κC]iridium(III) (abbreviation: [Ir(ppy)₂(4dppy)]), bis[2-(2-pyridinyl-κN)phenyl-κC][2-(4-methyl-5-phenyl-2-pyridinyl-κN)phenyl-κC], [2-d₃-methyl-8-(2-pyridinyl-κN)benzofuro[2,3-b]pyridine-κC]bis[2-(5-d₃-methyl-2-pyridinyl-κN²)phenyl-κC]iridium(III) (abbreviation: Ir(5mppy-d₃)₂(mbfpypy-d₃)), [2-(methyl-d₃)-8-[4-(1-methylethyl-1-d)-2-pyridinyl-κN]benzofuro[2,3-b]pyridin-7-yl-κC]bis[5-(methyl-d₃)-2-[5-(methyl-d₃)-2-pyridinyl-N]phenyl-κC]iridium(III) (abbreviation: Ir(5mtpy-d6)₂(mbfpypy-iPr-d4)), {2-(methyl-d₃)-8-[4-(1-methylethyl-1-d)-2-pyridinyl-κN]benzofuro[2,3-b]pyridin-7-yl-κC}bis{5-(methyl-d₃)-2-[5-(methyl-d₃)-2-pyridinyl-κN]phenyl-κC}iridium(III) (abbreviation: Ir(5mtpy-d₆)₂(mbfpypy-iPr-d₄)), [2-d₃-methyl-(2-pyridinyl-κN)benzofuro[2,3-b]pyridine-κC]bis[2-(2-pyridinyl-κN)phenyl-κC]iridium(III) (abbreviation: Ir(ppy)₂(mbfpypy-d₃)), and [2-(4-methyl-5-phenyl-2-pyridinyl-κN)phenyl-κC]bis[2-(2-pyridinyl-κN)phenyl-κC]iridium(III) (abbreviation: Ir(ppy)₂(mdppy)); organometallic complexes such as bis(2,4-diphenyl-1,3-oxazolato-N,C^{2′})iridium(III) acetylacetonate (abbreviation: [Ir(dpo)₂(acac)]), bis{2-[4′-(perfluorophenyl)phenyl]pyridinato-N,C^{2′}}iridium(III) acetylacetonate (abbreviation: [Ir(p-PF-ph)₂(acac)]), and bis(2-phenylbenzothiazolato-N,C^{2′})iridium(III) acetylacetonate (abbreviation: [Ir(bt)₂(acac)]); and a rare earth metal complex such as tris(acetylacetonato) (monophenanthroline)terbium(III) (abbreviation: [Tb(acac)₃(Phen)]).

<<Phosphorescent Substance (from 570 nm to 750 nm: Yellow or Red)>>

As examples of a phosphorescent substance which emits yellow or red light and whose emission spectrum has a peak wavelength in the wavelength ranging from 570 nm to 750 nm, inclusive, the following substances can be given.

Examples of a phosphorescent substance include organometallic complexes having a pyrimidine ring, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and (dipivaloylmethanato)bis[4,6-di(naphthalen-1-yl)pyrimidinato]iridium(III) (abbreviation: [Ir(d1npm)₂(dpm)]); organometallic complexes having a pyrazine ring, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), bis{4,6-dimethyl-2-[3-(3,5-dimethylphenyl)-5-phenyl-2-pyrazinyl-κN]phenyl-κC}(2,6-dimethyl-3,5-heptanedionato-κ²O,O′)iridium(III) (abbreviation: [Ir(dmdppr-P)₂(dibm)]), bis{4,6-dimethyl-2-[5-(4-cyano-2,6-dimethylphenyl)-3-(3,5-dimethylphenyl)-2-pyrazinyl-κN]phenyl-κC}(2,2,6,6-tetramethyl-3,5-heptanedionato-κ²O,O′)iridium(III) (abbreviation: [Ir(dmdppr-dmCP)₂(dpm)]), bis{2-[5-(2,6-dimethylphenyl)-3-(3,5-dimethylphenyl)-2-pyrazinyl-κN]-4,6-dimethylphenyl-κC}(2,2′,6,6′-tetramethyl-3,5-heptanedionato-κ²O,O′)iridium(III) (abbreviation: [Ir(dmdppr-dmp)₂(dpm)]), (acetylacetonato)bis(2-methyl-3-phenylquinoxalinato-N,C^{2′})iridium(III) (abbreviation: [Ir(mpq)₂(acac)]), (acetylacetonato)bis(2,3-diphenylquinoxalinato-N,C^{2′})iridium(III) (abbreviation: [Ir(dpq)₂(acac)]), and (acetylacetonato)bis[2,3-bis(4-fluorophenyl)quinoxalinato]iridium(III) (abbreviation: [Ir(Fdpq)₂(acac)]); organometallic complexes having a pyridine ring, such as tris(1-phenylisoquinolinato-N,C^{2′})iridium(III) (abbreviation: [Ir(piq)₃]), bis(1-phenylisoquinolinato-N,C^{2′})iridium(III) acetylacetonate (abbreviation: [Ir(piq)₂(acac)]), and bis[4,6-dimethyl-2-(2-quinolinyl-κN)phenyl-κC](2,4-pentanedionato-κ²O,O′)iridium(III) (abbreviation: [Ir(dmpqn)₂(acac)]); a platinum complex such as 2,3,7,8,12,13,17,18-octaethyl-21H,23H-porphyrin platinum(II) (abbreviation: [PtOEP]); and rare earth metal complexes such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]).

### <<TADF Material>>

Any of materials described below can be used as the TADF material. The TADF material is a material that has a small difference between its S₁ and T₁ levels (preferably less than or equal to 0.20 eV), enables up-conversion of a triplet excited state into a singlet excited state (i.e., reverse intersystem crossing) using a little thermal energy, and efficiently exhibits light (fluorescent light) from the singlet excited state. The thermally activated delayed fluorescence is efficiently obtained under the condition where the difference in energy between the triplet excitation energy level and the singlet excitation energy level is greater than or equal to 0.00 eV and less than or equal to 0.20 eV, preferably greater than or equal to 0.00 eV and less than or equal to 0.10 eV. Note that delayed fluorescent light by the TADF material refers to light emission having a spectrum similar to that of normal fluorescent light and an extremely long lifetime. The lifetime is longer than or equal to 1×10⁻⁶ seconds or longer than or equal to 1×10⁻³ seconds.

Note that the TADF material can be also used as an electron-transport material, a hole-transport material, or a host material.

Examples of the TADF material include fullerene, a derivative thereof, an acridine derivative such as proflavine, and eosin. Other examples thereof include a metal-containing porphyrin, containing a metal such as a porphyrin containing magnesium (Mg), zinc (Zn), cadmium (Cd), tin (Sn), platinum (Pt), indium (In), or palladium (Pd). Examples of the metal-containing porphyrin include a protoporphyrin-tin fluoride complex (abbreviation: SnF₂(Proto IX)), a mesoporphyrin-tin fluoride complex (abbreviation: SnF₂(Meso IX)), a hematoporphyrin-tin fluoride complex (abbreviation: SnF₂(Hemato IX)), a coproporphyrin tetramethyl ester-tin fluoride complex (abbreviation: SnF₂(Copro III-4Me)), an octaethylporphyrin-tin fluoride complex (abbreviation: SnF₂(OEP)), an etioporphyrin-tin fluoride complex (abbreviation: SnF₂(Etio I)), and an octaethylporphyrin-platinum chloride complex (abbreviation: PtCl₂OEP).

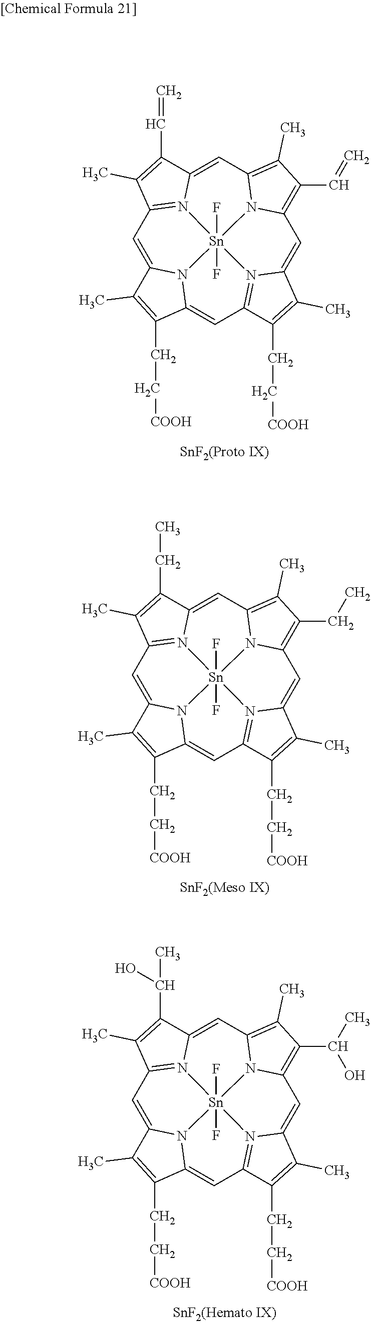

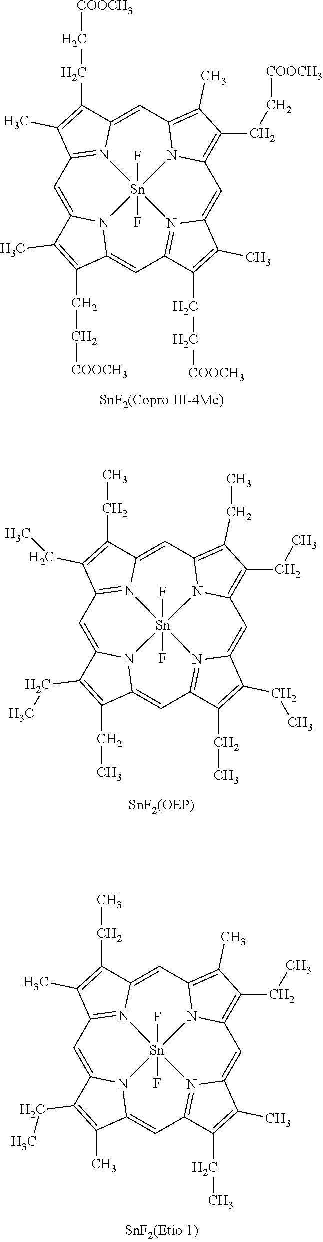

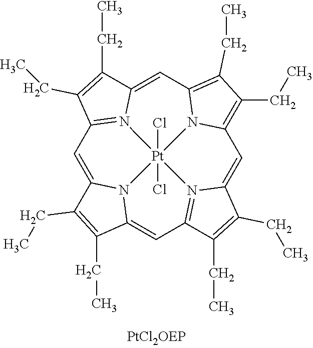

Additionally, a heteroaromatic compound having a π-electron rich heteroaromatic compound and a π-electron deficient heteroaromatic compound may be used, such as 2-(biphenyl-4-yl)-4,6-bis(12-phenylindolo[2,3-a]carbazol-11-yl)-1,3,5-triazine (abbreviation: PIC-TRZ), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), 10-phenyl-10H,10′H-spiro[acridin-9,9′-anthracen]-10′-one (abbreviation: ACRSA), 4-(9′-phenyl-3,3′-bi-9H-carbazol-9-yl)benzofuro[3,2-d]pyrimidine (abbreviation: 4PCCzBfpm), 4-[4-(9′-phenyl-3,3′-bi-9H-carbazol-9-yl)phenyl]benzofuro[3,2-d]pyrimidine (abbreviation: 4PCCzPBfpm), or 9-[3-(4,6-diphenyl-1,3,5-triazin-2-yl)phenyl]-9′-phenyl-2,3′-bi-9H-carbazole (abbreviation: mPCCzPTzn-02).

Note that a substance in which a π-electron rich heteroaromatic compound is directly bonded to a π-electron deficient heteroaromatic compound is particularly preferable because both the donor property of the π-electron rich heteroaromatic compound and the acceptor property of the π-electron deficient heteroaromatic compound are improved and the energy difference between the singlet excited state and the triplet excited state becomes small. As the TADF material, a TADF material in which the singlet and triplet excited states are in thermal equilibrium (TADF100) may be used. Since such a TADF material enables a short emission lifetime (excitation lifetime), the efficiency of a light-emitting device in a high-luminance region can be less likely to decrease.

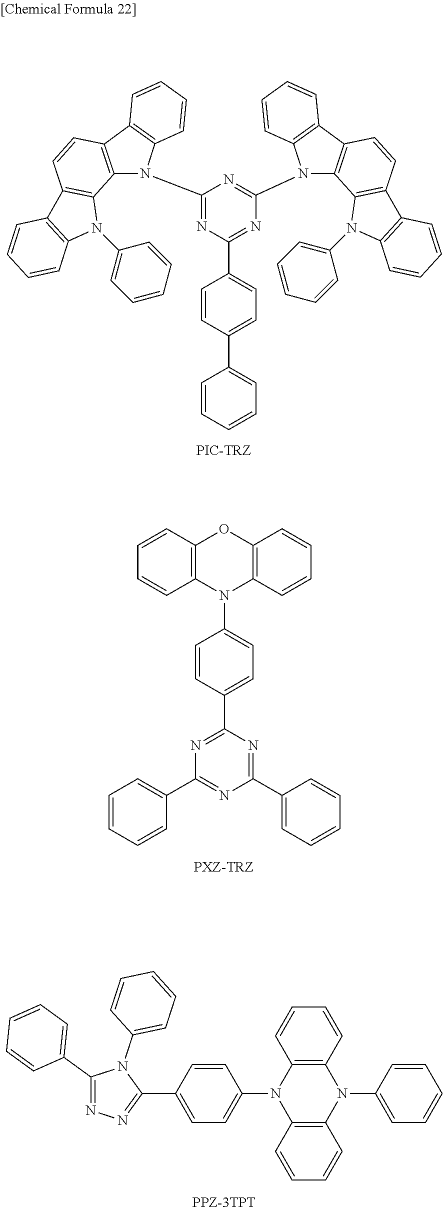 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-c3ccc(N4c5ccccc5Oc5ccccc54)cc3)n2)cc1 ; c1ccc(-c2nnc(-c3ccc(N4c5ccccc5N(c5ccccc5)c5ccccc54)cc3)n2-c2ccccc2)cc1]

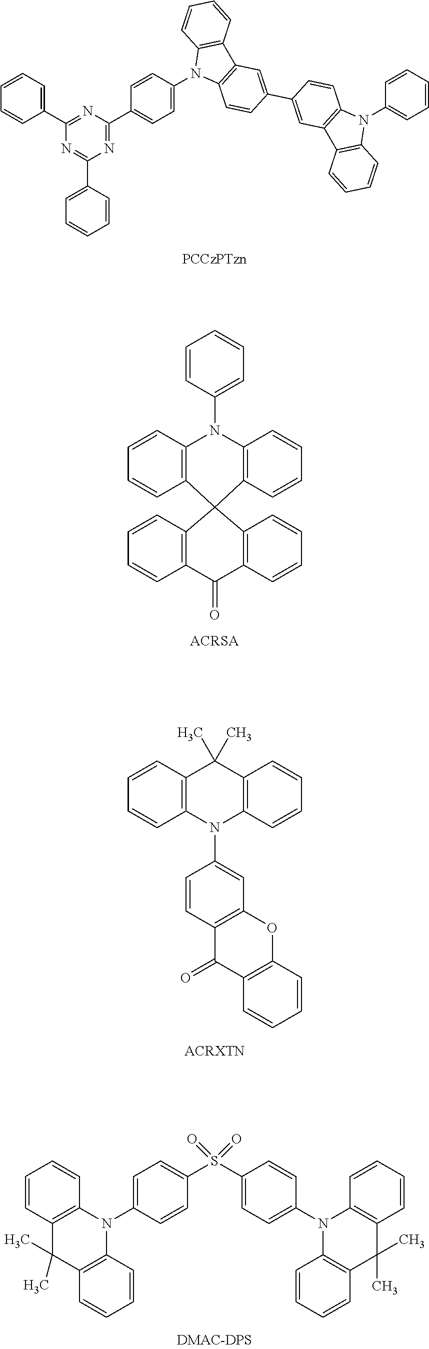 [SMILES: c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1 ; O=C1c2ccccc2C2(c3ccccc31)c1ccccc1N(c1ccccc1)c1ccccc12 ; CC1(C)c2ccccc2N(c2ccc3c(=O)c4ccccc4oc3c2)c2ccccc21 ; CC1(C)c2ccccc2N(c2ccc(S(=O)(=O)c3ccc(N4c5ccccc5C(C)(C)c5ccccc54)cc3)cc2)c2ccccc21]

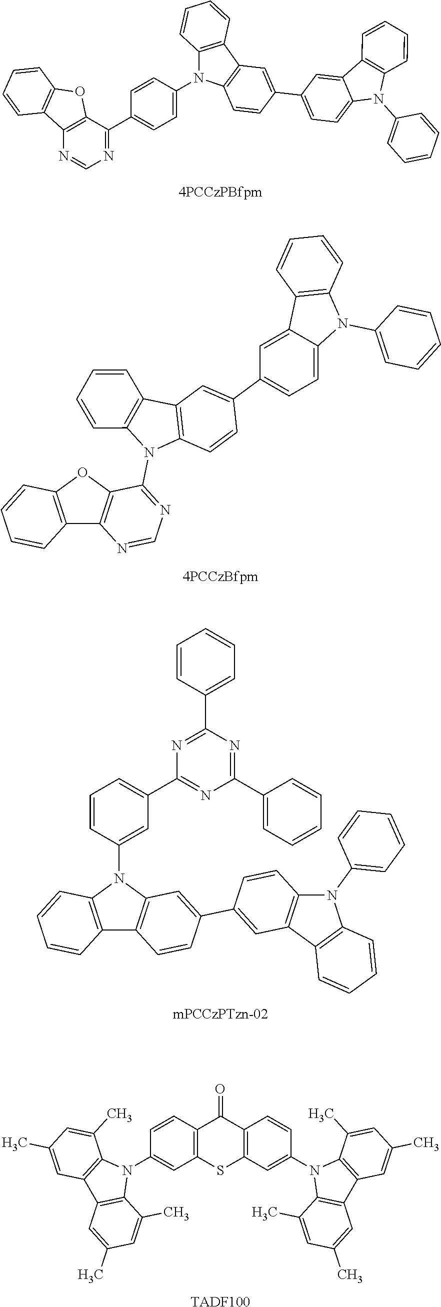

In addition to the above, another example of a material having a function of converting triplet excitation energy into light is a nano-structure of a transition metal compound having a perovskite structure. In particular, a nano-structure of a metal halide perovskite material is preferable. The nano-structure is preferably a nanoparticle or a nanorod.

As the organic compound (e.g., the host material) used in combination with the above-described light-emitting substance (guest material) in the light-emitting layers (113, 113a, and 113b), one or more kinds selected from substances having a larger energy gap than the light-emitting substance (guest material) can be used.

### <<Host Material for Fluorescent Light>>

In the case where the light-emitting substance used in the light-emitting layers (113, 113a, and 113b) is a fluorescent substance, an organic compound (a host material) used in combination with the fluorescent substance is preferably an organic compound that has a high energy level in a singlet excited state and has a low energy level in a triplet excited state or an organic compound having a high fluorescence quantum yield. Therefore, the hole-transport material (described above) and the electron-transport material (described below) shown in this embodiment, for example, can be used as long as they are organic compounds that satisfy such a condition. In addition, the first organic compound can be used.

In terms of a preferred combination with the light-emitting substance (fluorescent substance), examples of the organic compound (host material), some of which are mentioned in the above specific examples, include fused polycyclic aromatic compounds such as an anthracene derivative, a tetracene derivative, a phenanthrene derivative, a pyrene derivative, a chrysene derivative, and a dibenzo[g,p]chrysene derivative.

Specific examples of the organic compound (host material) that is preferably used in combination with the fluorescent substance include 9-phenyl-3-[4-(10-phenyl-9-anthryl)phenyl]-9H-carbazole (abbreviation: PCzPA), 3,6-diphenyl-9-[4-(10-phenyl-9-anthryl)phenyl]-9H-carbazole (abbreviation: DPCzPA), 3-[4-(1-naphthyl)phenyl]-9-phenyl-9H-carbazole (abbreviation: PCPN), 9,10-diphenylanthracene (abbreviation: DPAnth), N,N-diphenyl-9-[4-(10-phenyl-9-anthryl)phenyl]-9H-carbazol-3-amine (abbreviation: CzA1PA), 4-(10-phenyl-9-anthryl)triphenylamine (abbreviation: DPhPA), YGAPA, PCAPA, N,9-diphenyl-N-{4-[4-(10-phenyl-9-anthryl)phenyl]phenyl}-9H-carbazol-3-amine (abbreviation: PCAPBA), N-(9,10-diphenyl-2-anthryl)-N,9-diphenyl-9H-carbazol-3-amine (abbreviation: 2PCAPA), 6,12-dimethoxy-5,11-diphenylchrysene, N,N,N′,N′,N″,N″,N′″,N′″-octaphenyldibenzo[g,p]chrysene-2,7,10,15-tetraamine (abbreviation: DBC1), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]benzo[b]naphtho[1,2-d]furan (abbreviation: 2mBnfPPA), 9-phenyl-10-[4′-(9-phenyl-9H-fluoren-9-yl)biphenyl-4-yl]anthracene (abbreviation: FLPPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9-(1-naphthyl)-10-(2-naphthyl)anthracene (abbreviation: α,β-ADN), 2-(10-phenylanthracen-9-yl)dibenzofuran, 2-(10-phenyl-9-anthracenyl)benzo[b]naphtho[2,3-d]furan (abbreviation: Bnf(II)PhA), 9-(1-naphthyl)-10-[4-(2-naphthyl)phenyl]anthracene (abbreviation: αN-QNPAnth), 2,9-di(1-naphthyl)-10-phenylanthracene (abbreviation: 2αN-αNPhA), 9-(1-naphthyl)-10-[3-(1-naphthyl)phenyl]anthracene (abbreviation: αN-mαNPAnth), 9-(2-naphthyl)-10-[3-(1-naphthyl)phenyl]anthracene (abbreviation: βN-mαNPAnth), 9-(1-naphthyl)-10-[4-(1-naphthyl)phenyl]anthracene (abbreviation: αN-αNPAnth), 9-(2-naphthyl)-10-[4-(2-naphthyl)phenyl]anthracene (abbreviation: βN-βNPAnth), 2-(1-naphthyl)-9-(2-naphthyl)-10-phenylanthracene (abbreviation: 2αN-βNPhA), 9-(2-naphthyl)-10-[3-(2-naphthyl)phenyl]anthracene (abbreviation: βN-mβNPAnth), 1-{4-[10-(biphenyl-4-yl)-9-anthracenyl]phenyl}-2-ethyl-1H-benzimidazole (abbreviation: EtBImPBPhA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 1,3,5-tri(1-pyrenyl)benzene (abbreviation: TPB3), 5,12-diphenyltetracene, and 5,12-bis(biphenyl-2-yl)tetracene.

### <<Host Material for Phosphorescent Light>>

In the case where the light-emitting substance used in the light-emitting layers (113, 113a, and 113b) is a phosphorescent substance, an organic compound having triplet excitation energy (an energy difference between a ground state and a triplet excited state) which is higher than that of the light-emitting substance is preferably selected as the organic compound (host material) used in combination with the phosphorescent substance. Note that when a plurality of organic compounds (e.g., a first host material and a second host material (or an assist material)) are used in combination with a light-emitting substance so that an exciplex is formed, the plurality of organic compounds are preferably mixed with the phosphorescent substance. In addition, the first organic compound can be used.

With such a structure, light emission can be efficiently obtained by exciplex-triplet energy transfer (ExTET), which is energy transfer from an exciplex to a light-emitting substance. Note that a combination of the plurality of organic compounds that easily forms an exciplex is preferred, and it is particularly preferable to combine a compound that easily accepts holes (hole-transport material) and a compound that easily accepts electrons (electron-transport material).

In terms of a preferred combination with the light-emitting substance (phosphorescent substance), examples of the organic compounds (the host material and the assist material), some of which are mentioned in the above specific examples, include an aromatic amine (an organic compound having an aromatic amine skeleton), a carbazole derivative (an organic compound having a carbazole ring), a dibenzothiophene derivative (an organic compound having a dibenzothiophene ring), a dibenzofuran derivative (an organic compound having a dibenzofuran ring), an oxadiazole derivative (an organic compound having an oxadiazole ring), a triazole derivative (an organic compound having a triazole ring), a benzimidazole derivative (an organic compound having a benzimidazole ring), a quinoxaline derivative (an organic compound having a quinoxaline ring), a dibenzoquinoxaline derivative (an organic compound having a dibenzoquinoxaline ring), a pyrimidine derivative (an organic compound having a pyrimidine ring), a triazine derivative (an organic compound having a triazine ring), a pyridine derivative (an organic compound having a pyridine ring), a bipyridine derivative (an organic compound having a bipyridine ring), a phenanthroline derivative (an organic compound having a phenanthroline ring), a furodiazine derivative (an organic compound having a furodiazine ring), and zinc- or aluminum-based metal complexes.

Among the above organic compounds, specific examples of the aromatic amine and the carbazole derivative, which are organic compounds having a high hole-transport property, are the same as the specific examples of the hole-transport materials described above, and those materials are preferable as the host material.

Among the above organic compounds, specific examples of the dibenzothiophene derivative and the dibenzofuran derivative, which are organic compounds having a high hole-transport property, include 4-{3-[3-(9-phenyl-9H-fluoren-9-yl)phenyl]phenyl}dibenzofuran (abbreviation: mmDBFFLBi-II), 4,4′,4″-(benzene-1,3,5-triyl)tri(dibenzofuran) (abbreviation: DBF3P-II), DBT3P-II, 2,8-diphenyl-4-[4-(9-phenyl-9H-fluoren-9-yl)phenyl]dibenzothiophene (abbreviation: DBTFLP-III), 4-[4-(9-phenyl-9H-fluoren-9-yl)phenyl]-6-phenyldibenzothiophene (abbreviation: DBTFLP-IV), and 4-[3-(triphenylen-2-yl)phenyl]dibenzothiophene (abbreviation: mDBTPTp-II). Such derivatives are preferable as the host material.

Other examples of preferred host materials include metal complexes having an oxazole-based or thiazole-based ligand, such as bis[2-(2-benzoxazolyl)phenolato]zinc(II) (abbreviation: ZnPBO) and bis[2-(2-benzothiazolyl)phenolato]zinc(II) (abbreviation: ZnBTZ).

Among the above organic compounds, specific examples of the oxadiazole derivative, the triazole derivative, the benzimidazole derivative, the quinoxaline derivative, the dibenzoquinoxaline derivative, the quinazoline derivative, and the phenanthroline derivative, which are organic compounds having a high electron-transport property, include: an organic compound containing a heteroaromatic ring having an azole ring such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 3-(4-biphenylyl)-4-phenyl-5-(4-tert-butylphenyl)-1,2,4-triazole (abbreviation: TAZ), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), or 4,4′-bis(5-methylbenzoxazol-2-yl)stilbene (abbreviation: BzOs); an organic compound containing a heteroaromatic ring having a phenanthroline ring such as bathophenanthroline (abbreviation: BPhen), bathocuproine (abbreviation: BCP), 2,9-di(naphthalen-2-yl)-4,7-diphenyl-1,10-phenanthroline (abbreviation: NBPhen), or 2,2′-(1,3-phenylene)bis(9-phenyl-1,10-phenanthroline) (abbreviation: mPPhen2P); an organic compound containing a heteroaromatic ring having a dibenzoquinoxaline ring such as 2-[3-(dibenzothiophen-4-yl)phenyl]dibenzo[f,h]quinoxaline (abbreviation: 2mDBTPDBq-II); 2-[3′-(dibenzothiophen-4-yl)biphenyl-3-yl]dibenzo[f,h]quinoxaline (abbreviation: 2mDBTBPDBq-II); 2-[3′-(9H-carbazol-9-yl)biphenyl-3-yl]dibenzo[f,h]quinoxaline (abbreviation: 2mCzBPDBq); 2-[4-(3,6-diphenyl-9H-carbazol-9-yl)phenyl]dibenzo[f,h]quinoxaline (abbreviation: 2CzPDBq-III); 7-[3-(dibenzothiophen-4-yl)phenyl]dibenzo[f,h]quinoxaline (abbreviation: 7mDBTPDBq-II); 6-[3-(dibenzothiophen-4-yl)phenyl]dibenzo[f,h]quinoxaline (abbreviation: 6mDBTPDBq-II); 2-{4-[9,10-di(2-naphthyl)-2-anthryl]phenyl}-1-phenyl-1H-benzimidazole (abbreviation: ZADN); and 2-[4′-(9-phenyl-9H-carbazol-3-yl)-3,1′-biphenyl-1-yl]dibenzo[f,h]quinoxaline (abbreviation: 2mpPCBPDBq). Such organic compounds are preferable as the host material.

Among the above organic compounds, specific examples of the pyridine derivative, the diazine derivative (e.g., the pyrimidine derivative, the pyrazine derivative, and the pyridazine derivative), the triazine derivative, and the furodiazine derivative, which are organic compounds having a high electron-transport property, include organic compounds containing a heteroaromatic ring having a diazine ring such as 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 3,5-bis[3-(9H-carbazol-9-yl)phenyl]pyridine (abbreviation: 35DCzPPy), 1,3,5-tri[3-(3-pyridyl)phenyl]benzene (abbreviation: TmPyPB), 9,9′-[pyrimidine-4,6-diylbis(biphenyl-3,3′-diyl)]bis(9H-carbazole) (abbreviation: 4,6mCzBP2Pm), 2-[3′-(9,9-dimethyl-9H-fluoren-2-yl)biphenyl-3-yl]-4,6-diphenyl-1,3,5-triazine (abbreviation: mFBPTzn), 8-(biphenyl-4-yl)-4-[3-(dibenzothiophen-4-yl)phenyl]-[1]benzofuro[3,2-d]pyrimidine (abbreviation: 8BP-4mDBtPBfpm), 9-[3′-(dibenzothiophen-4-yl)biphenyl-3-yl]naphtho[1′,2′: 4,5]furo[2,3-b]pyrazine (abbreviation: 9mDBtBPNfpr), 9-[3′-(dibenzothiophen-4-yl)biphenyl-4-yl]naphtho[1′,2′: 4,5]furo[2,3-b]pyrazine (abbreviation: 9pmDBtBPNfpr), 11-[3′-(dibenzothiophen-4-yl)biphenyl-3-yl]phenanthro[9′,10′: 4,5]furo[2,3-b]pyrazine (abbreviation: 11mDBtBPPnfpr), 11-[3′-(dibenzothiophen-4-yl)biphenyl-4-yl]phenanthro[9′,10′: 4,5]furo[2,3-b]pyrazine, 11-[3′-(9H-carbazol-9-yl)biphenyl-3-yl]phenanthro[9′,10′: 4,5]furo[2,3-b]pyrazine, 12-(9′-phenyl-3,3′-bi-9H-carbazol-9-yl)phenanthro[9′,10′: 4,5]furo[2,3-b]pyrazine (abbreviation: 12PCCzPnfpr), 9-[3′-(9-phenyl-9H-carbazol-3-yl)biphenyl-4-yl]naphtho[1′,2′: 4,5]furo[2,3-b]pyrazine (abbreviation: 9pmPCBPNfpr), 9-(9′-phenyl-3,3′-bi-9H-carbazol-9-yl)naphtho[1′,2′: 4,5]furo[2,3-b]pyrazine (abbreviation: 9PCCzNfpr), 10-(9′-phenyl-3,3′-bi-9H-carbazol-9-yl)naphtho[1′,2′: 4,5]furo[2,3-b]pyrazine (abbreviation: 10PCCzNfpr), 9-[3′-(6-phenylbenzo[b]naphtho[1,2-d]furan-8-yl)biphenyl-3-yl]naphtho[1′,2′: 4,5]furo[2,3-b]pyrazine (abbreviation: 9mBnfBPNfpr), 9-{3-[6-(9,9-dimethylfluoren-2-yl)dibenzothiophen-4-yl]phenyl}naphtho[1′,2′: 4,5]furo[2,3-b]pyrazine (abbreviation: 9mFDBtPNfpr), 9-[3′-(6-phenyldibenzothiophen-4-yl)biphenyl-3-yl]naphtho[1′,2′: 4,5]furo[2,3-b]pyrazine (abbreviation: 9mDBtBPNfpr-02), 9-[3-(9′-phenyl-3,3′-bi-9H-carbazol-9-yl)phenyl]naphtho[1′,2′: 4,5]furo[2,3-b]pyrazine (abbreviation: 9mPCCzPNfpr), 9-{(3′-[2,8-diphenyldibenzothiophen-4-yl)biphenyl-3-yl}naphtho[1′,2′: 4,5]furo[2,3-b]pyrazine, 11-[3′-(2,8-diphenyldibenzothiophen-4-yl)biphenyl-3-yl]phenanthro[9′,10′: 4,5]furo[2,3-b]pyrazine, 5-[3-(4,6-diphenyl-1,3,5-triazin-2-yl)phenyl]-7,7-dimethyl-5H,7H-indeno[2,1-b]carbazole (abbreviation: mINc(II)PTzn), 2-[3′-(triphenylen-2-yl)biphenyl-3-yl]-4,6-diphenyl-1,3,5-triazine (abbreviation: mTpBPTzn), 2-(biphenyl-4-yl)-4-phenyl-6-(9,9′-spirobi[9H-fluoren]-2-yl)-1,3,5-triazine (abbreviation: BP-SFTzn), 2,6-bis(4-naphthalen-1-ylphenyl)-4-[4-(3-pyridyl)phenyl]pyrimidine (abbreviation: 2,4NP-6PyPPm), 3-[9-(4,6-diphenyl-1,3,5-triazin-2-yl)-2-dibenzofuranyl]-9-phenyl-9H-carbazole (abbreviation: PCDBfTzn), 2-(biphenyl-3-yl)-4-phenyl-6-{8-[(1,1′: 4′,1″-terphenyl)-4-yl]-1-dibenzofuranyl}-1,3,5-triazine (abbreviation: mBP-TPDBfTzn), 6-(biphenyl-3-yl)-4-[3,5-bis(9H-carbazol-9-yl)phenyl)-2-phenylpyrimidine (abbreviation: 6mBP-4Cz2PPm), and 4-[3,5-bis(9H-carbazol-9-yl)phenyl]-2-phenyl-6-(biphenyl-4-yl)pyrimidine (abbreviation: 6BP-4Cz2PPm), and those materials are preferable as the host material.

Among the above organic compounds, specific examples of metal complexes that are organic compounds having a high electron-transport property include zinc- or aluminum-based metal complexes, such as tris(8-quinolinolato)aluminum(III) (abbreviation: Alq), tris(4-methyl-8-quinolinolato)aluminum(III) (abbreviation: Almq₃), bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), and bis(8-quinolinolato)zinc(II) (abbreviation: Znq), and metal complexes having a quinoline ring or a benzoquinoline ring. Such metal complexes are preferable as the host material.

Moreover, high-molecular compounds such as poly(2,5-pyridinediyl) (abbreviation: PPy), poly[(9,9-dihexylfluorene-2,7-diyl)-co-(pyridine-3,5-diyl)](abbreviation: PF-Py), and poly[(9,9-dioctylfluorene-2,7-diyl)-co-(2,2′-bipyridine-6,6′-diyl)] (abbreviation: PF-BPy) are preferable as the host material.

Furthermore, the following organic compounds having a diazine ring, which have bipolar properties, a high hole-transport property and a high electron-transport property, can be used as the host material: 9-phenyl-9′-(4-phenyl-2-quinazolinyl)-3,3′-bi-9H-carbazole (abbreviation: PCCzQz), 2-[4′-(9-phenyl-9H-carbazol-3-yl)-3,1′-biphenyl-1-yl]dibenzo[f,h]quinoxaline (abbreviation: 2mpPCBPDBq), 5-[3-(4,6-diphenyl-1,3,5-triazin-2-yl)phenyl]-7,7-dimethyl-5H,7H-indeno[2,1-b]carbazole (abbreviation: mINc(II)PTzn), 11-[4-(biphenyl-4-yl)-6-phenyl-1,3,5-triazin-2-yl]-11,12-dihydro-12-phenyl-indolo[2,3-a]carbazole (abbreviation: BP-Icz(II)Tzn), and 7-[4-(9-phenyl-9H-carbazol-2-yl)quinazolin-2-yl]-7H-dibenzo[c,g]carbazole (abbreviation: PC-cgDBCzQz).

### <Electron-Transport Layer>

The electron-transport layers (114, 114a, and 114b) transport electrons, which are injected from the second electrode 102 and the charge-generation layers (106, 106a, and 106b) by electron-injection layers (115, 115a, and 115b) described later, to the light-emitting layers (113, 113a, and 113b). The heat resistance of the light-emitting device of one embodiment of the present invention can be improved by including a stacked structure of electron-transport layers. The electron-transport material used in the electron-transport layers (114, 114a, and 114b) is preferably a substance having an electron mobility of 1×10⁻⁶ cm²/Vs or higher in the case where the square root of the electric field strength [V/cm] is 600. Note that any other substance can also be used as long as the substance has an electron-transport property higher than a hole-transport property. The electron-transport layers (114, 114a, and 114b) can function even with a single-layer structure and may have a stacked-layer structure including two or more layers. When a photolithography process is performed over the electron-transport layer containing the above-described mixed material, which has heat resistance, an adverse effect of the thermal process on the device characteristics can be reduced.

### <<Electron-Transport Material>>

As the electron-transport material that can be used for the electron-transport layers (114, 114a, and 114b), an organic compound having a high electron-transport property can be used, and for example, a heteroaromatic compound can be used. The term heteroaromatic compound refers to a cyclic compound containing at least two different kinds of elements in a ring. Examples of cyclic structures include a three-membered ring, a four-membered ring, a five-membered ring, a six-membered ring, and the like, among which a five-membered ring and a six-membered ring are particularly preferred. The elements contained in the heteroaromatic compound are preferably one or more of nitrogen, oxygen, and sulfur, in addition to carbon. In particular, a heteroaromatic compound containing nitrogen (a nitrogen-containing heteroaromatic compound) is preferred, and any of materials having a high electron-transport property (electron-transport materials), such as a nitrogen-containing heteroaromatic compound and a π-electron deficient heteroaromatic compound including the nitrogen-containing heteroaromatic compound, is preferably used.

Note that the electron-transport material can be preferably different from the materials used in the light-emitting layer. Not all excitons formed by recombination of carriers in the light-emitting layer can contribute to light emission and some excitons are diffused into a layer in contact with the light-emitting layer or a layer in the vicinity of the light-emitting layer. In order to avoid this phenomenon, the energy level (the lowest singlet excitation level or the lowest triplet excitation level) of a material used for the layer in contact with the light-emitting layer or the layer in the vicinity of the light-emitting layer is preferably higher than that of a material used for the light-emitting layer. Therefore, when a material different from the material of the light-emitting layer is used as the electron-transport material, a light-emitting device with high efficiency can be obtained.

The heteroaromatic compound is an organic compound including at least one heteroaromatic ring.

The heteroaromatic ring includes any one of a pyridine ring, a diazine ring, a triazine ring, an azole ring, an oxazole ring, a thiazole ring, and the like. A heteroaromatic ring having a diazine ring includes a heteroaromatic ring having a pyrimidine ring, a pyrazine ring, a pyridazine ring, or the like. A heteroaromatic ring having an azole ring includes a heteroaromatic ring having an imidazole ring, a triazole ring, or an oxadiazole ring.

The heteroaromatic ring includes a fused heteroaromatic ring having a fused ring structure. Examples of the fused heteroaromatic ring include a quinoline ring, a benzoquinoline ring, a quinoxaline ring, a dibenzoquinoxaline ring, a quinazoline ring, a benzoquinazoline ring, a dibenzoquinazoline ring, a phenanthroline ring, a furodiazine ring, and a benzimidazole ring.

Examples of the heteroaromatic compound having a five-membered ring structure, which is a heteroaromatic compound containing carbon and one or more of nitrogen, oxygen, sulfur, and the like, include a heteroaromatic compound having an imidazole ring, a heteroaromatic compound having a triazole ring, a heteroaromatic compound having an oxazole ring, a heteroaromatic compound having an oxadiazole ring, a heteroaromatic compound having a thiazole ring, and a heteroaromatic compound having a benzimidazole ring.

Examples of the heteroaromatic compound having a six-membered ring structure, which is a heteroaromatic compound containing carbon and one or more of nitrogen, oxygen, sulfur, and the like, include a heteroaromatic compound having a heteroaromatic ring, such as a pyridine ring, a diazine ring (a pyrimidine ring, a pyrazine ring, a pyridazine ring, or the like), a triazine ring, or an azole ring. Other examples include a heteroaromatic compound having a bipyridine structure, a heteroaromatic compound having a terpyridine structure, and the like, which are included in examples of a heteroaromatic compound in which pyridine rings are connected.

Examples of the heteroaromatic compound having a fused ring structure partly including the above six-membered ring structure include a heteroaromatic compound having a fused heteroaromatic ring such as a quinoline ring, a benzoquinoline ring, a quinoxaline ring, a dibenzoquinoxaline ring, a phenanthroline ring, a furodiazine ring (including a structure in which an aromatic ring is fused to a furan ring of a furodiazine ring), or a benzimidazole ring.

Specific examples of the above-described heteroaromatic compound having a five-membered ring structure (an aazole ring (including an imidazole ring, a triazole ring, or an oxadiazole ring), an oxazole ring, a thiazole ring, or a benzimidazole ring) include 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 3-(4-biphenylyl)-4-phenyl-5-(4-tert-butylphenyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), and 4,4′-bis(5-methylbenzoxazol-2-yl)stilbene (abbreviation: BzOs).

Specific examples of the above-described heteroaromatic compound having a six-membered ring structure (including a heteroaromatic ring having a pyridine ring, a diazine ring, a triazine ring, or the like) include: a heteroaromatic compound including a heteroaromatic ring having a pyridine ring, such as 3,5-bis[3-(9H-carbazol-9-yl)phenyl]pyridine (abbreviation: 35DCzPPy) or 1,3,5-tri[3-(3-pyridyl)phenyl]benzene (abbreviation: TmPyPB); a heteroaromatic compound including a heteroaromatic ring having a triazine ring, such as 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 5-[3-(4,6-diphenyl-1,3,5-triazin-2-yl)phenyl]-7,7-dimethyl-5H,7H-indeno[2,1-b]carbazole (abbreviation: mINc(II)PTzn), 2-[3′-(triphenylen-2-yl)biphenyl-3-yl]-4,6-diphenyl-1,3,5-triazine (abbreviation: mTpBPTzn), 2-(biphenyl-4-yl)-4-phenyl-6-(9,9′-spirobi[9H-fluoren]-2-yl)-1,3,5-triazine (abbreviation: BP-SFTzn), 2,6-bis(4-naphthalen-1-ylphenyl)-4-[4-(3-pyridyl)phenyl]pyrimidine (abbreviation: 2,4NP-6PyPPm), 3-[9-(4,6-diphenyl-1,3,5-triazin-2-yl)-2-dibenzofuranyl]-9-phenyl-9H-carbazole (abbreviation: PCDBfTzn), 2-(biphenyl-3-yl)-4-phenyl-6-{8-[(1,1′: 4′,1″-terphenyl)-4-yl]-1-dibenzofuranyl}-1,3,5-triazine (abbreviation: mBP-TPDBfTzn), 2-{3-[3-(dibenzothiophen-4-yl)phenyl]phenyl}-4,6-diphenyl-1,3,5-triazine (abbreviation: mDBtBPTzn), or mFBPTzn; and a heteroaromatic compound including a heteroaromatic ring having a diazine (pyrimidine) ring, such as 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 4,6mCzBP2Pm, 6-(biphenyl-3-yl)-4-[3,5-bis(9H-carbazol-9-yl)phenyl]-2-phenylpyrimidine (abbreviation: 6mBP-4Cz2PPm), 4-[3,5-bis(9H-carbazol-9-yl)phenyl]-2-phenyl-6-(biphenyl-4-yl)pyrimidine (abbreviation: 6BP-4Cz2PPm), 4-[3-(dibenzothiophen-4-yl)phenyl]-8-(naphthalen-2-yl)-[1]benzofuro[3,2-d]pyrimidine (abbreviation: 8βN-4mDBtPBfpm), 8BP-4mDBtPBfpm, 9mDBtBPNfpr, 9pmDBtBPNfpr, 3,8-bis[3-(dibenzothiophen-4-yl)phenyl]benzofuro[2,3-b]pyrazine (abbreviation: 3,8mDBtP2Bfpr), 4,8-bis[3-(dibenzothiophen-4-yl)phenyl]-[1]benzofuro[3,2-d]pyrimidine (abbreviation: 4,8mDBtP2Bfpm), 8-[3′-(dibenzothiophen-4-yl)biphenyl-3-yl]naphtho[1′,2′: 4,5]furo[3,2-d]pyrimidine (abbreviation: 8mDBtBPNfpm), or 8-[(2,2′-binaphthalen)-6-yl]-4-[3-(dibenzothiophen-4-yl)phenyl]-[1]benzofuro[3,2-d]pyrimidine (abbreviation: 8(βN2)-4mDBtPBfpm). Note that the above aromatic compounds including a heteroaromatic ring include a heteroaromatic compound having a fused heteroaromatic ring.

Other examples include heteroaromatic compounds including a heteroaromatic ring having a diazine (pyrimidine) ring, such as 2,2′-(pyridine-2,6-diyl)bis(4-phenylbenzo[h]quinazoline) (abbreviation: 2,6(P-Bqn)2Py), 2,2′-(2,2′-bipyridine-6,6′-diyl)bis(4-phenylbenzo[h]quinazoline) (abbreviation: 6,6′(P-Bqn)2BPy), 2,2′-(pyridine-2,6-diyl)bis{4-[4-(2-naphthyl)phenyl]-6-phenylpyrimidine} (abbreviation: 2,6(NP—PPm)2Py), or 6-(biphenyl-3-yl)-4-[3,5-bis(9H-carbazol-9-yl)phenyl]-2-phenylpyrimidine (abbreviation: 6mBP-4Cz2PPm), and a heteroaromatic compound including a heteroaromatic ring having a triazine ring, such as 2,4,6-tris[3′-(pyridin-3-yl)biphenyl-3-yl]-1,3,5-triazine (abbreviation: TmPPPyTz), 2,4,6-tris(2-pyridyl)-1,3,5-triazine (abbreviation: 2Py3Tz), or 2-[3-(2,6-dimethyl-3-pyridinyl)-5-(9-phenanthrenyl)phenyl]-4,6-diphenyl-1,3,5-triazine (abbreviation: mPn-mDMePyPTzn).

Specific examples of the above-described heteroaromatic compound having a fused ring structure partly including a six-membered ring structure (the heteroaromatic compound having a fused ring structure) include a heteroaromatic compound having a quinoxaline ring, such as bathophenanthroline (abbreviation: BPhen), bathocuproine (abbreviation: BCP), 2,9-di(naphthalen-2-yl)-4,7-diphenyl-1,10-phenanthroline (abbreviation: NBPhen), 2,2′-(1,3-phenylene)bis(9-phenyl-1,10-phenanthroline) (abbreviation: mPPhen2P), 2,2′-(pyridin-2,6-diyl)bis(4-phenylbenzo[h]quinazoline) (abbreviation: 2,6(P-Bqn)₂Py), 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), or 2mpPCBPDBq.

For the electron-transport layers (114, 114a, and 114b), any of the metal complexes given below can be used as well as the heteroaromatic compounds described above. Examples of the metal complexes include a metal complex having a quinoline ring or a benzoquinoline ring, such as tris(8-quinolinolato)aluminum(III) (abbreviation: Alq₃), Almq₃, 8-quinolinolato-lithium (abbreviation: Liq), BeBq₂, bis(2-methyl-8-quinolinolato)(4-phenylphenolato)aluminum(III) (abbreviation: BAlq), or bis(8-quinolinolato)zinc(II) (abbreviation: Znq), and a metal complex having an oxazole ring or a thiazole ring, such as bis[2-(2-benzoxazolyl)phenolato]zinc(II) (abbreviation: ZnPBO) or bis[2-(2-benzothiazolyl)phenolato]zinc(II) (abbreviation: ZnBTZ).

High-molecular compounds such as poly(2,5-pyridinediyl) (abbreviation: PPy), poly[(9,9-dihexylfluorene-2,7-diyl)-co-(pyridine-3,5-diyl)] (abbreviation: PF-Py), and poly[(9,9-dioctylfluorene-2,7-diyl)-co-(2,2′-bipyridine-6,6′-diyl)] (abbreviation: PF-BPy) can be used as the electron-transport material.

Each of the electron-transport layers (114, 114a, and 114b) is not limited to a single layer and may be a stack of two or more layers each containing any of the above substances.

### <Electron-Injection Layer>

The electron-injection layers (115, 115a, and 115b) contain a substance having a high electron-injection property. The electron-injection layers (115, 115a, and 115b) are layers for increasing the efficiency of electron injection from the second electrode 102 and are preferably formed using a material whose value of the LUMO level has a small difference (0.50 eV or less) from the work function of a material used for the second electrode 102. Thus, the electron-injection layer 115 can be formed using an alkali metal, an alkaline earth metal, or a compound thereof, such as lithium, cesium, lithium fluoride (LiF), cesium fluoride (CsF), calcium fluoride (CaF₂), 8-quinolinolato-lithium (abbreviation: Liq), 2-(2-pyridyl)phenolatolithium (abbreviation: LiPP), 2-(2-pyridyl)-3-pyridinolatolithium (abbreviation: LiPPy), 4-phenyl-2-(2-pyridyl)phenolatolithium (abbreviation: LiPPP), an oxide of lithium (LiOₓ), or cesium carbonate. A rare earth metal such as ytterbium (Yb) and a compound of a rare earth metal such as erbium fluoride (ErF₃) can also be used. It is also possible to use a compound including a 1,3,4,6,7,8-tetrahydro-2H-pyrimido[1,2-a]pyrimidine skeleton, such as 1-(9,9′-spirobi[9H-fluoren]-2-yl)-1,3,4,6,7,8-hexahydro-2H-pyrimido[1,2-a]pyrimidine (abbreviation: 2hppSF), 1,1′-(9,9′-spirobi[9H-fluorene]-2,7-diyl)bis(1,3,4,6,7,8-hexahydro-2H-pyrimido[1,2-a]pyrimidine) (abbreviation: 2,7hpp2SF), or 1,1′-pyridine-2,6-diyl-bis(1,3,4,6,7,8-hexahydro-2H-pyrimido[1,2-a]pyrimidine) (abbreviation: hpp2Py). For the electron-injection layers (115, 115a, and 115b), a plurality of kinds of materials given above may be mixed or stacked as films. Electride may also be used for the electron-injection layers (115, 115a, and 115b). Examples of the electrode include a substance in which electrons are added at high concentration to calcium oxide-aluminum oxide. Any of the substances used for the electron-transport layers (114, 114a, and 114b), which are given above, can also be used.

A mixed material in which an organic compound and an electron donor (donor) are mixed may also be used for the electron-injection layers (115, 115a, and 115b). Such a mixed material is excellent in an electron-injection property and an electron-transport property because electrons are generated in the organic compound by the electron donor. The organic compound here is preferably a material excellent in transporting the generated electrons; specifically, for example, the above-described electron-transport materials used for the electron-transport layers (114, 114a, and 114b), such as a metal complex and a heteroaromatic compound, can be used. As the electron donor, a substance showing an electron-donating property with respect to an organic compound is preferably used. Specifically, an alkali metal, an alkaline earth metal, and a rare earth metal are preferable, and lithium, cesium, magnesium, calcium, erbium, ytterbium, and the like are given. In addition, an alkali metal oxide and an alkaline earth metal oxide are preferable, and lithium oxide, calcium oxide, barium oxide, and the like are given. Alternatively, a Lewis base such as magnesium oxide can be used. Further alternatively, an organic compound such as tetrathiafulvalene (abbreviation: TTF) can be used. Alternatively, a stack of two or more of these materials may be used.

A mixed material in which an organic compound and a metal are mixed may also be used for the electron-injection layers (115, 115a, and 115b). The organic compound used here preferably has a LUMO level higher than or equal to −3.60 eV and lower than or equal to −2.30 eV. Moreover, a material having an unshared electron pair is preferable.

Thus, as the organic compound used in the above mixed material, a mixed material obtained by mixing a metal and the heteroaromatic compound given above as the material that can be used for the electron-transport layer may be used. Preferred examples of the heteroaromatic compound include materials having an unshared electron pair, such as a heteroaromatic compound having a five-membered ring structure (e.g., an imidazole ring, a triazole ring, an oxazole ring, an oxadiazole ring, a thiazole ring, or a benzimidazole ring), a heteroaromatic compound having a six-membered ring structure (e.g., a pyridine ring, a diazine ring (including a pyrimidine ring, a pyrazine ring, a pyridazine ring, or the like), a triazine ring, a bipyridine ring, or a terpyridine ring), and a heteroaromatic compound having a fused ring structure partly including a six-membered ring structure (e.g., a quinoline ring, a benzoquinoline ring, a quinoxaline ring, a dibenzoquinoxaline ring, or a phenanthroline ring). Since the materials are specifically described above, description thereof is omitted here.

As a metal used for the above mixed material, a transition metal that belongs to Group 5, Group 7, Group 9, or Group 11 or a material that belongs to Group 13 in the periodic table is preferably used, and examples thereof include Ag, Cu, Al, and In. Here, the organic compound forms a SOMO with the transition metal.

To amplify light obtained from the light-emitting layer 113b, for example, the optical path length between the second electrode 102 and the light-emitting layer 113b is preferably less than one fourth of the wavelength λ of light emitted from the light-emitting layer 113b. In that case, the optical path length can be adjusted by changing the thickness of the electron-transport layer 114b or the electron-injection layer 115b.

When the two EL layers (103a and 103b) are provided and the charge-generation layer 106 is provided between the two EL layers as in the light-emitting device in FIG. 1E, a structure in which a plurality of EL layers are stacked between the pair of electrodes (the structure is also referred to as a tandem structure) can be obtained.

### <Charge-Generation Layer>

The charge-generation layer 106 has a function of injecting electrons into the EL layer 103a and injecting holes into the EL layer 103b when voltage is applied between the first electrode (anode) 101 and the second electrode (cathode) 102. The charge-generation layer 106 may be either a p-type layer in which an electron acceptor (acceptor) is added to a hole-transport material or an electron-injection buffer layer in which an electron donor (donor) is added to an electron-transport material. Alternatively, both of these layers may be stacked. Furthermore, an electron-relay layer may be provided between the p-type layer and the electron-injection buffer layer. Note that forming the charge-generation layer 106 with the use of any of the above materials can inhibit an increase in driving voltage in the stack of the EL layers.

In the case where the charge-generation layer 106 is a p-type layer in which an electron acceptor is added to a hole-transport material, which is an organic compound, any of the materials described in this embodiment can be used as the hole-transport material. Examples of the electron acceptor include 7,7,8,8-tetracyano-2,3,5,6-tetrafluoroquinodimethane (abbreviation: F₄-TCNQ) and chloranil. Other examples include oxides of metals that belong to Group 4 to Group 8 of the periodic table. Specific examples include vanadium oxide, niobium oxide, tantalum oxide, chromium oxide, molybdenum oxide, tungsten oxide, manganese oxide, and rhenium oxide. Any of the above-described acceptor materials may be used. Furthermore, a mixed film obtained by mixing materials of a p-type layer or a stack of films containing the respective materials may be used.

In the case where the charge-generation layer 106 an electron-injection buffer layer in which an electron donor is added to an electron-transport material, any of the materials described in this embodiment can be used as the electron-transport material. As the electron donor, it is possible to use an alkali metal, an alkaline earth metal, a rare earth metal, a metal belonging to Group 2 or Group 13 of the periodic table, or an oxide or a carbonate thereof. Specifically, lithium (Li), cesium (Cs), magnesium (Mg), calcium (Ca), ytterbium (Yb), indium (In), lithium oxide (Li₂O), cesium carbonate, or the like is preferably used. An alkali metal compound such as Liq may be used. An organic compound such as tetrathianaphthacene may be used as the electron donor. An organic compound including a 1,3,4,6,7,8-tetrahydro-2H-pyrimido[1,2-a]pyrimidine skeleton, such as 2hppSF, 2,7hpp2SF, or hpp2Py may be used as the electron donor. When any of these organic compounds is used as the electron donor, the electron-transport material to be combined with the electron donor is preferably an organic compound including a heteroaromatic ring having a phenanthroline ring, such as bathophenanthroline (abbreviation: BPhen), bathocuproine (abbreviation: BCP), 2,9-di(naphthalen-2-yl)-4,7-diphenyl-1,10-phenanthroline (abbreviation: NBphen), or 2,2′-(1,3-phenylene)bis(9-phenyl-1,10-phenanthroline) (abbreviation: mPPhen2P), in which case driving voltage of the light-emitting device can be decreased.

When an electron-relay layer is provided between the p-type layer and the electron-injection buffer layer in the charge-generation layer 106, the electron-relay layer contains at least a substance having an electron-transport property and has a function of preventing an interaction between the electron-injection buffer layer and the p-type layer and transferring electrons smoothly. The LUMO level of the substance having an electron-transport property in the electron-relay layer is preferably between the LUMO level of the acceptor substance in the p-type layer and the LUMO level of the substance having an electron-transport property in the electron-transport layer in contact with the charge-generation layer 106. Specifically, the LUMO level of the substance having an electron-transport property in the electron-relay layer can be higher than or equal to −5.00 eV, further preferably higher than or equal to −5.00 eV and lower than or equal to −3.00 eV. Note that as the substance having an electron-transport property in the electron-relay layer, a phthalocyanine-based material or a metal complex having a metal-oxygen bond and an aromatic ligand is preferably used.

Note that in terms of light extraction efficiency, the charge-generation layer 106 preferably has a property of transmitting visible light (specifically, the charge-generation layer 106 preferably has a visible light transmittance of 40% or more). The charge-generation layer 106 functions even if it has lower conductivity than the first electrode 101 or the second electrode 102.

Although FIG. 1E illustrates the structure in which two EL layers 103 are stacked, three or more EL layers may be stacked with charge-generation layers each provided between two adjacent EL layers.

### <Cap Layer>

Although not illustrated in FIGS. 1A to 1F, a cap layer may be provided over the second electrode 102 of the light-emitting device. For example, a material with high refractive index can be used for the cap layer. When the cap layer is provided over the second electrode 102, extraction efficiency of light emitted from the second electrode 102 can be improved.

Specific examples of a material that can be used for the cap layer include 5,5′-diphenyl-2,2′-di-5H-[1]benzothieno[3,2-c]carbazole (abbreviation: BisBTc), and 4,4′,4″-(benzene-1,3,5-triyl)tri(dibenzothiophene) (abbreviation: DBT3P-II).

### <Substrate>

The light-emitting device described in this embodiment can be formed over a variety of substrates. Note that the type of substrate is not limited to a certain type. Examples of the substrate include semiconductor substrates (e.g., a single crystal substrate or a silicon substrate), an SOI substrate, a glass substrate, a quartz substrate, a plastic substrate, a metal substrate, a stainless steel substrate, a substrate including stainless steel foil, a tungsten substrate, a substrate including tungsten foil, a flexible substrate, an attachment film, and paper and a base material film which include a fibrous material.

Examples of the glass substrate include a barium borosilicate glass substrate, an aluminoborosilicate glass substrate, and a soda lime glass substrate. Examples of the flexible substrate, the attachment film, and the base material film include plastics typified by polyethylene terephthalate (PET), polyethylene naphthalate (PEN), and polyether sulfone (PES), a synthetic resin such as acrylic resin, polypropylene, polyester, polyvinyl fluoride, polyvinyl chloride, polyamide, polyimide, aramid, epoxy resin, an inorganic vapor deposition film, and paper.

For fabrication of the light-emitting device in this embodiment, a gas phase method such as an evaporation method or a liquid phase method such as a spin coating method or an ink-jet method can be used. When an evaporation method is used, a physical vapor deposition method (PVD method) such as a sputtering method, an ion plating method, an ion beam evaporation method, a molecular beam evaporation method, or a vacuum evaporation method, a chemical vapor deposition method (CVD method), or the like can be used. Specifically, the layers having various functions (the hole-injection layer 111, the hole-transport layer 112, the light-emitting layer 113, the electron-transport layer 114, and the electron-injection layer 115) included in the EL layers of the light-emitting device can be formed by an evaporation method (e.g., a vacuum evaporation method), a coating method (e.g., a dip coating method, a die coating method, a bar coating method, a spin coating method, or a spray coating method), a printing method (e.g., an ink-jet method, screen printing (stencil), offset printing (planography), flexography (relief printing), gravure printing, or micro-contact printing), or the like.

In the case where a film formation method such as the coating method or the printing method is employed, a high-molecular compound (e.g., an oligomer, a dendrimer, or a polymer), a middle-molecular compound (a compound between a low-molecular compound and a high-molecular compound with a molecular weight of 400 to 4000), an inorganic compound (e.g., a quantum dot material), or the like can be used. The quantum dot material can be a colloidal quantum dot material, an alloyed quantum dot material, a core-shell quantum dot material, a core quantum dot material, or the like.

Materials that can be used for the layers (the hole-injection layer 111, the hole-transport layer 112, the light-emitting layer 113, the electron-transport layer 114, and the electron-injection layer 115) included in the EL layer 103 of the light-emitting device described in this embodiment are not limited to the materials described in this embodiment, and other materials can be used in combination as long as the functions of the layers are fulfilled.

The structures described in this embodiment can be used in combination with any of the structures described in the other embodiments as appropriate.

### Embodiment 3

This embodiment will describe a light-emitting and light-receiving apparatus 700 as a specific example of a light-emitting apparatus of one embodiment of the present invention and an example of the manufacturing method. Note that the light-emitting and light-receiving apparatus 700 includes both a light-emitting device and a light-receiving device, and can also be referred to as a light-emitting apparatus including a light-receiving device or a light-receiving apparatus including a light-emitting device. In addition, the light-emitting and light-receiving apparatus 700 can be used for a display portion of an electronic appliance or the like, and thus can also be referred to as a display panel or a display apparatus.

### <Structure Example of Light-Emitting and Light-Receiving Apparatus 700>

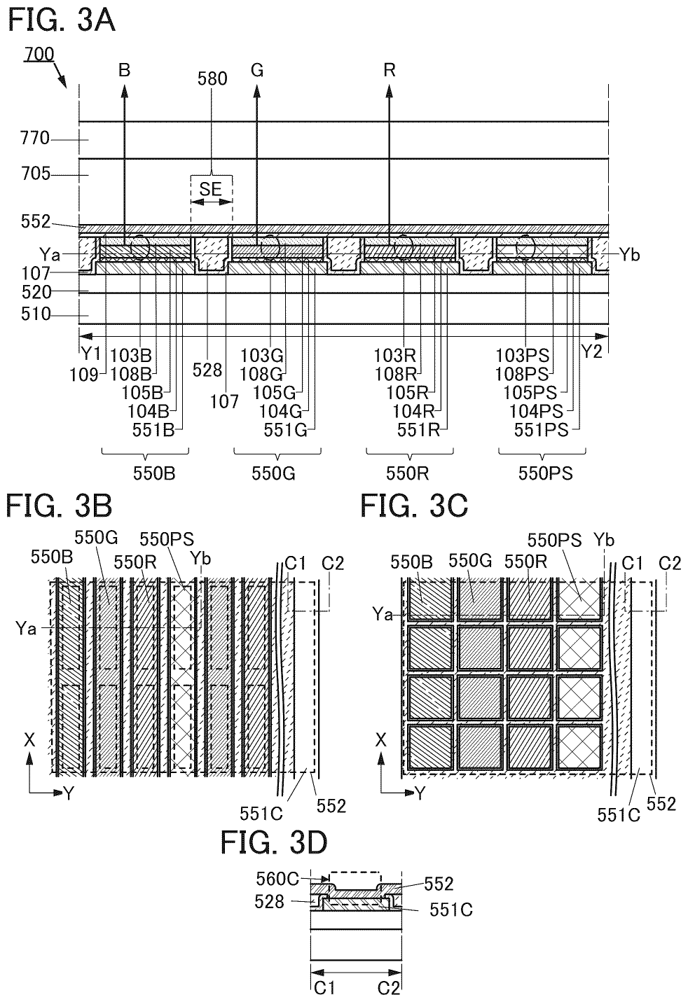

The light-emitting and light-receiving apparatus 700 illustrated in FIG. 3A includes a light-emitting device 550B, a light-emitting device 550G, a light-emitting device 550R, and a light-receiving device 550PS that are formed over a functional layer 520 over a first substrate 510. The functional layer 520 includes, for example, driver circuits such as a gate driver and a source driver that are composed of a plurality of transistors, and wirings that electrically connect these components. These driver circuits are electrically connected to the light-emitting device 550B, the light-emitting device 550G, the light-emitting device 550R, and the light-receiving device 550PS, for example, to drive them. The light-emitting and light-receiving apparatus 700 includes an insulating layer 705 over the functional layer 520 and the devices (the light-emitting devices and the light-receiving device), and the insulating layer 705 has a function of attaching a second substrate 770 and the functional layer 520.

At least one of the light-emitting devices 550B, 550G, and 550R has the device structure described in the foregoing embodiment. In addition, the structure of the EL layer 103 (see FIG. 1A) differs between the light-emitting devices. For example, a light-emitting layer 105B of the EL layer 103B can emit blue light, a light-emitting layer 105G of an EL layer 103G can emit green light, and a light-emitting layer 105R of an EL layer 103R can emit red light. For example, the light-emitting layer 105B can have the structure of the light-emitting layer described in the foregoing embodiment, and the light-emitting layers 105G and 105R can be formed using known materials.

Although the case where the devices (a plurality of light-emitting devices and a light-receiving device) are formed separately is described in this embodiment, part of an EL layer of a light-emitting device (a hole-injection layer, a hole-transport layer, and an electron-transport layer) and part of an active layer of a light-receiving device (the hole-injection layer, the hole-transport layer, and the electron-transport layer) may be formed using the same material at the same time in the manufacturing process.

In this specification and the like, a structure in which light-emitting layers in light-emitting devices of different colors (e.g., blue (B), green (G), and red (R)) and a light-receiving layer in a light-receiving device are separately formed or separately patterned is sometimes referred to as a side-by-side (SBS) structure. Although the light-emitting device 550B, the light-emitting device 550G, the light-emitting device 550R, and the light-receiving device 550PS are arranged in this order in the light-emitting and light-receiving apparatus 700 illustrated in FIG. 3A, one embodiment of the present invention is not limited to this structure.

In FIG. 3A, the light-emitting device 550B includes an electrode 551B, an electrode 552, and the EL layer 103B interposed between the electrode 551B and the electrode 552. The light-emitting device 550G includes an electrode 551G, the electrode 552, and the EL layer 103G interposed between the electrode 551G and the electrode 552. The light-emitting device 550R includes an electrode 551R, the electrode 552, and the EL layer 103R interposed between the electrode 551R and the electrode 552. The EL layers (103B, 103G, and 103R) each have a stacked-layer structure of layers having different functions including their respective light-emitting layers (105B, 105G, and 105R). Note that a specific structure of each layer of the light-emitting device is described in the foregoing embodiment, but is not limited.

In FIG. 3A, the light-receiving device 550PS includes an electrode 551PS, the electrode 552, and a light-receiving layer 103PS interposed between the electrode 551PS and the electrode 552. The light-receiving layer 103PS has a stacked-layer structure of layers having different functions including an active layer 105PS. The active layer 105PS contains a semiconductor. Examples of the semiconductor include inorganic semiconductors such as silicon and organic semiconductors such as organic compounds. Specific structures of other layers in the light-receiving device can be similar to the structures of the corresponding layers in the light-emitting device.

FIG. 3A illustrates a case where the EL layer 103B includes a hole-injection/transport layer 104B, the light-emitting layer 105B, an electron-transport layer 108B, and an electron-injection layer 109; the EL layer 103G includes a hole-injection/transport layer 104G, the light-emitting layer 105G, an electron-transport layer 108G, and the electron-injection layer 109; the EL layer 103R includes a hole-injection/transport layer 104R, the light-emitting layer 105R, an electron-transport layer 108R, and the electron-injection layer 109; and the light-receiving layer 103PS includes a hole-injection/transport layer 104PS, the active layer 105PS, an electron-transport layer 108PS, and the electron-injection layer 109. However, the present invention is not limited thereto.

In FIG. 3A, the electron-injection layer 109 and the electrode 552 are layers (common layers) shared by the devices (the light-emitting device 550B, the light-emitting device 550G, the light-emitting device 550R, and the light-receiving device 550PS).

Hereinafter, for simplicity, the light-emitting device 550B, the light-emitting device 550G, and the light-emitting device 550R are collectively referred to as a light-emitting device 550; the electrode 551B, the electrode 551G, and the electrode 551R are collectively referred to as an electrode 551; the EL layer 103B, the EL layer 103G, and the EL layer 103R are collectively referred to as the EL layer 103; the hole-injection/transport layer 104B, the hole-injection/transport layer 104G, and the hole-injection/transport layer 104R are collectively referred to as a hole-injection/transport layer 104; the light-emitting layer 105B, the light-emitting layer 105G, and the light-emitting layer 105R are collectively referred to as a light-emitting layer 105; and the electron-transport layer 108B, the electron-transport layer 108G, and the electron-transport layer 108R are collectively referred to as an electron-transport layer 108, in some cases.

As illustrated in FIG. 3A, an insulating layer 107 may be formed on the side surfaces (or end portions) of the hole-injection/transport layer 104, the light-emitting layer 105, and the electron-transport layer 108 included in the EL layer 103, and the side surfaces (or end portions) of the hole-injection/transport layer 104PS, the active layer 105PS, and the electron-transport layer 108PS included in the light-receiving layer 103PS. The insulating layer 107 is formed in contact with the side surfaces (or end portions) of the EL layer 103 and the light-receiving layer 103PS. This can inhibit entry of oxygen, moisture, or constituent elements thereof into the inside through the side surfaces of the EL layer 103 and the light-receiving layer 103PS. Note that the insulating layer 107 continuously covers the side surfaces of part of the EL layer 103 and part of the light-receiving layer 103PS of adjacent devices. For example, in FIG. 3A, the side surfaces of parts of the EL layer 103B of the light-emitting device 550B and the EL layer 103G of the light-emitting device 550G are covered with the continuous insulating layer 107.

As illustrated in FIG. 3A, a partition 528 is provided between the devices. Note that the electron-injection layer 109 and the electrode 552 that are common layers shared by the devices are provided continuously without being divided by the partition 528. Thus, the partition 528 can be regarded as being provided in a region surrounded by the electron-injection layer 109 and the insulating layer 107. In addition, the partitions 528 are positioned along the side surfaces (or end portions) of the electrode 551, part of the EL layer 103 (the hole-injection/transport layer 104, the light-emitting layer 105, and the electron-transport layer 108), and part of the light-receiving layer 103PS (the hole-injection/transport layer 104PS, the active layer 105PS, and the electron-transport layer 108PS) with the insulating layer 107 therebetween.

In each of the EL layer 103 and the light-receiving layer 103PS, particularly the hole-injection layer, which is included in the hole-transport region between the anode and the light-emitting layer and between the anode and the active layer, often has high conductivity; thus, a hole-injection layer formed as a layer shared by adjacent devices might cause crosstalk. Thus, as described in this structure example, part of the EL layer 103 (the hole-injection/transport layer 104, the light-emitting layer 105, and the electron-transport layer 108) and part of the light-receiving layer 103PS (the hole-injection/transport layer 104PS, the active layer 105PS, and the electron-transport layer 108PS) are separated, and the insulating layer 107 and the partition 528 are provided therebetween, so that crosstalk between adjacent devices can be inhibited.

Furthermore, a depression portion generated between adjacent devices can be flattened by provision of the partition 528. When the depression portion is flattened, disconnection of the electron-injection layer 109 and the electrode 552 formed over the EL layer 103 and the light-receiving layer 103PS can be inhibited.

For the insulating layer 107, aluminum oxide, magnesium oxide, hafnium oxide, gallium oxide, indium gallium zinc oxide, silicon nitride, or silicon nitride oxide can be used, for example. Some of the above-described materials may be stacked to form the insulating layer 107. The insulating layer 107 can be formed by a sputtering method, a CVD method, a molecular beam epitaxy (MBE) method, a pulsed laser deposition (PLD) method, an atomic layer deposition (ALD) method, or the like and is formed preferably by an ALD method, which achieves good coverage.

Examples of an insulating material used to form the partition 528 include organic materials such as an acrylic resin, a polyimide resin, an epoxy resin, an imide resin, a polyamide resin, a polyimide-amide resin, a silicone resin, a siloxane resin, a benzocyclobutene-based resin, a phenol resin, and precursors of these resins. Other examples include organic materials such as polyvinyl alcohol (PVA), polyvinyl butyral, polyvinyl pyrrolidone, polyethylene glycol, polyglycerin, pullulan, water-soluble cellulose, and an alcohol-soluble polyamide resin. A photosensitive resin such as a photoresist can also be used. Examples of the photosensitive resin include positive-type materials and negative-type materials.

With the use of the photosensitive resin, the partition 528 can be fabricated by only light exposure and developing steps. The partition 528 may be fabricated using a negative photosensitive resin (e.g., a resist material). In the case where an insulating layer containing an organic material is used as the partition 528, a material absorbing visible light is suitably used. When such a material absorbing visible light is used for the partition 528, light emission from the EL layer can be absorbed by the partition 528, leading to a reduction in light leakage (stray light) to an adjacent EL layer or light-receiving layer. Accordingly, a light-emitting and light-receiving apparatus with high display quality can be provided.

For example, the difference between the top-surface level of the partition 528 and the top-surface level of the EL layer 103 or the light-receiving layer 103PS is preferably 0.5 times or less, further preferably 0.3 times or less the thickness of the partition 528. The partition 528 may be provided such that the top-surface level of the EL layer 103 or the light-receiving layer 103PS is higher than the top-surface level of the partition 528, for example. Alternatively, the partition 528 may be provided such that the top-surface level of the partition 528 is higher than the top-surface level of the light-emitting layer of the EL layer 103B, the EL layer 103G, and the EL layer 103R or the active layer of the light-receiving layer 103PS, for example.

When crosstalk occurs between devices in a light-emitting and light-receiving apparatus with a high resolution exceeding 1000 ppi, a color gamut that the light-emitting and light-receiving apparatus can reproduce is narrowed. In a light-emitting and light-receiving apparatus with a high resolution more than 1000 ppi, preferably more than 2000 ppi, further preferably more than 5000 ppi, the insulating layer 107 and the partition 528 are provided between part of the EL layer 103 (the hole-injection/transport layer 104, the light-emitting layer 105B, and the electron-transport layer 108) and part of the light-receiving layer 103PS (the hole-injection/transport layer 104, the active layer 105PS, and the electron-transport layer 108), whereby the light-emitting and light-receiving apparatus can display bright colors.

FIGS. 3B and 3C are each a schematic top view of the light-emitting and light-receiving apparatus 700 taken along the dashed-dotted line Ya-Yb in the cross-sectional view of FIG. 3A. That is, the devices are arranged in a matrix. Note that FIG. 3B illustrates what is called a stripe arrangement, in which the light-emitting devices of the same color or the light-receiving devices are arranged in the X-direction. FIG. 3C illustrates a structure in which the light-emitting devices of the same color or the light-receiving devices are arranged in the X-direction and separated by patterning for each pixel. Note that the arrangement method of the light-emitting devices is not limited thereto; another method such as a delta, zigzag, PenTile, or diamond arrangement can also be used.

Note that part of the EL layer 103 (the hole-injection/transport layer 104, the light-emitting layer 105B, and the electron-transport layer 108) and part of the light-receiving layer 103PS (the hole-injection/transport layer 104, the active layer 105PS, and the electron-transport layer 108) are processed by patterning using a photolithography method for separation, so that a light-emitting and light-receiving apparatus (display panel) with a high resolution can be manufactured. The end portions (side surfaces) of the layers of part of the EL layer 103 and the layers of part of the light-receiving layer 103PS processed by patterning using a photolithography method have substantially one surface (or are positioned on substantially the same plane). In this case, the widths (SE) of spaces 580 between the EL layers and between the EL layer and the light-receiving layer are each preferably 5 μm or less, further preferably 1 μm or less.

FIG. 3D is a schematic cross-sectional view taken along the dashed-dotted line C1-C2 in FIGS. 3B and 3C. FIG. 3D illustrates a connection portion 560C where a connection electrode 551C and the electrode 552 are electrically connected to each other. In the connection portion 560C, the electrode 552 is provided over and in contact with the connection electrode 551C. The partition 528 is provided to cover an end portion of the connection electrode 551C.

### <Manufacturing Method Example of Light-Emitting and Light-Receiving Apparatus>

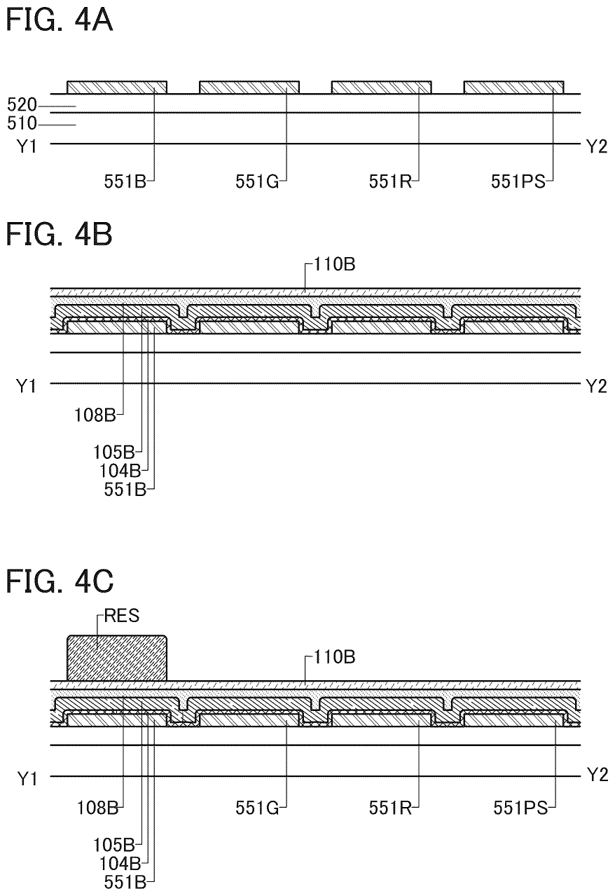

The electrode 551B, the electrode 551G, the electrode 551R, and the electrode 551PS are formed as illustrated in FIG. 4A. For example, a conductive film is formed over the functional layer 520 over the first substrate 510 and processed into predetermined shapes by a photolithography method.

The conductive film can be formed by any of a sputtering method, a CVD method, an MBE method, a vacuum evaporation method, a PLD method, an ALD method, and the like. Examples of the CVD method include a plasma-enhanced chemical vapor deposition (PECVD) method and a thermal CVD method. An example of a thermal CVD method is a metal organic CVD (MOCVD) method.

The conductive film may be processed by a nanoimprinting method, a sandblasting method, a lift-off method, or the like as well as a photolithography method described above. Alternatively, island-shaped thin films may be directly formed by a film formation method using a shielding mask such as a metal mask.

There are two typical examples of photolithography methods. In one of the methods, a resist mask is formed over a thin film to be processed, the thin film is processed by etching or the like, and then the resist mask is removed. In the other method, a photosensitive thin film is formed and then processed into a desired shape by light exposure and development. The former method involves heat treatment steps such as pre-applied bake (PAB) after resist application and post-exposure bake (PEB) after light exposure. In one embodiment of the present invention, a lithography method is used not only for processing of a conductive film but also for processing of a thin film used for formation of an EL layer (a film made of an organic compound or a film partly containing an organic compound).

As light for exposure in a photolithography method, it is possible to use light with the i-line (wavelength: 365 nm), light with the g-line (wavelength: 436 nm), light with the h-line (wavelength: 405 nm), or light in which the i-line, the g-line, and the h-line are mixed. Alternatively, ultraviolet light, KrF laser light, ArF laser light, or the like can be used. Exposure may be performed by liquid immersion exposure technique. As the light for exposure, extreme ultraviolet (EUV) light or X-rays may also be used. Instead of the light for exposure, an electron beam can be used. It is preferable to use EUV, X-rays, or an electron beam because extremely minute processing can be performed. Note that a photomask is not needed when light exposure is performed by scanning with a beam such as an electron beam.

For etching of a thin film using a resist mask, a dry etching method, a wet etching method, a sandblasting method, or the like can be used.

Subsequently, as illustrated in FIG. 4B, the hole-injection/transport layer 104B, the light-emitting layer 105B, and the electron-transport layer 108B are formed over the electrode 551B, the electrode 551G, the electrode 551R, and the electrode 551PS. Note that the hole-injection/transport layer 104B, the light-emitting layer 105B, and the electron-transport layer 108B can be formed by a vacuum evaporation method, for example. Furthermore, a sacrificial layer 110B is formed over the electron-transport layer 108B. For the formation of the hole-injection/transport layer 104B, the light-emitting layer 105B, and the electron-transport layer 108B, any of the materials described in the above embodiments can be used, for example.

For the sacrificial layer 110B, it is preferable to use a film highly resistant to etching treatment performed on the hole-injection/transport layer 104B, the light-emitting layer 105B, and the electron-transport layer 108B, i.e., a film having high etching selectivity with respect to the hole-injection/transport layer 104B, the light-emitting layer 105B, and the electron-transport layer 108B. The sacrificial layer 110B preferably has a stacked-layer structure of a first sacrificial layer and a second sacrificial layer that have different etching selectivities. For the sacrificial layer 110B, it is possible to use a film that can be removed by a wet etching method, which causes less damage to the EL layer 103B. In wet etching, oxalic acid or the like can be used as an etching material. Note that in this specification and the like, a sacrificial layer may be called a mask layer.

For the sacrificial layer 110B, an inorganic film such as a metal film, an alloy film, a metal oxide film, a semiconductor film, or an inorganic insulating film can be used, for example. The sacrificial layer 110B can be formed by any of a variety of film formation methods such as a sputtering method, an evaporation method, a CVD method, and an ALD method.

For the sacrificial layer 110B, a metal material such as gold, silver, platinum, magnesium, nickel, tungsten, chromium, molybdenum, iron, cobalt, copper, palladium, titanium, aluminum, yttrium, zirconium, or tantalum or an alloy material containing the metal material can be used, for example. It is particularly preferable to use a low-melting-point material such as aluminum or silver.

A metal oxide such as indium gallium zinc oxide (also referred to as In—Ga—Zn oxide or IGZO) can be used for the sacrificial layer 110B. It is also possible to use indium oxide, indium zinc oxide (In—Zn oxide), indium tin oxide (In—Sn oxide), indium titanium oxide (In—Ti oxide), indium tin zinc oxide (In—Sn—Zn oxide), indium titanium zinc oxide (In—Ti—Zn oxide), indium gallium tin zinc oxide (In—Ga—Sn—Zn oxide), or the like. Alternatively, indium tin oxide containing silicon can also be used, for example.

An element M (M is one or more of aluminum, silicon, boron, yttrium, copper, vanadium, beryllium, titanium, iron, nickel, germanium, zirconium, molybdenum, lanthanum, cerium, neodymium, hafnium, tantalum, tungsten, and magnesium) may be used instead of gallium. In particular, M is preferably one or more of gallium, aluminum, and yttrium.

For the sacrificial layer 110B, an inorganic insulating material such as aluminum oxide, hafnium oxide, or silicon oxide can be used.

The sacrificial layer 110B is preferably formed using a material that can be dissolved in a solvent chemically stable with respect to at least the electron-transport layer 108B that is in the uppermost position. Specifically, a material that can be dissolved in water or alcohol can be suitably used for the sacrificial layer 110B. In formation of the sacrificial layer 110B, it is preferable that application of such a material dissolved in a solvent such as water or alcohol be performed by a wet process and followed by heat treatment for evaporating the solvent. At this time, the heat treatment is preferably performed under a reduced-pressure atmosphere, in which case the solvent can be removed at a low temperature in a short time and thermal damage to the hole-injection/transport layer 104B, the light-emitting layer 105B, and the electron-transport layer 108B can be accordingly reduced.

In the case where the sacrificial layer 110B having a stacked-layer structure is formed, the stacked-layer structure can include the first sacrificial layer formed using any of the above-described materials and the second sacrificial layer thereover.

The second sacrificial layer in that case is a film used as a hard mask for etching of the first sacrificial layer. In processing the second sacrificial layer, the first sacrificial layer is exposed. Thus, a combination of films having greatly different etching rates is selected for the first sacrificial layer and the second sacrificial layer. Thus, a film that can be used for the second sacrificial layer can be selected in accordance with the etching conditions of the first sacrificial layer and those of the second sacrificial layer.

For example, in the case where the second sacrificial layer is etched by dry etching using a fluorine-containing gas (also referred to as a fluorine-based gas), the second sacrificial layer can be formed using silicon, silicon nitride, silicon oxide, tungsten, titanium, molybdenum, tantalum, tantalum nitride, an alloy containing molybdenum and niobium, an alloy containing molybdenum and tungsten, or the like. Here, a film of a metal oxide such as IGZO or ITO can be given as an example of a film having a high etching selectivity to the second sacrificial layer (i.e., a film with a low etching rate) in the dry etching using the fluorine-based gas, and can be used for the first sacrificial layer.

Note that the material for the second sacrificial layer is not limited to the above and can be selected from a variety of materials in accordance with the etching conditions of the first sacrificial layer and those of the second sacrificial layer. For example, any of the films that can be used for the first sacrificial layer can be used for the second sacrificial layer.

For the second sacrificial layer, a nitride film can be used, for example. Specifically, it is possible to use a nitride such as silicon nitride, aluminum nitride, hafnium nitride, titanium nitride, tantalum nitride, tungsten nitride, gallium nitride, or germanium nitride.

Alternatively, an oxide film can be used for the second sacrificial layer. Typically, it is possible to use a film of an oxide or an oxynitride such as silicon oxide, silicon oxynitride, aluminum oxide, aluminum oxynitride, hafnium oxide, or hafnium oxynitride.

Next, as illustrated in FIG. 4C, a resist is applied onto the sacrificial layer 110B, and the resist having a desired shape (a resist mask RES) is formed by a photolithography method. Such a method involves heat treatment steps such as pre-applied bake (PAB) after the resist application and post-exposure bake (PEB) after light exposure. The temperature reaches approximately 100° C. during the PAB, and approximately 120° C. during the PEB, for example. Therefore, the light-emitting device should be resistant to such high treatment temperatures.

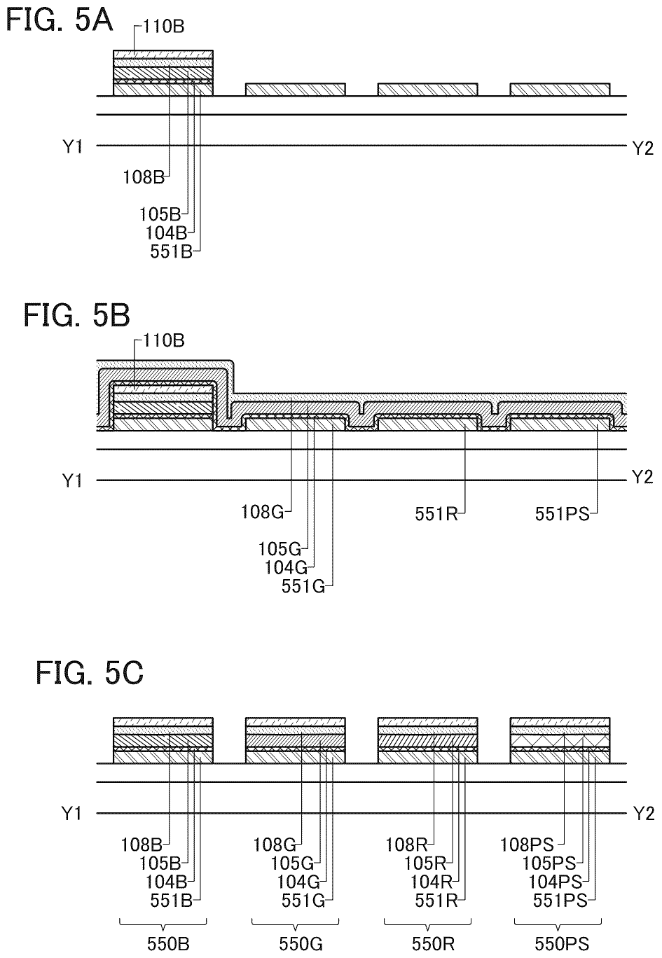

Next, part of the sacrificial layer 110B that is not covered with the resist mask RES is removed by etching using the resist mask RES, the resist mask RES is removed, and then the hole-injection/transport layer 104B, the light-emitting layer 105B, and the electron-transport layer 108B that are not covered with the sacrificial layer are partly removed by etching, so that the hole-injection/transport layer 104B, the light-emitting layer 105B, and the electron-transport layer 108B are processed to have side surfaces (or have their side surfaces exposed) over the electrode 551B or have belt-like shapes extending in the direction intersecting the sheet of the diagram. Note that dry etching can be used for the etching. Note that in the case where the sacrificial layer 110B has the aforementioned stacked-layer structure of the first sacrificial layer and the second sacrificial layer, the hole-injection/transport layer 104B, the light-emitting layer 105B, and the electron-transport layer 108B may be processed into a predetermined shape in the following manner: part of the second sacrificial layer is etched using the resist mask RES, the resist mask RES is then removed, and part of the first sacrificial layer is etched using the second sacrificial layer as a mask. The structure illustrated in FIG. 5A is obtained through these etching steps.

Subsequently, as illustrated in FIG. 5B, the hole-injection/transport layer 104G, the light-emitting layer 105G, and the electron-transport layer 108G are formed over the sacrificial layer 110B, the electrode 551G, the electrode 551R, and the electrode 551PS. The hole-injection/transport layer 104G, the light-emitting layer 105G, and the electron-transport layer 108G can be formed using any of the materials described in the above embodiments, for example. Note that the hole-injection/transport layer 104G, the light-emitting layer 105G, and the electron-transport layer 108G can be formed by a vacuum evaporation method, for example.

Hereinafter, in a manner similar to formation of the hole-injection/transport layer 104B, the light-emitting layer 105B, the electron-transport layer 108B, and the sacrificial layer 110B, the hole-injection/transport layer 104G, the light-emitting layer 105G, the electron-transport layer 108G, and a sacrificial layer 110G are formed over the electrode 551G, the hole-injection/transport layer 104R, the light-emitting layer 105R, the electron-transport layer 108R, and a sacrificial layer 110R are formed over the electrode 551R, and the hole-injection/transport layer 104PS, the active layer 105PS, the electron-transport layer 108PS, and a sacrificial layer 110PS are formed over the electrode 551PS, whereby the structure illustrated in FIG. 5C is obtained.

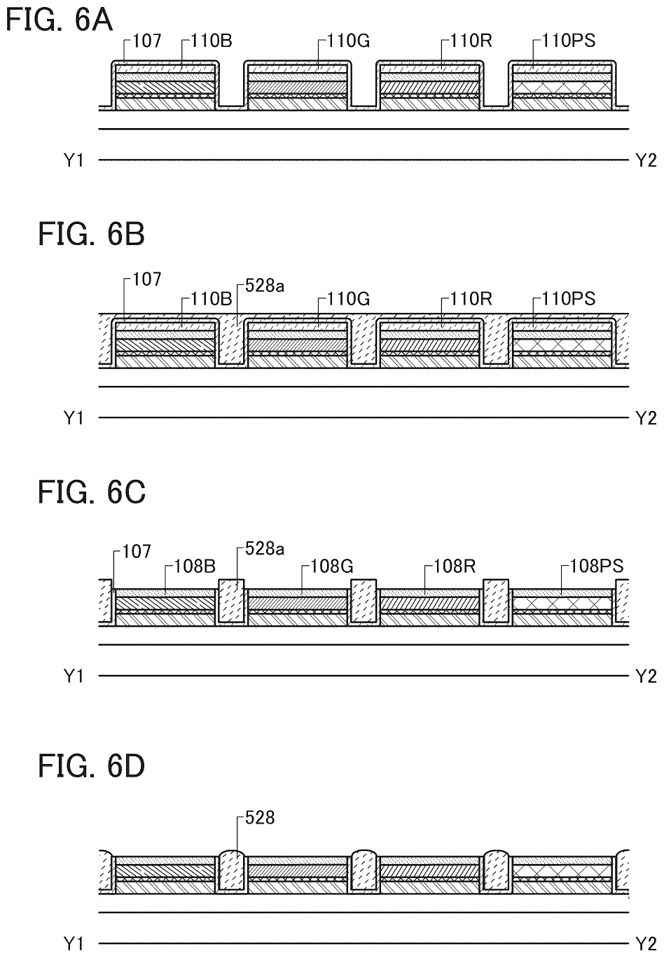

Next, as illustrated in FIG. 6A, the insulating layer 107 is formed over the sacrificial layers 110B, 110G, 110R, and HOPS.

Note that the insulating layer 107 can be formed by an ALD method, for example. In this case, as illustrated in FIG. 6A, the insulating layer 107 is formed to be in contact with the side surfaces (end portions) of the hole-injection/transport layers (104B, 104G, 104R, and 104PS), the light-emitting layers (105R, 105G, and 105R), the active layer 105PS, and the electron-transport layers (108B, 108G, 108R, and 108PS) of the devices. This can inhibit entry of oxygen, moisture, or constituent elements thereof into the inside through the side surfaces of the layers.

Next, as illustrated in FIG. 6B, a resin film 528a is formed over the insulating layer 107. As the resin film 528a, for example, a negative photosensitive resin or a positive photosensitive resin can be used.

Then, as illustrated in FIG. 6C, part of the resin film 528a, part of the insulating layer 107, and the sacrificial layers (110B, 110G, 110R, and HOPS) are removed to expose the top surfaces of the electron-transport layers (108B, 108G, 108R, and 108PS).

Next, heat treatment is performed to process an upper edge portion of the resin film 528a into a curved shape, so that the partition 528 is formed, as illustrated in FIG. 6D. When the upper edge portion of the partition 528 has a curved shape, good coverage with the electron-injection layer 109 to be formed later can be obtained. For example, in the case of using a positive photosensitive acrylic resin as a material for the resin film 528a, the partition 528 is preferably formed so as to have a curved surface with a curvature radius (greater than or equal to 0.2 μm and less than or equal to 3 μm) at the upper edge portion.

Next, the electron-injection layer 109 is formed over the insulating layer 107, the electron-transport layers (108B, 108G, 108R, and 108PS), and the partition 528. The electron-injection layer 109 can be formed using any of the materials described in the above embodiments. The electron-injection layer 109 is formed by a vacuum evaporation method, for example.

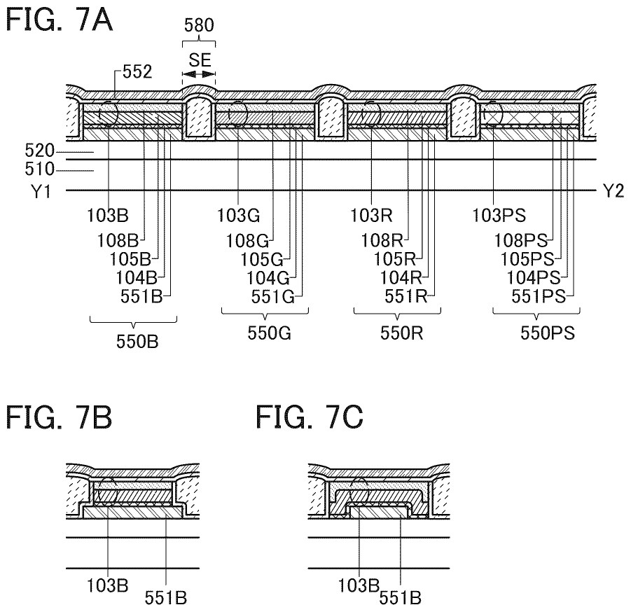

Next, as illustrated in FIG. 7A, the electrode 552 is formed over the electron-injection layer 109. The electrode 552 is formed by a vacuum evaporation method, for example.

Through the above steps, the EL layer 103B, the EL layer 103G, the EL layer 103R, and the light-receiving layer 103PS in the light-emitting device 550B, the light-emitting device 550G, the light-emitting device 550R, and the light-receiving device 550PS can be processed to be separated from each other.

Pattern formation by a photolithography method is performed in separate processing of the EL layer 103 and the light-receiving layer 103PS in the above manner, so that a light-emitting and light-receiving apparatus (display panel) with a high resolution can be manufactured. The end portions (side surfaces) of the layers of the EL layer and the light-receiving layer processed by patterning using a photolithography method have substantially one surface (or are positioned on substantially the same plane). The pattern formation by a photolithography method can inhibit crosstalk between adjacent light-emitting devices and between the light-emitting device and the light-receiving device. In addition, the space 580 is provided between the end portions (side surfaces) of adjacent devices processed by patterning using a photolithography method. In FIG. 7C, when the space 580 is denoted by a distance SE between the EL layers of adjacent light-emitting devices, decreasing the distance SE can increase the aperture ratio and the resolution. By contrast, as the distance SE is increased, the effect of the difference in the manufacturing process between the adjacent light-emitting devices becomes permissible, which leads to an increase in manufacturing yield. Since the light-emitting device manufactured according to this specification is suitable for a miniaturization process, the distance SE between the EL layers of adjacent light-emitting devices can be longer than or equal to 0.5 μm and shorter than or equal to 5 μm, preferably longer than or equal to 1 μm and shorter than or equal to 3 μm, further preferably longer than or equal to 1 μm and shorter than or equal to 2.5 μm, still further preferably longer than or equal to 1 μm and shorter than or equal to 2 μm. Typically, the distance SE is preferably longer than or equal to 1 μm and shorter than or equal to 2 μm (e.g., 1.5 μm or a neighborhood thereof).

In this specification and the like, a device formed using a metal mask or a fine metal mask (FMM) is sometimes referred to as a device having a metal mask (MM) structure. In this specification and the like, a device formed without using a metal mask or an FMM is sometimes referred to as a device having a metal maskless (MML) structure. Since a light-emitting and light-receiving apparatus having the MML structure is formed without using a metal mask, the pixel arrangement, the pixel shape, and the like can be designed more flexibly than in a light-emitting and light-receiving apparatus having the FMM structure or the MM structure.

Note that the island-shaped EL layers of the light-emitting and light-receiving apparatus having the MML structure are formed by not patterning using a metal mask but processing after deposition of an EL layer. Thus, a light-emitting and light-receiving apparatus with a higher resolution or a higher aperture ratio than a conventional one can be achieved. Moreover, EL layers can be formed separately for each color, which enables extremely clear images; thus, a light-emitting and light-receiving apparatus with a high contrast and high display quality can be achieved. Furthermore, provision of a sacrificial layer over an EL layer can reduce damage on the EL layer during the manufacturing process and increase the reliability of the light-emitting device.

In FIG. 3A and FIG. 7A, the width of the EL layer 103 is substantially equal to that of the electrode 551 in the light-emitting device 550, and the width of the light-receiving layer 103PS is substantially equal to that of the electrode 551PS in the light-receiving device 550PS; however, one embodiment of the present invention is not limited thereto.

In the light-emitting device 550, the width of the EL layer 103 may be smaller than that of the electrode 551. In the light-receiving device 550PS, the width of the light-receiving layer 103PS may be smaller than that of the electrode 551PS. FIG. 7B illustrates an example in which the width of the EL layer 103B is smaller than that of the electrode 551B in the light-emitting device 550B.

In the light-emitting device 550, the width of the EL layer 103 may be larger than that of the electrode 551. In the light-receiving device 550PS, the width of the light-receiving layer 103PS may be larger than that of the electrode 551PS. FIG. 7C illustrates an example in which the width of the EL layer 103B is larger than that of the electrode 551R in the light-emitting device 550B.

The structures described in this embodiment can be used in combination with any of the structures described in the other embodiments as appropriate.

### Embodiment 4

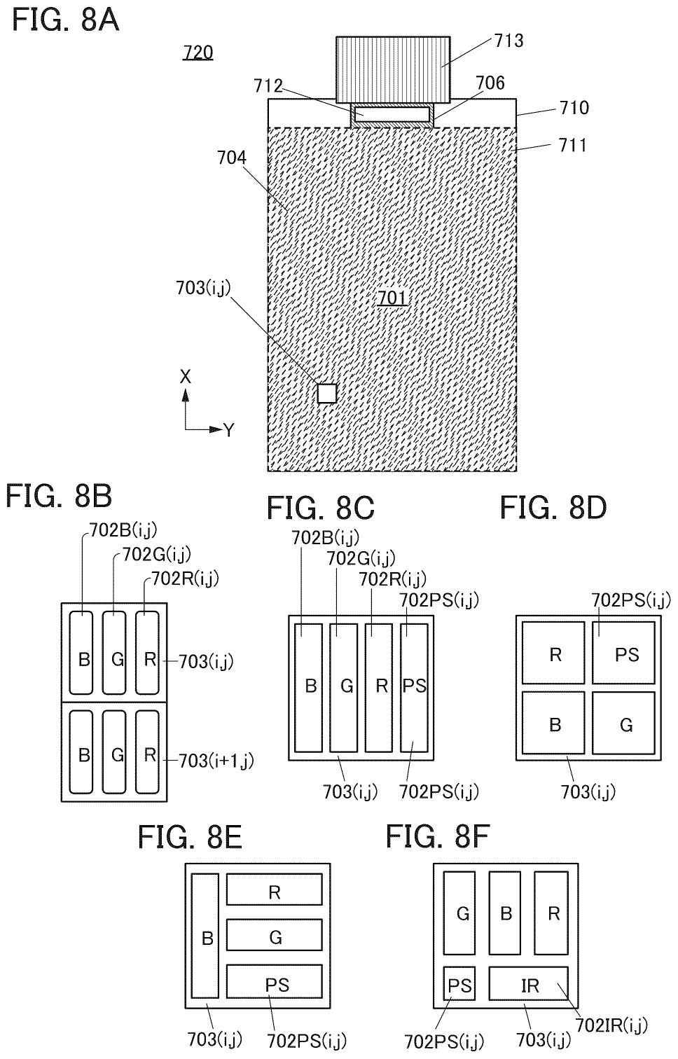

In this embodiment, an apparatus 720 is described with reference to FIGS. 8A to 8F, FIGS. 9A to 9C, and FIG. 10. The apparatus 720 illustrated in FIGS. 8A to 8F, FIGS. 9A to 9C, and FIG. 10 includes any of the light-emitting devices described in the above embodiment and therefore is a light-emitting apparatus. Furthermore, the apparatus 720 described in this embodiment can be used in a display unit of an electronic device or the like and therefore can also be referred to as a display panel or a display device. Moreover, when the apparatus 720 includes the light-emitting device as a light source and a light-receiving device that can receive light from the light-emitting device, the apparatus 720 can be referred to as a light-emitting and light-receiving apparatus. Note that the light-emitting apparatus, the display panel, the display device, and the light-emitting and light-receiving apparatus each include at least a light-emitting device.

Furthermore, the light-emitting apparatus, the display panel, the display device, and the light-emitting and light-receiving apparatus of this embodiment can each have high definition or a large size. Therefore, the light-emitting apparatus, the display panel, the display device, and the light-emitting and light-receiving apparatus of this embodiment can be used, for example, in display units of electronic devices such as a digital camera, a digital video camera, a digital photo frame, a mobile phone, a portable game console, a smart phone, a wristwatch terminal, a tablet terminal, a portable information terminal, and an audio reproducing apparatus, in addition to display units of electronic devices with a relatively large screen, such as a television device, a desktop or laptop personal computer, a monitor of a computer or the like, digital signage, and a large game machine such as a pachinko machine.

FIG. 8A is a top view of the apparatus 720 (e.g., the light-emitting apparatus, the display panel, the display device, and the light-emitting and light-receiving apparatus).

In FIG. 8A, the apparatus 720 has a structure in which a substrate 710 and a substrate 711 are attached to each other. In addition, the apparatus 720 includes a display region 701, a circuit 704, a wiring 706, and the like. Note that the display region 701 includes a plurality of pixels. As illustrated in FIG. 8B, a pixel 703(i, j) illustrated in FIG. 8A and a pixel 703(i+1, j) are adjacent to each other.

Furthermore, in the example of the apparatus 720 illustrated in FIG. 8A, the substrate 710 is provided with an integrated circuit (IC) 712 by a chip on glass (COG) method, a chip on film (COF) method, or the like. As the IC 712, an IC including a scan line driver circuit, a signal line driver circuit, or the like can be used, for example. In the example illustrated in FIG. 8A, an IC including a signal line driver circuit is used as the IC 712, and a scan line driver circuit is used as the circuit 704.

The wiring 706 has a function of supplying signals and power to the display region 701 and the circuit 704. The signals and power are input to the wiring 706 from the outside through a flexible printed circuit (FPC) 713 or to the wiring 706 from the IC 712. Note that the apparatus 720 is not necessarily provided with the IC. The IC may be mounted on the FPC by a COF method or the like.

FIG. 8B illustrates the pixel 703(i, j) and the pixel 703(i+1, j) of the display region 701. A plurality of kinds of subpixels including light-emitting devices that emit different color light from each other can be included in the pixel 703(i, j). Alternatively, a plurality of subpixels including light-emitting devices that emit the same color light may be included in addition to those described above. Three kinds of subpixels can be included, for example. The three subpixels can be of three colors of red (R), green (G), and blue (B) or of three colors of yellow (Y), cyan (C), and magenta (M), for example. Alternatively, the pixel can include four kinds of subpixels. The four subpixels can be of four colors of R, G, B, and white (W) or of four colors of R, G, B, and Y, for example. Specifically, the pixel 703(i, j) can consist of a subpixel 702B(i, j) for blue display, a subpixel 702G(i, j) for green display, and a subpixel 702R(i, j) for red display.

The subpixel may include not only a light-emitting device but also a light-receiving device. Note that when the subpixel includes a light-receiving device, the apparatus 720 is also referred to as a light-emitting and light-receiving apparatus.

FIGS. 8C to 8F illustrate various layout examples of the pixel 703(i, j) including a subpixel 702PS(i, j) including a light-receiving device. The pixel arrangement in FIG. 8C is stripe arrangement, and the pixel arrangement in FIG. 8D is matrix arrangement. The pixel arrangement in FIG. 8E has a structure where three subpixels (the subpixels R, G, and PS) are vertically arranged next to one subpixel (the subpixel B). In the pixel arrangement in FIG. 8F, the vertically oriented three subpixels G, B, and R are arranged laterally, and the subpixel PS and the horizontally oriented subpixel IR are arranged laterally below the three subpixels. Note that the wavelength of light detected by the subpixel 702PS(i, j) is not particularly limited; however, the light-receiving device included in the subpixel 702PS(i, j) preferably has sensitivity to light emitted from the light-emitting device included in the subpixel 702R(i, j), the subpixel 702G(i, j), the subpixel 702B(i,j), or the subpixel 702IR(i,j). For example, the light-receiving device preferably detects one or more kinds of light in blue, violet, bluish violet, green, yellowish green, yellow, orange, red, and infrared wavelength ranges, for example.

Furthermore, as illustrated in FIG. 8F, a subpixel 702IR(i, j) that emits infrared rays may be added to any of the above-described sets of subpixels in the pixel 703(i, j). Specifically, the subpixel that emits light including light with a wavelength ranging from 650 nm to 1000 nm, inclusive, may be used in the pixel 703(i, j).

Note that the arrangement of subpixels is not limited to the structures illustrated in FIGS. 8A to 8F and a variety of arrangement methods can be employed. The arrangement of subpixels may be stripe arrangement, S stripe arrangement, matrix arrangement, delta arrangement, Bayer arrangement, or pentile arrangement, for example.

Furthermore, top surfaces of the subpixels may have a triangular shape, a quadrangular shape (including a rectangular shape and a square shape), a polygonal shape such as a pentagonal shape, a polygonal shape with rounded corners, an elliptical shape, or a circular shape, for example. The top surface shape of a subpixel herein refers to a top surface shape of a light-emitting region of a light-emitting device.

Furthermore, in the case where not only a light-emitting device but also a light-receiving device is included in a pixel, the pixel has a light-receiving function and thus can detect a contact or approach of an object while displaying an image. For example, an image can be displayed by using all the subpixels included in a light-emitting apparatus; or light can be emitted by some of the subpixels as a light source and an image can be displayed by using the remaining subpixels.

Note that the light-receiving area of the subpixel 702PS(i, j) is preferably smaller than the light-emitting areas of the other subpixels. A smaller light-receiving area leads to a narrower image-capturing range, inhibits a blur in a captured image, and improves the definition. Thus, by using the subpixel 702PS(i, j), high-resolution or high-definition image capturing is possible. For example, image capturing for personal authentication with the use of a fingerprint, a palm print, the iris, the shape of a blood vessel (including the shape of a vein and the shape of an artery), a face, or the like is possible by using the subpixel 702PS(i, j).

Moreover, the subpixel 702PS(i, j) can be used in a touch sensor (also referred to as a direct touch sensor), a near touch sensor (also referred to as a hover sensor, a hover touch sensor, a contactless sensor, or a touchless sensor), or the like. For example, the subpixel 702PS(i, j) preferably detects infrared light. Thus, touch sensing is possible even in a dark place.

Here, the touch sensor or the near touch sensor can detect an approach or contact of an object (e.g., a finger, a hand, or a pen). The touch sensor can detect the object when the light-emitting and light-receiving apparatus and the object come in direct contact with each other. Furthermore, the near touch sensor can detect the object even when the object is not in contact with the light-emitting and light-receiving apparatus. For example, the light-emitting and light-receiving apparatus can preferably detect the object when the distance between the light-emitting and light-receiving apparatus and the object is more than or equal to 0.1 mm and less than or equal to 300 mm, preferably more than or equal to 3 mm and less than or equal to 50 mm. With this structure, the light-emitting and light-receiving apparatus can be controlled without the object directly contacting with the light-emitting and light-receiving apparatus. In other words, the light-emitting and light-receiving apparatus can be controlled in a contactless (touchless) manner. With the above-described structure, the light-emitting and light-receiving apparatus can be controlled with a reduced risk of being dirty or damaged, or without direct contact between the object and a dirt (e.g., dust, bacteria, or a virus) attached to the light-emitting and light-receiving apparatus.

For high-resolution image capturing, the subpixel 702PS(i, j) is preferably provided in every pixel included in the light-emitting and light-receiving apparatus. Meanwhile, in the case where the subpixel 702PS(i, j) is used in a touch sensor, a near touch sensor, or the like, high accuracy is not required as compared to the case of capturing an image of a fingerprint or the like; accordingly, the subpixel 702PS(i, j) is provided in some subpixels in the light-emitting and light-receiving apparatus. When the number of subpixels 702PS(i, j) included in the light-emitting and light-receiving apparatus is smaller than the number of subpixels 702R(i, j) or the like, higher detection speed can be achieved.

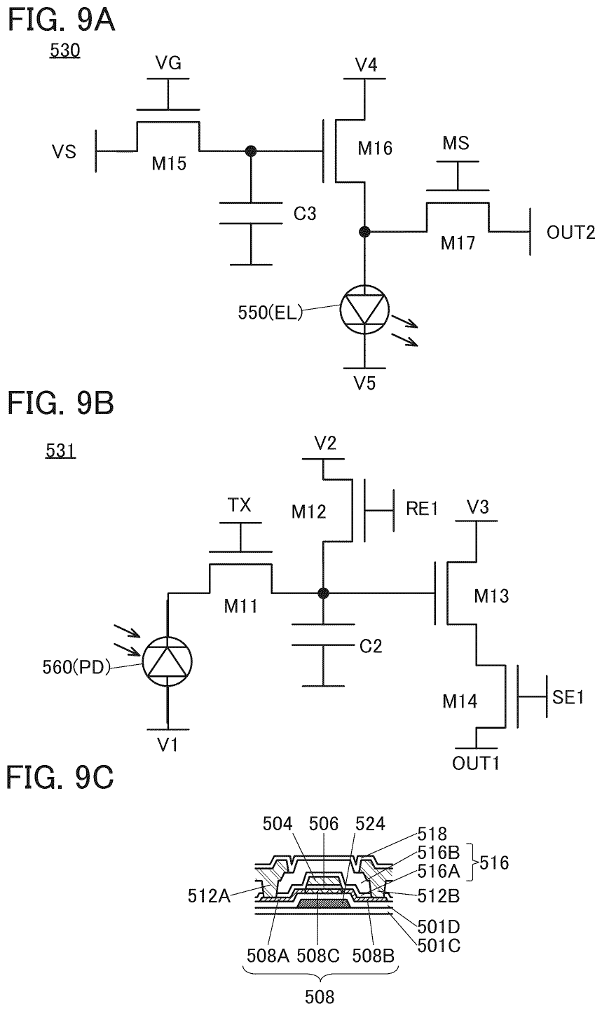

Next, an example of a pixel circuit of a subpixel included in the light-emitting device is described with reference to FIG. 9A. A pixel circuit 530 illustrated in FIG. 9A includes a light-emitting device (EL) 550, a transistor M15, a transistor M16, a transistor M17, and a capacitor C3. Note that a light-emitting diode can be used as the light-emitting device (EL) 550. In particular, the light-emitting device described in the above embodiment is preferably used as the light-emitting device (EL) 550.

In FIG. 9A, a gate of the transistor M15 is electrically connected to a wiring VG, one of a source and a drain of the transistor M15 is electrically connected to a wiring VS, and the other of the source and the drain of the transistor M15 is electrically connected to one electrode of the capacitor C3 and a gate of the transistor M16. One of a source and a drain of the transistor M16 is electrically connected to a wiring V4, and the other is electrically connected to an anode of the light-emitting device (EL) 550 and one of a source and a drain of the transistor M17. A gate of the transistor M17 is electrically connected to a wiring MS, and the other of the source and the drain of the transistor M17 is electrically connected to a wiring OUT2. A cathode of the light-emitting device (EL) 550 is electrically connected to a wiring V5.

A constant potential is supplied to the wiring V4 and the wiring V5. In the light-emitting device (EL) 550, the anode side can have a high potential and the cathode side can have a lower potential than the anode side. The transistor M15 is controlled by a signal supplied to the wiring VG and functions as a selection transistor for controlling a selection state of the pixel circuit 530. The transistor M16 functions as a driving transistor that controls a current flowing through the light-emitting device (EL) 550 in accordance with a potential supplied to the gate of the transistor M16. When the transistor M15 is on, a potential supplied to the wiring VS is supplied to the gate of the transistor M16, and the luminance of the light-emitting device (EL) 550 can be controlled in accordance with the potential. The transistor M17 is controlled by a signal supplied to the wiring MS and has a function of outputting a potential between the transistor M16 and the light-emitting device (EL) 550 to the outside through the wiring OUT2.

Here, a transistor in which a metal oxide (an oxide semiconductor) is used in a semiconductor layer where a channel is formed is preferably used as the transistors M11, M12, M13, and M14 included in the pixel circuit 531 in FIG. 9B and the transistors M15, M16, and M17 included in the pixel circuit 530 in FIG. 9A.

A transistor using a metal oxide having a wider band gap and a lower carrier density than silicon can achieve an extremely low off-state current. Such a low off-state current enables retention of charges accumulated in a capacitor that is connected in series to the transistor for a long time. Therefore, it is particularly preferable to use a transistor containing an oxide semiconductor as the transistors M11, M12, and M15 each of which is connected in series to a capacitor C2 or the capacitor C3. When each of the other transistors also includes an oxide semiconductor, manufacturing cost can be reduced.

Alternatively, transistors using silicon as a semiconductor in which a channel is formed can be used as the transistors M11 to M17. In particular, it is preferable to use silicon with high crystallinity such as single crystal silicon or polycrystalline silicon because high field-effect mobility can be achieved and higher-speed operation can be performed.

Alternatively, a transistor containing an oxide semiconductor may be used as at least one of the transistors M11 to M17, and transistors containing silicon may be used as the other transistors.

Next, an example of a pixel circuit of a subpixel including a light-receiving device is described with reference to FIG. 9B. The pixel circuit 531 illustrated in FIG. 9B includes a light-receiving device (PD) 560, the transistor M11, the transistor M12, the transistor M13, the transistor M14, and the capacitor C2. For example, a photodiode is used as the light-receiving device (PD) 560.

In FIG. 9B, an anode of the light-receiving device (PD) 560 is electrically connected to a wiring V1, and a cathode of the light-receiving device (PD) 560 is electrically connected to one of a source and a drain of the transistor M11. A gate of the transistor M11 is electrically connected to a wiring TX, and the other of the source and the drain of the transistor M11 is electrically connected to one electrode of the capacitor C2, one of a source and a drain of the transistor M12, and a gate of the transistor M13. A gate of the transistor M12 is electrically connected to a wiring RE1, and the other of the source and the drain of the transistor M12 is electrically connected to a wiring V2. One of a source and a drain of the transistor M13 is electrically connected to a wiring V3, and the other of the source and the drain of the transistor M13 is electrically connected to one of a source and a drain of the transistor M14. A gate of the transistor M14 is electrically connected to a wiring SE1, and the other of the source and the drain of the transistor M14 is electrically connected to a wiring OUT1.

A constant potential is supplied to the wiring V1, the wiring V2, and the wiring V3. When the light-receiving device (PD) 560 is driven with a reverse bias, the wiring V2 is supplied with a potential higher than the potential of the wiring V1. The transistor M12 is controlled by a signal supplied to the wiring RE1 and has a function of resetting the potential of a node connected to the gate of the transistor M13 to a potential supplied to the wiring V2. The transistor M11 is controlled by a signal supplied to the wiring TX and has a function of controlling the timing at which the potential of the node changes, in accordance with a current flowing through the light-receiving device (PD) 560. The transistor M13 functions as an amplifier transistor for outputting a signal corresponding to the potential of the node. The transistor M14 is controlled by a signal supplied to the wiring SE1 and functions as a selection transistor for reading an output corresponding to the potential of the node by an external circuit connected to the wiring OUT1.

Although n-channel transistors are illustrated in FIGS. 9A and 9B, p-channel transistors can be used instead.

The transistors included in the pixel circuit 530 and the transistors included in the pixel circuit 531 are preferably formed side by side over the same substrate. Preferably, the transistors included in the pixel circuit 530 and the transistors included in the pixel circuit 531 are periodically arranged in one region, in particular.

One or more layers including the transistor and/or the capacitor are preferably provided to overlap with the light-receiving device (PD) 560 or the light-emitting device (EL) 550. Thus, the effective area of each pixel circuit can be reduced, and a high-resolution light-receiving unit or display unit can be achieved.

FIG. 9C illustrates an example of a specific structure of a transistor that can be used in the pixel circuit described with reference to FIGS. 9A and 9B. As the transistor, a bottom-gate transistor, a top-gate transistor, or the like can be used as appropriate.

The transistor illustrated in FIG. 9C includes a semiconductor film 508, a conductive film 504, an insulating film 506, a conductive film 512A, and a conductive film 512B. The transistor is formed over an insulating film 501C, for example. The transistor also includes an insulating film 516 (an insulating film 516A and an insulating film 516B) and an insulating film 518.

The semiconductor film 508 includes a region 508A electrically connected to the conductive film 512A and a region 508B electrically connected to the conductive film 512B. The semiconductor film 508 includes a region 508C between the region 508A and the region 508B.

The conductive film 504 includes a region overlapping with the region 508C and has a function of a gate electrode.

The insulating film 506 includes a region positioned between the semiconductor film 508 and the conductive film 504. The insulating film 506 has a function of a first gate insulating film.

The conductive film 512A has one of a function of a source electrode and a function of a drain electrode, and the conductive film 512B has the other thereof.

A conductive film 524 can be used in the transistor. The semiconductor film 508 is sandwiched between the conductive film 504 and a region included in the conductive film 524. The conductive film 524 has a function of a second gate electrode. An insulating film 501D is positioned between the semiconductor film 508 and the conductive film 524 and has a function of a second gate insulating film.

The insulating film 516 functions as, for example, a protective film covering the semiconductor film 508. Specifically, a film including a silicon oxide film, a silicon oxynitride film, a silicon nitride oxide film, a silicon nitride film, an aluminum oxide film, a hafnium oxide film, an yttrium oxide film, a zirconium oxide film, a gallium oxide film, a tantalum oxide film, a magnesium oxide film, a lanthanum oxide film, a cerium oxide film, or a neodymium oxide film can be used as the insulating film 516, for example.

For the insulating film 518, a material that has a function of inhibiting diffusion of oxygen, hydrogen, water, an alkali metal, an alkaline earth metal, and the like is preferably used. Specifically, the insulating film 518 can be formed using silicon nitride, silicon oxynitride, aluminum nitride, or aluminum oxynitride, for example. In each of silicon oxynitride and aluminum oxynitride, the number of nitrogen atoms contained is preferably larger than the number of oxygen atoms contained.

Note that in a step of forming the semiconductor film used in the transistor of the pixel circuit, the semiconductor film used in the transistor of the driver circuit can be formed. A semiconductor film having the same composition as the semiconductor film used in the transistor of the pixel circuit can be used in the driver circuit, for example.

The semiconductor film 508 preferably contains indium, M (M is one or more of gallium, aluminum, silicon, boron, yttrium, tin, copper, vanadium, beryllium, titanium, iron, nickel, germanium, zirconium, molybdenum, lanthanum, cerium, neodymium, hafnium, tantalum, tungsten, and magnesium), and zinc, for example. Specifically, M is preferably one or more of aluminum, gallium, yttrium, and tin.

In particular, an oxide containing In, Ga, and Zn (also referred to as IGZO) is preferably used as the semiconductor film 508. Alternatively, it is preferable to use an oxide containing In, Sn, and Zn. Further alternatively, it is preferable to use an oxide containing In, Ga, Sn, and Zn. Further alternatively, it is preferable to use an oxide containing In, Al, and Zn (also referred to as IAZO). Further alternatively, it is preferable to use an oxide containing In, Al, Ga, and Zn (also referred to as IAGZO).

When the semiconductor film is an In-M-Zn oxide, the atomic ratio of In is preferably greater than or equal to the atomic ratio of Min the In-M-Zn oxide. Examples of the atomic ratio of the metal elements in such an In-M-Zn oxide are In:M:Zn=1:1:1, 1:1:1.2, 1:3:2, 1:3:4, 2:1:3, 3:1:2, 4:2:3, 4:2:4.1, 5:1:3, 5:1:6, 5:1:7, 5:1:8, 6:1:6, and 5:2:5 and a composition in the vicinity of any of the above atomic ratios. Note that the vicinity of the atomic ratio includes ±30% of an intended atomic ratio.

For example, when the atomic ratio is described as In:Ga:Zn=4:2:3 or a composition in the vicinity thereof, the case is included where the atomic proportion of Ga is greater than or equal to 1 and less than or equal to 3 and the atomic proportion of Zn is greater than or equal to 2 and less than or equal to 4 with the atomic proportion of In being 4. In addition, when the atomic ratio is described as In:Ga:Zn=5:1:6 or a composition in the vicinity thereof, the case is included where the atomic proportion of Ga is greater than 0.1 and less than or equal to 2 and the atomic proportion of Zn is greater than or equal to 5 and less than or equal to 7 with the atomic proportion of In being 5. Furthermore, when the atomic ratio is described as In:Ga:Zn=1:1:1 or a composition in the vicinity thereof, the case is included where the atomic proportion of Ga is greater than 0.1 and less than or equal to 2 and the atomic proportion of Zn is greater than 0.1 and less than or equal to 2 with the atomic proportion of In being 1.

There is no particular limitation on the crystallinity of a semiconductor material used in the transistor, and an amorphous semiconductor or a semiconductor having crystallinity (a microcrystalline semiconductor, a polycrystalline semiconductor, a single crystal semiconductor, or a semiconductor partly including crystal regions) can be used. It is preferable to use a semiconductor having crystallinity, in which case deterioration of transistor characteristics can be suppressed.

In the case of using a metal oxide in a semiconductor film 508, the apparatus 720 includes a light-emitting device including a metal oxide in its semiconductor film and having a metal maskless (MML) structure. With this structure, the leakage current that might flow through the transistor and the leakage current that might flow between adjacent light-emitting devices (also referred to as a lateral leakage current, a side leakage current, or the like) can become extremely low. With the structure, a viewer can observe any one or more of the image crispness, the image sharpness, a high chroma, and a high contrast ratio in an image displayed on the display device. When the leakage current that might flow through the transistor and the lateral leakage current that might flow between light-emitting devices are extremely low, display with little leakage of light at the time of black display (what is called black floating) (such display is also referred to as deep black display) can be achieved.

Alternatively, silicon may be used for the semiconductor film 508. Examples of silicon include single crystal silicon, polycrystalline silicon, and amorphous silicon. In particular, a transistor containing low-temperature polysilicon (LTPS) in its semiconductor layer (hereinafter also referred to as an LTPS transistor) is preferably used. The LTPS transistor has high field-effect mobility and excellent frequency characteristics.

With the use of transistors using silicon such as LTPS transistors, a circuit required to be driven at a high frequency (e.g., a source driver circuit) can be formed on the same substrate as the display unit. This allows simplification of an external circuit mounted on the light-emitting apparatus and a reduction in costs of parts and mounting costs.

The structure of the transistors used in the display panel may be selected as appropriate depending on the size of the screen of the display panel. For example, single crystal Si transistors can be used in the display panel with a screen diagonal greater than or equal to 0.1 inches and less than or equal to 3 inches. In addition, LTPS transistors can be used in the display panel with a screen diagonal greater than or equal to 0.1 inches and less than or equal to 30 inches, preferably greater than or equal to 1 inch and less than or equal to 30 inches. In addition, an LTPO structure (where an LTPS transistor and an OS transistor are used in combination) can be used for the display panel with a screen diagonal greater than or equal to 0.1 inches and less than or equal to 50 inches, preferably greater than or equal to 1 inch and less than or equal to 50 inches. In addition, OS transistors (transistors each including a metal oxide in a semiconductor where a channel is formed) can be used in the display panel with a screen diagonal greater than or equal to 0.1 inches and less than or equal to 200 inches, preferably greater than or equal to 50 inches and less than or equal to 100 inches.

With the use of single crystal Si transistors, an increase in screen size is extremely difficult due to the size of a single crystal Si substrate. Furthermore, since a laser crystallization apparatus is used in the manufacturing process, LTPS transistors are unlikely to respond to an increase in screen size (typically to a screen diagonal greater than 30 inches). By contrast, since the manufacturing process does not necessarily require a laser crystallization apparatus or the like or can be performed at a relatively low temperature (typically, lower than or equal to 450° C.), OS transistors can be applied to a display panel with a relatively large area (typically, a screen diagonal greater than or equal to 50 inches and less than or equal to 100 inches). In addition, LTPO can be applied to a display panel with a size (typically, a screen diagonal greater than or equal to 1 inch and less than or equal to 50 inches) midway between the size of a display panel using LTPS transistors and the size of a display panel using OS transistors.

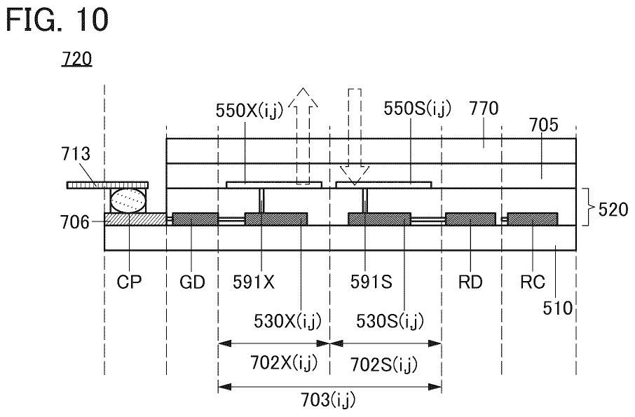

Next, a cross-sectional view of a light-emitting and light-receiving apparatus is shown. FIG. 10 is a cross-sectional view of the apparatus illustrated in FIG. 8A.

FIG. 10 is a cross-sectional view of part of a region including the FPC 713 and the wiring 706 and part of the display region 701 including the pixel 703(i, j).

In FIG. 10, the apparatus 720 includes the functional layer 520 between the first substrate 510 and the second substrate 770. The functional layer 520 includes, as well as the above-described transistors (M11, M12, M13, M14, M15, M16, and M17), the capacitors (C2 and C3), and the like described with reference to FIGS. 9A to 9C, wirings (VS, VG, V1, V2, V3, V4, and V5) electrically connected to these components, for example. FIG. 10 illustrates a non-limiting example of the functional layer 520 that includes a pixel circuit 530X(i, j), a pixel circuit 530S(i, j), and a circuit GD.

Furthermore, each pixel circuit included in the functional layer 520 is electrically connected to a light-emitting device or a light-receiving device. For example, in FIG. 10, the pixel circuit 530X(i, j) and the pixel circuit 530S(i, j) included in the functional layer 520 are electrically connected to a light-emitting device 550X(i, j) and a light-receiving device 550S(i, j), respectively, in FIG. 10 formed over the functional layer 520. Concretely, the light-emitting device 550X(i, j) is electrically connected to the pixel circuit 530X(i, j) through a wiring 591X, and the light-receiving device 550S(i, j) is electrically connected to the pixel circuit 530S(i, j) through a wiring 591S. The insulating layer 705 is provided over the functional layer 520, the light-emitting devices, and the light-receiving device, and has a function of attaching the second substrate 770 and the functional layer 520.

As the second substrate 770, a substrate where touch sensors are arranged in a matrix can be used. For example, a substrate provided with capacitive touch sensors or optical touch sensors can be used as the second substrate 770. Thus, the light-emitting and light-receiving apparatus of one embodiment of the present invention can be used as a touch panel.

The structures described in this embodiment can be used in appropriate combination with any of the structures described in the other embodiments.

### Embodiment 5

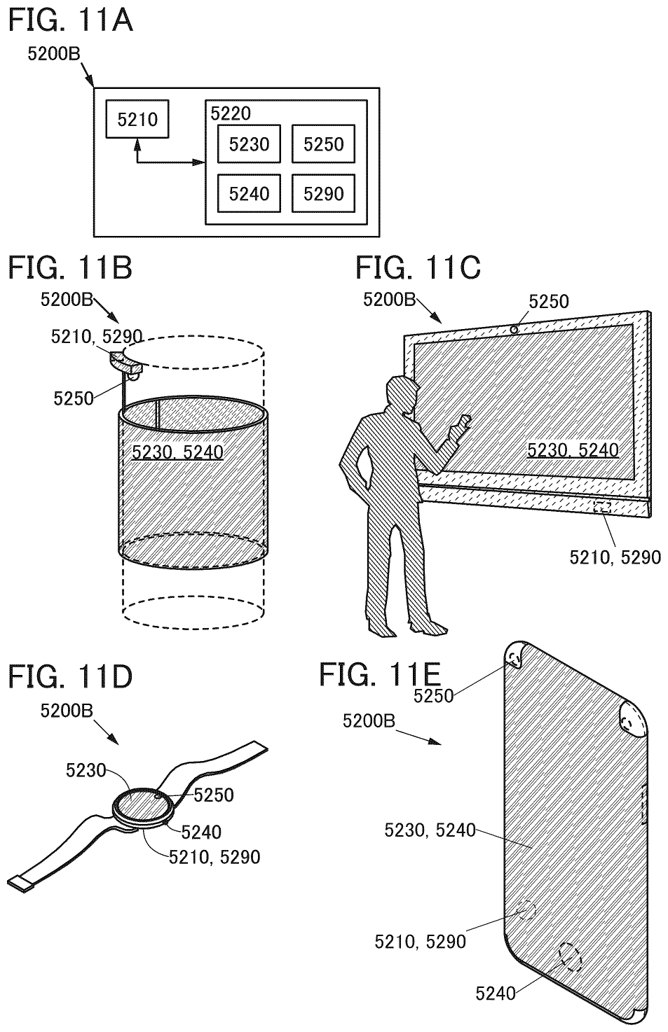

This embodiment will describe structures of electronic devices of embodiments of the present invention with reference to FIGS. 11A to 11E, FIGS. 12A to 12E, and FIGS. 13A and 13B.

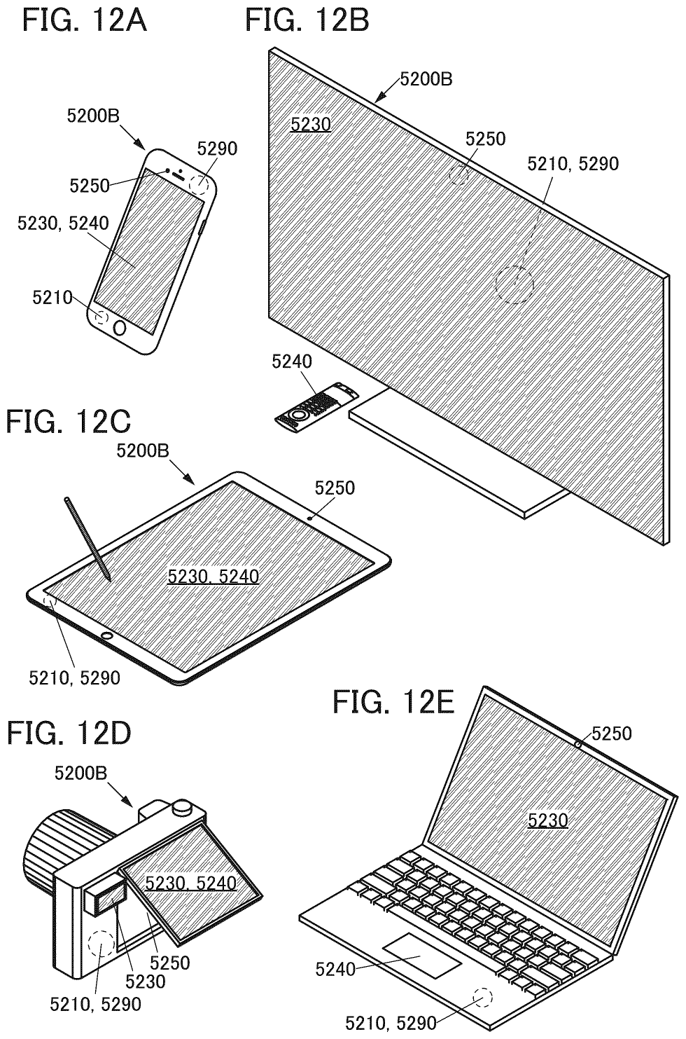

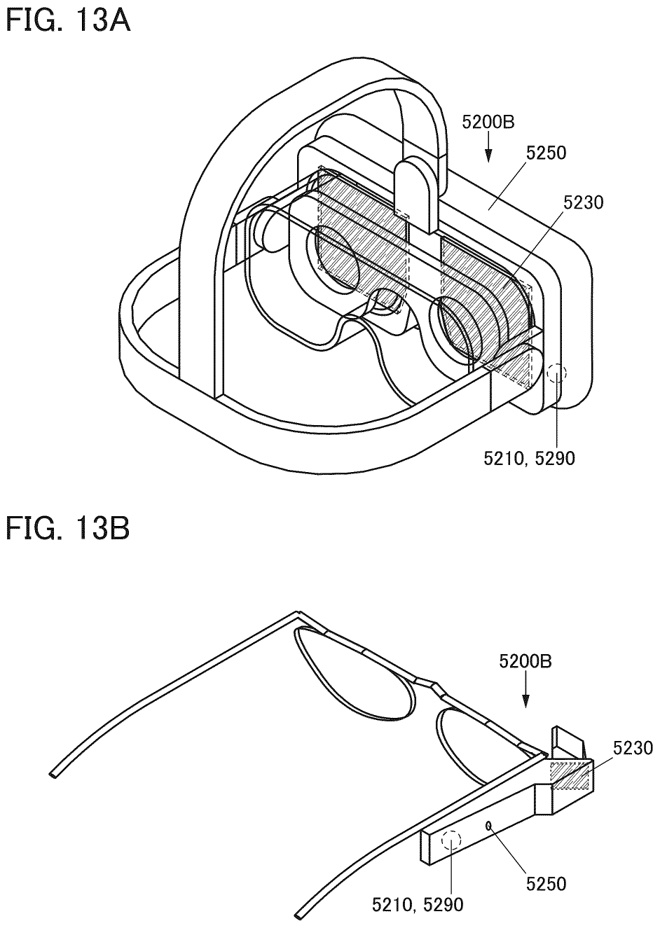

FIGS. 11A to 11E, FIGS. 12A to 12E, and FIGS. 13A and 13B each illustrate a structure of an electronic device of one embodiment of the present invention. FIG. 11A is a block diagram of an electronic device, and FIGS. 11B to 11E are perspective views illustrating structures of the electronic device. FIGS. 12A to 12E are perspective views illustrating structures of electronic devices. FIGS. 13A and 13B are perspective views illustrating structures of electronic devices.

An electronic device 5200B described in this embodiment includes an arithmetic device 5210 and an input/output device 5220 (see FIG. 11A).

The arithmetic device 5210 has a function of receiving handling data and a function of supplying image data on the basis of the handling data.

The input/output device 5220 includes a display unit 5230, an input unit 5240, a sensor unit 5250, and a communication unit 5290, and has a function of supplying handling data and a function of receiving image data. The input/output device 5220 also has a function of supplying sensing data, a function of supplying communication data, and a function of receiving communication data.

The input unit 5240 has a function of supplying handling data. For example, the input unit 5240 supplies handling data on the basis of handling by a user of the electronic device 5200B.

Specifically, a keyboard, a hardware button, a pointing device, a touch sensor, an illuminance sensor, an imaging device, an audio input device, an eye-gaze input device, an attitude sensing device, or the like can be used as the input unit 5240.

The display unit 5230 includes a display panel and has a function of displaying image data. For example, the display panel described in Embodiment 3 can be used for the display unit 5230.

The sensor unit 5250 has a function of supplying sensing data. For example, the sensor unit 5250 has a function of sensing a surrounding environment where the electronic device is used and supplying the sensing data.

Specifically, an illuminance sensor, an imaging device, an attitude sensing device, a pressure sensor, a human motion sensor, or the like can be used as the sensor unit 5250.

The communication unit 5290 has a function of receiving and supplying communication data. For example, the communication unit 5290 has a function of being connected to another electronic device or a communication network by wireless communication or wired communication. Specifically, the communication unit 5290 has a function of wireless local area network communication, telephone communication, near field communication, or the like.

FIG. 11B illustrates an electronic device having an outer shape along a cylindrical column or the like. An example of such an electronic device is digital signage. The display panel of one embodiment of the present invention can be used for the display unit 5230. The electronic device may have a function of changing its display method in accordance with the illuminance of a usage environment. The electronic device has a function of changing the displayed content when sensing the existence of a person. Thus, for example, the electronic device can be provided on a column of a building. The electronic device can display advertising, guidance, or the like.

FIG. 11C illustrates an electronic device having a function of generating image data on the basis of the path of a pointer used by the user. Examples of such an electronic device include an electronic blackboard, an electronic bulletin board, and digital signage. Specifically, a display panel with a diagonal size of 20 inches or longer, preferably 40 inches or longer, further preferably 55 inches or longer can be used. A plurality of display panels can be arranged and used as one display region. Alternatively, a plurality of display panels can be arranged and used as a multiscreen.

FIG. 11D illustrates an electronic device that is capable of receiving data from another device and displaying the data on the display unit 5230. An example of such an electronic device is a wearable electronic device. Specifically, the electronic device can display several options, and the user can choose some from the options and send a reply to the data transmitter. As another example, the electronic device has a function of changing its display method in accordance with the illuminance of a usage environment. Thus, for example, power consumption of the wearable electronic device can be reduced. As another example, the wearable electronic device can display an image so as to be suitably used even in an environment under strong external light, e.g., outdoors in fine weather.

FIG. 11E illustrates an electronic device including the display unit 5230 having a surface gently curved along a side surface of a housing. An example of such an electronic device is a mobile phone. The display unit 5230 includes a display panel that has a function of displaying images on the front surface, the side surfaces, the top surface, and the rear surface, for example. Thus, a mobile phone can display data on not only its front surface but also its side surfaces, top surface, and rear surface, for example.

FIG. 12A illustrates an electronic device that is capable of receiving data via the Internet and displaying the data on the display unit 5230. An example of such an electronic device is a smartphone. For example, the user can check a created message on the display unit 5230 and send the created message to another device. As another example, the electronic device has a function of changing its display method in accordance with the illuminance of a usage environment. Thus, power consumption of the smartphone can be reduced. As another example, it is possible to obtain a smartphone which can display an image such that the smartphone can be suitably used in an environment under strong external light, e.g., outdoors in fine weather.

FIG. 12B illustrates an electronic device that can use a remote controller as the input unit 5240. An example of such an electronic device is a television system. For example, data received from a broadcast station or via the Internet can be displayed on the display unit 5230. The electronic device can take an image of the user with the sensor unit 5250 and transmit the image of the user. The electronic device can acquire a viewing history of the user and provide it to a cloud service. The electronic device can acquire recommendation data from a cloud service and display the data on the display unit 5230. A program or a moving image can be displayed on the basis of the recommendation data. As another example, the electronic device has a function of changing its display method in accordance with the illuminance of a usage environment. Accordingly, for example, it is possible to obtain a television system which can display an image such that the television system can be suitably used even under strong external light entering the room from the outside in fine weather.

FIG. 12C illustrates an electronic device that is capable of receiving an educational material via the Internet and displaying it on the display unit 5230. An example of such an electronic device is a tablet computer. The user can input an assignment with the input unit 5240 and send it via the Internet. The user can obtain a corrected assignment or the evaluation from a cloud service and have it displayed on the display unit 5230. The user can select a suitable educational material on the basis of the evaluation and have it displayed.

For example, an image signal can be received from another electronic device and displayed on the display unit 5230. When the electronic device is placed on a stand or the like, the display unit 5230 can be used as a sub-display. Thus, for example, it is possible to obtain a tablet computer which can display an image such that the tablet computer is favorably used even in an environment under strong external light, e.g., outdoors in fine weather.

FIG. 12D illustrates an electronic device including a plurality of display units 5230. An example of such an electronic device is a digital camera. For example, the display unit 5230 can display an image that the sensor unit 5250 is capturing. A captured image can be displayed on the sensor unit. A captured image can be decorated using the input unit 5240. A message can be attached to a captured image. A captured image can be transmitted via the Internet. The electronic device has a function of changing shooting conditions in accordance with the illuminance of a usage environment. Accordingly, for example, it is possible to obtain a digital camera that can display a subject such that an image is favorably viewed even in an environment under strong external light, e.g., outdoors in fine weather.

FIG. 12E illustrates an electronic device in which the electronic device of this embodiment is used as a master to control another electronic device used as a slave. An example of such an electronic device is a portable personal computer. For example, part of image data can be displayed on the display unit 5230 and another part of the image data can be displayed on a display unit of another electronic device. Image signals can be supplied. Data written from an input unit of another electronic device can be obtained with the communication unit 5290. Thus, a large display region can be utilized in the case of using a portable personal computer, for example.

FIG. 13A illustrates an electronic device including the sensor unit 5250 that senses an acceleration or a direction. An example of such an electronic device is a goggles-type electronic device. The sensor unit 5250 can supply data on the position of the user or the direction in which the user faces. The electronic device can generate image data for the right eye and image data for the left eye in accordance with the position of the user or the direction in which the user faces. The display unit 5230 includes a display region for the right eye and a display region for the left eye. Thus, a virtual reality image that gives the user a sense of immersion can be displayed on the goggles-type electronic device, for example.

FIG. 13B illustrates an electronic device including an imaging device and the sensor unit 5250 that senses an acceleration or a direction. An example of such an electronic device is a glasses-type electronic device. The sensor unit 5250 can supply data on the position of the user or the direction in which the user faces. The electronic device can generate image data in accordance with the position of the user or the direction in which the user faces. Accordingly, the data can be shown together with a real-world scene, for example. Alternatively, an augmented reality image can be displayed on the glasses-type electronic device.

Note that this embodiment can be combined with any of the other embodiments in this specification as appropriate.

### Embodiment 6

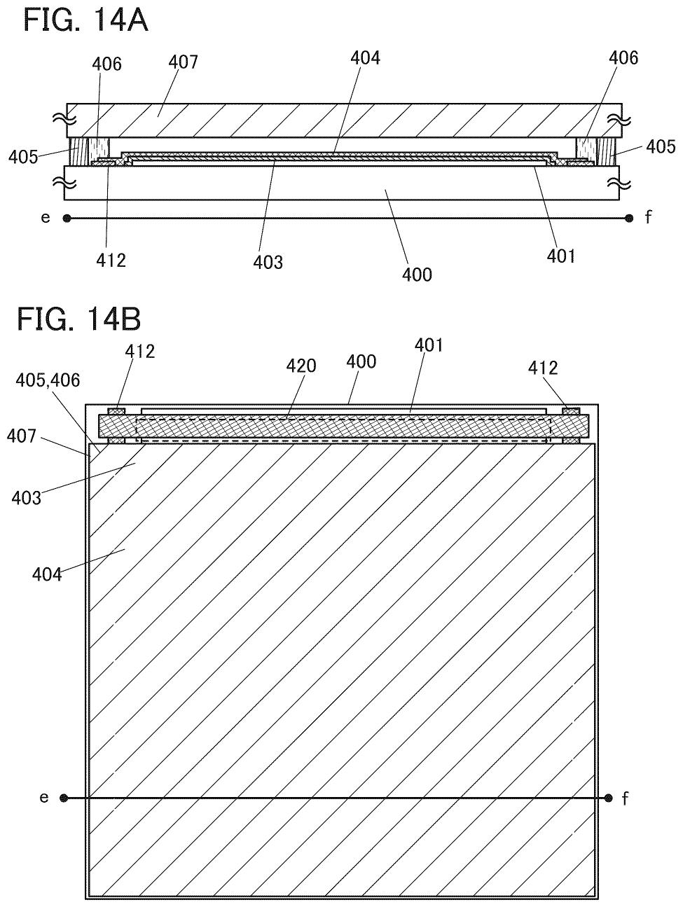

This embodiment will describe a structure in which any of the light-emitting devices described in the above embodiment is used as a lighting device with reference to FIGS. 14A and 14B. FIG. 14A illustrates a cross section taken along the line e-f in a top view of the lighting device in FIG. 14B.

In the lighting device in this embodiment, a first electrode 401 is formed over a substrate 400 that is a support and has a light-transmitting property. The first electrode 401 corresponds to the first electrode 101 in the above embodiment. When light is extracted from the first electrode 401 side, the first electrode 401 is formed using a material having a light-transmitting property.

A pad 412 for applying voltage to a second electrode 404 is provided over the substrate 400.

An EL layer 403 is formed over the first electrode 401. The structure of the EL layer 403 corresponds to the structure of the EL layer 103 in Embodiment 2. Refer to the corresponding description for these structures.

The second electrode 404 is formed to cover the EL layer 403. The second electrode 404 corresponds to the second electrode 102 in the above embodiment. The second electrode 404 is formed using a material having high reflectance when light is extracted from the first electrode 401 side. The second electrode 404 is connected to the pad 412 so that voltage is applied to the second electrode 404.

As described above, the lighting device described in this embodiment includes a light-emitting device including the first electrode 401, the EL layer 403, and the second electrode 404. Since the light-emitting device has high emission efficiency, the lighting device in this embodiment can have low power consumption.

The substrate 400 provided with the light-emitting device having the above structure and a sealing substrate 407 are fixed and sealed with sealing materials 405 and 406, whereby the lighting device is completed. It is possible to use only either the sealing material 405 or the sealing material 406. In addition, the inner sealing material 406 (not illustrated in FIG. 14B) can be mixed with a desiccant that enables moisture to be adsorbed, increasing the reliability.

When parts of the pad 412 and the first electrode 401 are extended to the outside of the sealing materials 405 and 406, the extended parts can serve as external input terminals. An IC chip 420 mounted with a converter or the like may be provided over the external input terminals.

### Embodiment 7

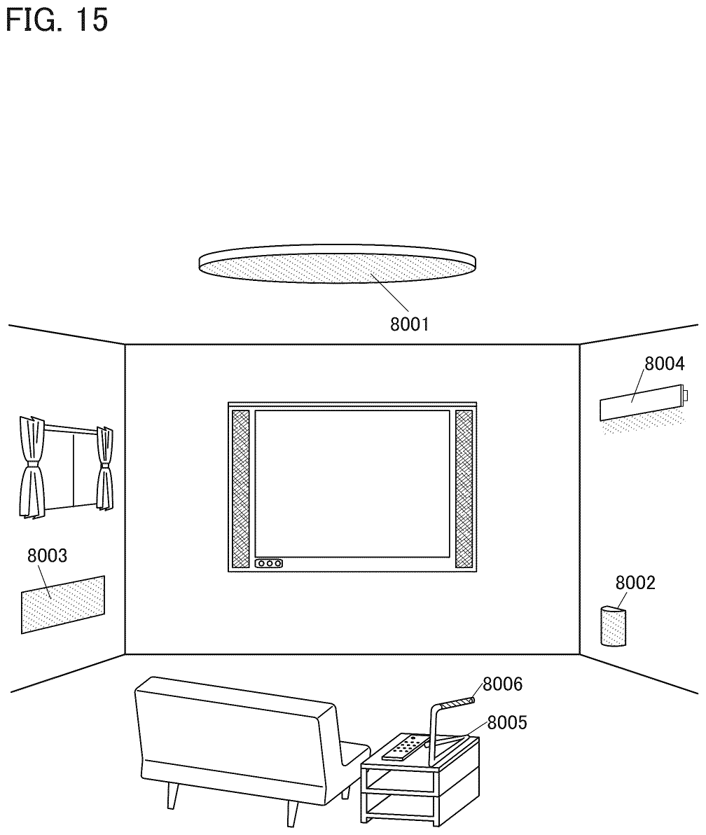

This embodiment will describe application examples of lighting devices fabricated using the light-emitting apparatus of one embodiment of the present invention or the light-emitting device, which is part of the light-emitting apparatus with reference to FIG. 15.

A ceiling light 8001 can be used as an indoor lighting device. Examples of the ceiling light 8001 include a direct-mount light and an embedded light. Such lighting devices are fabricated using the light-emitting apparatus and a housing and a cover in combination. Application to a cord pendant light (light that is suspended from a ceiling by a cord) is also possible.

A foot light 8002 lights a floor so that safety on the floor can be improved. For example, it can be effectively used in a bedroom, on a staircase, and on a passage. In such cases, the size and shape of the foot light can be changed in accordance with the dimensions and structure of a room. The foot light can be a stationary lighting device using the light-emitting apparatus and a support in combination.

A sheet-like lighting 8003 is a thin sheet-like lighting device. The sheet-like lighting, which is attached to a wall when used, is space-saving and thus can be used for a wide variety of uses. Furthermore, the area of the sheet-like lighting can be easily increased. The sheet-like lighting can also be used on an object such as a wall or a housing that has a curved surface.

A lighting device 8004 in which the direction of light from a light source is controlled to be only a desired direction can be used.

A desk lamp 8005 includes a light source 8006. As the light source 8006, the light-emitting apparatus of one embodiment of the present invention or the light-emitting device, which is part of the light-emitting apparatus, can be used.

Besides the above examples, when the light-emitting apparatus of one embodiment of the present invention or the light-emitting device, which is part of the light-emitting apparatus, is used as part of furniture in a room, a lighting device that functions as the furniture can be obtained.

As described above, a variety of lighting devices that include the light-emitting apparatus can be obtained. Note that these lighting devices are also embodiments of the present invention.

The structures described in this embodiment can be used in appropriate combination with any of the structures described in the other embodiments.

### Example 1

### Synthesis Example 1

This example shows synthesis of 4,4′-(diphenylsilanediyl)bis{N-[4-(9H-carbazol-9-yl)phenyl]-N-phenylaniline} (abbreviation: PSiYGA2) (structural formula (100)), which is the organic compound of one embodiment of the present invention, and various measurement results of this compound. The structure of PSiYGA2 is shown below.

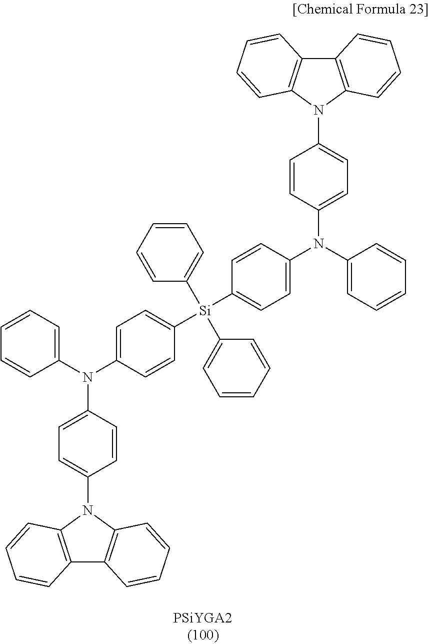 [SMILES: c1ccc(N(c2ccc(-n3c4ccccc4c4ccccc43)cc2)c2ccc([Si](c3ccccc3)(c3ccccc3)c3ccc(N(c4ccccc4)c4ccc(-n5c6ccccc6c6ccccc65)cc4)cc3)cc2)cc1]

Into a 300 mL three-neck flask were put 1.5 g (3.0 mmol) of bis(4-bromophenyl)diphenylsilane, 2.0 g (6.0 mmol) of N-[4-(9H-carbazol-9-yl)phenyl]-N-phenylaniline, 1.0 g (10 mmol) of sodium tert-butoxide (abbreviation: tBuONa), and 0.1 mg (0.1 mmol) of bis(dibenzylideneacetone)palladium(0) (abbreviation: Pd(dba)₂), and the air in the flask was replaced with nitrogen. Then, 100 mL of dehydrated xylene was added to this mixture. After the mixture was degassed while being stirred under reduced pressure, 0.8 mL (0.4 mmol) of tri(tert-butyl)phosphine (abbreviation: (tBu)₃P) (10 wt % hexane solution) was added thereto. This mixture was stirred while being heated under a nitrogen stream at 120° C. for 6 hours to be reacted. After the reaction, 150 mL of toluene was added to this reaction mixture solution, and this suspension was filtered through Florisil and Celite. The resulting filtrate was concentrated, purified by silica gel column chromatography (a developing solvent: a mixed solution of toluene and hexane), and then recrystallized to give 2.1 g of a white powder which is a target substance in a yield of 70%. The reaction scheme of this synthesis method is illustrated below in (a-1).

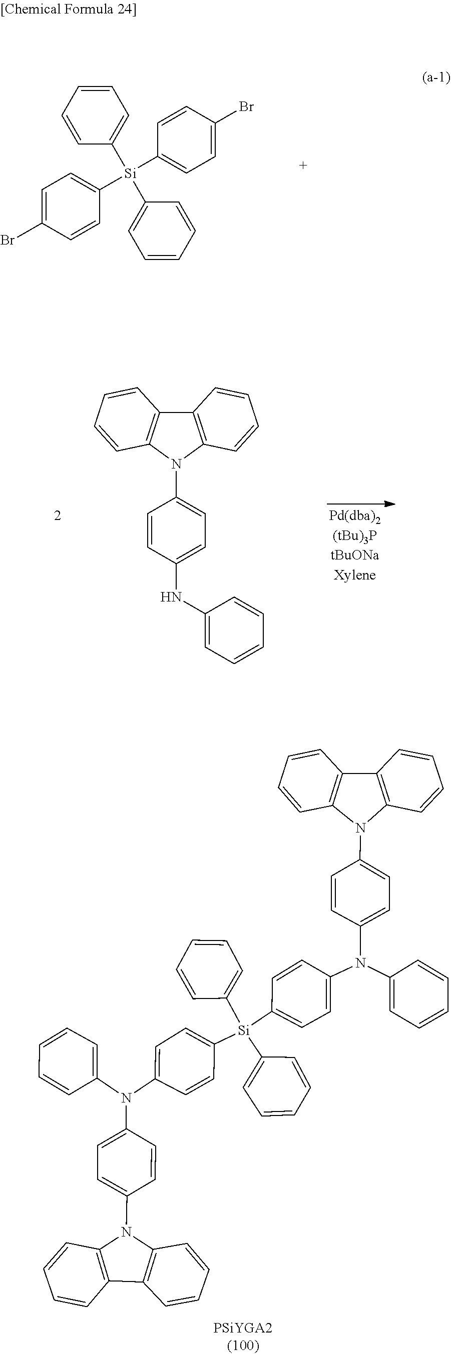 [SMILES: Brc1ccc([Si](c2ccccc2)(c2ccccc2)c2ccc(Br)cc2)cc1 ; c1ccc(Nc2ccc(-n3c4ccccc4c4ccccc43)cc2)cc1 ; c1ccc(N(c2ccc(-n3c4ccccc4c4ccccc43)cc2)c2ccc([Si](c3ccccc3)(c3ccccc3)c3ccc(N(c4ccccc4)c4ccc(-n5c6ccccc6c6ccccc65)cc4)cc3)cc2)cc1]

Silica gel thin layer chromatography (TLC) using a developing solvent (ethyl acetate and hexane in a ratio of 1:5) gave the following Rf values: the target substance, 0.49; and bis(4-bromophenyl)diphenylsilane, 0.41

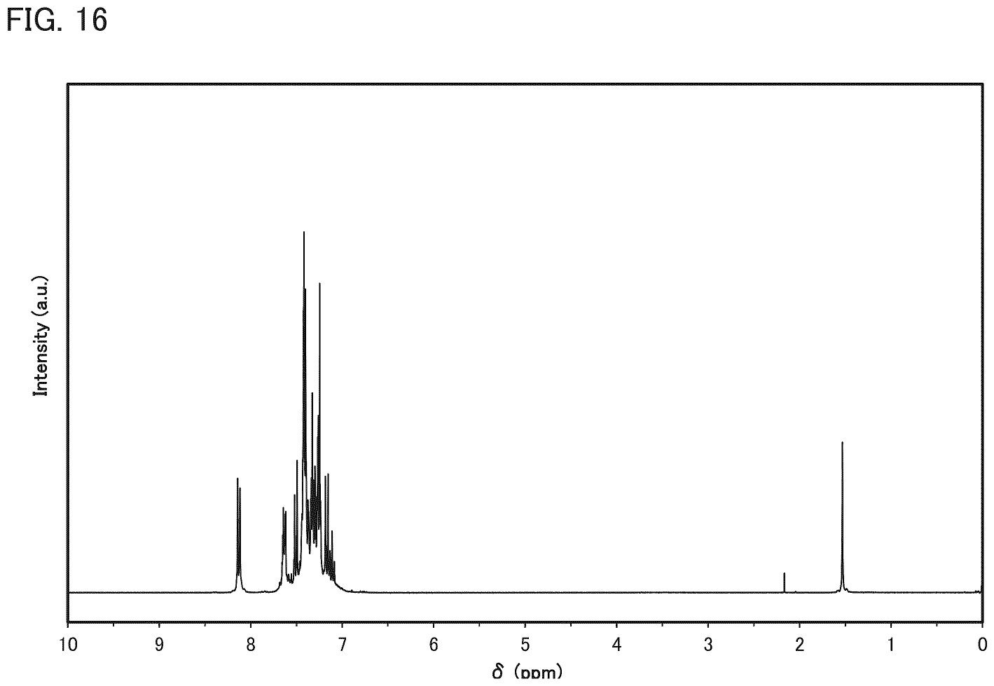

FIG. 16 shows a ¹H NMR spectrum of PSiYGA2 after the purification by sublimation. Results of ¹H NMR measurement are shown below. The results reveal that PSiYGA2 was obtained.

¹H NMR (CDCl₃, 300 MHz): δ (ppm)=7.11 (t, J=7.2 Hz, 2H), 7.17 (d, J=8.4, 4H), 7.25-7.44 (m, 34H), 7.51 (d, J=8.4, 4H), 7.61-7.65 (m, 4H), 8.13 (d, J=7.8, 4H).

Next, PSiYGA2 obtained in this example was analyzed by liquid chromatography mass spectrometry (LC/MS).

In the LC/MS analysis, liquid chromatography (LC) separation was performed with UltiMate 3000 by Thermo Fisher Scientific K.K., and mass spectrometry (MS) was performed with Q Exactive by Thermo Fisher Scientific K.K.

The LC separation was performed with a given column at a column temperature of 40° C. under the following solution sending conditions: an appropriately selected solvent (an aqueous acetonitrile solution); a sample prepared by dissolving PSiYGA2 in an organic solvent at a given concentration; and 5.0 μL injection amount.

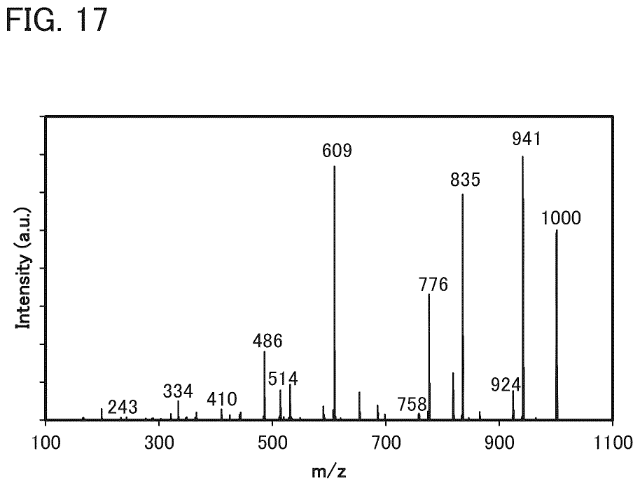

MS/MS of the m/ [symbol] of 1000.40 corresponding to the exact mass of PSiYGA2 was performed by a parallel reaction monitoring (PRM) method. For PRM, the mass range of a target ion was set to m/ [symbol] =1000.40±2.0 (isolation window=4) and detection was performed in a positive mode. The measurement was performed with energy (normalized collision energy: NCE) accelerating the target ion in a collision cell set to 40. The MS spectrum obtained by MS/MS is shown in FIG. 17.

FIG. 17 shows that product ions of PSiYGA2 are mainly detected at m/ [symbol] of around 243, around 334, around 410, around 486, around 514, around 609, around 758, around 776, around 835, around 924, and around 941.

The product ion at m/ [symbol] of 243 corresponds to a product ion C₁₈H₁₃N^{⋅+} derived from a carbazolyl phenyl group. The product ion at m/ [symbol] of 334 corresponds to a product ion C₂₄H₁₈N₂^{⋅+} derived from a carbazolyl diphenylamino group. The product ion at m/ [symbol] of 410 corresponds to a product ion C₃₀H₂₂N₂^{⋅+} derived from a carbazolyl triphenylamino group. The product ion at m/ [symbol] of 486 corresponds to a product ion C₃₆H₂₆N₂^{⋅+} derived from a carbazolyl triphenylamino group with an additional phenyl group. The product ion at m/ [symbol] of 514 corresponds to a product ion C₃₆H₂₆N₂Si^{⋅⋅⋅+} obtained by elimination of a carbazolyl triphenylamino group and a phenyl group from PSiYGA2. The product ion at m/ [symbol] of 609 corresponds to a product ion C₄₂H₃₃N₂OSi⁺ obtained by elimination of a carbazolyl triphenylamino group from PSiYGA2 and addition of water. The product ion at m/ [symbol] of 758 corresponds to a product ion C₅₄H₄₀N₃Si^{⋅⋅+} obtained by elimination of a carbazolyl group and a phenyl group from PSiYGA2. The product ion at m/ [symbol] of 776 corresponds to a product ion C₅₄H₄₂N₃OSi⁺ obtained by elimination of a carbazolyl group and a phenyl group from PSiYGA2 and addition of water. The product ion at m/ [symbol] of 835 corresponds to a product ion C₆₀H₄₅N₃Si^{⋅+} obtained by elimination of a 9H-carbazolyl group from PSiYGA2. The product ion at m/ [symbol] of 924 corresponds to a product ion C₆₆H₄₈N₄Si^{⋅+} obtained by elimination of a phenyl group from PSiYGA2. The product ion at m/ [symbol] of 941 corresponds to a product ion C₆₆H₄₉N₄OSi^{⋅+} obtained by elimination of a phenyl group from PiYGA2 and addition of water.

The above results indicate that PSiYGA2 includes a carbazole skeleton and a benzene skeleton. Note that the above m/ [symbol] values±1 may be detected as protonation and deprotonation products of the product ions.

As described above, one of the features of the general formula (G1) in which Q is silicon is detection of the m/z values corresponding to the product ions of a silanol group (SiOH) formed by elimination of the first or second partial structure (aryl group) bonded to silicon and addition of water thereto.

### Example 2

### Synthesis Example 2

This example shows synthesis of 4,4′-(propane-2,2-diyl)bis{N-[4-(9H-carbazol-9-yl)phenyl]-N-phenylaniline} (abbreviation: YGABmP) (structural formula (114)) and various measurement results of this compound.

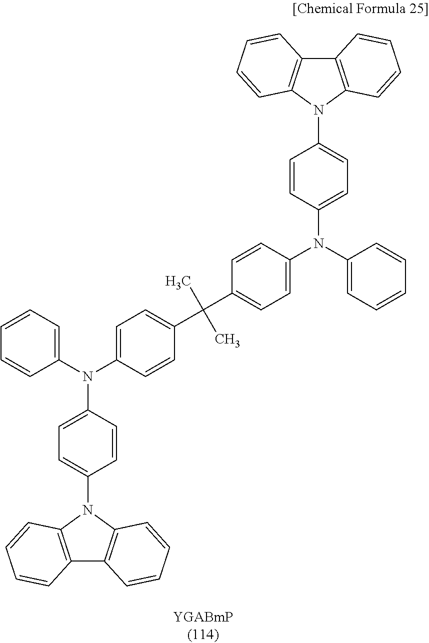 [SMILES: CC(C)(c1ccc(N(c2ccccc2)c2ccc(-n3c4ccccc4c4ccccc43)cc2)cc1)c1ccc(N(c2ccccc2)c2ccc(-n3c4ccccc4c4ccccc43)cc2)cc1]

### (Synthesis Method of YGABmP)

Into a 100 mL three-neck flask were put 2.2 g (5.0 mmol) of 4,4′-(propane-2,2-diyl)bis(iodobenzene), 3.5 g (11 mmol) of N-[4-(9H-carbazol-9-yl)phenyl]-N-phenylaniline, 4.0 g (40 mmol) of sodium tert-butoxide (abbreviation: tBuONa), and 0.2 mg (0.4 mmol) of bis(dibenzylideneacetone)palladium(0) (abbreviation: Pd(dba)₂), and the air in the flask was replaced with nitrogen. Then, 20 mL of dehydrated xylene was added to this mixture. After the mixture was degassed while being stirred under reduced pressure, 0.8 mL (0.4 mmol) of tri(tert-butyl)phosphine (abbreviation: (tBu)₃P) (10 wt % hexane solution) was added thereto. This mixture was stirred while being heated under a nitrogen stream at 120° C. for 6 hours to be reacted. After the reaction, 150 mL of toluene was added to this reaction mixture solution, and this suspension was filtered through Florisil and Celite. The resulting filtrate was concentrated, purified by silica gel column chromatography (a developing solvent: a mixed solution of toluene and hexane), and then recrystallized to give 2.1 g of a white powder which is a target substance in a yield of 70%. The reaction scheme is illustrated below in (b-1).

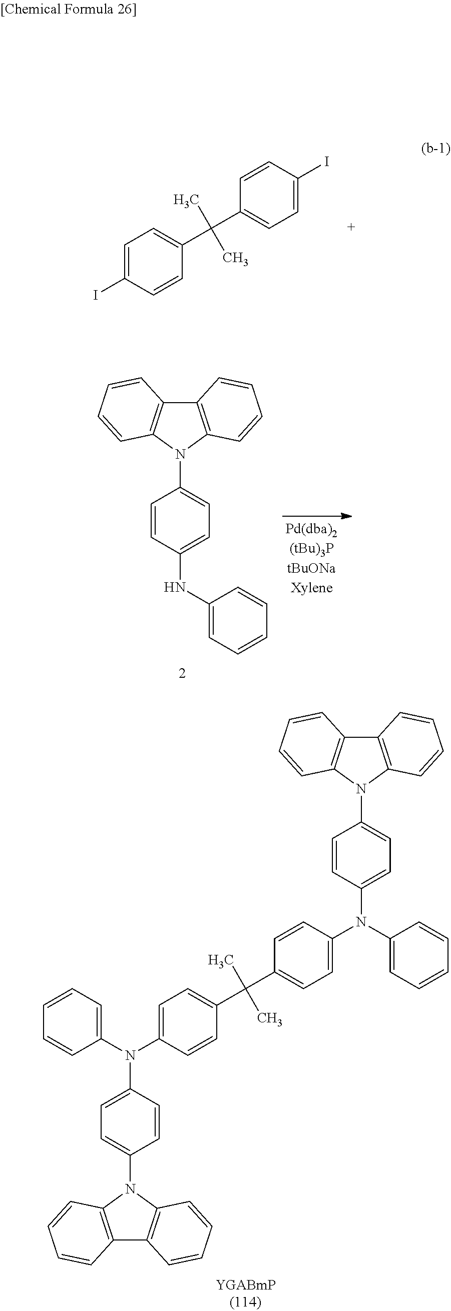 [SMILES: CC(C)(c1ccc(I)cc1)c1ccc(I)cc1 ; c1ccc(Nc2ccc(-n3c4ccccc4c4ccccc43)cc2)cc1 ; CC(C)(c1ccc(N(c2ccccc2)c2ccc(-n3c4ccccc4c4ccccc43)cc2)cc1)c1ccc(N(c2ccccc2)c2ccc(-n3c4ccccc4c4ccccc43)cc2)cc1]

Silica gel thin layer chromatography (TLC) using a developing solvent (ethyl acetate and hexane in a ratio of 1:10) gave the following Rf values: the target substance, 0.24; 4,4′-(propane-2,2-diyl)bis(iodobenzene), 0.73; and N-[4-(9H-carbazol-9-yl)phenyl]-N-phenylaniline, 0.20.

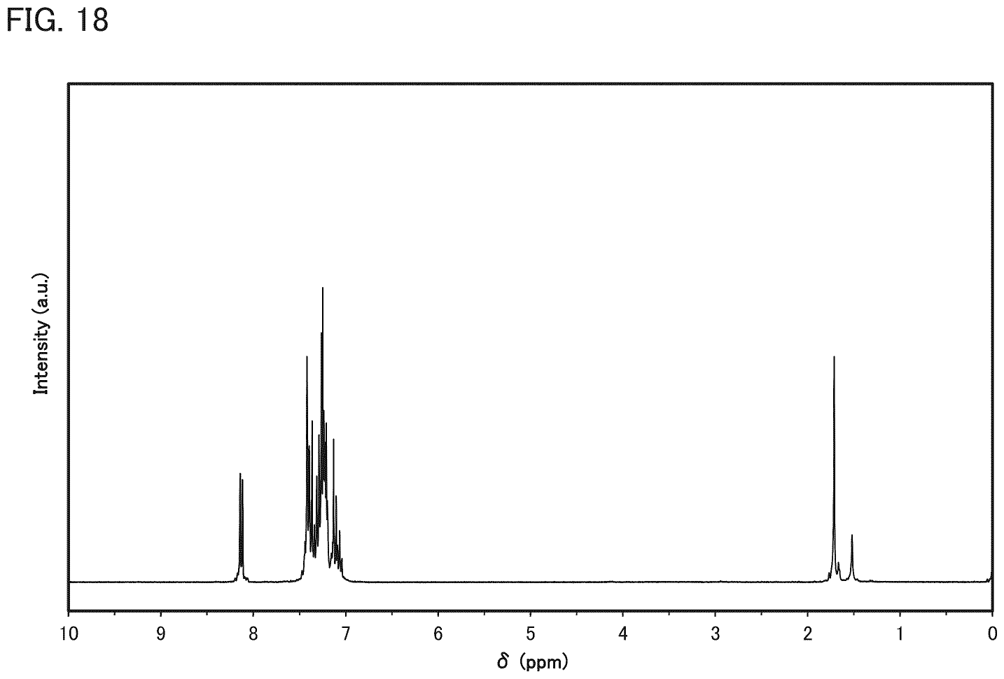

FIG. 18 shows a ¹H NMR spectrum of the obtained organic compound. Results of ¹H NMR measurement are shown below. The results reveal that YGABmP, which was the target substance, was obtained.

¹H NMR (CDCl₃, 300 MHz): δ (ppm)=2.81 (s, 6H), 7.04-7.44 (m, 38H), 8.13 (d, J=7.8 Hz, 4H).

Next, YGABmP obtained in this example was analyzed by LC/MS.

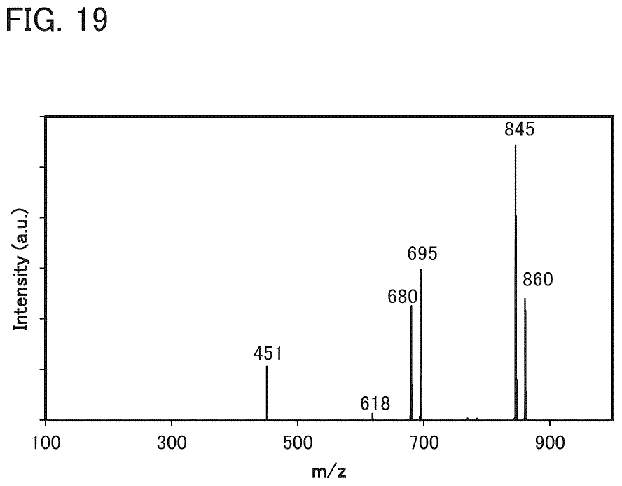

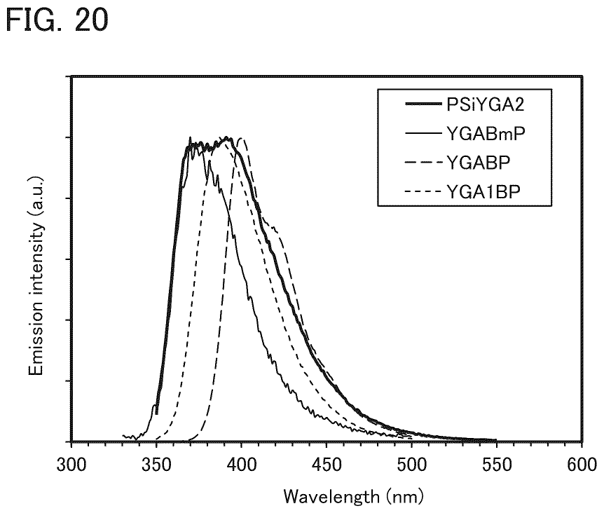

As in Example 1, MS/MS of the m/ [symbol] of 860.39 corresponding to the exact mass of YGABmP was performed by a PRM method. For PRM, the mass range of a target ion was set to m/ [symbol] =860.39±2.0 (isolation window=4) and detection was performed in a positive mode. Measurement was performed with energy (normalized collision energy: NCE) accelerating the target ion in a collision cell set to 30. The MS spectrum obtained by MS/MS is shown in FIG. 19.

FIG. 19 shows that product ions of YGABmP are mainly detected at m/ [symbol] of around 451, around 618, around 680, around 695, and around 845.

The product ion at m/ [symbol] of 451 corresponds to a product ion C₃₃H₂₇N₂^{⋅+} obtained by elimination of a carbazolyl diphenylamino group and a phenyl group from YGABmP. The product ion at m/ [symbol] of 618 corresponds to a product ion C₄₅H₃₆N₃^{⋅+} obtained by elimination of a carbazolyl group and a phenyl group from YGABmP. The product ion at m/ [symbol] of 680 corresponds to a product ion C₅₀H₃₈N₃^{⋅+} obtained by elimination of a carbazolyl group and a methyl group from YGABmP. The product ion at m/ [symbol] of 695 corresponds to a product ion C₅₁H₄₁N₃^{⋅+} obtained by elimination of a carbazolyl group [symbol] from YGABmP. The product ion at m/ [symbol] of 845 corresponds to a product ion C₆₂H₄₅N₄^{⋅+} obtained by elimination of a methyl group from YGABmP.

The above results indicate that YGABmP includes a carbazole skeleton and a benzene skeleton. Note that the above m/z values±1 may be detected as protonation and deprotonation products of the product ions. As described above, one of the features of the general formula (G1) in which Q is carbon is detection of the m/ [symbol] values corresponding to the product ions formed by elimination of the first or second partial structure (aryl group) bonded to carbon.

Next, measurement results of the T_{g} and emission spectra of PSiYGA2, whose synthesis method is shown in Example 1, YGABmP, whose synthesis method is shown in this example, and comparative compounds are described. The comparative compounds used were N-(4-biphenyl)-4-(carbazol-9-yl)phenylaniline (abbreviation: YGA1BP) and N,N′-bis[4-(carbazol-9-yl)phenyl]-N,N′-diphenylbiphenyl-4,4′-diamine (abbreviation: YGABP), each of which does not have a sigma bond while having the same unit as PSiYGA2 and YGABmP. The following are the chemical formulae of PSiYGA2, YGABmP, YGA1BP, and YGABP in addition to Unit A that these compounds include in common.

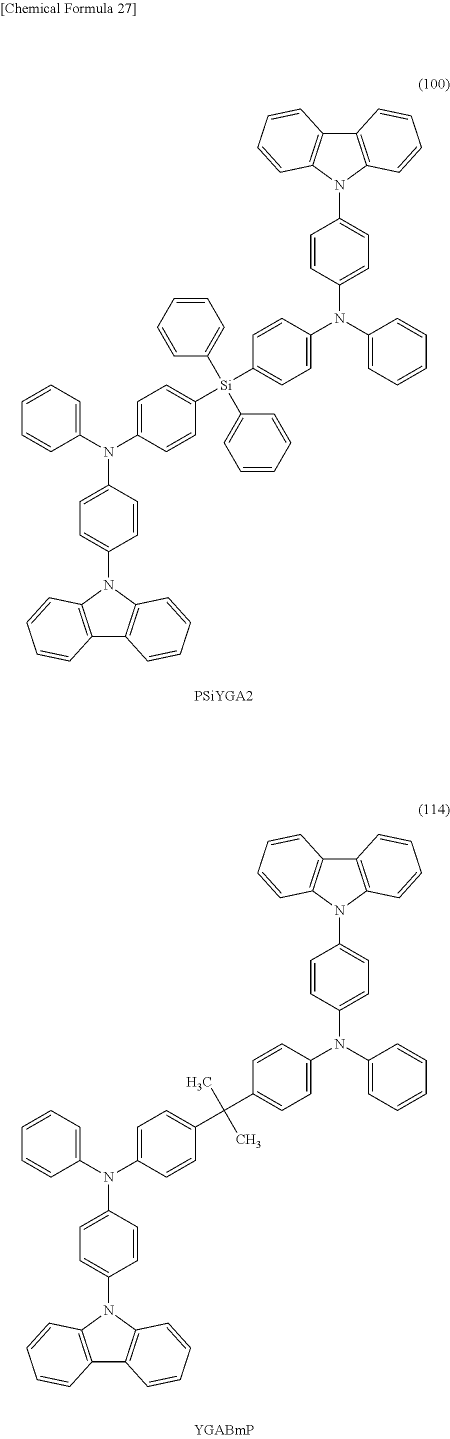 [SMILES: c1ccc(N(c2ccc(-n3c4ccccc4c4ccccc43)cc2)c2ccc([Si](c3ccccc3)(c3ccccc3)c3ccc(N(c4ccccc4)c4ccc(-n5c6ccccc6c6ccccc65)cc4)cc3)cc2)cc1 ; CC(C)(c1ccc(N(c2ccccc2)c2ccc(-n3c4ccccc4c4ccccc43)cc2)cc1)c1ccc(N(c2ccccc2)c2ccc(-n3c4ccccc4c4ccccc43)cc2)cc1]

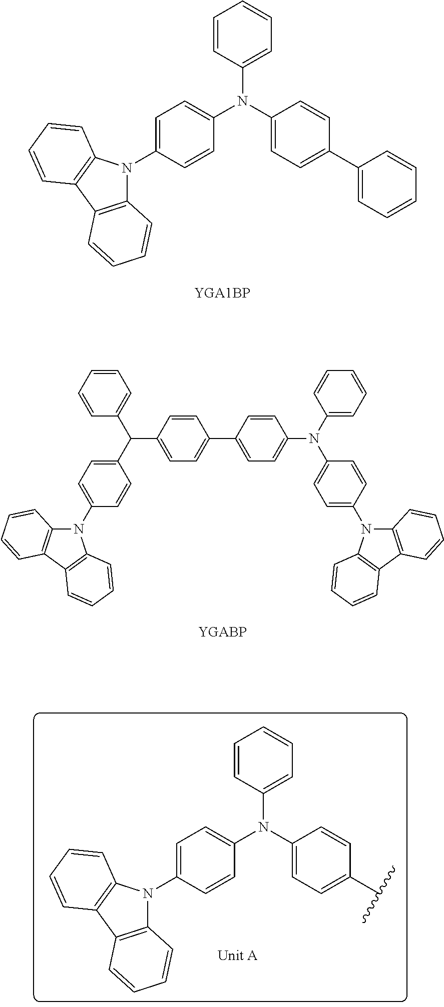 [SMILES: c1ccc(-c2ccc(N(c3ccccc3)c3ccc(-n4c5ccccc5c5ccccc54)cc3)cc2)cc1 ; c1ccc(C(c2ccc(-c3ccc(N(c4ccccc4)c4ccc(-n5c6ccccc6c6ccccc65)cc4)cc3)cc2)c2ccc(-n3c4ccccc4c4ccccc43)cc2)cc1 ; *c1ccc(N(c2ccccc2)c2ccc(-n3c4ccccc4c4ccccc43)cc2)cc1]

Emission spectra of toluene solutions of the compounds were measured with a fluorescence spectrophotometer (FS920, Hamamatsu Photonics K.K.). FIG. 20 shows the emission spectra of the toluene solutions of PSiYGA2, YGABmP, YGA1BP, and YGABP. The S₁ level was energy of the wavelength (emission edge) of the intersection of the horizontal axis (wavelength) or the base line and a tangent to the emission spectrum at a point where the slope of the spectrum at a peak on the shorter wavelength side has a maximum value.

The T_{g} of each compound was measured with a differential scanning calorimeter (DSC, Pyris 1 DSC, or DSC8500, PerkinElmer, Inc.). The temperature of a powder sample of PSiYGA2 was raised to 370° C. (higher than or equal to the melting point) and then decreased to −5° C. or lower at 50° C./min or higher. Then, the temperature was raised to 370° C. at 40° C./min and the T_{g} was read from the obtained DSC curve. The T_{g} of each of the other compounds was similarly measured.

The peak wavelength and the wavelength of the short-wavelength emission edge, the S₁ level calculated from the emission edge, and the measured T_{g} of each compound are listed in the following table.

TABLE 1

| | Peak wavelength | Emission | S₁ | T_{g} |
|---|---|---|---|---|
| | (nm) | edge (nm) | level (eV) | (° C.) |
| PSiYGA2 | 373 | 350 | 3.54 | 144 |
| YGABmP | 375 | 350 | 3.54 | 140 |
| YGA1BP | 388 | 362 | 3.43 | 82 |
| YGABP | 400, 420 | 382 | 3.25 | 144 |

FIG. 20 and the above table show that the peak wavelengths and the wavelengths of the short-wavelength emission edges of PSiYGA2 and YGABmP are shorter than those of YGA1BP.

The above table also shows that PSiYGA2 and YGABmP each have a higher S₁ level than YGA1BP and YGABP. This is because PSiYGA2 and YGABmP each have a structure in which two Units A are bonded through a sigma bond while YGA1BP has a structure in which a phenyl group is directly bonded to Unit A and YGABP has a structure in which two Units A are directly bonded to each other.

The above table also reveals that the T_{g} of each of PSiYGA2 and YGABmP is higher than or equal to 140° C., which is equivalent to that of YGABP and significantly higher than that of YGA1BP. This is probably because, like YGABP, PSiYGA2 and YGABmP each have a sufficiently high molecular weight by having two Units A.

The above results reveal that PSiYGA2 and YGABmP of one embodiment of the present invention are materials each having a high S₁ level and a high T_{g}.

### Example 3

### Synthesis Example 3

This example shows synthesis of diphenyl-bis{4-[3-(9-phenyl-9H-carbazol-3-yl)-9H-carbazol-9-yl)phenyl}silane (abbreviation: PCCz2PSi) (structural formula (101)), which is the organic compound of one embodiment of the present invention, and various measurement results of this compound. The structure of PCCz2PSi is shown below.

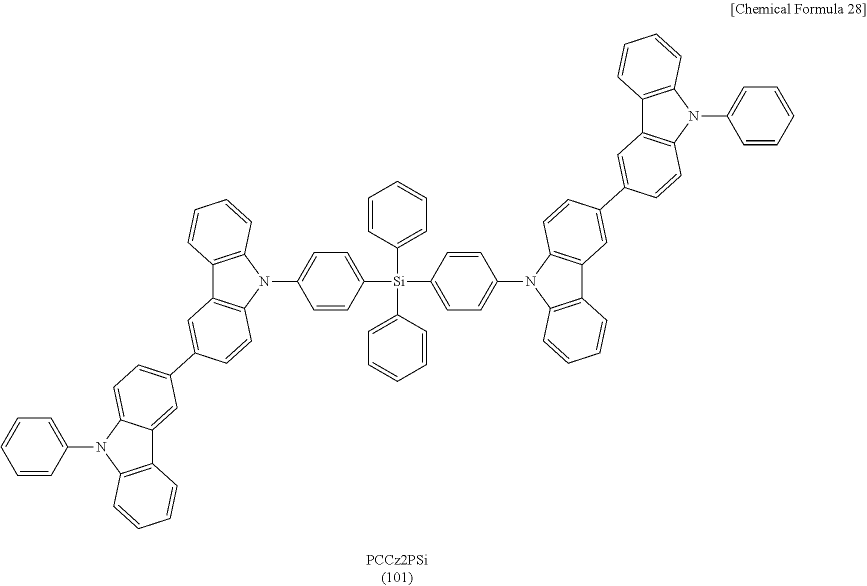 [SMILES: c1ccc(-n2c3ccccc3c3cc(-c4ccc5c(c4)c4ccccc4n5-c4ccc([Si](c5ccccc5)(c5ccccc5)c5ccc(-n6c7ccccc7c7cc(-c8ccc9c(c8)c8ccccc8n9-c8ccccc8)ccc76)cc5)cc4)ccc32)cc1]

### <Synthesis of PCCz2PSi>

Into a 50 mL three-neck flask were put 0.44 g (0.89 mmol) of bis(4-bromophenyl)-diphenylsilane, 0.73 g (1.8 mmol) of 3-(9-phenyl-9H-carbazol-3-yl)-9H-carbazole, and 0.74 g (5.4 mmol) of potassium carbonate (abbreviation: K₂CO₃). To the mixture, 9.0 mL of dehydrated xylene was added, and this mixture was degassed by being stirred under reduced pressure. To this mixture were added 4.0 mg (18 μmol) of palladium(II) acetate (abbreviation: Pd(OAc)₂) and 18 mg (89 μmol) of tri-t-butylphosphine (abbreviation: P(tBu)₃), and the mixture was stirred at 140° C. under a nitrogen stream for 10 hours. After stirring, the mixture was cooled down to room temperature. This mixture was subjected to extraction with toluene. Then, the solution after the extraction was concentrated to give an oily substance. The oily substance was purified by silica gel column chromatography (hexane was first used and toluene and hexane in a ratio of 1:2 were then used as developing solvents). The obtained fraction was concentrated to give an oily substance. The oily substance was purified by high performance liquid column chromatography (with chloroform as a developing solvent). The obtained fraction was concentrated to give an oily substance. Hexane was added to the oily substance, irradiation with ultrasonic waves was performed, and a precipitated solid was collected by suction filtration, whereby 0.66 g of a target white solid was obtained in a yield of 65%. The synthesis scheme of PCCz2PSi is shown in (b-1) below.

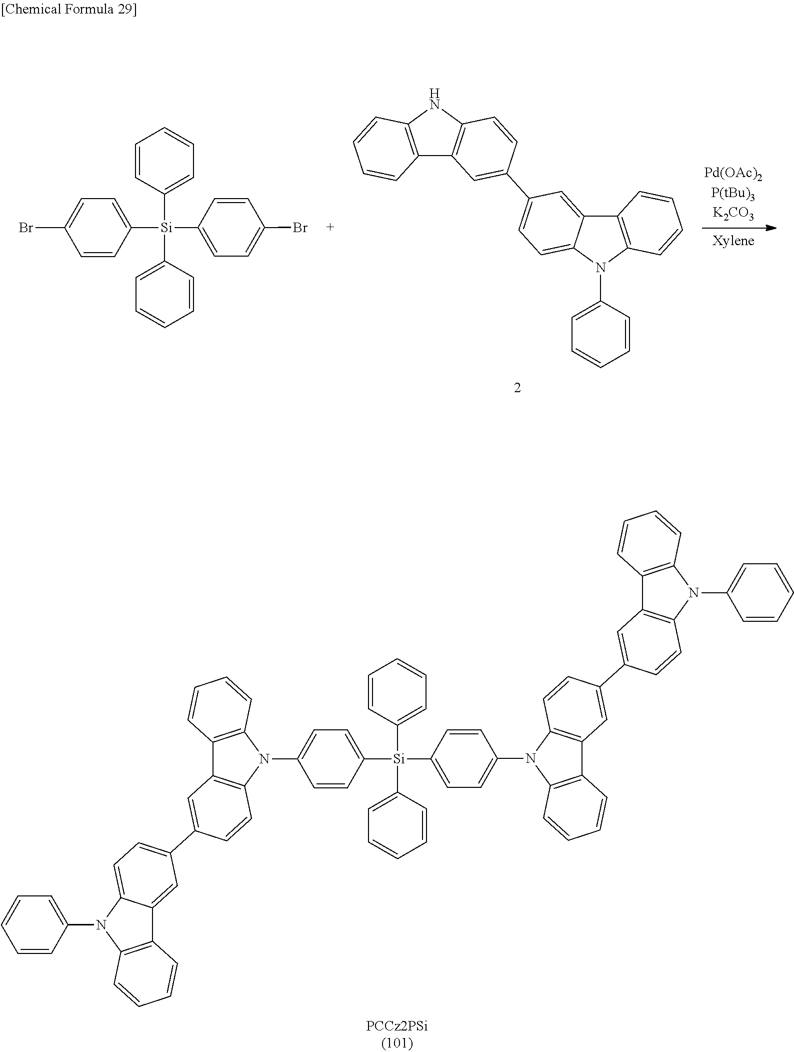 [SMILES: Brc1ccc([Si](c2ccccc2)(c2ccccc2)c2ccc(Br)cc2)cc1 ; c1ccc(-n2c3ccccc3c3cc(-c4ccc5[nH]c6ccccc6c5c4)ccc32)cc1 ; c1ccc(-n2c3ccccc3c3cc(-c4ccc5c(c4)c4ccccc4n5-c4ccc([Si](c5ccccc5)(c5ccccc5)c5ccc(-n6c7ccccc7c7cc(-c8ccc9c(c8)c8ccccc8n9-c8ccccc8)ccc76)cc5)cc4)ccc32)cc1]

By a train sublimation method, 0.66 g of the obtained white solid was purified. In the purification by sublimation, heating was performed for 17 hours at an argon flow rate of 5 mL/min, a pressure of 3.2 Pa, and a heating temperature of 455° C. As a result, a target white solid (0.38 g, at a collection rate of 58%) was obtained.

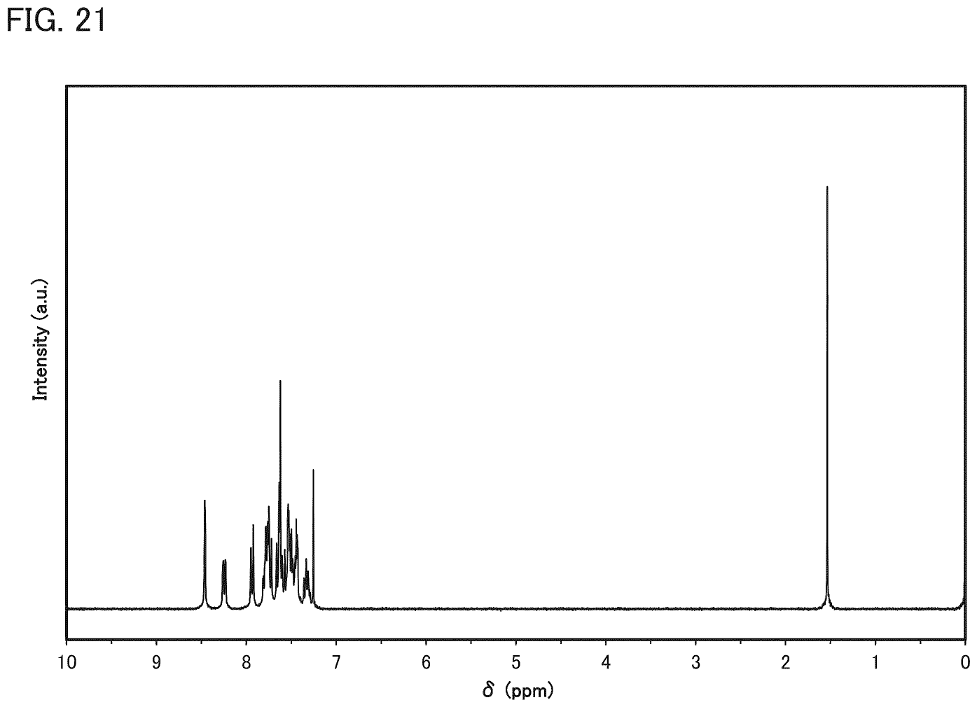

FIG. 21 shows a ¹H NMR spectrum of the organic compound obtained by the purification by sublimation. Results of ¹H NMR measurement are shown below. The results reveal that PCCz2PSi was obtained.

¹H NMR (CDCl₃, 300 MHz): δ=8.46 (s, 4H), 8.24 (d, J=6.3 Hz, 4H), 7.94 (d, J=7.8 Hz, 4H), 7.81-7.72 (m, 12H), 7.66-7.40 (m, 28H), 7.36-7.29 (m, 4H).

Next, measurement results of the T_{g} and emission spectra of PCCz2PSi whose synthesis method is shown in this example and a comparative compound are described. The comparative compound prepared was 9,9′-diphenyl-9H,9′H-3,3′-bicarbazole (abbreviation: PCCP), which has the same unit as PCCz2PSi but does not have a sigma bond. The following are the chemical formulae of PCCz2PSi and PCCP in addition to Unit B that these compounds include in common.

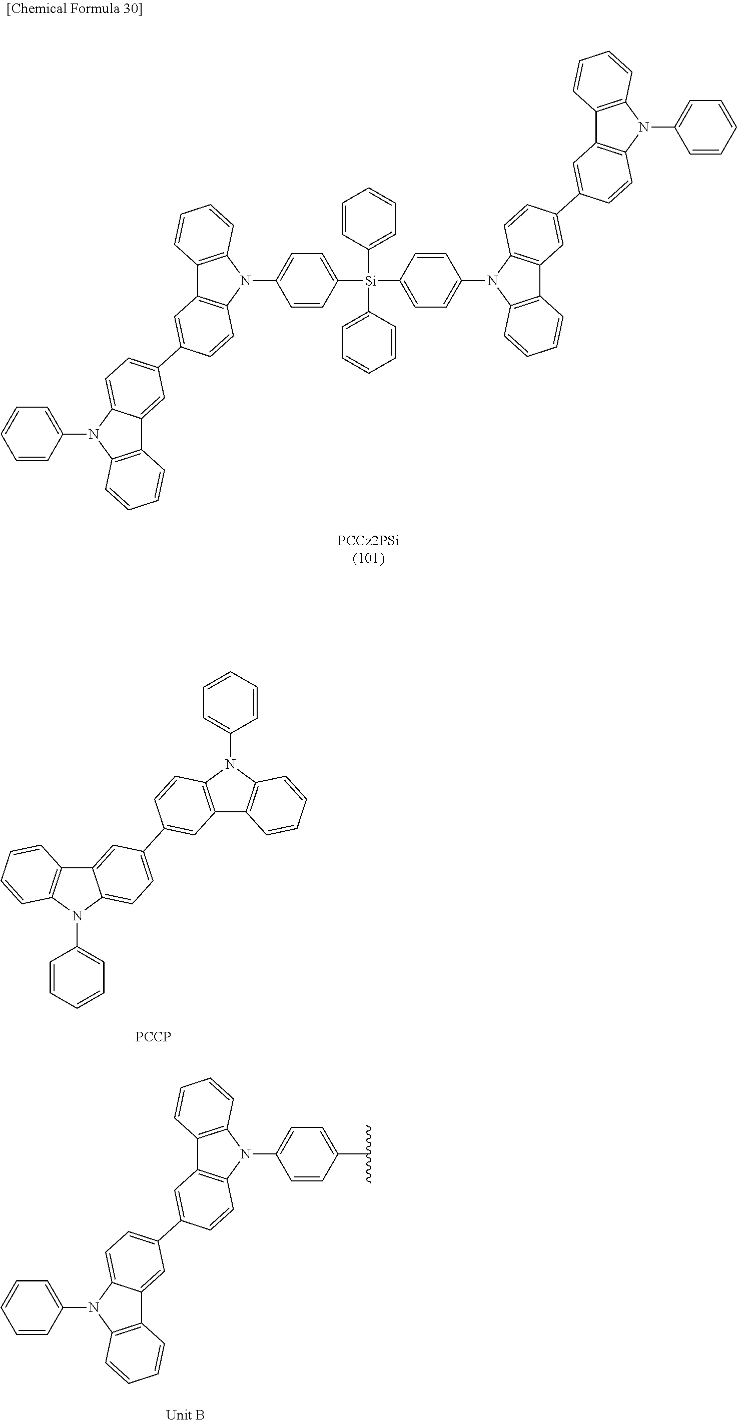

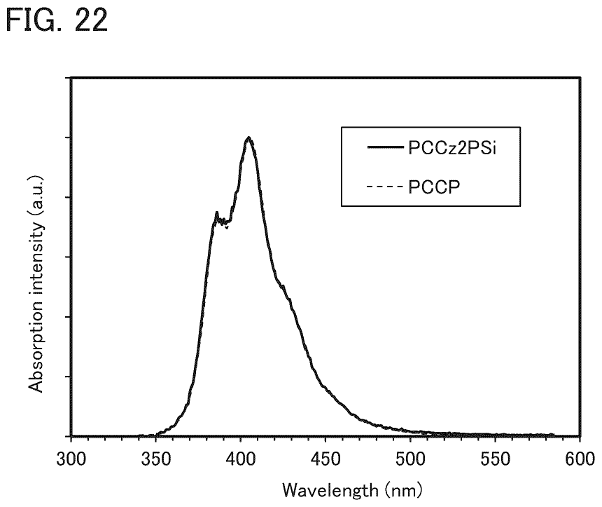

Emission spectra of toluene solutions of the compounds were measured with a fluorescence spectrophotometer (FS920, Hamamatsu Photonics K.K.). The emission spectra are shown in FIG. 22. The S₁ level of each compound was calculated in a manner similar to that in Example 2. The T_{g} of each compound was also measured in a manner similar to that in Example 2. The peak wavelength and the short-wavelength emission edge read from the emission spectrum, the S₁ level calculated from the emission edge, and the measured T_{g} of each compound are listed in the following table.

TABLE 2

| | Peak wavelength | Emission | S₁ | T_{g} |
|---|---|---|---|---|
| | (nm) | edge (nm) | level (eV) | (° C.) |
| PCCz2PSi | 390, 407 | 369 | 3.36 | 193 |
| PCCP | 390, 407 | 369 | 3.36 | 101 |

FIG. 22 and the above table show that the emission spectra of PCCz2PSi and PCCP overlap with each other and the peak wavelengths, the short-wavelength emission edges, and the S₁ levels of PCCz2PSi and PCCP are equivalent to each other.

The above table also reveals that the T_{g} of PCCz2PSi is higher than or equal to 190° C., which is significantly higher than that of PCCP. This is probably because the molecular weight is sufficiently increased with the structure in which two Units B are bonded to each other through a sigma bond.

The above results reveal that PCCz2PSi of one embodiment of the present invention is a material having a high S₁ level and a high T_{g}.

### Example 4

### Synthesis Example 4

This example shows synthesis of 9,9″-[3,3′-(diphenylsilyl)diphenyl]bis(3,9′-bi-9H-carbazole) (abbreviation: mCzCz2PSi) (structural formula (102)), which is the organic compound of one embodiment of the present invention, and various measurement results of this compound. The structure of mCzCz2PSi is shown below.

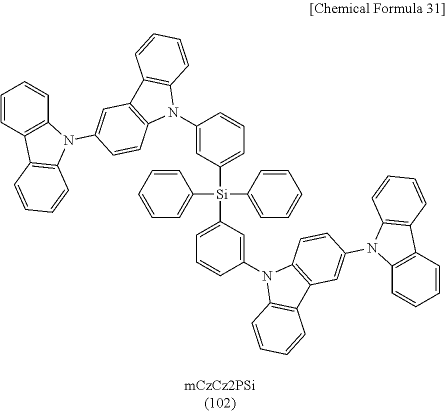 [SMILES: c1ccc([Si](c2ccccc2)(c2cccc(-n3c4ccccc4c4cc(-n5c6ccccc6c6ccccc65)ccc43)c2)c2cccc(-n3c4ccccc4c4cc(-n5c6ccccc6c6ccccc65)ccc43)c2)cc1]

Into a 200 mL three-neck flask were put 1.5 g (3.0 mmol) of bis(3-bromophenyl)diphenylsilane, 2.4 g (7.2 mmol) of 9H-3,9′-bicarbazole, 1.7 g (18 mmol) of sodium tert-butoxide, and 0.15 g (0.36 mmol) of 2-dicyclohexylphosphino-2′,6′-dimethoxybiphenyl (abbreviation: SPhos). To the mixture was added 20 mL of xylene and the mixture was degassed by being stirred while the pressure was reduced. To the mixture was added 0.17 g (0.18 mmol) of tris(dibenzylideneacetone)dipalladium(0) and the mixture was stirred while being heated at 140° C. under a nitrogen stream for 7 hours. After the stirring, toluene was added to the mixture, and the mixture was suction-filtered through Florisil, Celite, and alumina to give a filtrate. The obtained filtrate was concentrated to give a solid. This solid was purified by silica gel column chromatography (developing solvent: hexane:ethyl acetate in a ratio of 9:1). The obtained solid was reprecipitated with ethyl acetate/ethanol to give 2.6 g of a white solid in a yield of 86%.

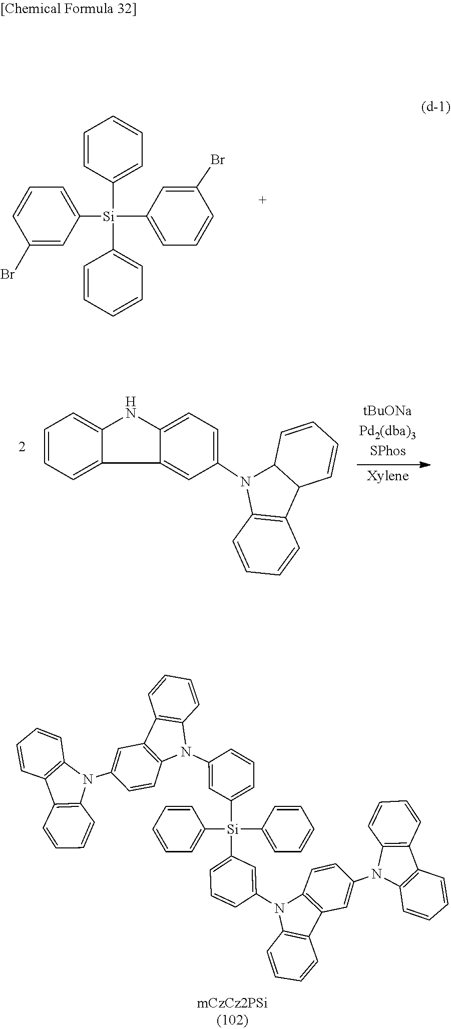 [SMILES: Brc1cccc([Si](c2ccccc2)(c2ccccc2)c2cccc(Br)c2)c1 ; C1=CC2c3ccccc3N(c3ccc4[nH]c5ccccc5c4c3)C2C=C1 ; c1ccc([Si](c2ccccc2)(c2cccc(-n3c4ccccc4c4cc(-n5c6ccccc6c6ccccc65)ccc43)c2)c2cccc(-n3c4ccccc4c4cc(-n5c6ccccc6c6ccccc65)ccc43)c2)cc1]

By a train sublimation method, 2.5 g of the obtained solid was purified. In the purification by sublimation, heating was performed at an argon flow rate of 15 mL/min, a pressure of 3.8 Pa, and a heating temperature of 380° C. After the sublimation purification, 2.2 g of a white solid was obtained at a collection rate of 89%.

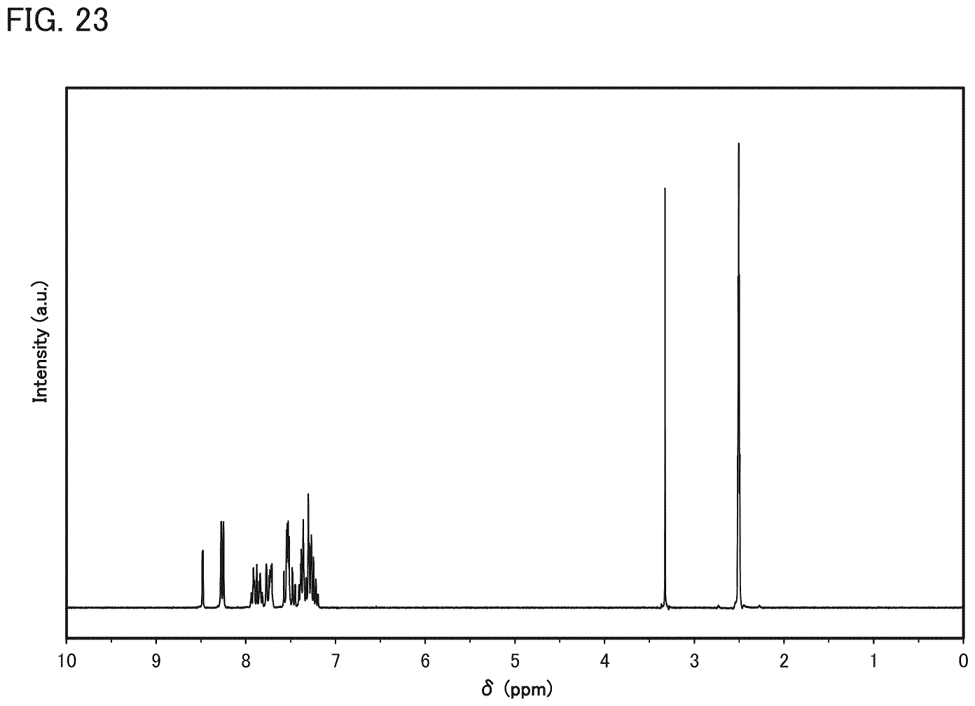

FIG. 23 shows a ¹H NMR spectrum of the organic compound obtained by the purification by sublimation. Results of ¹H NMR measurement are shown below. The results reveal that mCzCz2PSi was obtained.

¹H NMR (DMSO-d₆, 300 MHz): δ=7.19-7.41 (m, 18H), 7.55 (dd, J1=8.4 Hz, J2=1.8 Hz, 2H), 7.51-7.58 (m, 8H), 7.70-7.95 (m, 12H), 8.26 (d, J1=7.5 Hz, 6H), 8.48 (d, J1=2.4 Hz, 2H).

Next, mCzCz2PSi obtained in this example was analyzed by LC/MS.

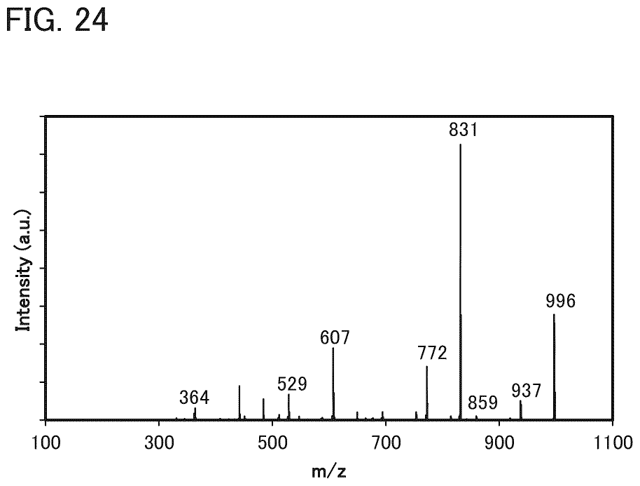

As in Example 1, MS/MS of the m/z of 996.36 corresponding to the exact mass of mCzCz2PSi was performed by a PRM method. For PRM, the mass range of a target ion was set to m/ [symbol] =996.36±2.0 (isolation window=4) and detection was performed in a positive mode. The measurement was performed with energy (normalized collision energy: NCE) accelerating the target ion in a collision cell set to 50. The MS spectrum obtained by MS/MS is shown in FIG. 24.

FIG. 24 shows that product ions of mCzCz2PSi are mainly detected at m/ [symbol] of around 364, around 529, around 607, around 772, around 831, around 859, and around 937.

[symbol] The product ion at m/ [symbol] of 364 corresponds to a product ion C₂₂H₁₆N₄OSi^{⋅+} obtained by elimination of a carbazolyl group, a phenyl-bicarbazolyl group, and a phenyl group from mCzCz2PSi and addition of water. The product ion at m/ [symbol] of 529 corresponds to a product ion C₃₆H₂₅N₂OSi^{⋅+} obtained by elimination of a phenyl-bicarbazolyl group and a phenyl group from mCzCz2PSi and addition of water. The product ion at m/ [symbol] of 607 corresponds to a product ion C₄₂H₃₁N₂OSi^{⋅+} obtained by elimination of a phenyl-bicarbazolyl group from mCzCz2PSi and addition of water. The product ion at m/ [symbol] of 772 corresponds to a product ion C₄₂H₃₁N₂OSi^{⋅+} obtained by elimination of a carbazolyl group and a phenyl group from mCzCz2PSi and addition of water. The product ion at m/ [symbol] of 831 corresponds to a product ion C₆₀H₄₁N₃Si⁺ obtained by elimination of a carbazolyl group from mCzCz2PSi and addition of hydrogen. The product ion at m/ [symbol] of 859 corresponds to a product ion C₆₀H₃₉N₄OSi⁺ obtained by elimination of two phenyl groups from mCzCz2PSi and addition of water. The product ion at m/ [symbol] of 937 corresponds to a product ion C₆₆H₄₅N₄OSi⁺ obtained by elimination of a phenyl group from mCzCz2PSi and addition of water.

The above results indicate that mCzCz2PSi includes a carbazole skeleton and a benzene skeleton. Note that the above m/ [symbol] values±1 may be detected as protonation and deprotonation products of the product ions.

As described above, one of the features of the general formula (G1) in which Q is silicon is detection of the m/ [symbol] values corresponding to the product ions of a silanol group (SiOH) formed by elimination of the first or second partial structure (aryl group) bonded to silicon and addition of water thereto.

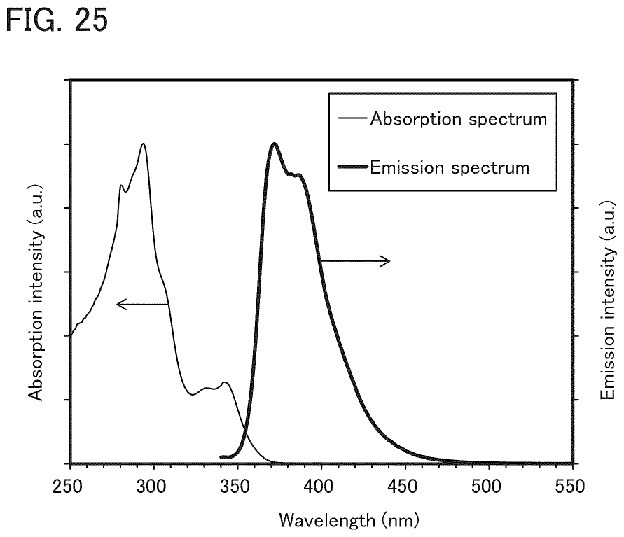

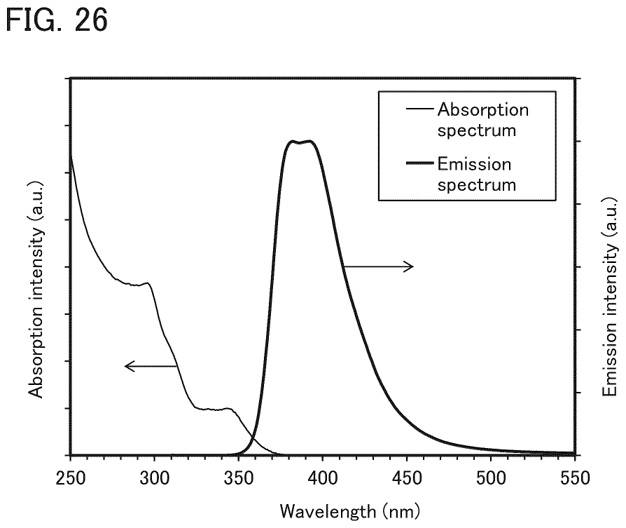

Next, the measurement results of the absorption and emission spectra of a toluene solution of mCzCz2PSi are shown in FIG. 25. The absorption spectrum of the toluene solution was measured with an ultraviolet-visible light spectrophotometer (V-770DS, JASCO Corporation), and the spectrum of toluene alone in a quartz cell was subtracted. Furthermore, the absorption and emission spectra of the thin film are shown in FIG. 26. The solid thin film was formed over a quartz substrate by a vacuum evaporation method. The absorption spectrum of the thin film was measured with a spectrophotometer (U-4100 Spectrophotometer, Hitachi High-Tech Corporation). The emission spectrum was measured with a fluorescence spectrophotometer (FP-8600, JASCO Corporation).

As shown in FIG. 25, the toluene solution of mCzCz2PSi has absorption peaks at 342 nm, 330 nm, 305 nm, and 294 nm and emission wavelength peaks at 372 nm and 387 nm (excitation wavelength: 330 nm). As shown in FIG. 26, the thin film of mCzCz2PSi has absorption peaks at 343 nm and 294 nm and emission wavelength peaks at 382 nm and 392 nm (excitation wavelength: 300 nm).

The comparison results of the T_{g} and emission spectra of mCzCz2PSi, whose synthesis method is shown in this example, and 9-[3-(triphenylsilyl)phenyl]-3,9′-bi-9H-carbazole (abbreviation: PSiCzCz) having the same unit as mCzCz2PSi are described. The following are the chemical formulae of mCzCz2PSi and PSiCzCz in addition to Unit C that these compounds include in common.

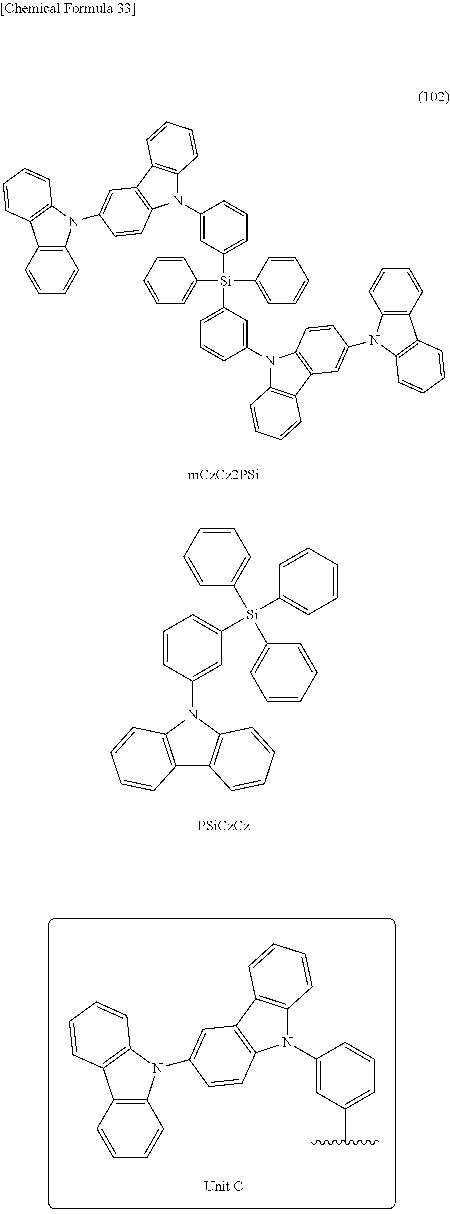 [SMILES: c1ccc([Si](c2ccccc2)(c2cccc(-n3c4ccccc4c4cc(-n5c6ccccc6c6ccccc65)ccc43)c2)c2cccc(-n3c4ccccc4c4cc(-n5c6ccccc6c6ccccc65)ccc43)c2)cc1 ; c1ccc([Si](c2ccccc2)(c2ccccc2)c2cccc(-n3c4ccccc4c4ccccc43)c2)cc1 ; *c1cccc(-n2c3ccccc3c3cc(-n4c5ccccc5c5ccccc54)ccc32)c1]

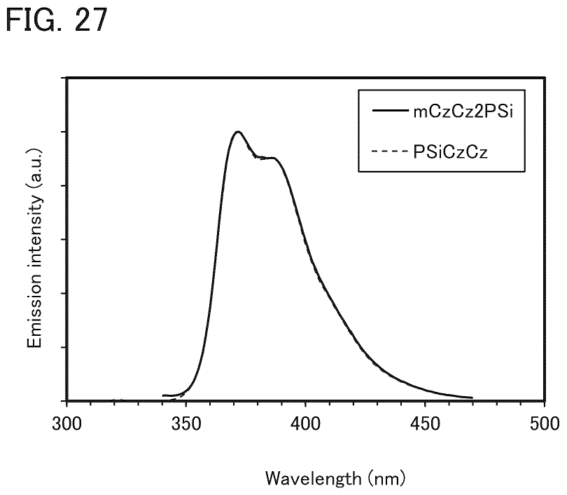

The results of the emission spectra of toluene solutions of the compounds measured with a fluorescence spectrophotometer (FP-8600, JASCO Corporation) are summarized in FIG. 27. Note that the S₁ level was energy of the wavelength (emission edge) of intersection of the horizontal axis (wavelength) or the base line and a tangent to the emission spectrum at a point where the slope of the spectrum at a peak on the shorter wavelength side has a maximum value.

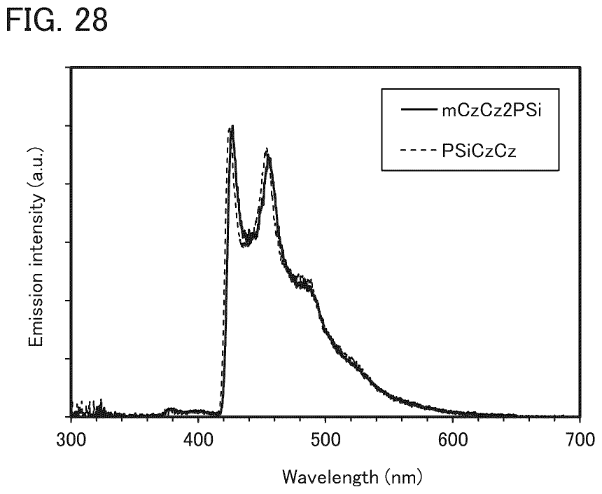

The emission spectra of thin films of the compounds at 10 K were measured. Specifically, phosphorescence spectra were measured in the following way: after the thin films were irradiated with excitation light (325 nm), 0.02 seconds were allowed to elapse, and light emission was then detected. FIG. 28 shows the emission spectra (phosphorescence spectra). Note that the T₁ level was energy of the wavelength (emission edge) of intersection of the horizontal axis (wavelength) or the base line and a tangent to the emission spectrum (phosphorescence spectrum) at a point where the slope of the spectrum at a peak on the shorter wavelength side has a maximum value.

The T_{g} of each compound was also measured in a manner similar to that in Example 2. The peak wavelength and the short-wavelength emission edge read from the emission spectra in FIG. 27, the S₁ level calculated from the emission edge, the T₁ level which is energy of a shorter-wavelength rising (onset) of the emission spectrum read from the emission spectra (phosphorescence spectra) in FIG. 28, and the measured T_{g} of each compound are listed in the following table.

TABLE 3

| | Peak | Emission | S₁ | T₁ | |
|---|---|---|---|---|---|
| | wavelength | edge | level | level | Tg |
| | (nm) | (nm) | (eV) | (eV) | (° C.) |
| mCzCz2PSi | 372 | 356 | 3.48 | 3.0 | 166 |
| PSiCzCz | 371 | 356 | 3.48 | 3.0 | 115 |

FIG. 27 and the above table show that the fluorescence emission spectra of mCzCz2PSi and PSiCzCz overlap with each other and the peak wavelengths, the short-wavelength emission edges, and the S₁ levels of mCzCz2PSi and PSiCzCz are equivalent to each other.

The above table also reveals that the T_{g} of mCzCz2PSi is higher than or equal to 160° C., which is significantly higher than that of PSiCzCz. This is probably because the molecular weight is sufficiently increased with the structure in which two Units C are bonded to each other through a sigma bond, as compared with PSiCzCz having only one Unit C.

FIG. 28 and the above table show that the energy of a shorter-wavelength onset of the emission spectrum (phosphorescence spectrum) of each of mCzCz2PSi and PSiCzCz is 3.0 eV. Moreover, the emission spectra of mCzCz2PSi and PSiCzCz substantially overlap with each other. Accordingly, it can be said that the T₁ levels of mCzCz2PSi and PSiCzCz are equivalent to each other. Note that the spectra observed at around 370 nm to around 420 nm are derived from fluorescence.

The above results reveal that mCzCz2PSi of one embodiment of the present invention is a material having a high S₁ level and a high T_{g}. The results in Examples 2 to 4 reveal that the organic compounds of one embodiment of the present invention are each a material having a high S₁ level, a high T₁ level, and a high T_{g}.

Since the organic compound of one embodiment of the present invention has a high T_{g} of 100° C. or higher, a light-emitting device including the organic compound can have favorable characteristics even when stored or driven at high temperature. Furthermore, in the process for the fabrication of the light-emitting device, a layer including the organic compound can maintain favorable film quality even after high-temperature treatment, which also enables the light-emitting device to have favorable characteristics.

### Example 5

This example shows fabrication of the light-emitting device 1, a blue fluorescent light-emitting device fabricated using PSiYGA2, which is the organic compound of one embodiment of the present invention, and the comparative light-emitting device 2 and various measurement results of the devices. Structural formulae of the organic compounds used for the light-emitting device 1 and the comparative light-emitting device 2 are shown below. Furthermore, device structures of the light-emitting device 1 and the comparative light-emitting device 2 are shown in Table 4.

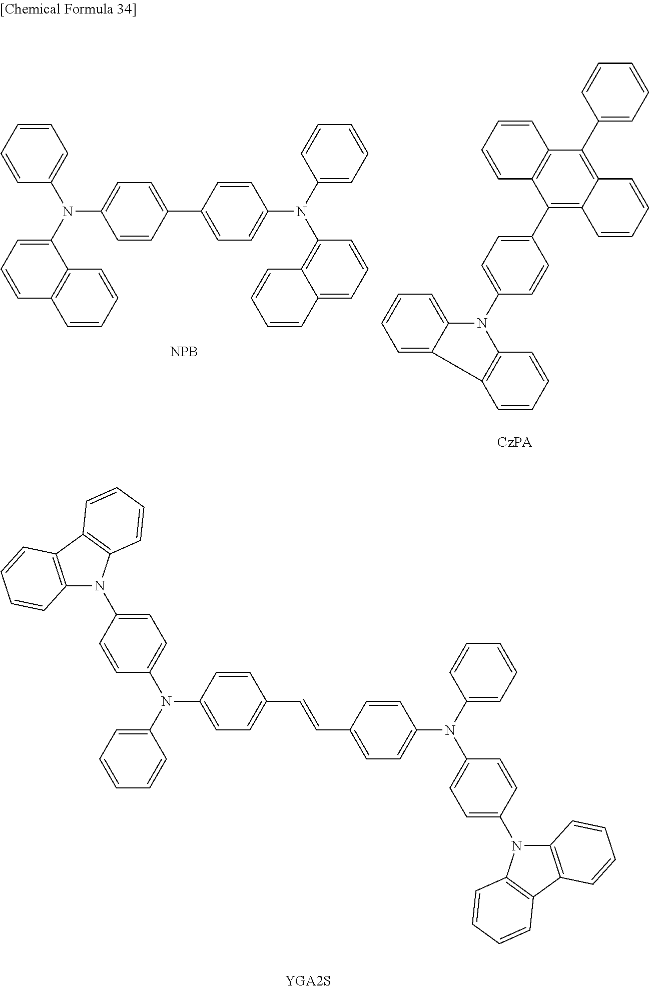

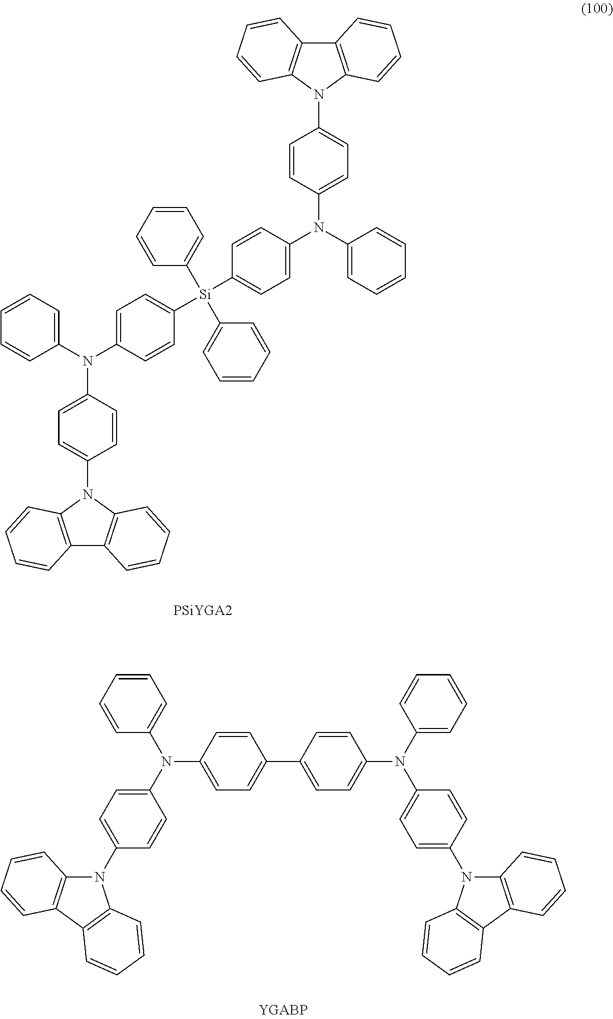 [SMILES: c1ccc(N(c2ccc(-n3c4ccccc4c4ccccc43)cc2)c2ccc([Si](c3ccccc3)(c3ccccc3)c3ccc(N(c4ccccc4)c4ccc(-n5c6ccccc6c6ccccc65)cc4)cc3)cc2)cc1 ; c1ccc(N(c2ccc(-c3ccc(N(c4ccccc4)c4ccc(-n5c6ccccc6c6ccccc65)cc4)cc3)cc2)c2ccc(-n3c4ccccc4c4ccccc43)cc2)cc1]

TABLE 4

| | | |
|---|---|---|
| | | Comparative |
| | Light-emitting | light-emitting |
| Thickness | device 1 | device 2 |

| Second electrode | | 200 nm | Al |
|---|---|---|---|
| Electron-injection layer | | 1 nm | LiF |
| Electron- | 2 | 20 nm | Bphen |
| transport layer | 1 | 10 nm | Alq₃ |

| Light-emitting layer | 30 nm | CzPA:YGA2S (1:0.05) | |
|---|---|---|---|
| Hole-transport layer | 10 nm | PSiYGA2 | YGABP |

| | | |
|---|---|---|
| Hole-injection layer | 50 nm | NPB:MoO₃ (4:1) |
| First electrode | 110 nm | ITSO |

### <<Fabrication of Light-Emitting Device 1>>

In the light-emitting device 1 described in this example, the hole-injection layer, the hole-transport layer, a light-emitting layer, the electron-transport layer (a first electron-transport layer and the second electron-transport layer), and the electron-injection layer are stacked in this order over the first electrode formed over the substrate, and the second electrode is stacked over the electron-injection layer.

First, the first electrode was formed over the substrate. The electrode area was set to 4 mm² (2 mm×2 mm). A glass substrate was used as the substrate. As the first electrode, an indium tin oxide containing silicon oxide (ITSO) was deposited to a thickness of 110 nm by a sputtering method. In this example, the first electrode serves as an anode.

For pretreatment, a surface of the substrate was washed with water, baking was performed at 200° C. for one hour, and then UV ozone treatment was performed for 370 seconds. After that, the substrate was transferred into a vacuum evaporation apparatus where the pressure had been reduced to approximately 1×10⁻⁴ Pa, and was subjected to vacuum baking at 170° C. for 30 minutes in a heating chamber of the vacuum evaporation apparatus, and then the substrate was cooled down for approximately 30 minutes.

Next, the hole-injection layer was formed over the first electrode. The hole-injection layer was formed to a thickness of 50 nm by co-evaporation of 4,4′-bis[N-(1-naphthyl)-N-phenylamino]biphenyl (abbreviation: NPB) and molybdenum oxide (MoO₃) at a weight ratio of 4:1 (=NPB:MoO₃).

Then, the hole-transport layer was formed over the hole-injection layer. As the hole-transport layer, PSiYGA2 was evaporated to a thickness of 10 nm.

Next, the light-emitting layer was formed over the hole-transport layer. The light-emitting layer was formed to a thickness of 30 nm by co-evaporation of 9-[4-(10-phenyl-9-anthracenyl)phenyl]-9H-carbazole (abbreviation: CzPA) and N,N′-bis[4-(9H-carbazol-9-yl)phenyl]-N,N′-diphenyl-4,4′-stilbenediamine (abbreviation: YGA2S) at a weight ratio of 1:0.05 (=CzPA:YGA2S).

After that, the electron-transport layer was formed over the light-emitting layer. The electron-transport layer was formed as follows: the first electron-transport layer was first formed to a thickness of 10 nm by co-evaporation of tris(8-quinolinolato)aluminum(III) (abbreviation: Alq₃); then, the second electron-transport layer was formed by evaporation of bathophenanthroline (abbreviation: BPhen) to a thickness of 20 nm.

Then, the electron-injection layer was formed over the electron-transport layer. The electron-injection layer was deposited to a thickness of 1 nm by evaporation of lithium fluoride (LiF).

Next, the second electrode was formed over the electron-injection layer. The second electrode was formed to a thickness of 200 nm by evaporation of aluminum (Al). In this example, the second electrode serves as a cathode.

Through the above process, the light-emitting device 1 was fabricated. Next, a method for fabricating the comparative light-emitting device 2 will be described.

<<Fabrication of comparative light-emitting device 2>>

The comparative light-emitting device 2 was fabricated in the same manner as the light-emitting device 1, except that PSiYGA2 used in the hole-transport layer of the light-emitting device 1 was replaced with YGABP.

The light-emitting device 1 and the comparative light-emitting device 2 were each sealed using a glass substrate in a glove box containing a nitrogen atmosphere so as not to be exposed to the air (a sealing material was applied to surround the devices and UV treatment and heat treatment at 80° C. for one hour were performed at the time of sealing). Then, the initial characteristics of the light-emitting devices were measured.

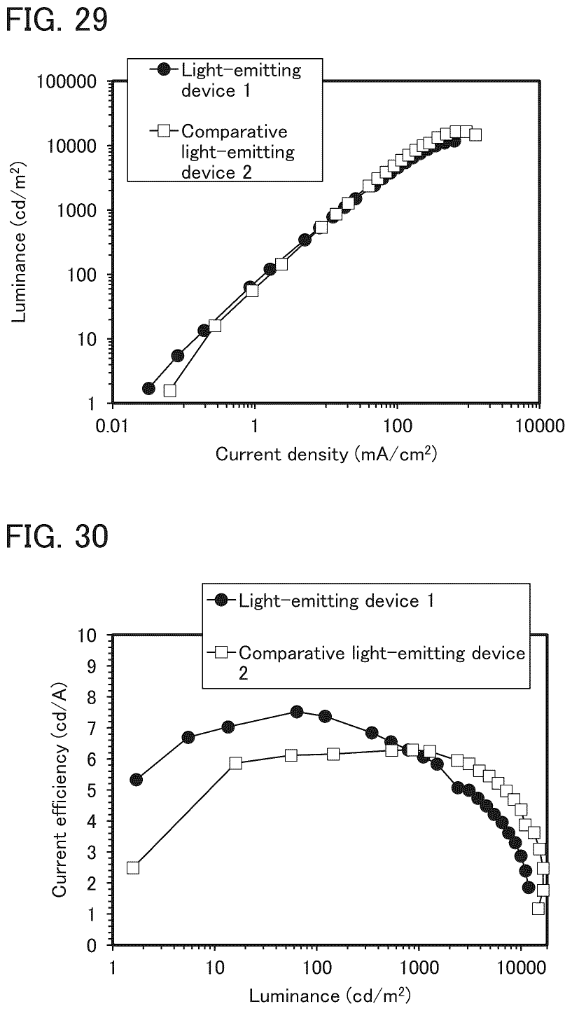

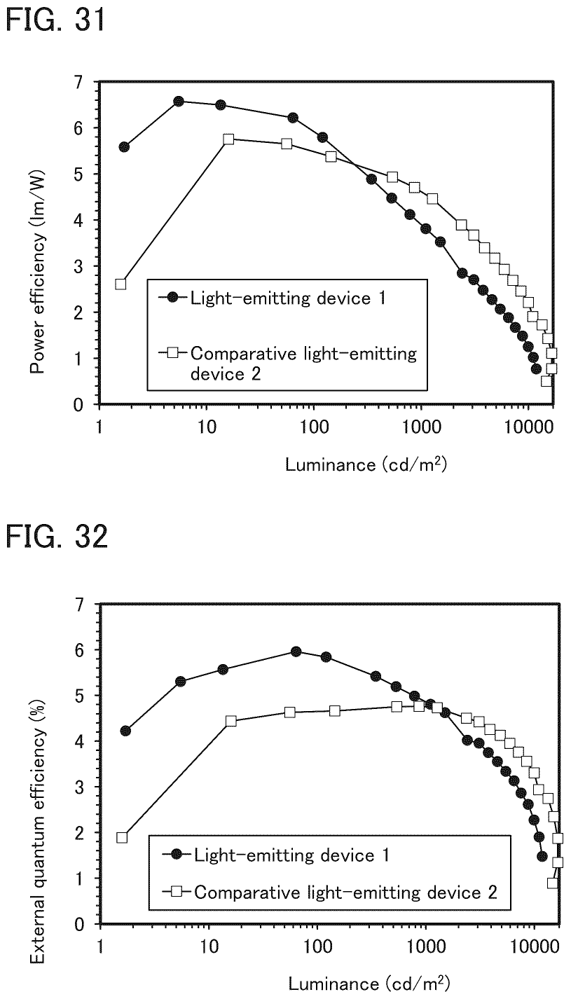

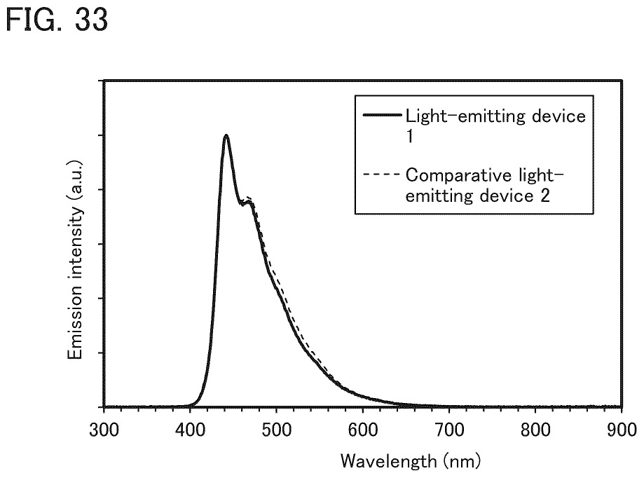

FIG. 29 shows the luminance-current density characteristics of the light-emitting device 1 and the comparative light-emitting device 2; FIG. 30, the current efficiency-luminance characteristics; FIG. 31, the power efficiency-luminance characteristics; FIG. 32, the external quantum efficiency-luminance characteristics; and FIG. 33, the electroluminescence spectra measured at a current density of 2.5 mA/cm².

Table 5 shows the main characteristics of the light-emitting device 1 and the comparative light-emitting device 2 at a luminance of approximately 100 cd/m². Luminance and CIE chromaticity were measured with a luminance colorimeter (BM-5A, TOPCON TECHNOHOUSE CORPORATION), and electroluminescence spectra were measured with a multi-channel spectrometer (PMA-11, Hamamatsu Photonics K.K.) at normal temperature. The external quantum efficiency described in this example was calculated from measurement on the front assuming a perfectly diffusing surface (also referred to as a Lambertian surface).

TABLE 5

| | | Current | Current | External |
|---|---|---|---|---|
| | Voltage | density | efficiency | quantum |
| | (V) | (mA/cm²) | (cd/A) | efficiency (%) |
| Light-emitteing device 1 | 4.0 | 1.6 | 7.4 | 5.8 |
| Comparative light- | 3.6 | 2.3 | 6.2 | 4.7 |
| emitteing device 2 | | | | |

FIG. 29 to FIG. 33 and the above table reveal that the light-emitting device 1 exhibits blue light emission derived from YGA2S and has favorable emission characteristics. In addition, the light-emitting device 1 was found to have higher current efficiency and higher external quantum efficiency than the comparative light-emitting device 2. The light-emitting device 1 was found to have higher maximum current efficiency and higher maximum external quantum efficiency than the comparative light-emitting device 2.

A reason for the above is that PSiYGA2 used in the hole-transport layer in the light-emitting device 1 has a higher S₁ level than YGABP used in the hole-transport layer in the comparative light-emitting device 2, as described in Example 2. It can be said that owing to PSiYGA2 having a higher S₁ level in the hole-transport layer, the light-emitting device 1 has higher emission efficiency than the comparative light-emitting device 2 including YGABP having a lower S₁ level in the hole-transport layer.

Thus, the use of the organic compound of one embodiment of the present invention for the hole-transport layer adjacent to the light-emitting layer can provide the light-emitting device with high emission efficiency.

### Example 6

This example shows fabrication of the light-emitting device 3, a blue fluorescent light-emitting device fabricated using YGABmP, which is the organic compound of one embodiment of the present invention, and the comparative light-emitting device 4 and various measurement results of the devices. Structural formulae of the organic compounds used for the light-emitting device 3 and the comparative light-emitting device 4 are shown below. Furthermore, device structures of the light-emitting device 3 and the comparative light-emitting device 4 are shown in Table 6.

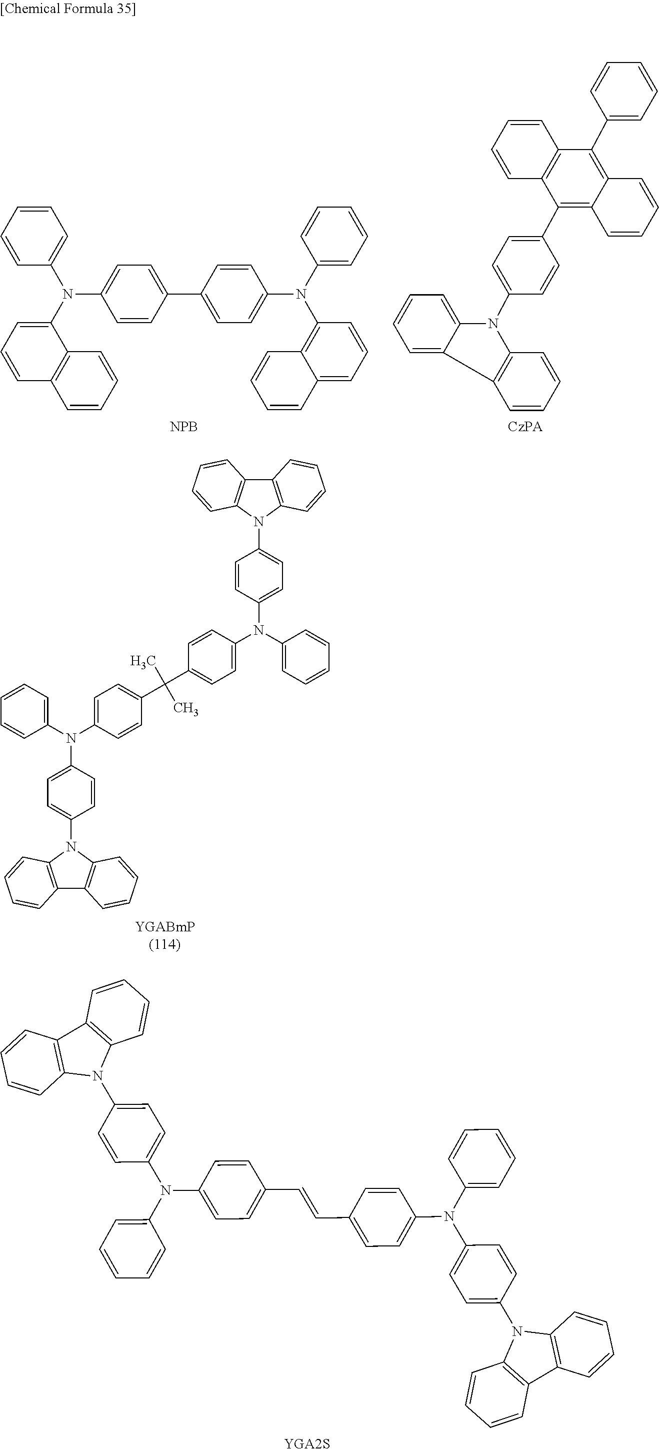

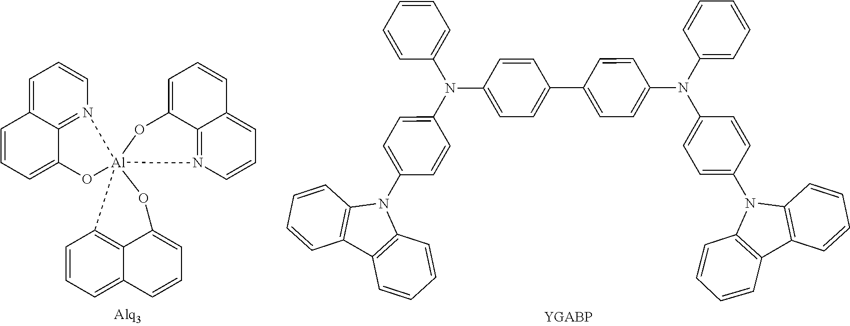

TABLE 6

| | | Light- | Comparative |
|---|---|---|---|
| | | emitting | light-emitting |
| | Thickness | device 3 | device 4 |
| Second electrode | 200 nm | Al | |
| Electron-injection layer | 20 nm | Alq₃:Li (1:0.01) | |
| Electron-transport layer | 10 nm | Alq₃ | |
| Light-emitting layer | 30 nm | CzPA:YGA2S (1:0.05) | |
| Hole-transport layer | 10 nm | YGABmP | YGABP |

| | | |
|---|---|---|
| Hole-injection layer | 50 nm | NPB:MoOx (4:1) |
| First electrode | 110 nm | ITSO |

### <<Fabrication of Light-Emitting Device 3>>

The light-emitting device 3 is different from the light-emitting device 1 in that YGABmP replaces PSiYGA2 used in the hole-transport layer of the light-emitting device 1 described in the above example. The light-emitting device 3 is different from the light-emitting device 1 also in the materials used for the electron-transport layer and the electron-injection layer. Specifically, the light-emitting device 3 was fabricated in a manner similar to that of the light-emitting device 1 except that YGABmP was evaporated to a thickness of 10 nm to form the hole-transport layer, Alq₃ was evaporated to a thickness of 10 nm to form the electron-transport layer after formation of the light-emitting layer, and Alq₃ and lithium (Li) were co-evaporated to a thickness of 20 nm at a weight ratio of 1:0.01 (=Alq₃:Li) to form the electron-injection layer.

### <<Fabrication of Comparative Light-Emitting Device 4>>

The comparative light-emitting device 4 is different from the light-emitting device 3 in that YGABP replaces YGABmP used in the hole-transport layer of the light-emitting device 3. The other components were formed in the same manner as those in the light-emitting device 3.

The light-emitting device 3 and the comparative light-emitting device 4 were each sealed using a glass substrate in a glove box containing a nitrogen atmosphere so as not to be exposed to the air (a sealing material was applied to surround the devices and UV treatment and heat treatment at 80° C. for one hour were performed at the time of sealing). Then, the initial characteristics of the light-emitting devices were measured.

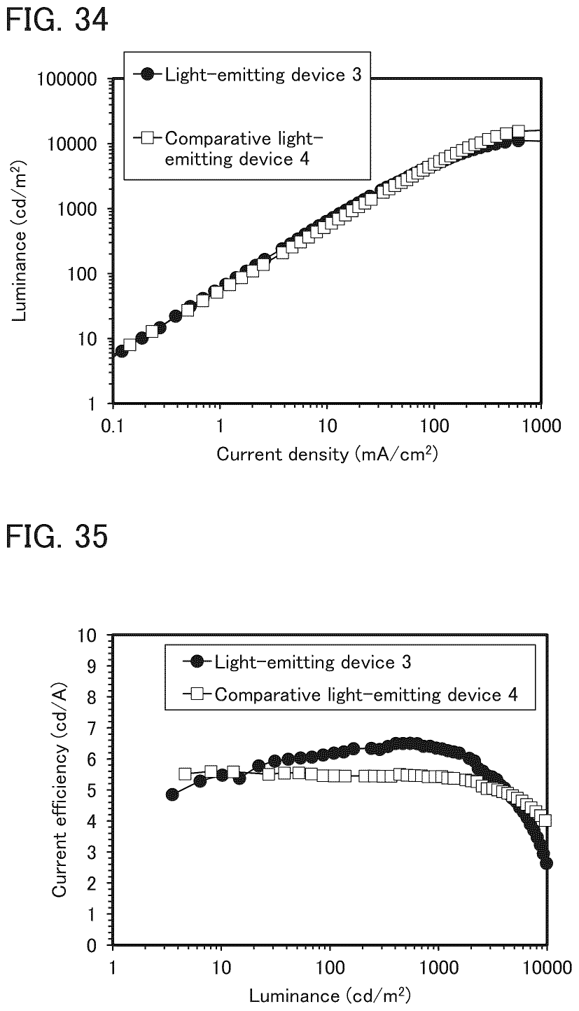

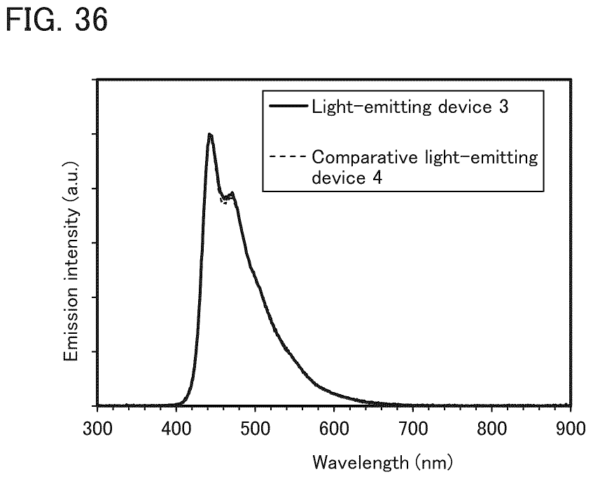

FIG. 34 shows the luminance-current density characteristics of the light-emitting device 3 and the comparative light-emitting device 4; FIG. 35, the current efficiency-luminance characteristics; and FIG. 36, the electroluminescence spectra measured at a current density of 2.5 mA/cm².

Table 7 shows the main characteristics of the light-emitting device 3 and the comparative light-emitting device 4 at a luminance of approximately 1000 cd/m². Luminance and CIE chromaticity were measured with a luminance colorimeter (BM-5A, TOPCON TECHNOHOUSE CORPORATION), and electroluminescence spectra were measured with a multi-channel spectrometer (PMA-11, Hamamatsu Photonics K.K.) at normal temperature.

TABLE 7

| | | Current | Current |
|---|---|---|---|
| | Voltage | density | efficiency |
| | (V) | (mA/cm²) | (cd/A) |
| Light-emitting device 3 | 5.6 | 14.9 | 6.4 |
| Comparative light-emitting device 4 | 5.4 | 19.6 | 5.4 |

FIG. 34 to FIG. 36 and the above table reveal that the light-emitting device 3 exhibits blue light emission derived from YGA2S and has favorable emission characteristics. In addition, the light-emitting device 3 was found to have higher current efficiency than the comparative light-emitting device 4. The light-emitting device 3 was found to have higher maximum current efficiency than the comparative light-emitting device 4.

A reason for the above is that YGABmP used in the hole-transport layer in the light-emitting device 3 has a higher S₁ level than YGABP used in the hole-transport layer in the comparative light-emitting device 4, as described in Example 2. It can be said that owing to YGABmP having a higher S₁ level in the hole-transport layer, the light-emitting device 3 has higher emission efficiency than the comparative light-emitting device 4 including YGABP having a lower S₁ level in the hole-transport layer.

Thus, the use of the organic compound of one embodiment of the present invention for the hole-transport layer adjacent to the light-emitting layer can provide the light-emitting device with high emission efficiency.

### Example 7

This example shows fabrication of the light-emitting device 5, the light-emitting device 7, and the light-emitting device 9, blue phosphorescent light-emitting devices fabricated using PCCz2PSi, which is the organic compound of one embodiment of the present invention, and comparative light-emitting devices (the comparative light-emitting device 6, the comparative light-emitting device 8, and the comparative light-emitting device 10) and various measurement results of the devices. Structural formulae of organic compounds used for each of the light-emitting devices are shown below. The device structures of the light-emitting devices are shown below in tables.

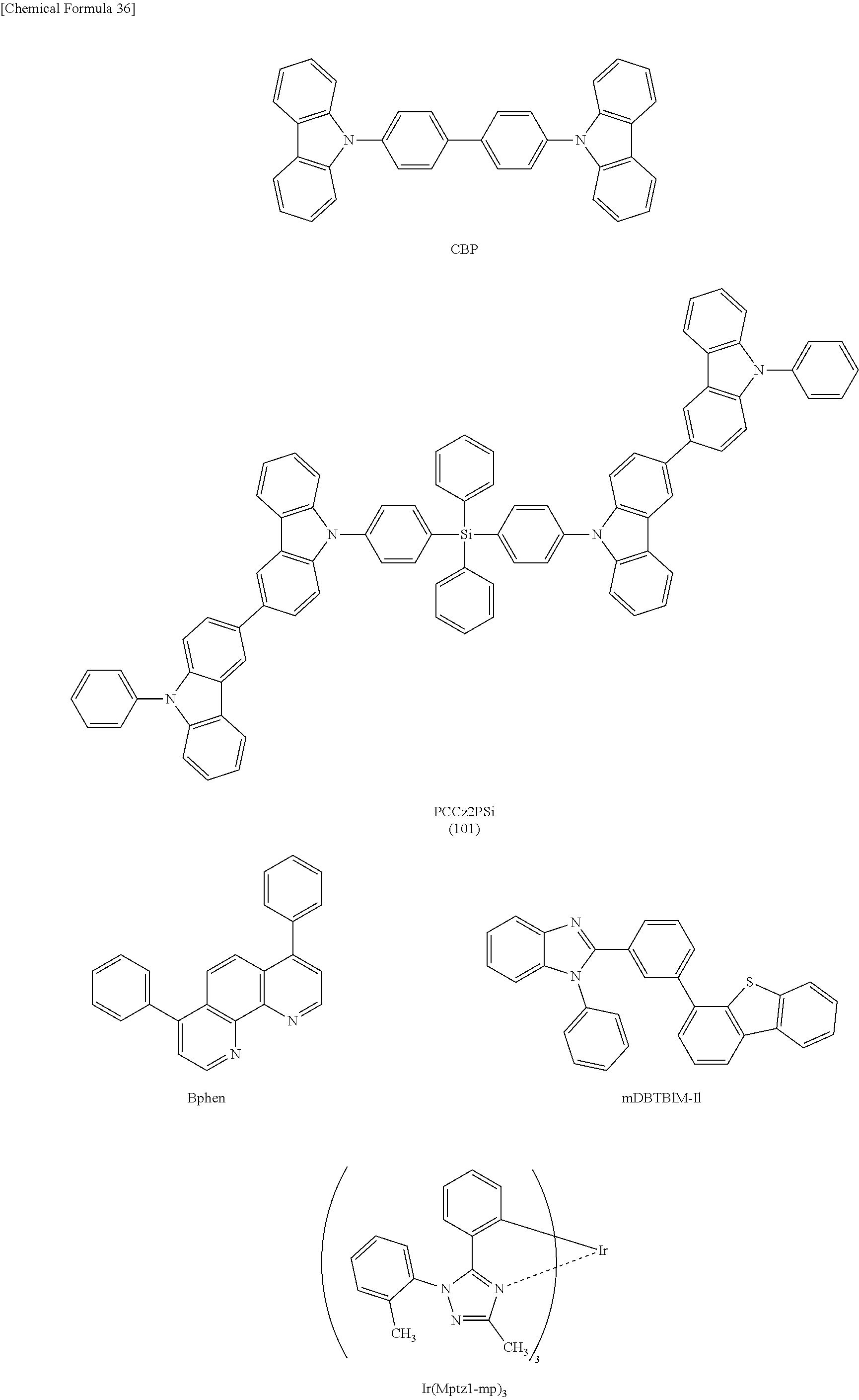

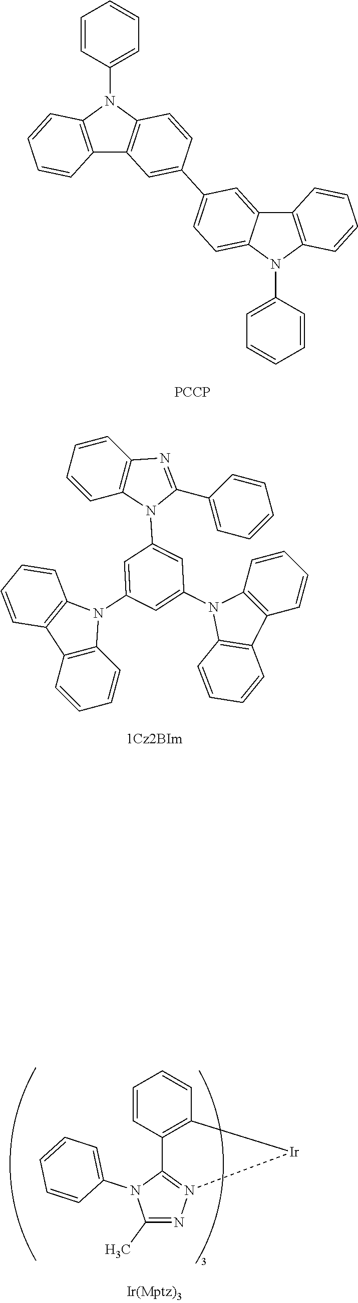

TABLE 8

| | | | | |
|---|---|---|---|---|
| | Light-emitting | Comparative light- | Light-emitting | Comparative light- |
| Thickness | device 5 | emitting device 6 | device 7 | emitting device 8 |

| Second electrode | | 200 nm | Al |
|---|---|---|---|
| Electron-injection layer | | 1 nm | LiF |
| Electron- | 2 | 15 nm | Bphen |
| transport layer | 1 | 10 nm | mDBTBIm-II:Ir(Mptz1-mp)₃ (1:0.06) |

| Light-emitting layer | 30 nm | 1Cz2BIm:PCCP:Ir(Mptz1-mp)₃ (1:0.25:0.06) | | | |
|---|---|---|---|---|---|
| Hole-transport layer | 20 nm | PCCz2PSi | PCCP | PCCz2PSi: | PCCP: |
| | | | | Ir(Mptz1-mp)₃ | Ir(Mptz1-mp)₃ |
| | | | | (1:0.25) | (1:0.25) |

| | | |
|---|---|---|
| Hole-injection layer | 60 nm | CBP:MoOx (4:2) |
| First electrode | 110 nm | ITSO |

TABLE 9

| | | |
|---|---|---|
| | | Comparative light- |
| Thickness | Light-emitting device 9 | emitting device 10 |

| Second electrode | | 200 nm | Al |
|---|---|---|---|
| Electron-injection layer | | 1 nm | LiF |
| Electron- | 2 | 15 nm | Bphen |
| transport layer | 1 | 10 nm | mDBTBIm-II:Ir(Mptz)₃ (1:0.06) |

| | | | |
|---|---|---|---|
| Light-emitting layer | 30 nm | 1Cz2BIm:PCCz2PSi:Ir(Mptz)₃ | 1Cz2BIm:PCCP:Ir(Mptz)₃ |
| | | (1:0.5:0.06) | (1:1:0.06) |

| | | |
|---|---|---|
| Hole-transport layer | 20 nm | PCCP:Ir(Mptz)₃ (1:0.25) |
| Hole-injection layer | 60 nm | CBP:MoOx (4:2) |
| First electrode | 110 nm | ITSO |

### <<Fabrication of Light-Emitting Device 5>>

In the light-emitting device 5 described in this example, the hole-injection layer, the hole-transport layer, the light-emitting layer, the electron-transport layer (the first electron-transport layer and the second electron-transport layer), and the electron-injection layer are stacked in this order over the first electrode formed over the substrate, and a second electrode is stacked over the electron-injection layer. The light-emitting device 5 includes PCCz2PSi in the hole-transport layer.

First, the first electrode was formed over the substrate. The electrode area was set to 4 mm² (2 mm×2 mm). A glass substrate was used as the substrate. As the first electrode, an indium tin oxide containing silicon oxide (ITSO) was deposited to a thickness of 110 nm by a sputtering method. In this example, the first electrode serves as an anode.

For pretreatment, a surface of the substrate was washed with water, baking was performed at 200° C. for one hour, and then UV ozone treatment was performed for 370 seconds. After that, the substrate was transferred into a vacuum evaporation apparatus where the pressure had been reduced to approximately 1×10⁻⁴ Pa, and was subjected to vacuum baking at 170° C. for 30 minutes in a heating chamber of the vacuum evaporation apparatus, and then the substrate was cooled down for approximately 30 minutes.

Next, the hole-injection layer was formed over the first electrode. The hole-injection layer was formed to a thickness of 60 nm by co-evaporation of 4,4′-di(N-carbazolyl)biphenyl (abbreviation: CBP) and molybdenum oxide (MoO₃) at a weight ratio of 4:2 (=CBP:MoO₃).

Then, the hole-transport layer was formed over the hole-injection layer. As the hole-transport layer, PCCz2PSi was evaporated to a thickness of 20 nm.

Next, the light-emitting layer was formed over the hole-transport layer. The light-emitting layer was formed to a thickness of 30 nm by co-evaporation of 1-[3,5-di(9H-carbazol-9-yl]phenyl)-2-phenylbenzimidazole (abbreviation: 1Cz2BIm), PCCP, and tris[3-methyl-1-(2-methylphenyl)-5-phenyl-1H-1,2,4-triazoleato]iridium(III) (abbreviation: Ir(Mptz1-mp)₃) at a weight ratio of 1:0.25:0.06 (=1Cz2BIm:PCCP:Ir(Mptz1-mp)₃).

After that, the electron-transport layer was formed over the light-emitting layer. The electron-transport layer was formed as follows: the first electron-transport layer was first formed to a thickness of 10 nm by co-evaporation of 2-[3-(dibenzothiophen-4-yl)phenyl]-1-phenyl-1H-benzimidazole (abbreviation: mDBTBIm-II) and Ir(Mptz1-mp)₃ at a weight ratio of 1:0.06 (=mDBTBIm-II:Ir(Mptz1-mp)₃); then, the second electron-transport layer was formed by evaporation of BPhen to a thickness of 15 nm.

Next, the electron-injection layer was formed by evaporation of lithium fluoride (LiF) to a thickness of 1 nm over the electron-transport layer, and then the second electrode was formed by evaporation of aluminum (Al) to a thickness of 200 nm. In this example, the second electrode serves as a cathode.

Through the above process, the light-emitting device 5 was fabricated. Next, methods for fabricating the comparative light-emitting device 6, the light-emitting device 7, the comparative light-emitting device 8, the light-emitting device 9, and the comparative light-emitting device 10 will be described.

### <<Fabrication of Comparative Light-Emitting Device 6>>

The comparative light-emitting device 6 was fabricated in the same manner as the light-emitting device 1, except that PCCz2PSi used in the hole-transport layer of the light-emitting device 5 was replaced with PCCP.

### <<Fabrication of Light-Emitting Device 7>>

The light-emitting device 7 was fabricated in a manner similar to that of the light-emitting device 5 except that the hole-transport layer was formed by co-evaporation of PCCz2PSi and Ir(Mptz1-mp)₃ at a weight ratio of 1:0.25 (=PCCz2PSi:Ir(Mptz1-mp)₃).

### <<Fabrication of the Comparative Light-Emitting Device 8>>

The comparative light-emitting device 8 was fabricated in the same manner as the light-emitting device 7, except that PCCz2PSi used in the hole-transport layer of the light-emitting device 7 was replaced with PCCP.

### <<Fabrication of Light-Emitting Device 9>>

The light-emitting device 9 was fabricated in a manner similar to that of the comparative light-emitting device 8 except that: tris(5-methyl-3,4-diphenyl-4H-1,2,4-triazolato)iridium(III) (abbreviation: Ir(Mptz)₃) replaces Ir(Mptz1-mp)₃ used in the hole-transport layer, the light-emitting layer, and the first electron-transport layer of the comparative light-emitting device 8; PCCz2PSi replaces PCCP used in the light-emitting layer in the comparative light-emitting device 8; and 1Cz2BIm, PCCz2PSi, and Ir(Mptz)₃ were co-evaporated at a weight ratio of 1:0.5:0.06 (=1Cz2BIm:PCCz2PSi:Ir(Mptz)₃). Thus, the light-emitting device 9 includes PCCz2PSi as a host of the light-emitting layer.

### <<Fabrication of Comparative Light-Emitting Device 10>>

The comparative light-emitting device 10 was fabricated in a manner similar to that of the light-emitting device 9 except that PCCz2PSi used in the light-emitting layer was replaced with PCCP and the light-emitting layer was formed by co-evaporation of 1Cz2BIm, PCCP, and Ir(Mptz)₃ at a weight ratio of 1:1:0.06 (=1Cz2BIm:PCCP:Ir (Mptz)₃).

This fabricated light-emitting devices were each sealed using a glass substrate in a glove box containing a nitrogen atmosphere so as not to be exposed to the air (a sealing material was applied to surround the devices and UV treatment was performed at the time of sealing). Then, the initial characteristics of the light-emitting devices were measured.

A table shows the main characteristics of the light-emitting devices at a luminance of approximately 1000 cd/m². Luminance and CIE chromaticity were measured with a luminance colorimeter (BM-5A, TOPCON TECHNOHOUSE CORPORATION), and electroluminescence spectra were measured with a multi-channel spectrometer (PMA-11, Hamamatsu Photonics K.K.) at normal temperature. The external quantum efficiency described in this example was calculated from measurement on the front assuming a perfectly diffusing surface (also referred to as a Lambertian surface).

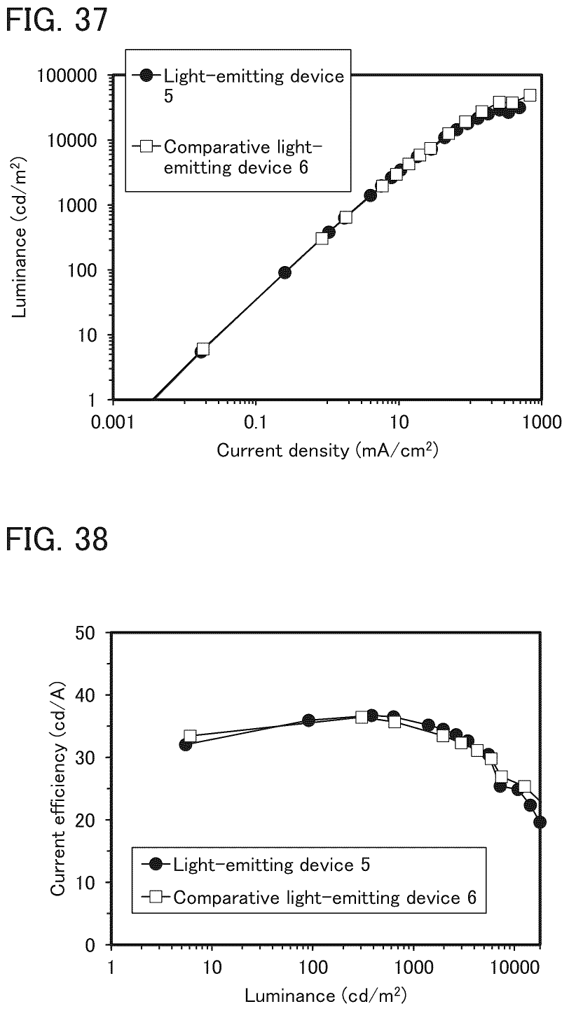

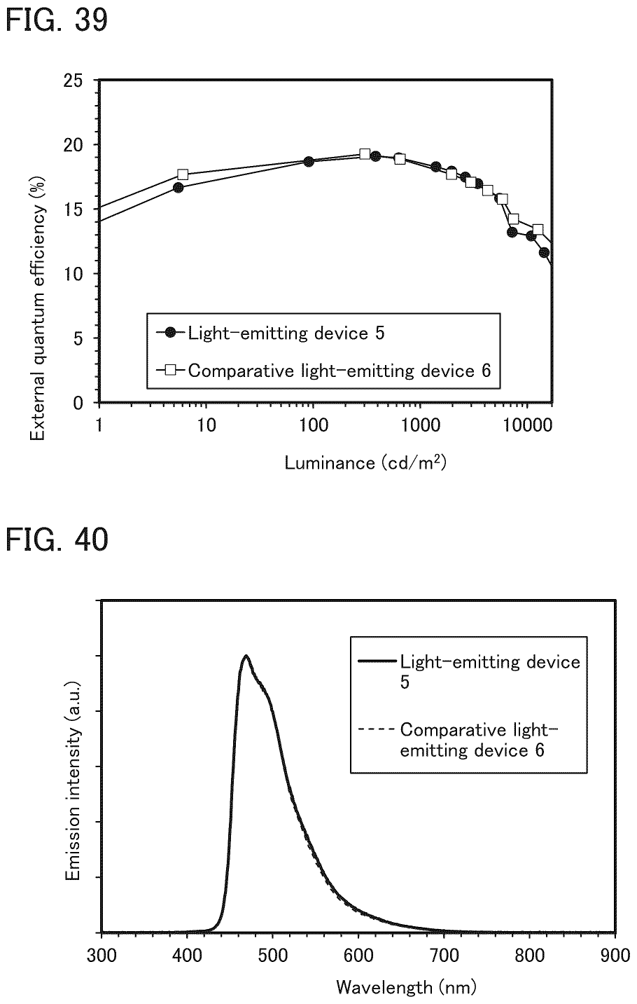

FIG. 37 shows the luminance-current density characteristics of the light-emitting device 5 and the comparative light-emitting device 6; FIG. 38, the current efficiency-luminance characteristics; FIG. 39, the external quantum efficiency-luminance characteristics; and FIG. 40, the electroluminescence spectra measured at a current density of 2.5 mA/cm². Furthermore, device structures of the light-emitting device 5 and the comparative light-emitting device 6 are shown in the following table.

TABLE 10

| | | | | | | External |
|---|---|---|---|---|---|---|
| | | Current | | | Current | quantum |
| | Voltage | density | Chromaticity | Chromaticity | efficiency | efficiency |
| | (V) | (mA/cm²) | x | y | (cd/A) | (%) |
| Light-emitting device 5 | 4.2 | 1.73 | 0.18 | 0.30 | 36.5 | 19.0 |
| Comparative light-emitting | 4.0 | 1.81 | 0.18 | 0.30 | 35.7 | 18.9 |
| device 6 | | | | | | |

FIG. 37 to FIG. 40 and the above table reveal that the light-emitting device 5 exhibits blue light emission derived from Ir(Mptz1-mp)₃, which is a light-emitting material, and has favorable emission characteristics. In addition, the light-emitting device 5 was found to have current efficiency and external quantum efficiency higher than or equal to those of the comparative light-emitting device 6. The light-emitting device 5 was found to have maximum current efficiency and higher maximum external quantum efficiency equivalent to those of the comparative light-emitting device 6.

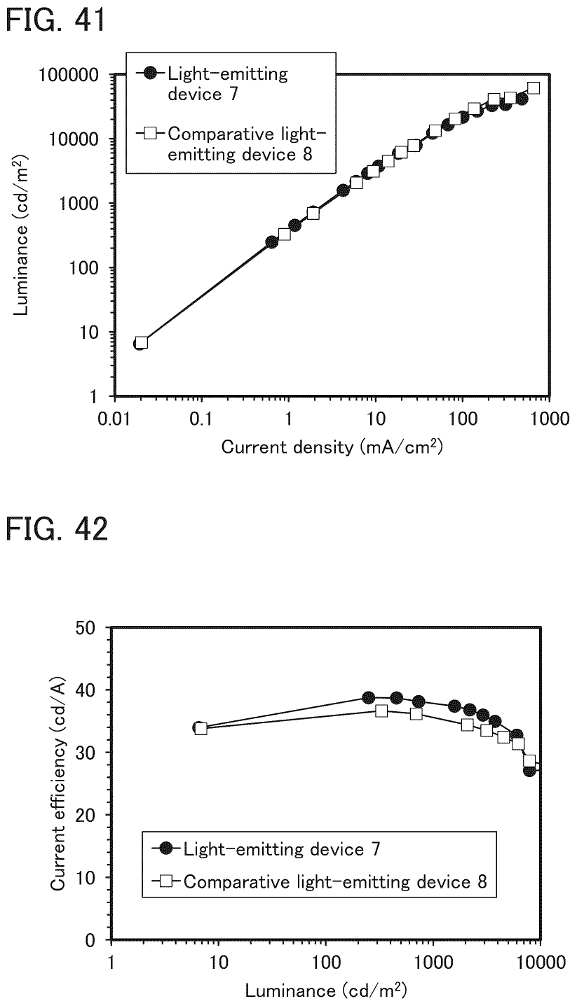

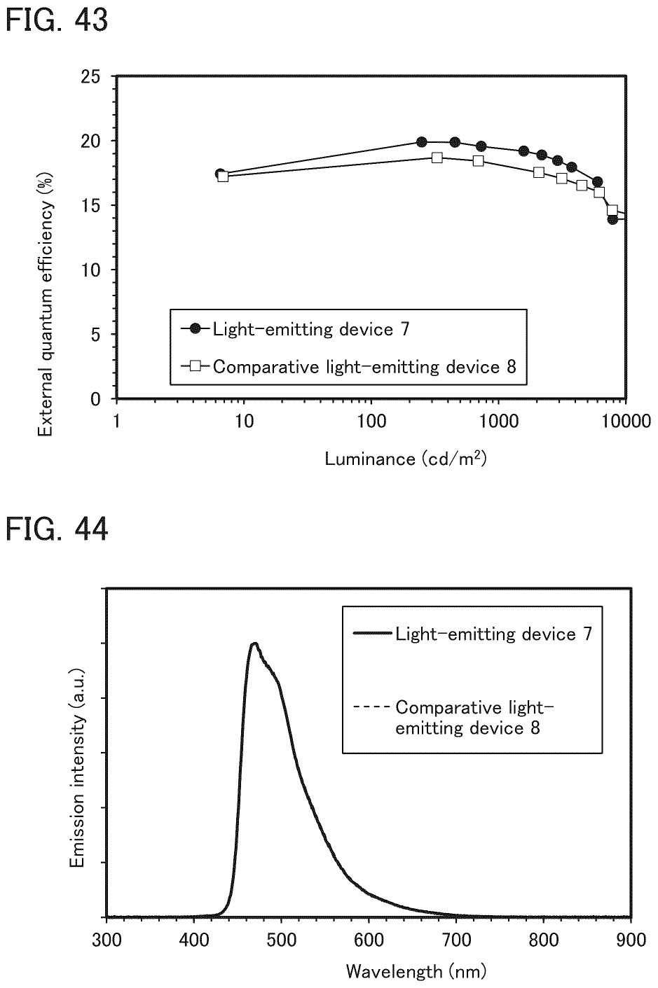

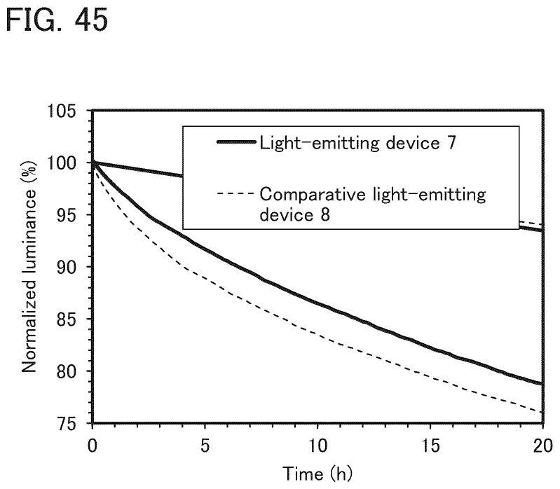

FIG. 41 shows the luminance-current density characteristics of the light-emitting device 7 and the comparative light-emitting device 8; FIG. 42, the current efficiency-luminance characteristics; FIG. 43, the external quantum efficiency-luminance characteristics; and FIG. 44, the electroluminescence spectra measured at a current density of 2.5 mA/cm². FIG. 45 shows luminance changes over driving time when the light-emitting device 7 and the comparative light-emitting device 8 are driven at a constant current of 2 mA (50 mA/cm²). Furthermore, device structures of the light-emitting device 7 and the comparative light-emitting device 8 are shown in the following table.

TABLE 11

| | | | | | | | External |
|---|---|---|---|---|---|---|---|
| | | Current | | | Current | Power | quantum |
| | Voltage | density | Chromaticity | Chromaticity | efficiency | efficiency | efficiency |
| | (V) | (mA/cm²) | x | y | (cd/A) | (lm/W) | (%) |
| Light-emitting device 7 | 4.2 | 1.92 | 0.18 | 0.31 | 38.1 | 28.5 | 19.6 |
| Comparative light- | 4.0 | 1.92 | 0.18 | 0.31 | 36.2 | 28.4 | 18.4 |
| emitting device 8 | | | | | | | |

FIG. 41 to FIG. 44 and the above table reveal that the light-emitting device 7 exhibits blue light emission derived from Ir(Mptz1-mp)₃ and favorable light-emitting characteristics. Moreover, the light-emitting device 7 was found to have higher external quantum efficiency than the comparative light-emitting device 8. In addition, the light-emitting device 7 was found to have power efficiency higher than or equal to that of the comparative light-emitting device 8. Furthermore, FIG. 45 reveals that the light-emitting device 7 shows less luminance change over driving time than the comparative light-emitting device 8. Moreover, the light-emitting device 7 was found to have higher maximum current efficiency and higher maximum external quantum efficiency than the comparative light-emitting device 8.

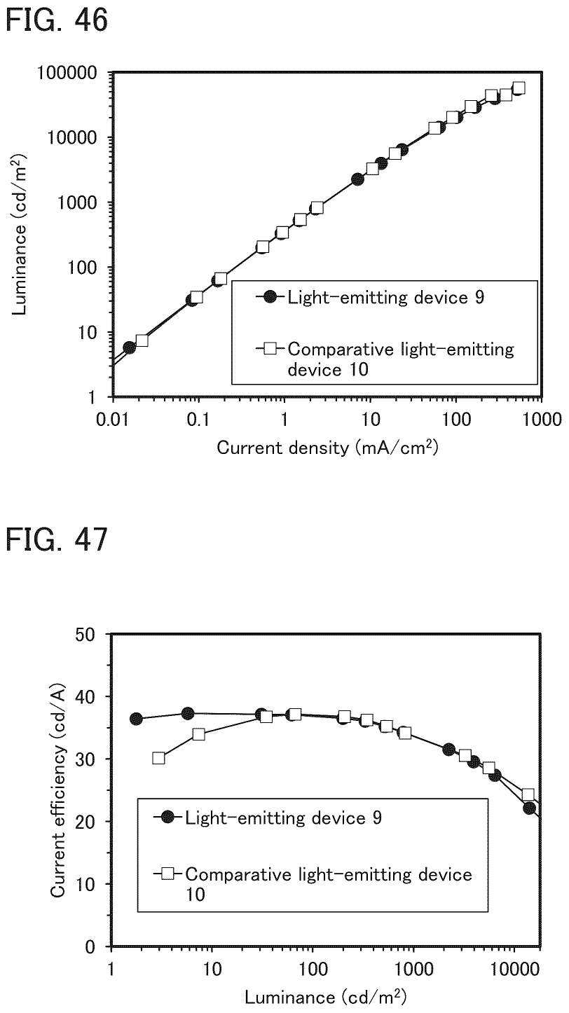

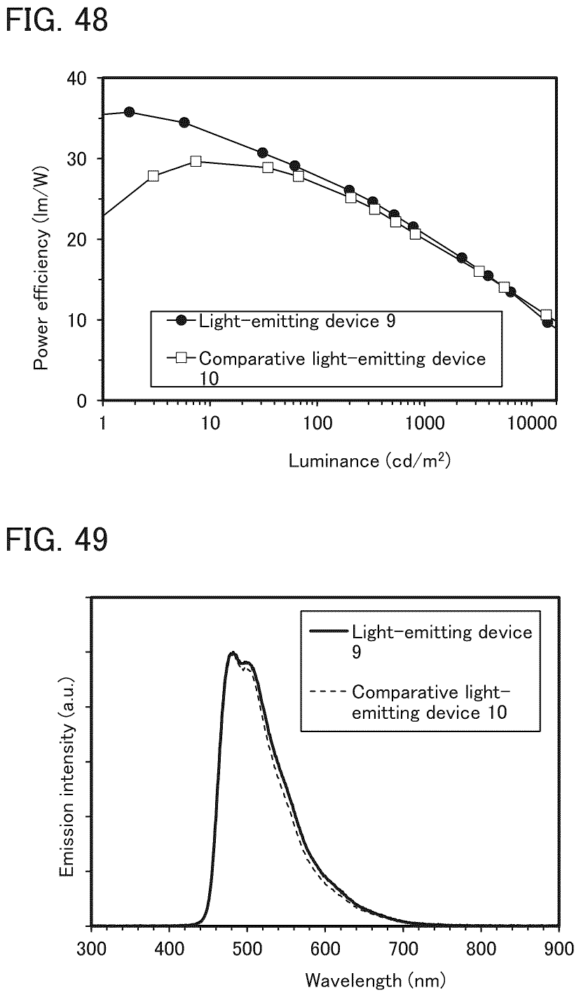

FIG. 46 shows the luminance-current density characteristics of the light-emitting device 9 and the comparative light-emitting device 10; FIG. 47, the current efficiency-luminance characteristics; FIG. 48, the power efficiency-luminance characteristics; and FIG. 49, the electroluminescence spectra measured at a current density of 2.5 mA/cm². Furthermore, device structures of the light-emitting device 9 and the comparative light-emitting device 10 are shown in the following table.

TABLE 12

| | | | | | Current | Power |
|---|---|---|---|---|---|---|
| | Voltage | Current density | Chromaticity | Chromaticity | efficiency | efficiency |
| | (V) | (mA/cm²) | x | y | (cd/A) | (lm/W) |
| Light-emitting device 9 | 5.0 | 2.30 | 0.22 | 0.41 | 34.3 | 21.5 |
| Comparative light- | 5.2 | 2.41 | 0.21 | 0.40 | 34.2 | 20.6 |
| emitting device 10 | | | | | | |

FIG. 46 to FIG. 49 and the above table reveal that the light-emitting device 9 exhibits blue light emission derived from Ir(Mptz)₃ and favorable light-emitting characteristics. In addition, the light-emitting device 9 was found to have higher power efficiency than the comparative light-emitting device 10. In addition, the light-emitting device 9 was found to have current efficiency higher than or equal to the comparative light-emitting device 10. In addition, the light-emitting device 9 was found to have higher maximum current efficiency and higher maximum external quantum efficiency higher than or equal to the comparative light-emitting device 9. Note that the mixture ratio of the two kinds of host materials used in the light-emitting layer of each light-emitting device was adjusted so that the optimum carrier balance was achieved to maximize efficiency.

Thus, the use of the organic compound of one embodiment of the present invention for the light-emitting layer or the hole-transport layer adjacent to the light-emitting layer can provide the light-emitting device with high emission efficiency.

### Example 8

This example shows fabrication of the light-emitting device 11, a blue phosphorescent light-emitting device fabricated using mCzCz2PSi, which is the organic compound of one embodiment of the present invention, and the comparative light-emitting device 12 and various measurement results of the devices. Structural formulae of the organic compounds used for the light-emitting device 11 and the comparative light-emitting device 12 are shown below. Furthermore, device structures of the light-emitting device 11 and the comparative light-emitting device 12 are shown below in a table.

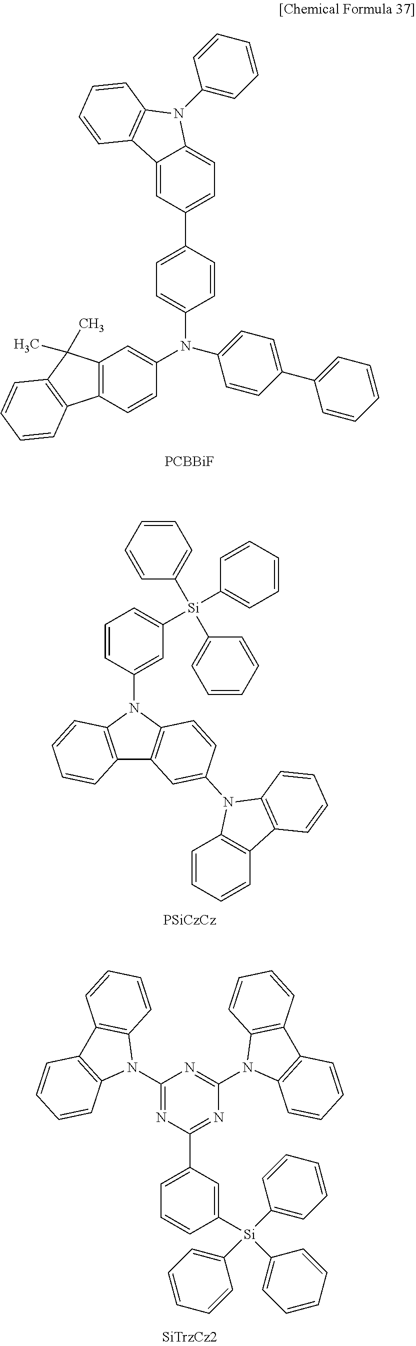

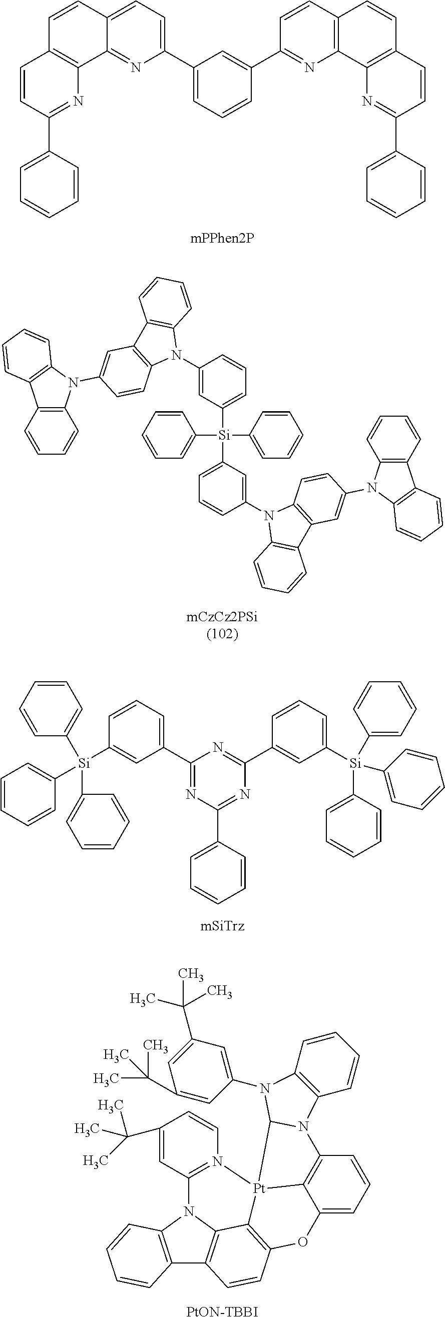

TABLE 13

| | | | | Comparative light- |
|---|---|---|---|---|
| | | Thickness | Light-emitting device 11 | emitting device 12 |
| Second electrode | | 200 nm | Al | |
| Electron-injection layer | | 1 nm | LiF | |
| Electron- | 2 | 20 nm | mPPhen2P | |
| transport layer | 1 | 5 nm | mSiTrz | |
| Light-emitting layer | | 35 nm | SiTrzCz2:mCzCz2PSi:PtON-TBBI | SiTrzCz2:PSiCzCz:PtON-TBBI |
| | | | (0.60:0.27:0.13) | (0.435:0.435:0.13) |

| | | | |
|---|---|---|---|
| Hole-transport | 2 | 5 nm | mCzCz2PSi |
| layer | 1 | 30 nm | PCBBiF |

| | | |
|---|---|---|
| Hole-injection layer | 10 nm | PCBBiF:OCHD-003 (1:0.03) |
| First electrode | 70 nm | ITSO |

### <<Fabrication of Light-Emitting Device 11>>

In the light-emitting device 11 described in this example, the hole-injection layer, the hole-transport layer (a first hole-transport layer and a second hole-transport layer), the light-emitting layer, the electron-transport layer (first and second electron-transport layers), and the electron-injection layer are stacked in this order over the first electrode formed over the substrate, and the second electrode is stacked over the electron-injection layer.

First, indium tin oxide containing silicon oxide (ITSO) was deposited over a glass substrate to a thickness of 70 nm by a sputtering method, whereby the first electrode was formed. The electrode area was set to 4 mm² (2 mm×2 mm). Note that the first electrode serves as an anode.

For pretreatment, a surface of the substrate was washed with water, baking was performed at 200° C. for one hour, and then UV ozone treatment was performed for 370 seconds. After that, the substrate was transferred into a vacuum evaporation apparatus where the pressure had been reduced to approximately 1×10⁻⁴ Pa, and was subjected to vacuum baking at 170° C. for 30 minutes in a heating chamber of the vacuum evaporation apparatus, and then the substrate was cooled down for approximately 30 minutes.

Then, the substrate provided with the first electrode was fixed to a holder provided in the vacuum evaporation apparatus such that the surface on which the first electrode was formed faced downward. Over the first electrode, N-(biphenyl-4-yl)-N-[4-(9-phenyl-9H-carbazol-3-yl)phenyl]-9,9-dimethyl-9H-fluoren-2-amine (abbreviation: PCBBiF) and a fluorine-containing electron acceptor material with a molecular weight of 672 (OCHD-003) were deposited by co-evaporation to a thickness of 10 nm such that the weight ratio of PCBBiF to OCHD-003 was 1:0.03, whereby the hole-injection layer was formed.

Next, over the hole-injection layer, the first hole-transport layer was formed by evaporation of PCBBiF to a thickness of 30 nm. Then, over the first hole-transport layer, the second hole-transport layer was formed by evaporation of mCzCz2PSi to a thickness of 5 nm.

Over the second hole-transport layer, 9,9′-{6-[3-(triphenylsilyl)phenyl]-1,3,5-triazine-2,4-diyl}bis(9H-carbazole) (abbreviation: SiTrzCz2), mCzCz2PSi, and (2-{3-[3-(3,5-di-tert-butylphenyl)benzimidazol-1-yl-2-ylidene-κC²]phenoxy-κC²}-9-(4-tert-butyl-2-pyridinyl-κN)carbazole-2,1-diyl-κC¹)platinum(II) (abbreviation: PtON-TBBI) were deposited by co-evaporation to a thickness of 35 nm such that the weight ratio of SiTrzCz2 to mCzCz2PSi and PtON-TBBI was 0.60:0.27:0.13.

Next, over the light-emitting layer, the first electron-transport layer was formed by evaporation of 2-phenyl-4,6-bis[3-(triphenylsilyl)phenyl]-1,3,5-triazine (abbreviation: mSiTrz) to a thickness of 5 nm; then, over the first electron-transport layer, the second electron-transport layer was formed by evaporation of 2,2′-(1,3-phenylene)bis(9-phenyl-1,10-phenanthroline) (abbreviation: mPPhen2P) to a thickness of 20 nm.

Next, lithium fluoride (LiF) was deposited over the second electron-transport layer by evaporation to a thickness of 1 nm to form the electron-injection layer, and then aluminum (Al) was deposited by evaporation to a thickness of 200 nm to form the second electrode.

### <<Fabrication of Comparative Light-Emitting Device 12>>

The comparative light-emitting device 12 is different from the light-emitting device 11 in that mCzCz2PSi used in the light-emitting layer in the light-emitting device 11 was replaced with PSiCzCz and co-evaporation was performed at a weight ratio of SiTrzCz2:PSiCzCz:PtON-TBBI=0.435:0.435:0.13. The other components were formed in the same manner as those in the light-emitting device 11.

The light-emitting device 11 and the comparative light-emitting device 12 were each sealed using a glass substrate in a glove box containing a nitrogen atmosphere so as not to be exposed to the air (a sealing material was applied to surround the devices and UV treatment and heat treatment at 80° C. for one hour were performed at the time of sealing). Then, the initial characteristics of the light-emitting devices were measured.

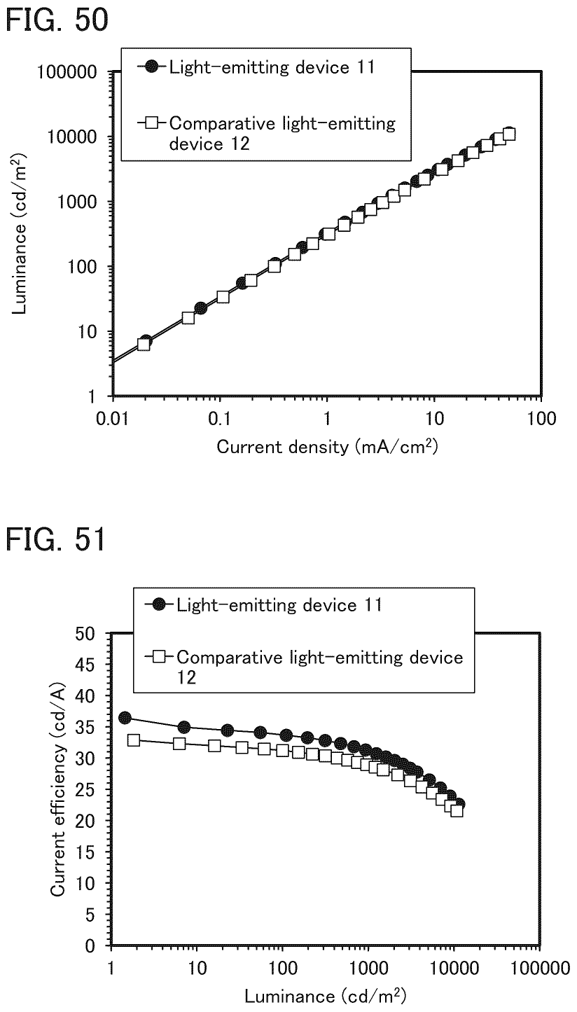

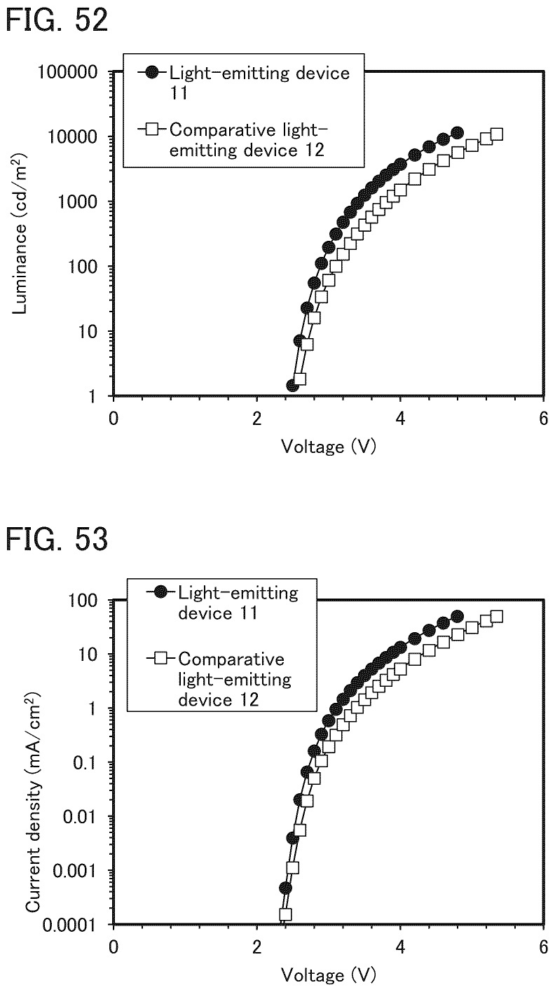

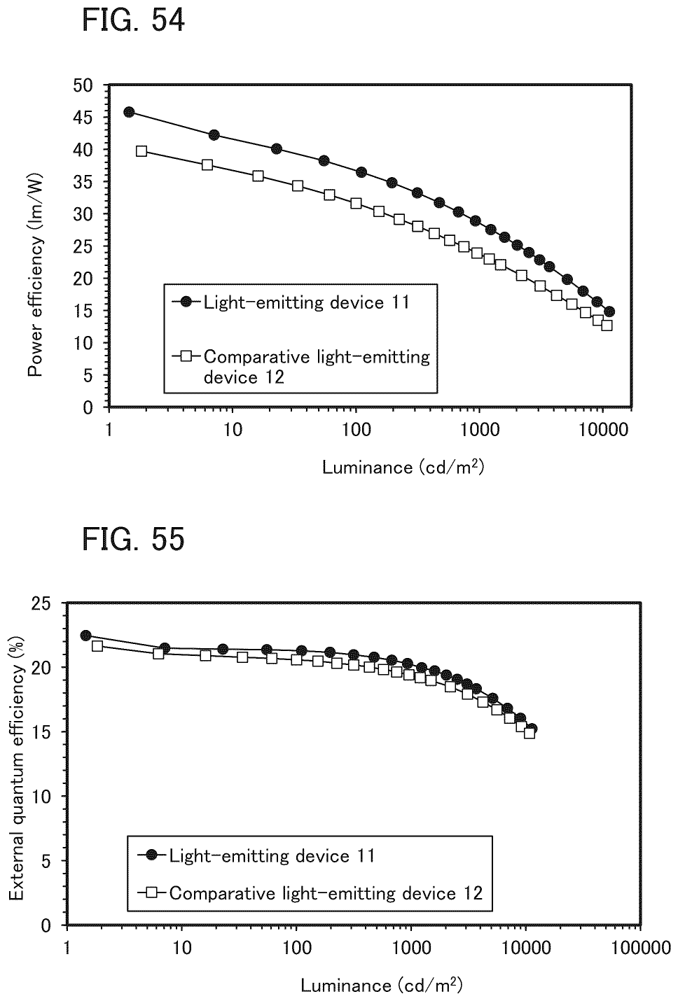

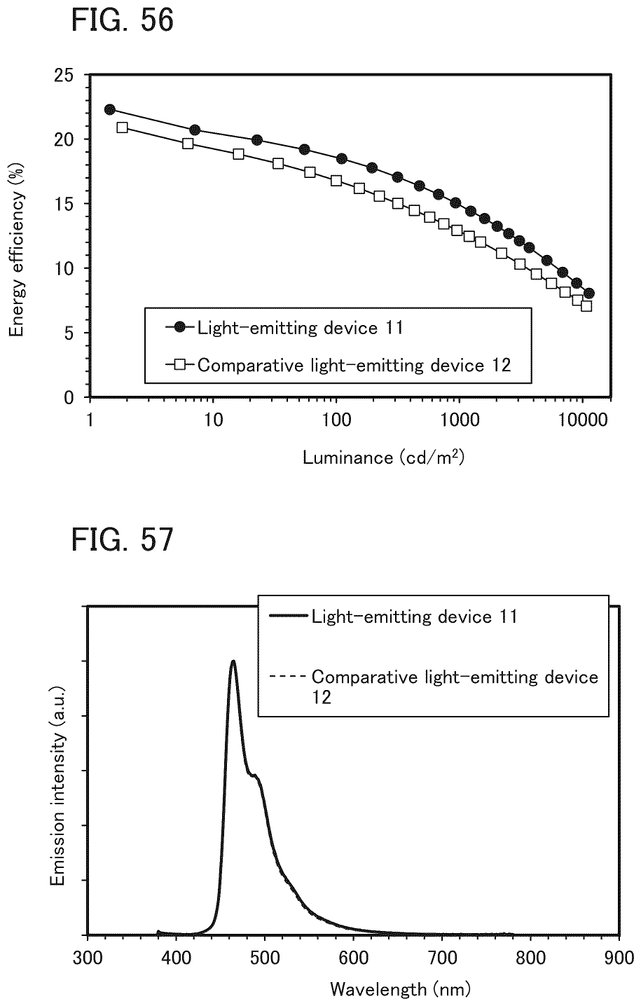

FIG. 50 shows the luminance-current density characteristics of the light-emitting device 11 and the comparative light-emitting device 12; FIG. 51, the current efficiency-luminance characteristics; FIG. 52, the luminance-voltage characteristics; FIG. 53, the current density-voltage characteristics; FIG. 54, the power efficiency-luminance characteristics; FIG. 55, the external quantum efficiency-luminance characteristics; FIG. 56, the energy efficiency-luminance characteristics; and FIG. 57, the electroluminescence spectra measured at a luminance of approximately 1000 cd/m².

Table 14 shows the main characteristics of the light-emitting device 11 and the comparative light-emitting device 12 at a luminance of approximately 1000 cd/m². Luminance, CIE chromaticity, and electroluminescence spectra were measured at normal temperature with a spectroradiometer (SR-UL1R, TOPCON TECHNOHOUSE CORPORATION). The external quantum efficiency described in this example was calculated from measurement on the front assuming a perfectly diffusing surface (also referred to as a Lambertian surface).

TABLE 14

| | | | | | | | External | |
|---|---|---|---|---|---|---|---|---|
| | | Current | | | Current | Power | quantum | Energy |
| | Voltage | density | Chromaticity | Chromaticity | efficiency | efficiency | efficiency | efficiency |
| | (V) | (mA/cm²) | x | y | (cd/A) | (lm/W) | (%) | (%) |
| Light-emitteing device 11 | 3.4 | 3.0 | 0.15 | 0.22 | 31 | 29 | 20 | 15 |
| Comparative light- | 3.8 | 3.3 | 0.14 | 0.21 | 29 | 24 | 19 | 13 |
| emitteing device 12 | | | | | | | | |

FIG. 50 to FIG. 57 and the above table reveal that the light-emitting device 11 exhibits blue light emission derived from PtON-TBBI and has favorable emission characteristics. In addition, the light-emitting device 11 was found to have lower driving voltage and higher current efficiency and higher external quantum efficiency than the comparative light-emitting device 12. The light-emitting device 11 was found to have higher maximum current efficiency, higher external energy conversion efficiency, higher power efficiency, and higher external energy conversion efficiency, than the comparative light-emitting device 12. Note that the mixture ratio of the two kinds of host materials used in the light-emitting layer of each light-emitting device was adjusted so that the optimum carrier balance was achieved to maximize efficiency.

Thus, the use of the organic compound of one embodiment of the present invention for the light-emitting layer can provide the light-emitting device with high emission efficiency.

### Example 9

This example shows, assuming the dissociation state of the organic compound represented by the general formula (G1) at the bond between Q and R¹ (Q-R₁ bond), results of estimation of an energy difference before and after the dissociation at the Q-R₁ bond as the bonding energy to examine the stability of the organic compound of one embodiment of the present invention.

Structural formulae of an organic compound (a1), an organic compound (a2), an organic compound (a3), and an organic compound (a4), each of which is the organic compound of one embodiment of the present invention used in this example, are shown below, where the assumed dissociation portions are shown by dotted lines. Note that the organic compound (a1) is the organic compound represented by the structural formula (102); the organic compound (a2), the structural formula (128); the organic compound (a3), the structural formula (129); and the organic compound (a4), the structural formula (117). Note that in the organic compounds (a1) to (a4), the groups other than Q, R¹, and R² of the general formula (G1) were the same for easier comparison between the bonding energies of the Q-R₁ bonds.

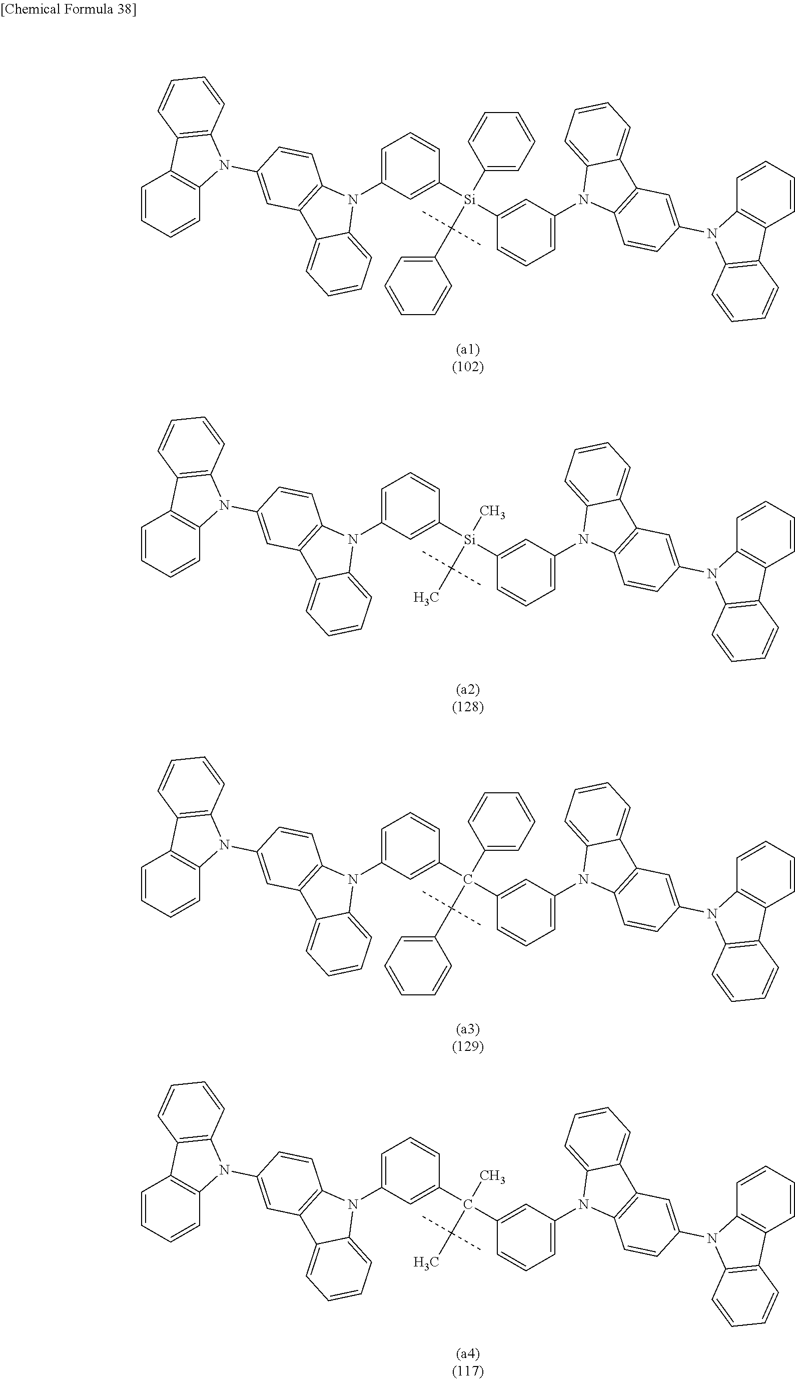

Structural formulae representing the states of the organic compounds (a1) to (a4) after the dissociation are shown below. This example assumes that dissociation occurs at the Q-R₁ bond of the organic compound (a1) to form a radical (b1) and a radical (c1), dissociation occurs at the Q-R₁ bond of the organic compound (a2) to form a radical (b2) and a radical (c2), dissociation occurs at the organic compound (a3) to form a radical (b3) and the radical (c1), and dissociation occurs at the organic compound (a4) to form a radical (b4) and a radical (c2).

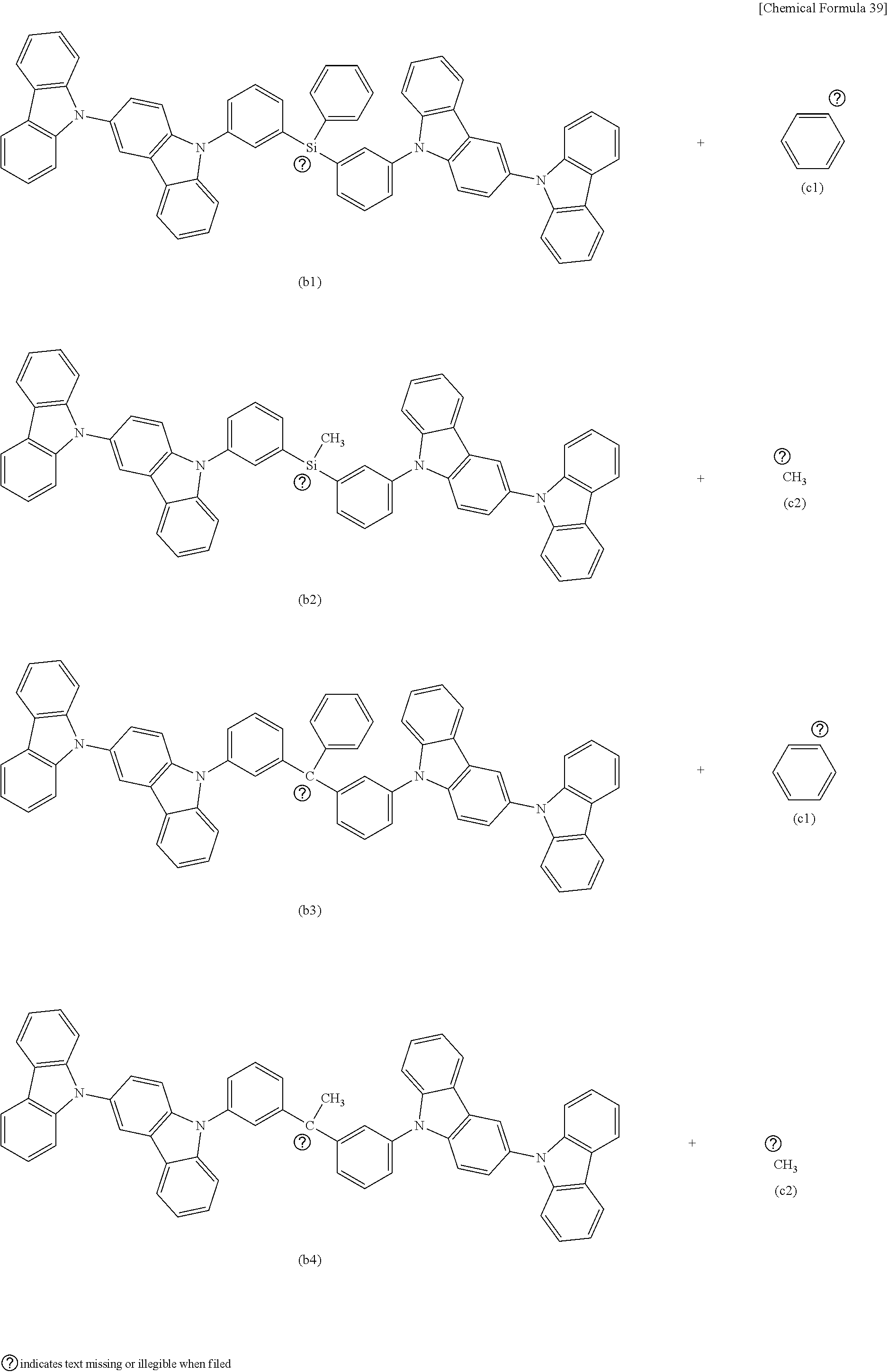

The most stable structures of the organic compound before the dissociation at the Q-R₁ bond and the two radicals after the dissociation of the organic compound at the Q-R₁ bond were each simulated by quantum chemical calculation to obtain zero-point-corrected energy. After the calculation, the bonding energy of the Q-R₁ bond was estimated as the difference between the zero-point-corrected energy of the organic compound before the dissociation at the Q-R₁ bond, which was set to 0 eV, and the sum of the zero-point-corrected energies of the two radicals after the dissociation at the Q-R₁ bond. For example, in the case where dissociation occurs at the Q-R₁ bond of the organic compound (a1) to form the radicals (b1) and (c1), the bonding energy of the Q-R₁ bond can be obtained by the calculation formula of {[zero-point-corrected energy of the radical (b1)]+[zero-point-corrected energy of the radical (c1)]}−[zero-point-corrected energy of (a1)]. When the bonding energy value of the Q-R₁ bond is larger, dissociation is less likely to occur at the Q-R₁ bond of the organic compound.

The most stable structures of the organic compounds and the radicals in the ground state were calculated by density functional theory (DFT). Here, vibration analysis was conducted on each of the most stable structures. In the DFT, the total energy of the molecules is represented as the sum of potential energy, electrostatic energy between electrons, electronic kinetic energy, and exchange-correlation energy including all the complicated interactions between electrons. Also in the DFT, an exchange-correlation interaction is approximated by a functional (a function of another function) of one electron potential represented in terms of electron density; thus, electron states can be obtained with high accuracy. As a basis function, 6-311G was applied to all the atoms. Furthermore, to improve calculation accuracy, the p function and the d function as polarization basis sets were added to hydrogen atoms and atoms other than hydrogen atoms, respectively. As a functional, B3LYP was used. Gaussian 16 was used as a quantum chemistry computational program.

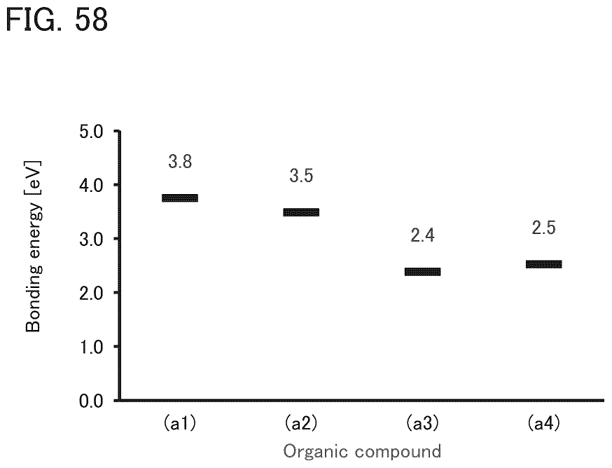

FIG. 58 shows the results of obtaining the bonding energy of the Q-R₁ bond on the assumption that dissociation occurred at the Q-R₁ bond of each of the organic compounds (a1) to (a4) to form the above two radicals.

As shown in FIG. 58, the bonding energies of the Q-R₁ bonds of the organic compounds (a1) and (a2) are higher than those of the organic compounds (a3) and (a4). These results reveal that dissociation is less likely to occur at the Q-R₁ bond of each of the organic compounds (a1) and (a2) than that at the Q-R₁ bond of each of the organic compounds (a3) and (a4). In this example, the organic compounds (a1) and (a2) each include silicon as Q, and the organic compounds (a3) and (a4) each include carbon as Q. This suggests that in the case where Q is silicon, dissociation is less likely to occur at the Q-R₁ bond than in the case where Q is carbon. Consequently, the organic compound represented by the general formula (G1) including silicon as Q can have increased chemical stability, so that light-emitting device characteristics with a long lifetime can be expected. Furthermore, the organic compound represented by the general formula (G1) including silicon as Q can probably be suitable for the light-emitting device with high emission energy, such as a blue-light-emitting device having an emission wavelength of approximately 2.7 eV (460 nm).

Since a silicon atom has a larger atomic radius than a carbon atom, steric hindrance between the substituents bonded to Q can be effectively inhibited in the organic compound represented by the general formula (G1) in the case where Q is silicon, as compared with the case where Q is carbon, and accordingly the organic compound can be stabilized. It is for this reason that, in the case where Q is silicon, dissociation is less likely to occur at the Q-R₁ bond than in the case where Q is carbon.

As shown in FIG. 58, the bonding energy of the Q-R₁ bond of the organic compound (a2) is higher than that of the organic compound (a1). This reveals that, in the organic compound represented by the general formula (G1), when Q is silicon, dissociation at the Q-R₁ bond is unlikely to occur in the case where R¹ is an aryl group such as a phenyl group, as compared with the case where R¹ is an alkyl group such as a methyl group. Meanwhile, as shown in FIG. 58, the bonding energy of the Q-R₁ bond of the organic compound (a4) is higher than that of the organic compound (a3). This reveals that, in the organic compound represented by the general formula (G1), when Q is carbon, the dissociation is unlikely to occur in the case where R¹ is an alkyl group such as a methyl group, as compared with the case where R¹ is an aryl group such as a phenyl group. This is probably because, since a silicon atom has a larger atomic radius than a carbon atom, steric hindrance between substituents bonded to Q can be effectively inhibited in the case where R¹ is an alkyl group such as a methyl group, as compared with the case where R¹ is an aryl group such as a phenyl group, and accordingly the organic compound is stabilized. Thus, it is found that when Q is carbon in the organic compound represented by the general formula (G1), each of R¹ and R² preferably independently represents an alkyl group such as a methyl group.

This application is based on Japanese Patent Application Serial No. 2023-112138 filed with Japan Patent Office on Jul. 7, 2023, the entire contents of which are hereby incorporated by reference.

What is claimed is:

1. An organic compound represented by a general formula (G1): 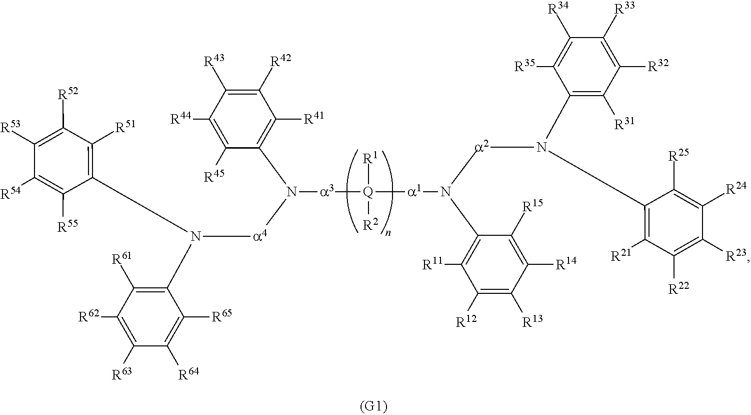
wherein:
Q represents carbon or silicon;
each of R¹ and R² independently represents an alkyl group having 1 to 6 carbon atoms, a substituted or unsubstituted cycloalkyl group having 6 to 8 carbon atoms, or a substituted or unsubstituted aryl group having 6 to 12 carbon atoms;
n represents an integer greater than or equal to 1 and less than or equal to 3;
each of α¹ to α⁴ independently represents a substituted or unsubstituted phenylene group or a substituted or unsubstituted biphenylene group;
at least one of R¹⁵, R²⁵, R³¹, R⁴⁵, R⁵⁵, and R⁶¹ represents a single bond;
each of the others of R¹⁵, R²⁵, R³¹, R⁴⁵, R⁵⁵, and R⁶¹ independently represents hydrogen, an alkyl group having 1 to 6 carbon atoms, or a substituted or unsubstituted aryl group having 6 to 12 carbon atoms;
each of R¹¹ to R¹⁴, R²¹ to R²⁴, R³² to R³⁵, R⁴¹ to R⁴⁴, R⁵¹ to R⁵⁴, and R⁶² to R⁶⁵ independently represents hydrogen, an alkyl group having 1 to 6 carbon atoms, a substituted or unsubstituted cycloalkyl group having 6 to 8 carbon atoms, or a substituted or unsubstituted aryl group having 6 to 12 carbon atoms;
R¹⁵ is bonded to α² to form a ring when R¹⁵ is a single bond;
R⁴⁵ is bonded to α⁴ to form a ring when R⁴⁵ is a single bond;
R²⁵ and R³¹ are bonded to each other to form a ring when R²⁵ and R³¹ are each a single bond;
R⁵⁵ and R⁶¹ are bonded to each other to form a ring when R⁵⁵ and R⁶¹ are each a single bond;
α² is a substituted or unsubstituted biphenylene group and bonded to one of R²⁵ and R³¹ to form a ring when the one of R²⁵ and R³¹ is a single bond and the other of R²⁵ and R³¹ is hydrogen, an alkyl group having 1 to 6 carbon atoms, or a substituted or unsubstituted aryl group having 6 to 12 carbon atoms; and
α⁴ is a substituted or unsubstituted biphenylene group and bonded to one of R⁵⁵ and R⁶¹ to form a ring when the one of R⁵⁵ and R⁶¹ is a single bond and the other of R⁵⁵ and R⁶¹ is hydrogen, an alkyl group having 1 to 6 carbon atoms, or a substituted or unsubstituted aryl group having 6 to 12 carbon atoms.

2. The organic compound according to claim 1,
wherein each of α¹ and α³ independently represents a substituted or unsubstituted m-phenylene group.

3. The organic compound according to claim 1,
wherein the organic compound is represented by a general formula (G2-1): 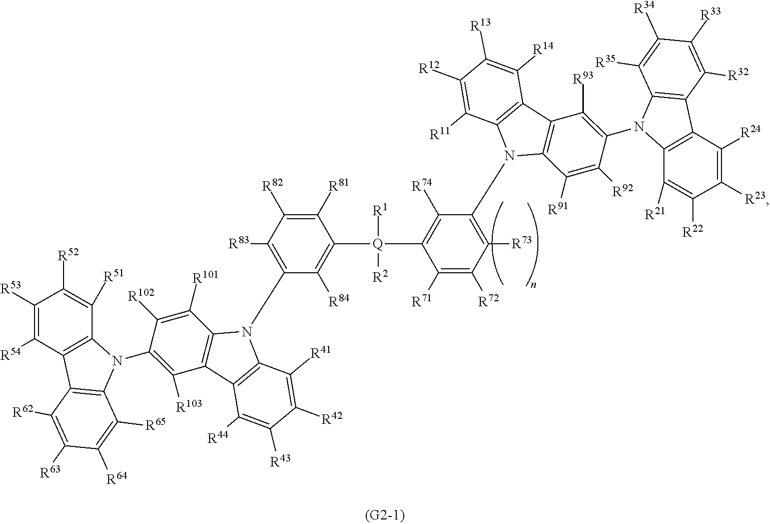
wherein:
Q represents carbon or silicon;
each of R¹ and R² independently represents an alkyl group having 1 to 6 carbon atoms, a substituted or unsubstituted cycloalkyl group having 6 to 8 carbon atoms, or a substituted or unsubstituted aryl group having 6 to 12 carbon atoms;
n represents an integer greater than or equal to 1 and less than or equal to 3; and
each of R¹¹ to R¹⁴, R²¹ to R²⁴, R³² to R³⁵, R⁴¹ to R⁴⁴, R⁵¹ to R⁵⁴, R⁶² to R⁶⁵, R⁷¹ to R⁷⁴, R⁸¹ to R⁸⁴, R⁹¹ to R⁹³, and R¹⁰¹ to R¹⁰³ independently represents hydrogen, an alkyl group having 1 to 6 carbon atoms, or a substituted or unsubstituted aryl group having 6 to 12 carbon atoms.

4. The organic compound according to claim 1,
wherein the organic compound is represented by a general formula (G1-2-2): 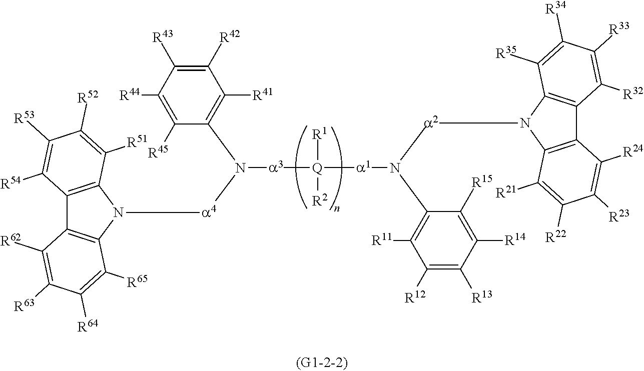
wherein:
Q represents carbon or silicon;
each of R¹ and R² independently represents an alkyl group having 1 to 6 carbon atoms, a substituted or unsubstituted cycloalkyl group having 6 to 8 carbon atoms, or a substituted or unsubstituted aryl group having 6 to 12 carbon atoms;
n represents an integer greater than or equal to 1 and less than or equal to 3;
each of α¹ to α⁴ independently represents a substituted or unsubstituted phenyl group or a substituted or unsubstituted biphenyl group; and
each of R¹¹ to R¹⁵, R²¹ to R²⁴, R³² to R³⁵, R⁴¹ to R⁴⁵, R⁵¹ to R⁵⁴, and R⁶² to R⁶⁵ independently represents hydrogen, an alkyl group having 1 to 6 carbon atoms, or a substituted or unsubstituted aryl group having 6 to 12 carbon atoms.

5. The organic compound according to claim 1,
wherein the organic compound is represented by a general formula (G2-3): 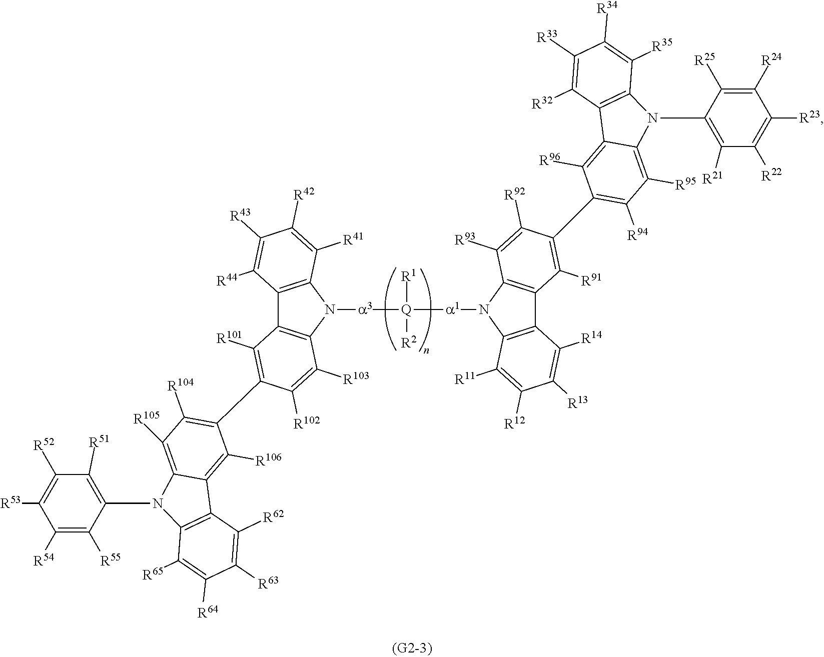
wherein:
Q represents carbon or silicon;
each of R¹ and R² independently represents an alkyl group having 1 to 6 carbon atoms, a substituted or unsubstituted cycloalkyl group having 6 to 8 carbon atoms, or a substituted or unsubstituted aryl group having 6 to 12 carbon atoms;
n represents an integer greater than or equal to 1 and less than or equal to 3;
each of α¹ and α³ independently represents a substituted or unsubstituted phenyl group or a substituted or unsubstituted biphenyl group; and
each of R¹¹ to R¹⁴, R²¹ to R²⁵, R³² to R³⁵, R⁴¹ to R⁴⁴, R⁵¹ to R⁵⁵, R⁶² to R⁶⁵, R⁹¹ to R⁹⁶, and R¹⁰¹ to R¹⁰⁶ independently represents hydrogen, an alkyl group having 1 to 6 carbon atoms, or a substituted or unsubstituted aryl group having 6 to 12 carbon atoms.

6. The organic compound according to claim 1,
wherein a glass transition temperature of the organic compound is higher than or equal to 100° C.

7. The organic compound according to claim 1,
wherein the organic compound is represented by any one of a structural formula (100), a structural formula (101), a structural formula (102), and a structural formula (114):

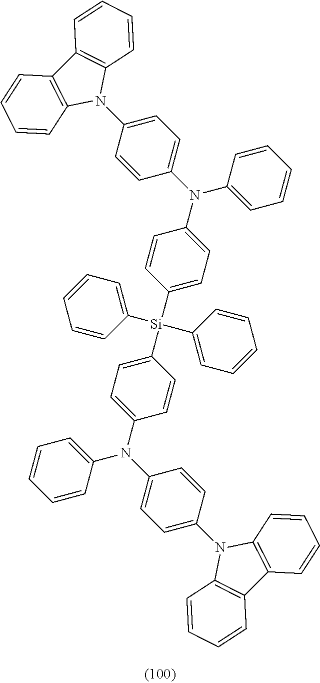 [SMILES: c1ccc(N(c2ccc(-n3c4ccccc4c4ccccc43)cc2)c2ccc([Si](c3ccccc3)(c3ccccc3)c3ccc(N(c4ccccc4)c4ccc(-n5c6ccccc6c6ccccc65)cc4)cc3)cc2)cc1]

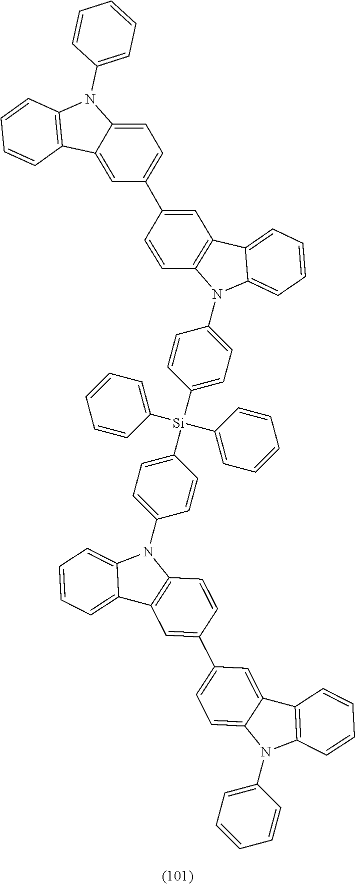 [SMILES: c1ccc(-n2c3ccccc3c3cc(-c4ccc5c(c4)c4ccccc4n5-c4ccc([Si](c5ccccc5)(c5ccccc5)c5ccc(-n6c7ccccc7c7cc(-c8ccc9c(c8)c8ccccc8n9-c8ccccc8)ccc76)cc5)cc4)ccc32)cc1]

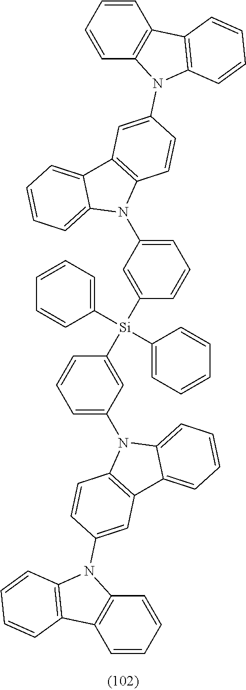 [SMILES: c1ccc([Si](c2ccccc2)(c2cccc(-n3c4ccccc4c4cc(-n5c6ccccc6c6ccccc65)ccc43)c2)c2cccc(-n3c4ccccc4c4cc(-n5c6ccccc6c6ccccc65)ccc43)c2)cc1]

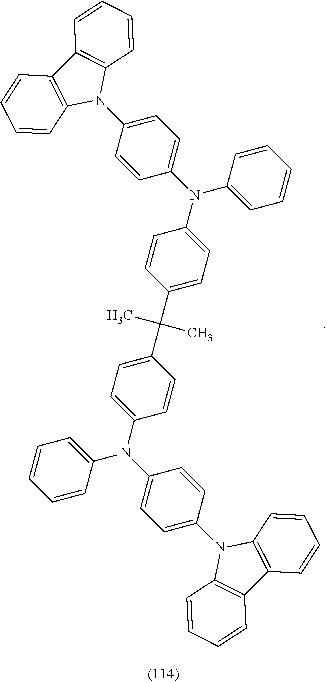 [SMILES: CC(C)(c1ccc(N(c2ccccc2)c2ccc(-n3c4ccccc4c4ccccc43)cc2)cc1)c1ccc(N(c2ccccc2)c2ccc(-n3c4ccccc4c4ccccc43)cc2)cc1]

8. A light-emitting device comprising:
a first electrode;
a second electrode; and
an organic compound layer,
wherein the organic compound layer is between the first electrode and the second electrode, and
wherein the organic compound layer comprises the organic compound according to claim 1.

9. A light-emitting device comprising:
a first electrode;
a second electrode; and
an organic compound layer,
wherein the organic compound layer is between the first electrode and the second electrode,
wherein the organic compound layer comprises a plurality of layers each comprising an organic compound and comprises at least one light-emitting layer, and
wherein, among the plurality of layers each comprising the organic compound, any one of layers from a layer in contact with the first electrode to at least the light-emitting layer comprises the organic compound according to claim 1.

10. A light-emitting device comprising:
a first electrode;
a second electrode; and
an organic compound layer,
wherein the organic compound layer is between the first electrode and the second electrode,
wherein the organic compound layer comprises a plurality of layers comprising an organic compound and comprises at least one light-emitting layer,
wherein the light-emitting layer comprises the organic compound according to claim 1 and a second organic compound, and
wherein the second organic compound comprises a π-electron deficient heteroaromatic ring.

11. A light-emitting apparatus comprising:
the light-emitting device according to claim 8; and
a transistor or a substrate.

12. An electronic device comprising:
the light-emitting apparatus according to claim 11; and
a sensor unit, an input unit, or a communication unit.

13. A lighting device comprising:
the light-emitting apparatus according to claim 11; and
a housing.
